SEQUENCE LISTING (205 sequences)

SEQ ID NO: 1 - DNA, 953 bp, Homo sapiens
ctctgccatgatcattttcaacctgagcagttacaatccaggacccttcattctggtagggatcccaggcctggagcaattccatgtgtggattggaattcccttctgtatcatctacattgtagctgttgtgggaaactgcatccttctctacctcattgtggtggagcatagtcttcatgaacccatgttcttctttctctccatgctggccatgactgacctcatcttgtccacagctggtgtgcctaaaacactcagtatcttttggctaggggctcgcgaaatcacattcccaggatgccttacacaaatgttcttccttcactataactttgtcctggattcagccattctgatggccatggcatttgatcgctatgtagctatctgttctcccttgagatataccaccatcttgactcccaagaccatcatcaagagtgctatgggcatctcctttcgaagcttctgcatcatcctgccagatgtattcttgctgacatgcctgcctttctgcaggacacgcatcataccccacacatactgtgagcatataggtgttgcccagctcgcctgtgctgatatctccatcaacttctggtatggcttttgtgttcccatcatgacagtcatctcagatgtgattctcattgctgtttcctacgcacacatcctctgtgctgtcttttgccttccctcccaagatgcccgccagaaagccctcggcacttgtggttctcatgtctgtgtcatcctcatgttttatacacctgcctttttctccatcctcgcccatcgctttggacacaatgtctctcgcaccttccacatcatgtttgccaatctctacattgttatcccacctgcactcaaccccatggtttacggagtgaagaccaagcagatcagagataaggttatacttttgttttctaagggtacaggatgat

SEQ ID NO: 2 - protein, 316 aa, Homo sapiens
SAMIIFNLSSYNPGPFILVGIPGLEQFHVWIGIPFCIIYIVAVVGNCILLYLIVVEHSLHEPMFFFLSMLAMTDLILSTAGVPKTLSIFWLGAREITFPGCLTQMFFLHYNFVLDSAILMAMAFDRYVAICSPLRYTTILTPKTIIKSAMGISFRSFCIILPDVFLLTCLPFCRTRIIPHTYCEHIGVAQLACADISINFWYGFCVPIMTVISDVILIAVSYAHILCAVFCLPSQDARQKALGTCGSHVCVILMFYTPAFFSILAHRFGHNVSRTFHIMFANLYIVIPPALNPMVYGVKTKQIRDKVILLFSKGTG

SEQ ID NO: 3 - DNA, 953 bp, Homo sapiens
ctctgccatgatcattttcaacctgagcagttacaatccaggacccttcattctggtagggatcccaggcctggagcaattccatgtgtggattggaattcccttctgtatcatctacattgtagctgttgtgggaaactgcatccttctctacctcattgtggtggagcatagtcttcatgaacccatgttcttctttctctccatgctggccatgactgacctcatcttgtccacagctggtgtgcctaaaacactcagtatcttttggctaggggctcgcgaaatcacattcccaggatgccttacacaaatgttcttccttcactataactttgtcctggattcagccattctgatggccatggcatttgatcgctatgtagctatctgttctcccttgagatataccaccatcttgactcccaagaccatcatcaagagtgctatgggcatctcctttcgaagcttctgcatcatcctgccagatgtattcttgctgacatgcctgcctttctgcaggacacgcatcataccccacacatactgtgagcatataggtgttgcccggctcgcctgtgctgatatctccatcaacttctggtatggcttttgtgttcccatcatgacagtcatctcagatgtgattctcattgctgtttcctacgcacacatcctctgtgctgtcttttgccttccctcccaagatgcccgccagaaagccctcggcacttgtggttctcatgtctgtgtcatcctcatgttttatacacctgcctttttctccatcctcgcccatcgctttggacacaatgtctctcgcaccttccacatcatgtttgccaatctctacattgttatcccacctgcactcaaccccatggtttacggagtgaagaccaagcagatcagagataaggttatacttttgttttctaagggtacaggatgat

SEQ ID NO: 4 - protein, 314 aa, Homo sapiens
MIIFNLSSYNPGPFILVGIPGLEQFHVWIGIPFCIIYIVAVVGNCILLYLIVVEHSLHEPMFFFLSMLAMTDLILSTAGVPKTLSIFWLGAREITFPGCLTQMFFLHYNFVLDSAILMAMAFDRYVAICSPLRYTTILTPKTIIKSAMGISFRSFCIILPDVFLLTCLPFCRTRIIPHTYCEHIGVARLACADISINFWYGFCVPIMTVISDVILIAVSYAHILCAVFCLPSQDARQKALGTCGSHVCVILMFYTPAFFSILAHRFGHNVSRTFHIMFANLYIVIPPALNPMVYGVKTKQIRDKVILLFSKGTG

SEQ ID NO: 5 - DNA, 953 bp, Homo sapiens
ctctgccatgatcattttcaacctgagcagttacaatccaggacccttcattctggtagggatcccaggcctggagcaattccatgtgtggattggaattcccttctgtatcatctacattgtagctgttgtgggaaactgcatccttctctacctcattgtggtggagcatagtcttcatgaacccatgttcttctttctctccatgctggccatgactgacctcatcttgtccacagctggtgtgcctaaaacactcagtatcttttggctaggggctcgcgaaatcacattcccaggatgccttacacaaatgttcttccttcactataactttgtcctggattcagccattctgatggccatggcatttgatcactatgtagctatctgttctcccttgagatataccaccatcttgactcccaagaccatcatcaagagtgctatgggcatctcctttcgaagcttctgcatcatcctgccagatgtattcttgctgacatgcctgcctttctgcaggacacgcatcataccccacacatactgtgagcatatgggtgttgcccagctcgcctgtgctgatatctccatcaacttctggtatggcttttgtgttcccatcatgacagtcatctcagatgtgattctcattgctgtttcctacgcacacatcctctgtgctgtcttttgccttccctcccaagatgcccgccagaaggccctcggcacttgtggttctcatgtctgtgtcatcctcatgttttatacacctgcctttttctccatcctcgcccatcgctttggacacaatgtctctcgcaccttccacatcatgtttgccaatctctacattgttatcccacctgcactcaaccccatggtttacggagtgaagaccaagcagatcagagataaggttatacttttgttttctaagggtacaggatgat

SEQ ID NO: 6 - protein, 314 aa, Homo sapiens
MIIFNLSSYNPGPFILVGIPGLEQFHVWIGIPFCIIYIVAVVGNCILLYLIVVEHSLHEPMFFFLSMLAMTDLILSTAGVPKTLSIFWLGAREITFPGCLTQMFFLHYNFVLDSAILMAMAFDHYVAICSPLRYTTILTPKTIIKSAMGISFRSFCIILPDVFLLTCLPFCRTRIIPHTYCEHMGVAQLACADISINFWYGFCVPIMTVISDVILIAVSYAHILCAVFCLPSQDARQKALGTCGSHVCVILMFYTPAFFSILAHRFGHNVSRTFHIMFANLYIVIPPALNPMVYGVKTKQIRDKVILLFSKGTG

SEQ ID NO: 7 - DNA, 965 bp, Homo sapiens
ccccttgtctcctcacacaatgaccctgggatccctgggaaacagcagcagcagcgtttctgctaccttcctgctgagggcatccctgggctggagcgcatgcacatctggatctccatcccactgtgcttcatgtatctggtttccatcccgggcaactgcacaattctttttatcattaaaacagagcgctcacttcatgaacctatgtatctcttcctgtccatgctggctctgattgacctgggtctctccctttgcactctccctacagtcctgggcatcttttgggttggagcacgagaaattagccatgatgcctgctttgctcagctctttttcattcactgcttctccttcctcgagtcctctgtgctactgtctatggcctttgaccgctttgtggctatctgccaccccttgcactatgtttccattctcaccaacacagtcattggcaggattggcctggtctctctgggtcgtagtgtagcactcatttttccattaccttttatgctcaaaagattcccctattgtggctccccagttctctcacattcttattgtctccaccaagaagtgatgaaattggcctgtgccgacatgaaggccaacagcatctacggcatgtttgtcatcgtctctacagtgggtatagactcactgctcatcctcttctcttatgctctgatcctgcgcaccgtgctgtccatcgcctccagggctgagagattcaaggcccttaacacctgtgtttcccacatctgtgctgtgctgctcttctacactcccatgattggcctctctgtcatccatcgctttggaaagcaggcaccccacctggtccaggtggtcatgggtttcatgtatcttctctttcctcctgtgatgaatcccattgtctacagtgtgaagaccaaacagatccgggatcgagtgacgcatgccttttgttactaact

SEQ ID NO: 8 - protein, 314 aa, Homo sapiens
MTLGSLGNSSSSVSATFLLSGIPGLERMHIWISIPLCFMYLVSIPGNCTILFIIKTERSLHEPMYLFLSMLALIDLGLSLCTLPTVLGIFWVGAREISHDACFAQLFFIHCFSFLESSVLLSMAFDRFVAICHPLHYVSILTNTVIGRIGLVSLGRSVALIFPLPFMLKRFPYCGSPVLSHSYCLHQEVMKLACADMKANSIYGMFVIVSTVGIDSLLILFSYALILRTVLSIASRAERFKALNTCVSHICAVLLFYTPMIGLSVIHRFGKQAPHLVQVVMGFMYLLFPPVMNPIVYSVKTKQIRDRVTHAFCY

SEQ ID NO: 9 - DNA, 966 bp, Homo sapiens
ccccttgtctcctcacacaatgaccctgggatccctgggaaacagcagcagcagcgtttctgctaccttcctgctgagtggcatccctgggctggagcgcatgcacatctggatctccatcccactgtgcttcatgtatctggtttccatcccgggcaactgcacaattctttttatcattaaaacagagcgctcacttcatgaacctatgtatctcttcctgtccatgctggctctgattgacctgggtctctccctttgcactctccctacagtcctgggcatcttttgggttggagcacgacaaattagccatgatgcctgctttgctcagctctttttcattcactgcttctccttcctcgagtcctctgtgctactgtctatggcctttgaccgctttgtggctatctgccaccccttgcactatgtttccattctcaccaacacagtcattggcaggattggcctggtctctctgggtcgtagtgtagcactcatttttccattaccttttatgctcaaaagattcccctattgtggctccccagttctctcacattcttattgtctccaccaagaagtgatgaaattggcctgtgccgacatgaaggccaacagcatctacggcatgtttgtcatcgtctctacagtgggtatagactcactgctcatcctcttctcttatgctctgatcctgcgcaccgtgctgtccatcgcctccagggctgagagattcaaggcccttaacacctgtgtttcccacatctgtgctgtgctgctcttctacactcccatgattggcctctctgtcatccatcgctttggaaagcaggcaccccacctggtccaggtggtcatgggtttcatgtatcttctctttcctcctgtgatgaatcccattgtctacagtgtgaagaccaaacagatccgggatcgagtgacgcatgccttttgttactaact

SEQ ID NO: 10 - protein, 314 aa, Homo sapiens
MTLGSLGNSSSSVSATFLLSGIPGLERMHIWISIPLCFMYLVSIPGNCTILFIIKTERSLHEPMYLFLSMLALIDLGLSLCTLPTVLGIFWVGARQISHDACFAQLFFIHCFSFLESSVLLSMAFDRFVAICHPLHYVSILTNTVIGRIGLVSLGRSVALIFPLPFMLKRFPYCGSPVLSHSYCLHQEVMKLACADMKANSIYGMFVIVSTVGIDSLLILFSYALILRTVLSIASRAERFKALNTCVSHICAVLLFYTPMIGLSVIHRFGKQAPHLVQVVMGFMYLLFPPVMNPIVYSVKTKQIRDRVTHAFCY

SEQ ID NO: 11 - DNA, 1251 bp, Homo sapiens
caaaatgattatagctgacaaaatcaggaagtgtgttgttaacttcctgacttcttatatttcagagaacgaagagttgaaccatttaacatgaattgggtaaataagagtgtcccacaggagttcattctgttagttttctcagatcaaccatggctagagattccaccctttgtgatgtttctgttttcctatatcttgacaatctttggcaatctgacaataattcttgtgtcacatgtggatttcaaactccacacccctatgtacttttttcttagcaatctctcactcctggacctttgctataccacaagtacagttccacaaatgctggtaaacatatgcaacaccaggaaagtaatcagttatggtggctgtgtggcccagcttttcattttcctggccttgggttccacagaatgtcttctcctggccgtcatgtgctttgataggtttgtagctatttgtcggcctctccattactcaattatcatgcaccagaggctctgcttccagttggcagctgcatcctggattagtggctttagcaattcagtattacagtccacctggacacttaagatgccactgtgtggtcacaaagaagtggatcacttcttctgtgaagtccctgctctgctcaagttgtcctgtgttgacacaacagcaaatgaggctgaactattcttcatcagtgtgctattccttctaatacccgtgacactcatccttatatcgtatgcttttattgtccaagcagtgttgagaatccagtctgctgaaggtcaacgaaaggcatttgggacatgtggctcccatctaattgtggtgtcacttttttatggtacagctatctccatgtacctgcaaccaccttcacccagctccaaagaccggggaaagatggtttctctcttctgtggaatcattgcacccatgctgaatccccttatatatacacttaggaacaaagaggtaaaggaagcctttaaaaggttggttgcaaagagtcttcttaatcaagaaataagaaatatgcaaatgataagctttgctaaagacacagtgcttacttaccttactaacttctccgcaagttgtcctatttttgtcattactatagaaaactattgtaatctccctcaaagaaaatttccttgacaaaaagctatatttgtttctgttgcctaaacattttcattgaacaagcccccagaattggccttccaatgcaccaaaaactgtaat

SEQ ID NO: 12 - protein, 357 aa, Homo sapiens
MNWVNKSVPQEFILLVFSDQPWLEIPPFVMFLFSYILTIFGNLTIILVSHVDFKLHTPMYFFLSNLSLLDLCYTTSTVPQMLVNICNTRKVISYGGCVAQLFIFLALGSTECLLLAVMCFDRFVAICRPLHYSIIMHQRLCFQLAAASWISGFSNSVLQSTWTLKMPLCGHKEVDHFFCEVPALLKLSCVDTTANEAELFFISVLFLLIPVTLILISYAFIVQAVLRIQSAEGQRKAFGTCGSHLIVVSLFYGTAISMYLQPPSPSSKDRGKMVSLFCGIIAPMLNPLIYTLRNKEVKEAFKRLVAKSLLNQEIRNMQMISFAKDTVLTYLTNFSASCPIFVITIENYCNLPQRKFP

SEQ ID NO: 13 - DNA, 953 bp, Homo sapiens
gcctgaccaaaaagaatggcagccaaaaactcttctgtgacagagtttatcctcgaaggcttaacccaccagccgggactgcggatccccctcttcttcctgtttctgggtttctacacggtcaccgtggtggggaacctgggcttgataaccctgattgggctgaactctcacctgcacactcccatgtacttcttcctttttaacctctctttaatagatttctgtttctccactaccatcactcccaaaatgctgatgagttttgtctcaaggaagaacatcatttccttcacagggtgtatgactcagctcttcttcttctgcttctttgtcgtctctgagtccttcatcctgtcagcgatggcgtatgaccgctacgtggccatctgtaacccactgttgtacacagtcaccatgtcttgccaggtgtgtttgctccttttgttgggtgcctatgggatggggtttgctggggccatggcccacacaggaagcataatgaacctgaccttctgtgctgacaaccttgtcaatcatttcatgtgtgacatccttcctctccttgagctctcctgcaacagctcttacatgaatgagccggtggtctttattgtggtggctgttgacgttggaatgcccattgtcactgtctttatttcttatgccctcatcctctccagcattctacacaacagttctacagaaggcaggtccaaagcctttagtacttgcagttcccacataattgtagtttctcttttctttggttctggtgctttcatgtatctcaaacccctttccatcctgcccctcgagcaagggaaagtgtcctccctgttctataccataatagtccccgtgttaaacccattaatctatagcttgaggaacaaggatgtcaaagttgccctgaggagaactttgggcagaaaaatcttttcttaagaaag

SEQ ID NO: 14 - protein, 310 aa, Homo sapiens
MAAKNSSVTEFILEGLTHQPGLRIPLFFLFLGFYTVTVVGNLGLITLIGLNSHLHTPMYFFLFNLSLIDFCFSTTITPKMLMSFVSRKNIISFTGCMTQLFFFCFFVVSESFILSAMAYDRYVAICNPLLYTVTMSCQVCLLLLLGAYGMGFAGAMAHTGSIMNLTFCADNLVNHFMCDILPLLELSCNSSYMNEPVVFIVVAVDVGMPIVTVFISYALILSSILHNSSTEGRSKAFSTCSSHIIVVSLFFGSGAFMYLKPLSILPLEQGKVSSLFYTIIVPVLNPLIYSLRNKDVKVALRRTLGRKIFS

SEQ ID NO: 15 - DNA, 1103 bp, Homo sapiens
  misc_feature (51): where n is A, G, C or T
tgcagattttatcttctttctacctctgtgagtagaaggtgaggttctganagttctccccagctatgcctactgtaaaccacagtggcactagccacacagtcttccacttgctgggcatccctggcctacaggaccagcacatgtggatttctatcccattcttcatttcctatgtcaccgcccttcttgggaacagcctgctcatcttcattatcctcacaaagcgcagcctccatgaacccatgtacctcttcctctgcatgctggctggagcagacattgtcctctccacgtgcaccattcctcaggccttagctatcttctggttccgtgctggggacatctccctggatcgttgcatcactcagctcttcttcatccattccaccttcatctctgagtcagggatcttgctggtgatggcctttgaccactatattgccatatgctacccactgaggtacaccaccattcttacaaatgctctgatcaagaaaatttgtgtgactgtctctctgagaagttatggtacaattttccctatcatatttcttttaaaaagattgactttctgccagaataatattattccacacaccttttgtgaacacattggcctagccaaatatgcatgtaatgacattcgaataaacatttggtatgggttttccattctaatgtcgacggtggtcttagatgttgtactaatttttatttcctatatgctgattctccatgctgtcttccacatgccttctccagatgcttgccacaaagctctcaacacatttggctcccatgtctgcatcatcatcctcttttatgggtctggcatcttcacaatccttacccagaggtttggacgccacattccaccttgtatccacatcccgttggctaatgtctgcattctggctccacctatgctgaatcccattatttatgggatcaaaaccaagcaaatccaggaacaggtggttcagtttttgtttataaaacagaaaataactttggtttaagaactgagttttcagaatctctagctatctggtaagtgggtatgaaagtggtagatgggagaggtcagctgataccgtaga

SEQ ID NO: 16 - protein, 318 aa, Homo sapiens
MPTVNHSGTSHTVFHLLGIPGLQDQHMWISIPFFISYVTALLGNSLLIFIILTKRSLHEPMYLFLCMLAGADIVLSTCTIPQALAIFWFRAGDISLDRCITQLFFIHSTFISESGILLVMAFDHYIAICYPLRYTTILTNALIKKICVTVSLRSYGTIFPIIFLLKRLTFCQNNIIPHTFCEHIGLAKYACNDIRINIWYGFSILMSTVVLDVVLIFISYMLILHAVFHMPSPDACHKALNTFGSHVCIIILFYGSGIFTILTQRFGRHIPPCIHIPLANVCILAPPMLNPIIYGIKTKQIQEQVVQFLFIKQKITLV

SEQ ID NO: 17 - DNA, 972 bp, Homo sapiens
aaacttggacgatcgacatggaaattgtctccacaggaaacgaaactattactgaatttgtcctccttggcttctatgacatccctgaactgcatttcttgttttttattgtattcactgctgtctatgtcttcatcatcatagggaatatgctgattattgtagcagtggttagctcccagaggctccacaaacccatgtatattttcttggcgaatctgtccttcctggatattctctacacctccgcagtgatgccaaaaatgctggagggcttcctgcaagaagcaactatctctgtggctggttgcttgctccagttctttatcttcggctctctagccacagctgaatgcttactgctggctgtcatggcatatgaccgctacctggcaatttgctacccactccactacccactcctgatggggcccagacggtacatggggctggtggtcacaacctggctctctggatttgtggtagatggactggttgtggccctggtggcccagctgaggttctgtggccccaaccacattgaccagttttactgtgactttatgcttttcgtgggcctggcttgctcggatcccagagtggctcaggtgacaactctcattctgtctgtgttctgcctcactattccttttggactgattctgacatcttatgccagaattgtggtggcagtgctgagagttcctgctggggcaagcaggagaagggctttctccacatgctcctcccacctagctgtagtgaccacattctatggaacgctcatgatcttttatgttgcaccctctgctgtccattcccagctcctctccaaggtcttctccctgctctacactgtggtcacccctctcttcaatcctgtgatctataccatgaggaacaaggaggtgcatcaggcacttcggaagattctctgtatcaaacaaactgaaacacttgattgaaggagag

SEQ ID NO: 18 - protein, 315 aa, Homo sapiens
MEIVSTGNETITEFVLLGFYDIPELHFLFFIVFTAVYVFIIIGNMLIIVAVVSSQRLHKPMYIFLANLSFLDILYTSAVMPKMLEGFLQEATISVAGCLLQFFIFGSLATAECLLLAVMAYDRYLAICYPLHYPLLMGPRRYMGLVVTTWLSGFVVDGLVVALVAQLRFCGPNHIDQFYCDFMLFVGLACSDPRVAQVTTLILSVFCLTIPFGLILTSYARIVVAVLRVPAGASRRRAFSTCSSHLAVVTTFYGTLMIFYVAPSAVHSQLLSKVFSLLYTVVTPLFNPVIYTMRNKEVHQALRKILCIKQTETLD

SEQ ID NO: 19 - DNA, 1383 bp, Homo sapiens
tagaaatggaacgaccacaaagtgattttaaccaaactgaagttgctgaatttttcctcatgggattttcgaattcctgggatattcagattgtacatgctgctctattcttcctagtttacctggcagctgtcataggaaatctcctaatcatcatacttaccactctggatgttcacctccaaaccccaatgtatttctttttgagaaacttgtctttcttagatttttgttacatctctgtcacaattccaaaatctattgttagttccttgactcatgatacttccatttctttctttgggtgtgctctgcaagccttctttttcatggacttggcaactacggaggtagccatccttacagtgatgtcctatgaccgctatatggccatctgccggcctttacattatgaggtcatcataaaccaaggtgtctgtctgaggatgatggccatgtcgtggctcagtggggtgatctgtggattcatgcatgtgatagcaacattctcattaccattctgtgggcgcaatagaatacgtcaatttttctgtaatattccacagctcctaagcctcttagaccccaaagtaattaccattgagattggagtcatggtttttggtacaagtcttgtgataatctcctttgttgtaattactctctcctacatgtacattttttctgtcatcatgaggattccttctaaggagggtagatcaaaaacattttctacctgcattccacatcttgtggttgtaacactctttatgatatctggcagcattgcctatgtgaagccaatttcaaattctccccccgttctggatgttttcctgtctgcgttctacacagtcgtgcccccgaccctgaaccccgtcatctatagtctgaggaatagggacatgaaggcagccctgagaaggcagtgtggtccctgagaaggcagtgtggtatgctagatgaagaatttgattacggaccagactcttgaactcttgctctaatcaggcaatttgtaaactctctgggcttatattttcaattgattgctgagctctttcttgatttttaaattaaattcagctatagtttaccatactaaccatatttgggtgaattcaaccttagaatgcactaaaaggtacttcggggtgttcaaaacattagaacttgtgcttttattcatttttatgagttgtagatctcaaaattttataaattatattgtactcagtaatacaacatattaattaatataaaagtgaattactaatataaatgaattactaccgtatgtgtatattaattacagcagaacaagatcatatggattaaaaacaaccaaatggaaaataaacataaggtaacagtgtgtgctca

SEQ ID NO: 20 - protein, 311 aa, Homo sapiens
MERPQSDFNQTEVAEFFLMGFSNSWDIQIVHAALFFLVYLAAVIGNLLIIILTTLDVHLQTPMYFFLRNLSFLDFCYISVTIPKSIVSSLTHDTSISFFGCALQAFFFMDLATTEVAILTVMSYDRYMAICRPLHYEVIINQGVCLRMMAMSWLSGVICGFMHVIATFSLPFCGRNRIRQFFCNIPQLLSLLDPKVITIEIGVMVFGTSLVIISFVVITLSYMYIFSVIMRIPSKEGRSKTFSTCIPHLVVVTLFMISGSIAYVKPISNSPPVLDVFLSAFYTVVPPTLNPVIYSLRNRDMKAALRRQCGP

SEQ ID NO: 21 - DNA, 958 bp, Homo sapiens
aacatggaaagcaatcagacctggatcacagaagtcatcctgttgggattccaggtggacccagctctggagttgttcctctttgggtttttcttgctattctacagcttaaccctgatgggaaatgggattatcctggggctcatctacttggactctagactgcacacacccatgtatgtcttcctgtcacacctggccattgtggacatgtcctatgcctcgagtactgtccctaagatgctagcaaatcttgtgatgcacaaaaaagtcatctcctttgctccttgcatacttcagacttttttgtatttggcgtttgctattacagagtgtctgattttggtgatgatgtgctatgatcggtatgtggcaatctgtcaccccttgcaatacaccctcattatgaactggagagtgtgcactgtcctggcctcaacttgctggatatttagctttctcttggctctggtccatattactcttattctgaggctgcctttttgtggcccacaaaagatcaaccactttttctgtcaaatcatgtccgtattcaaattggcctgtgctgacactaggctcaaccaggtggtcctatttgcgggttctgcgttcatcttagtggggccgctctgcctggtgctggtctcctacttgcacatcctggtggccatcttgaggatccagtctggggagggccgcagaaaggccttctctacctgctcctcccacctctgcgtggtggggcttttctttggcagcgccattgtcatgtacatggcccccaggtcaaaccattctcaagaacggaggaagatcctttccctgttttacagccttttcaacccgatcctgaaccccctcatctacagccttaggaatgcagaggtgaaaggggctctaaagagagtcctttggaaacagagatcaatgtgaagaatcatttgagatatcctga

SEQ ID NO: 22 - protein, 310 aa, Homo sapiens
MESNQTWITEVILLGFQVDPALELFLFGFFLLFYSLTLMGNGIILGLIYLDSRLHTPMYVFLSHLAIVDMSYASSTVPKMLANLVMHKKVISFAPCILQTFLYLAFAITECLILVMMCYDRYVAICHPLQYTLIMNWRVCTVLASTCWIFSFLLALVHITLILRLPFCGPQKINHFFCQIMSVFKLACADTRLNQVVLFAGSAFILVGPLCLVLVSYLHILVAILRIQSGEGRRKAFSTCSSHLCVVGLFFGSAIVMYMAPRSNHSQERRKILSLFYSLFNPILNPLIYSLRNAEVKGALKRVLWKQRSM

SEQ ID NO: 23 - DNA, 968 bp, Homo sapiens
agctctactagaaatatggagagcagaaaccaatcaacagtgactgaatttatcttcactggattccctcagcttcaggatggtagtctcctgtacttctttcctttacttttcatctatacttttattatcattgataacttattaatcttctctgctgtaaggctggacacccatctccacaaccccatgtataattttatcagtatattttcctttctggagatctggtacaccacagccaccattcccaagatgctctccaacctcatcagtgaaaagaaggccatctcaatgactggctgcatcttgcagatgtatttcttccactcacttgaaaactcagaggggatcttgctgaccaccatggccattgacagatacgttgccatctgcaaccctcttcgctatcaaatgatcatgaccccccggctctgtgctcaactctctgcaggttcctgcctcttcggtttccttatcctgcttcccgagattgtgatgatttccacactgcctttctgtgggcccaaccaaatccatcagatcttctgtgacttggtccctgtgctaagcctggcctgtacagacacgtccatgattctgattgaggatgtgattcatgctgtgaccatcatcattaccttcctaatcattgccctgtcctatgtaagaattgtcactgtgatattgaggattccctcttctgaagggaggcaaaaggctttttctacctgtgcaggccaccccatggtcttcccgatattctttggcagtgtatcactcatgtacttgcgtttcagcgacacttatccaccagttttggacacagccattgcactgatgtttactgtacttgctccattcttcaatcccatcatttatagcctgagaaacaaggacatgaacaatgcgattaaaaaactgttctgtcttcaaaaagtgttgaacaagcctggaggttaatacag

SEQ ID NO: 24 - protein, 315 aa, Homo sapiens
MESRNQSTVTEFIFTGFPQLQDGSLLYFFPLLFIYTFIIIDNLLIFSAVRLDTHLHNPMYNFISIFSFLEIWYTTATIPKMLSNLISEKKAISMTGCILQMYFFHSLENSEGILLTTMAIDRYVAICNPLRYQMIMTPRLCAQLSAGSCLFGFLILLPEIVMISTLPFCGPNQIHQIFCDLVPVLSLACTDTSMILIEDVIHAVTIIITFLIIALSYVRIVTVILRIPSSEGRQKAFSTCAGHPMVFPIFFGSVSLMYLRFSDTYPPVLDTAIALMFTVLAPFFNPIIYSLRNKDMNNAIKKLFCLQKVLNKPGG

SEQ ID NO: 25 - DNA, 928 bp, Homo sapiens
gttttctgtcatctacatcaacgccatgataggaaatgtgctcattgtggtcaccatcactgccagcccatcactgagatcccccatgtactttttcctggcctatctctcctttattgatgcctgctattcctctgtcaatgcccctaagctgatcacagattcactctatgaaaacaagactatcttattcaatggatgtatgactcaagtctttggagaacattttttcagaggtgttgaggtcatcctacttactgtaatggcctatgaccactatgtggccatctgcaagcccttgcactataccaccatcatgaagaagcatgtttgtagcctgctagtgggagtgtcatgggtaggaggctttcttcatgcaaccatacagatcctcttcatctgtcaattacctttctgtggtcctaatgtcatagatcactttatgtgtgatctctacactttgatcaatcttgcctgcactaatacccacactctaggactcttcattgctgccaacagtgggttcatatgcctgttaaactgtctcttgctcctggtctcctgcgtggtcatactgtactccttaaagacccacagcttagaggcaaggcacgaagccctctctacctgtgtctcccacatcacagttgtcatcttatcctttataccctgcatatttgtgtacatgagacctccagctactttacccattgataaagcagttgctgtattctacactatgataacttctatgttaaaccccttaatctacaccttgaggaatgctcaaatgaaaaatgccattaggaaattgtgtagtaggaaagctatttcaagtgtcaaataaatgtgactggagcccaacatgattcaactgaggcaagggtcaaaaggacattttgggtaatgccagcaaaggaatacttat

SEQ ID NO: 26 - protein, 281 aa, Homo sapiens
FSVIYINAMIGNVLIVVTITASPSLRSPMYFFLAYLSFIDACYSSVNAPKLITDSLYENKTILFNGCMTQVFGEHFFRGVEVILLTVMAYDHYVAICKPLHYTTIMKKHVCSLLVGVSWVGGFLHATIQILFICQLPFCGPNVIDHFMCDLYTLINLACTNTHTLGLFIAANSGFICLLNCLLLLVSCVVILYSLKTHSLEARHEALSTCVSHITVVILSFIPCIFVYMRPPATLPIDKAVAVFYTMITSMLNPLIYTLRNAQMKNAIRKLCSRKAISSVK

SEQ ID NO: 27 - DNA, 971 bp, Homo sapiens
caatgatggaaatagccaatgtgagttctccagaagtctttgtcctcctgggcttctccgcacgaccctcactagaaactgtcctcttcatagttgtcttgagtttttacatggtatcgatcttgggcaatggcatcatcattctggtctcccatacagatgtgcacctccacacacctatgtacttctttcttgccaacctctccttcctggacatgagcttcaccacgagcattgtcccacagctcctggctaacctctggggaccacagaaaaccataagctatggagggtgtgtggtccagttctatatctcccattggctgggggcaaccgagtgtgtcctgctggccaccatgtcctatgaccgctacgctgccatctgcaggccactccattacactgtcattatgcatccacagctttgccttgggctagctttggcctcctggctggggggtctgaccaccagcatggtgggctccacgctcaccatgctcctaccgctgtgtgggaacaattgcatcgaccacttcttttgcgagatgcccctcattatgcaactggcttgtgtggataccagcctcaatgagatggagatgtacctggccagctttgtctttgttgtcctgcctctggggctcatcctggtctcttacggccacattgcccgggccgtgttgaagatcaggtcagcagaagggcggagaaaggcattcaacacctgttcttcccacgtggctgtggtgtctctgttttacgggagcatcatcttcatgtatctccagccagccaagagcacctcccatgagcagggcaagttcatagctctgttctacaccgtagtcactcctgcgctgaacccagttatttacaacctgaggaacacggaggtgaagagcgccctccggcacatggtattagagaactgctgtggctctgcaggcaagctggcgcaaatttagagactc

SEQ ID NO: 28 - protein, 320 aa, Homo sapiens
MMEIANVSSPEVFVLLGFSARPSLETVLFIVVLSFYMVSILGNGIIILVSHTDVHLHTPMYFFLANLSFLDMSFTTSIVPQLLANLWGPQKTISYGGCVVQFYISHWLGATECVLLATMSYDRYAAICRPLHYTVIMHPQLCLGLALASWLGGLTTSMVGSTLTMLLPLCGNNCIDHFFCEMPLIMQLACVDTSLNEMEMYLASFVFVVLPLGLILVSYGHIARAVLKIRSAEGRRKAFNTCSSHVAVVSLFYGSIIFMYLQPAKSTSHEQGKFIALFYTVVTPALNPVIYNLRNTEVKSALRHMVLENCCGSAGKLAQI

SEQ ID NO: 29 - DNA, 958 bp, Homo sapiens
ctatggagcagagcaattattccgtgtatgccgactttatccttctgggtttgttcagcaacgcccgtttcccctggcttctctttgccctcattctcctggtctttgtgacctccatagccagcaacgtggtcaagatcattctcatccacatagactcccgcctccacacccccatgtacttcctgctcagccagctctccctcagggacatcttgtatatttccaccattgtgcccaaaatgctggtcgaccaggtgatgagccagagagccatttcctttgcaggatgcactgcccaacacttcctctacttgaccttagcaggggctgagttcttcctcctaggactcatgtcctgtgatcgctacgtagccatctgcaaccctctgcactatcctgacctcatgagccgcaagatctgctggttgattgtggcggcagcctggctgggagggtctatcgatggtttcttgctcacccccgtcaccatgcagttccccttctgtgcctctcgggagatcaaccacttcttctgcgaggtgcctgcccttctgaagctctcctgcacggacacatcagcctacgagacagccatgtatgtctgctgtattatgatgctcctcatccctttctctgtgatctcgggctcttacacaagaattctcattactgtttataggatgagcgaggcagaggggaggcgaaaggctgtggccacctgctcctcacacatggtggttgtcagcctcttctatggggctgccatgtacacatacgtgctgcctcattcttaccacacccctgagcaggacaaagctgtatctgccttctacaccatcctcactcccatgctcaatccactcatttacagccttaggaacaaggatgtcacgggggccctacagaaggttgttgggaggtgtgtgtcctcaggaaaggtaaccactttctaaac

SEQ ID NO: 30 - protein, 317 aa, Homo sapiens [sequence omitted]

SEQ ID NO: 31 - DNA, 1013 bp, Homo sapiens
cctatgtgatgtgttatctttctcagctatgcctcagccttggggaacacactttacatatggggatggtgagacataccaatgagagcaacctagcaggtttcatccttttagggttttctgattatcctcagttacagaaggttctatttgtgctcatattgattctgtatttactaactattttggggaataccaccatcattctggtttctcgtctggaacccaagcctcatatgccgatgtatttcttcctttctcatctctccttcctgtaccgctgcttcaccagcagtgttattccccagctcctggtaaacctgtgggaacccatgaaaactatcgcctatggtggctgtttggttcacctttacaactcccatgccctgggatccactgagtgcgtcctcccggctctgatgtcctgtgaccgctatgtggctgtctgccgtcctctccattacactgtcttaatgcatatccatctctgcatggccttggcatctatggcatggctcagtggaatagccaccaccctggtacagtccaccctcaccctgcagctgcccttctgtgggcatcgccaagtggatcatttcatctgcgaggtccctgtgctcatcaagctggcttgtgtgggcaccacgtttaacgaggctgagctttttgtggctagtatccttttccttatagtgcctgtctcattcatcctggtctcctctggctacattgcccacgcagtgttgaggattaagtcagctaccgggagacagaaagcattcgggacctgcttctcccacctgacagtggtcaccatcttttatggaaccatcatcttcatgtatctgcagccagccaagagtagatccagggaccagggcaagtttgtttctctcttctacactgtggtaacccgcatgcttaaccctcttatttataccttgaggatcaaggaggtgaaaggggcattaaagaaagttctagcaaaggctctgggagtaaatattttatga

SEQ ID NO: 32 - protein, 334 aa, Homo sapiens
MCYLSQLCLSLGEHTLHMGMVRHTNESNLAGFILLGFSDYPQLQKVLFVLILILYLLTILGNTTIILVSRLEPKPHMPMYFFLSHLSFLYRCFTSSVIPQLLVNLWEPMKTIAYGGCLVHLYNSHALGSTECVLPALMSCDRYVAVCRPLHYTVLMHIHLCMALASMAWLSGIATTLVQSTLTLQLPFCGHRQVDHFICEVPVLIKLACVGTTFNEAELFVASILFLIVPVSFILVSSGYIAHAVLRIKSATGRQKAFGTCFSHLTVVTIFYGTIIFMYLQPAKSRSRDQGKFVSLFYTVVTRMLNPLIYTLRIKEVKGALKKVLAKALGVNIL

SEQ ID NO: 33 - DNA, 996 bp, Homo sapiens
catgctggactatgccctctccatttacaggtagctctactagaaatatggagagcagaaaccaatcaacagtgactgaatttatcttcactggattccctcagcttcaggatggtagtctcctgtacttctttcctttacttttcatctatacttttattatcattgataacttattaatcttctctgctgtaaggctggacacccatctccacaaccccatgtataattttatcagtatattttcctttctggagatctggtacaccacagccaccattcccaagatgctctccaacctcatcagtgaaaagaaggccatctcaatgactggctgcatcttgcagatgtatttcttccactcacttgaaaactcagaggggatcttgctgaccaccatggccattgacagatacgttgccatctgcaaccctcttcgctatcaaatgatcatgaccccccggctctgtgctcaactctctgcaggttcctgcctcttcggtttccttatcctgcttcccgagattgtgatgatttccacactgcctttctgtgggcccaaccaaatccatcagatcttctgtgacttggtccctgtgctaagcctggcctgtacagacacgtccatgattctgattgaggatgtgattcatgctgtgaccatcatcattaccttcctaatcattgccctgtcctatgtaagaattgtcactgtgatattgaggattccctcttctgaagggaggcaaaaggctttttctacctgtgcaggccacctcatggtcttcccgatattctttggcagtgtatcactcatgtacttgcgtttcagcgacacttatccaccagttttggacacagccattgcactgatgtttactgtacttgctccattcttcaatcccatcatttatagcctgagaaacaaggacatgaacaatgcgattaaaaaactgttctgtcttcaaaaagtgttgaacaagcctggaggttaat

SEQ ID NO: 34 - protein, 327 aa, Homo sapiens [sequence omitted]

SEQ ID NO: 35 - protein, 321 aa, Mus musculus
MNSKASMLGTNFTIIHPTVFILLGIPGLEQYHTWLSIPFCLMYIAAVLGNGALILVVLSERTLHEPMYVFLSMLAGTDILLSTTTVPKTLAIFWFHAGEIPFDACIAQMFFIHVAFVAESGILLAMAFDRYVAICTPLRYSAVLTPMAIGKMTLAIWGRSIGTIFPIIFLLKRLSYCRTNVIPHSYCEHIGVARLACADITVNIWYGFSVPMASVLVDVALIGISYTLILQAVFRLPSQDARHKALNTCGSHIGVILLFFIPSFFTFLTHRFGKNIPHHVHILLANLYVLVPPMLNPIIYGAKTKQIRDSMTRMLSVVWKS

SEQ ID NO: 36 - protein, 326 aa, Mus musculus
MKVASSFHNDTNPQDVWYVLIGIPGLEDLHSWIAIPICSMYIVAVIGNVLLIFLIVTERSLHEPMYFFLSMLALADLLLSTATAPKMLAIFWFHSRGISFGSCVSQMFFIHFIFVAESAILLAMAFDRYVAICYPLRYTTILTSSVIGKIGTAAVVRSFLICFPFIFLVYRLLYCGKHIIPHSYCEHMGIARLACDNITVNIIYGLTMALLSTGLDILLIIISYTMILRTVFQIPSWAARYKALNTCGSHICVILLFYTPAFFSFFAHRFGGKTVPRHIHILVANLYVVVPPMLNPIIYGVKTKQIQDRVVFLFSSVSTCQHDSRC

SEQ ID NO: 37 - protein, 318 aa, Mus musculus
  VARIANT (286): Where Xaa is Thr, Leu, Gln or Arg
MSPGNSSWIHPSSFLLLGIPGLEELQFWLGLPFGTVYLIAVLGNVIILFVIYLEHSLHQPMFYLLAILAVTDLGLSTATVPRALGIFWFGFHKIAFRDCVAQMFFIHLFTGIETFMLVAMAFDRYIAICNPLRYNTILTNRTICIIVGVGLFKNFILVFPLIFLILRLSFCGHNIIPHTYCEHMGIARLACVSIKVNVLFGLILISMILLDVVLSALSYAKILHAVFKLPSWEARLKALNTCGSHVCVILAFFTPAFFSFLTHRFGHNIPRYIHILLANLYVIIPXALNPIIYGVRTKQIQDRAVTILCNEVGQLADD

SEQ ID NO: 38 - protein, 318 aa, Homo sapiens
MSDSNLSDNHLPDTFFLTGIPGLEAAHFWIAIPFCAMYLVALVGNAALILVIAMDNALHAPMYLFLCLLSLTDLALSSTTVPKMLAILWLHAGEISFGGCLAQMFCVHSIYALESSILLAMAFDRYVAICNPLRYTTILNHAVIGRIGFVGLFRSVAIVSPFIFLLRRLPYCGHRVMTHTYCEHMGIARLACANITVNIVYGLTVALLAMGLDSILIAISYGFILHAVFHLPSHDAQHKALSTCGSHIGIILVFYIPAFFSFLTHRFGHHEVPKHVHIFLANLYVLVPPVLNPILYGARTKEIRSRLLKLLHLGKTSI

SEQ ID NO: 39 - protein, 339 aa, Mus musculus
MPEKMLSKLIAYLLLIESCRQTAQLVKGRRIWVDSRPHWPNTTHYRELEDQHVWIAIPFCSMYILALVGNGTILYIIITDRALHEPMYLFLCLLSITDLVLCSTTLPKMLAIFWLRSHVISYHGCLTQMFFVHAVFATESAVLLAMAFDRYVAICRPLHYTSILNAVVIGKIGLACVTRGLLFVFPFVILIERLPFCGHHIIPHTYCEHMGIAKLACASIKPNTIYGLTVALSVTGMDVVLIATSYILILQAVLRLPSKDAQFRAFSTCGAHICVILVFYIPAFFSFFTHRFGHHVPPQVHIILANLYLLVPPVLNPLVYGINTKQIRLRILDFFVKRR

SEQ ID NO: 40 - protein, 254 aa, Artificial Sequence
  Description of Artificial Sequence7tm_1, 7 transmembrane receptor domain sequence
GNLLVILVILRTKKLRTPTNIFLLNLAVADLLFLLTLPPWALYYLVGGDWVFGDALCKLVGALFVVNGYASILLLTAISIDRYLAIVHPLRYRRIRTPRRAKVLILLVWVLALLLSLPPLLFSWLRTVEEGNTTVCLIDFPEESVKRSYVLLSTLVGFVLPLLVILVCYTRILRTLRKRARSQRSLKRRSSSERKAAKMLLVVVVVFVLCWLPYHIVLLLDSLCLLSIWRVLPTALLITLWLAYVNSCLNPIIY

SEQ ID NO: 41 - protein, 320 aa, Homo sapiens
MSSCNFTHATFVLIGIPGLEKAHFWVGFPLLSMYVVAMFGNCIVVFIVRTERSLHAPMYLFLCMLAAIDLALSTSTMPKILALFWFDSREISFEACLTQMFFIHALSAIESTILLAMAFDRYVAICHPLRHAAVLNNTVTAQIGIVAVVRGSLFFFPLPLLIKRLAFCHSNVLSHSYCVHQDVMKLAYADTLPNVVYGLTAILLVMGVDVMFISLSYFLIIRTVLQLPSKSERAKAFGTCVSHIGVVLAFYVPLIGLSVVHRFGNSLHPIVRVVMGDIYLLLPPVINPIIYGAKTKQIRTRVLAMFKISCDKDLQAVGGK

SEQ ID NO: 42 - protein, 320 aa, Mus musculus
MSSCNFTHATFLLIGIPGLEEAHFWFGFPLLSMYAVALFGNCIVVFIVRTERSLHAPMYLFLCMLAAIDLALSTSTMPKILALFWFDSREITFDACLAQMFFIHTLSAIESTILLAMAFDRYVAICHPLRHAAVLNNTVTVQIGMVALVRGSLFFFPLPLLIKRLAFCHSNVLSHSYCVHQDVMKLAYTDTLPNVVYGLTAILLVMGVDVMFISLSYFLIIRTVLQLPSKSERAKAFGTCVSHISVVLAFYVPLIGLSVVHRFGNSLDPIVHVLMGDVYLLLPPVINPIIYGAKTKQIRTRVLAMFKISCDKDIEAGGNT

SEQ ID NO: 43 - protein, 319 aa, Mus musculus
MATSNSSTIVSSTFYLTGIPGYEEFHHWISIPFCFLYLVGITGNCMILHIVRTDPRLHEPMYYFLAMLSLTDMAMSLPTMMSLFRVLWSISREIQFNICVVQMFLIHTFSFTESSVLLAMALDRYVAICHPLRYATILTPKLIAKIGTAALLRSSILIIPLIARLAFFPFCGSHVLSHSYCLHQDMIRLACADIRFNVIYGLVLITLLWGMDSLGIFVSYVLILHSVLKIASREGRLKALNTCASHICAVLILYVPMIGLSIVHRFAKHSSPLIHIFMAHIYLLVPPVLNPIIYSVKTKQIREGILHLLCSPKISSITM

SEQ ID NO: 44 - protein, 320 aa, Rattus norvegicus
MSSCNFTHATFMLIGIPGLEEAHFWFGFPLLSMYAVALFGNCIVVFIVRTERSLHAPMYLFLCMLAAIDLALSTSTMPKILALFWFDSREITFDACLAQMFFIHALSAIESTILLAMAFDRYVAICHPLRHAAVLNNTVTVQIGMVALVRGSLFFFPLPLLIKRLAFCHSNVLSHSYCVHQDVMKLAYTDTLPNVVYGLTAILLVMGVDVMFISLSYFLIIRAVLQLPSKSERAKAFGTCVSHIGVVLAFYVPLIGLSVVHRFGNSLDPIVHVLMGDVYLLLPPVINPIIYGAKTKQIRTRVLAMFKISCDKDIEAGGNT

SEQ ID NO: 45 - protein, 312 aa, Homo sapiens
MGLFNVTHPAFFLLTGIPGLESSHSWLSGPLCVMYAVALGGNTVILQAVRVEPSLHEPMYYFLSMLSFSDVAISMATLPTVLRTFCLNARNITFDACLIQMFLIHFFSMMESGILLAMSFDRYVAICDPLRYATVLTTEVIAAMGLGAAARSFITLFPLPFLIKRLPICRSNVLSHSYCLHPDMMRLACADISINSIYGLFVLVSTFGMDLFFIFLSYVLILRSVMATASREERLKALNTCVSHILAVLAFYVPMIGVSTVHRFGKHVPCYIHVLMSNVYLFVPPVLNPLIYSAKTKEIRRAIFRMFHHIKI

SEQ ID NO: 46 - protein, 254 aa, Artificial Sequence; identical to SEQ ID NO: 40
  Description of Artificial Sequence7tm_1, 7 transmembrane receptor domain sequence

SEQ ID NO: 47 - protein, 357 aa, Homo sapiens; identical to SEQ ID NO: 12

SEQ ID NO: 48 - protein, 357 aa, Homo sapiens
MNWVNKSVPQEFILLVFSDQPWLEIPPFVMFLFSYILTIFGNLTIILVSHVDFKLHTPMYFFLSNLSLLDLCYTTSTVPQMLVNICNTRKVISYGGCVAQLFIFLALGSTECLLLAVMCFDRFVAICRPLHYSIIMHQRLCFQLAAASWISGFSNSVLQSTWTLKMPLCGHKEVDHFFCEVPALLKLSCVDTTANEAELFFISVLFLLIPVTLILISYAFIVQAVLRIQSAEGRRKAFGTCGSHLIVVSLFYGTAISMYLQPPSPSSKDRGKMVSLFCGIIAPMLNPLIYTLRNKEVKEAFKRLVAKSLLNQEIRNMQMISFAKDTVLTYLTNFSASCPIFVITIENYCNLPQRKFP

SEQ ID NO: 49 - protein, 310 aa, Homo sapiens
MNWVNKSVPQEFILLVFSDQPWLEIPPFVMFLFSYILTIFGNLTIILVSHVDFKLHTPMYFFLSNLSLLDLCYTTSTVPQMLVNICNTRKVISYGGCVAQLFIFLALGSTECLLLAVMCFDRFVAICRPLHYSIIMHQRLCFQLAAASWISGFSNSVLQSTWTLKMPLCGHKEVDHFFCEVPALLKLSCVDTTANEAELFFISVLFLLIPVTLILISYAFIVQAVLRIQSAEGQRKAFGTCGSHLIVVSLFYGTAISMYLQPPSPSSKDRGKMVSLFCGIIAPMLNPLIYTLRNKEVKEAFKRLVAKSLL

SEQ ID NO: 50 - protein, 313 aa, Rattus norvegicus
MSVANESISREFILLGFSDRPWLELPLFVVFLVSYILTIFGNMMIILVSRLDSKLHTPMYFFLTNLSLLDLCYTTSTVPQMLINICSTRKVISYGGCVVQLFIFLSLGSTECFLLGVMSLDRFLAICRPLHYSVIMHQRRCLHLAAACWISGFSNSVLQSTWTLQMPLCGHKEVDHFFCEVPALLKLSCVDTTANEAELFFISVLFLLIPVTLILISYAFIVQAVLKIRSAECRRKAFGTCGSHLIVVVLFYGTAIYMYLQPPSPSSKDRGKMVSLFYGIITPMLNPLIYTLRNEEVKGAFKRLMKRIILIGK

SEQ ID NO: 51 - protein, 313 aa, Homo sapiens
MNWVNDSIIQEFILLGFSDRPWLEFPLLVVFLISYTVTIFGNLTIILVSRLDTKLHTPMYFFLTNLSLLDLCYTTCTVPQMLVNLCSIRKVISYRGCVAQLFIFLALGATEYLLLAVMSFDRFVAICRPLHYSVIMHQRLCLQLAAASWVTGFSNSVWLSTLTLQLPLCDPYVIDHFLCEVPALLKLSCVETTANEAELFLVSELFHLIPLTLILISYAFIVRAVLRIQSAEGRQKAFGTCGSHLIVVSLFYSTAVSVYLQPPSPSSKDQGKMVSLFYGIIAPMLNPLIYTLRNKEVKEGFKRLVARVFLIKK

SEQ ID NO: 52 - protein, 254 aa, Artificial Sequence; identical to SEQ ID NO: 40
  Description of Artificial Sequence7tm_1, 7 transmembrane receptor domain sequence

SEQ ID NO: 53 - protein, 310 aa, Mus musculus
MTAKNSSVTEFILAGLTDQPGLRMPLFFLFLGFYMVTVVGNLGLISLIGLNSHLHTPMYFFLFNLSLIDFCYSSTISPKMLMSFISKKNIISHPGCMAQLFFFCFFVISESFILSAMAYDRYVAICNPLMYMVTMSPQVCLLLLFGVYLMGFVGAMAHTISMARLTFCADNIVNHYMCDILPLLEHSCTSTYVNELVVFIFVSFDIGVPIVTIFISYALILSSILHMHSTEGRSKAFSTCSSHMIVVCLFFGSGAFMYLQPPSVLSLDQGKVSSLFYTIVVPMLNPLIYSLRNKDVKVAVRKTLDRRIFS

SEQ ID NO: 54 - protein, 310 aa, Mus musculus
MATENASVPEFILAGLTDQPGLRMPLFFLFLGFYMVTMVGNLGLITLIGLNSHLHTPMYFFLFNLSLIDFCYSTVITPKMLVSFVSKKNIISYSGCMTQLFFFLFFVVSESFILSAMAYDRYVAICNPLMYTVTMSPQVCLLLLLGVYVMGFAGAMAHTAFMVKLTFCADKLVNHYMCDILPLLERSCTSTYVNELVVFIVVGIDIGVPTVTIFISYALILSSILRISSTEGRSKAFSTCSSHIIAVSLFFGSGAFMYLKPSSLLPMNQGKVSSLFYTIVVPMLNPLIYSLRNKDVKVALRKTLSRSSFS

SEQ ID NO: 55 - protein, 309 aa, Mus musculus
MTAKNSSVIEFILAGLTDQPGLRMPLFFLFLGFYMVTVVGNLGLISLIGLNSHLHTPMYFFLFNLSVIDFCYSSTIIPKMLMNFISRKNIISHSGCMTQLFFFCFFVVSESFILSAMAYDRYVAICNPLMYTVTMSPQVYLLLLLGVYVMGFSGAMAIQELMNLTFCADNLINHFMCDILPLLELSCNSTFINELVIFIVVAFDIGVPIVTIFISYALILSSILRMHSTEGRSKAFSTYSSHLIVVCLFFGSGAFMYLKPPSILPLDQGKVSSLFYMIVVPMLNPLIYSLRNKDVKVGLRKTLGKRILS

SEQ ID NO: 56 - protein, 304 aa, Rattus norvegicus
SVTEFILAGLTDQPGLRMPLFFLFLGFYMVTVVGNLIGLFLIGLNSHLHTPMYFFLFNLSVVDFCFSSTIIPKMLMSFISKKNIISHSGCMTQLFFFCFFVVSETFILSAMAYDRYVAICNPLMYTVTMSPQVCLLLLLGAYVMGFSEAMAHTGNLMNLTFCADNLVNHFMCDILPLLELSCNSTFINELVVFIVVAIDIAVPIVSIFISYALILSSILRMHSTEGRSKAFSTCSSHLIVVCLLFGSGAFMYLKLPSILPLDQGKVSSLFYTIVVPMLNPLIYSLRNKDVKVALRKTLGKIILS

SEQ ID NO: 57 - protein, 311 aa, Homo sapiens
MAAENSSFVTQFILAGLTDQPGVQIPLFFLFLGFYVVTVVGNLGLITLIRLNSHLHTPMYFFLYNLSFIDFCYSSVITPKMLMSFVLKKNSISYAGCMTQLFFFLFFVVSESFILSAMAYDRYVAICNPLLYMVTMSPQVCFLLLLGVYGMGFAGAMAHTACMMGVTFCANNLVNHYMCDILPLLECACTSTYVNELVVFVVVGIDIGVPTVTIFISYALILSSIFHIDSTEGRSKAFSTCSSHIIAVSLFFGSGAFMYLKPFSLLAMNQGKVSSLFYTTVVPMLNPLIYSLRNKDVKVALKKILNKNAFS

SEQ ID NO: 58 - protein, 241 aa, Artificial Sequence
  Description of Artificial Sequence7tm_1, 7 transmembrane receptor domain sequence
KLRTPTNIFLLNLAVADLLFLLTLPPWALYYLVGGDWVFGDALCKLVGALFVVNGYASILLLTAISIDRYLAIVHPLRYRRIRTPRRAKVLILLVWVLALLLSLPPLLFSWLRTVEEGNTTVCLIDFPEESVKRSYVLLSTLVGFVLPLLVILVCYTRILRTLRKRARSQRSLKRRSSSERKAAKMLLVVVVVFVLCWLPYHIVLLLDSLCLLSIWRVLPTALLITLWLAYVNSCLNPIIY

SEQ ID NO: 59 - protein, 321 aa, Mus musculus; identical to SEQ ID NO: 35

SEQ ID NO: 60 - protein, 326 aa, Mus musculus; identical to SEQ ID NO: 36

SEQ ID NO: 61 - protein, 339 aa, Mus musculus; identical to SEQ ID NO: 39

SEQ ID NO: 62 - protein, 269 aa, Homo sapiens
MSDSNLSDNHLPDTFFLTGIPGLEAAHFWIAIPFCAMYLVALVGNAALILVIAMDNALHAPMYLFLCLLSLTDLALSSTTVPKMLAILWLHAGEISFGGCLAQMFCVHSIYALESSILLAMAFDRYVAICNPLRYTTILNHAVIGRIGFVGLFRSVAIVSPFIFLLRRLPYCGHRVMTHTYCEHMGIARLACANITVNIVYGLTVALLAMGLDSILIAISYGFILHAVFHLPSHDAQHKALSTCGSHIGIILVFYIPAFFSFLTHRFGH

SEQ ID NO: 63 - protein, 318 aa, Mus musculus; identical to SEQ ID NO: 37
  VARIANT (286): Where Xaa is Thr, Leu, Gln or Arg

SEQ ID NO: 64 - protein, 254 aa, Artificial Sequence; identical to SEQ ID NO: 40
  Description of Artificial Sequence7tm_1, 7 transmembrane receptor domain sequence

SEQ ID NO: 65 - protein, 315 aa, Homo sapiens; identical to SEQ ID NO: 18

SEQ ID NO: 66 - protein, 315 aa, Homo sapiens
MEIVSTGNETITEFVLLGFYDIPELHFLFFIVFTAVYVFIIIGNMLIIVAVVSSQRLHKPMYIFLANLSFLDILYTSAVMPKMLEGFLQEATISVAGCLLQFFIFGSLATAECLLLAVMAYDRYLAICYPLHYPLLMGPRRYMGLVVTTWLSGFVVDGLVVALVTQLRFCGPNHIDQFYCDFMLFVGLACSDPRVAQVTTLILSVFCLTIPFGLILTSYARIVVAVLRVPAGASRRRAFSTCSSHLAVVTTFYGTLMIFYVAPSAVHSQLLSKVFSLLYTVVTPLFNPVIYTMRNKEVHQALRKILCIKQTETLD

SEQ ID NO: 67 - protein, 292 aa, Marmota marmota
PMYLFLGNLSFLEILYTSTVVPKMLEGFLQVAAISVTGCLTQFFIFGSLATAECFLLAVMAYDRFLAICYPLRYPLLMGPRWCMGLVVTAWLSGFMVDELVVVLMAQLRFCGSNRIDHFYCDFMPLVVLACSDPRVAQVTTFVLSVVFLTVPFGLILTSYARIVVTVLRVPAGASRRKAFSTCSSHLAVVSTFYGTLMVLYIVPSAVHSQLLSKVFALLYTVVTPIFNPIIYSFRNQEVKKALRRTLHLAQGQDANTKKSSRDGQDVKRALRRTLHLAQDQEANTNKGSKIG

SEQ ID NO: 68 - protein, 327 aa, Mus musculus
MERRNHTGRVSEFVLLGFPAPAPLRALLFFLSLLAYVLVLTENILIITAIRNHPTLHKPMYFFLANMSFLEIWYVTVTIPKMLAGFIGSEENHGQLISFEACMTQLYFFLGLGCTECVLLAVMAYDRYVAICHPLHYPVIVSSRLCVQMAAGSWAGGFGISMVKVFLISRLSYCGPNTINHFFCDVSPLLNLSCTDMSTAELTDFILAIFILLGPLSVTGASYMAITGAVMRIPSAAGRHKAFSTCASHLTVVIIFYAASIFIYARPKALSAFDTNKLVSVLYAVIVPLLNPIIYCLRNQEVKKALRRTLHLAQGQDANTKKSSRDG

SEQ ID NO: 69 - protein, 327 aa, Rattus norvegicus
  VARIANT (35): Where Xaa is Asp, Val, Ser or Gly
MERRNHSGRVSEFVLLGFPAPAPLRVLLFFLSLLXYVLVLTENMLIIIAIRNHPTLHKPMYFFLANMSFLEIWYVTVTIPKMLAGFIGSKENHGQLISFEACMTQLYFFLGLGCTECVLLAVMAYDRYVAICHPLHYPVIVSSRLCVQMAAGSWAGGFGISMVKVFLISRLSYCGPNTINHFFCDVSPLLNLSCTDMSTAELTDFVLAIFILLGPLSVTGASYMAITGAVMRIPSAAGRHKAFSTCASHLTVVIIFYAASIFIYARPKALSAFDTNKLVSVLYAVIVPLFNPIIYCLRNQDVKRALRRTLHLAQDQEANTNKGSKIG

SEQ ID NO: 70 - protein, 254 aa, Artificial Sequence; identical to SEQ ID NO: 40
  Description of Artificial Sequence7tm_1, 7 transmembrane receptor domain sequence

SEQ ID NO: 71 - protein, 321 aa, Homo sapiens
MVNLTSMSGFLLMGFSDERKLQILHALVFLVTYLLALTGNLLIITIITVDRRLHSPMYYFLKHLSLLDLCFISVTVPQSIANSLMGNGYISLVQCILQVFFFIALASSEVAILTVMSYDRYAAICQPLHYETIMDPRACRHAVIAVWIAGGLSGLMHAAINFSIPLCGKRVIHQFFCDVPQMLKLACSYEFINEIALAAFTTSAAFICLISIVLSYIRIFSTVLRIPSAEGRTKVFSTCLPHLFVATFFLSAAGFEFLRLPSDSSSTVDLVFSVFYTVIPPTLNPVIYSLRNDSMKAALRKMLSKEELPQRKMCLKAMFKL

SEQ ID NO: 72 - protein, 321 aa, Mus musculus
MTVKNITTMSGFLLMGFSDNRELQILYALLFLLTYLLGSAGNFIIITITTLDPQLQSPMYYFLKHLSILDLSSLSVTVPQYVDSSLAGSGYISYGQCMLQIFFFAAFAWGEVAILTVMSYDRYVAICLPLHYEVIMSPRKCTWAVTSVWLSSVIPGTLYIASIFSIRFCRAKIIHQLFCDVPQLLKLSCSNDHLVVIGMVSFMTAVAFACFVGIVISYVHIFSTVLRMPSAESRSKVFSTCLPHLFVVSLFLSTGSCAYLNTSSDSPTALEFLFSIFYTVLPPTLNPVIYSLRNETIKSVVRKLLLSSKFTVRIICPVATD

SEQ ID NO: 73 - protein, 304 aa, Mus musculus
MRGFLLMGFSDNRELQILHALFFLVAYLLGSAGNVIIITITTLDPQLQSPMYYFLKHLSILDLSSLSVTVPQYVDICLTQSGYISYAQCMLQIFFFTGFAWGEVAILTVMSYDRYVAVCLPLHYEVIMGPSKCRWAVTAVWLSSVIPGTLYIASIFSIRFCGDRIIHQFFCDVPQVLKLSCSDDYLVTVGVADFLSAVAFACFIGIVNSYVHIFSTVLRMPSAESRSKVFSTCLPHLFVVLLFLSTGIFAYLNPTSDSPTALQFLVSIFYTVLPPTLNPVIYSLRNETIKSVIRKLLLSSKFTG

SEQ ID NO: 74 - protein, 320 aa, Mus musculus
MIVENITTMSGFLLMGFSDNHELQILQALLFLVTYLVGSAGNVIIITITTLDPQLQSPMYYFLKHLSILDLSSLSVTVPQYVDSSLAQSGYISYAQCMLQIFFFTAFAWGELAILTVMSYDRYVAICLPLHYEVIMSPRKCTWAVATVWLSGGISGTLYITGTLFIRFCGDKIIHQFFCDVPQLLKLSCSNDHLVIMDMVSFLTAVSFACFTGIVISYVHIFSTVLRMPSAESRSKVFSTCLPHLFVVSLFLSTGAFAYLNLTSDSSTALEFLLSIFYTVLPPTLNPVIYSLRNETIKNVVRKLLLSTKFTVRIIFSCCF

SEQ ID NO: 75 - protein, 312 aa, Mus musculus
MIMENITTMSGFLLMGFSDNRELQILQALLFLVTYLVGSAGNFIIITITTLDPQLKSPMYYFLKHLSILDLSSLSVTVPQYVDSSLARSGYISYEQCMLQIFFFTCFAWDEMAILTVMSYDRYVAVCLPLHYEVIMSPRKCTWALAAVWLSGGVSGTLYTASTLSIRFCGDRIIHQFFCDVPQVLKLSCSNDYLLTIGVANILSAVAFACFIGIVISYVHIFSTVLRMPSAESRYKVFSTCLPHLFVVSLFLSTSTFAYLNPTADSPTALEFLFSILYTVLPPTINPVIYSLRNETIKSVVRKLLSSTKFTV

SEQ ID NO: 76 - protein, 254 aa, Artificial Sequence; identical to SEQ ID NO: 40
  Description of Artificial Sequence7tm_1, 7 transmembrane receptor domain sequence

SEQ ID NO: 77 - protein, 310 aa, Homo sapiens
MGDNITSITEFLLLGFPVGPRIQMLLFGLFSLFYVFTLLGNGTILGLISLDSRLHAPMYFFLSHLAVVDIAYACNTVPRMLVNLLHPAKPISFAGRMMQTFLFSTFAVTECLLLVVMSYDLYVAICHPLRYLAIMTWRVCITLAVTSWTTGVLLSLIHLVLLLPLPFCRPQKIYHFFCEILAVLKLACADTHINENMVLAGAISGLVGPLSTIVVSYMCILCAILQIQSREVQRKAFCTCFSHLCVIGLFYGTAIIMYVGPRYGNPKEQKKYLLLFHSLFNPMLNPLICSLRNSEVKNTLKRVLGVERAL

SEQ ID NO: 78 - protein, 310 aa, Homo sapiens
MGDNITSIREFLLLGFPVGPRIQMLLFGLFSLFYVFTLLGNGTILGLISLDSRLHAPMYFFLSHLAVVDIAYACNTVPRMLVNLLHPAKPISFAGRMMQTFLFSTFAVTECLLLVVMSYDLYVAICHPLRYLAIMTWRVCITLAVTSWTTGVLLSLIHLVLLLPLPFCRPQKIYHFFCEILAVLKLACADTHINENMVLAGAISGLVGPLSTIVVSYMCILCAILQIQSREVQRKAFRTCFSHLCVIGLVYGTAIIMYVGPRYGNPKEQKKYLLLFHSLFNPMLNPLICSLRNSEVKNTLKRVLGVERAL

SEQ ID NO: 79 - protein, 217 aa, Homo sapiens
IIDISYASNKVPKMLTNLGLNKRKTISFVPCTMQTFLYMAFAHTECLILVMMSYDRYMAICHPLQYSVIMRWGVCTVLAVTSWACGSLLALVHVVLILRLPFCGPHEINHFFCEILSVLKLACADTWLNQVVIFAASVFILVGPLCLVLVSYSRILAAILRIQSGEGRRKAFSTCSSHLCMVGLFFGSAIVMYMAPKSRHPEEQQKVLSLFYSLFNP

SEQ ID NO: 80 - protein, 217 aa, Homo sapiens
IIDISYASNNVPKMLTNLGLNKRKTISFVPCTMQTFLYMAFAHTECLILVMMSYDRYMAICHPLQYSVIMRWGVCTVLAVTSWACGSLLALVHVVLILRLPFCGPHEINHFFCEILSVLKLACADTWLNQVVIFAASVFILVGPLCLVLVSYSRILAAILGIQSGEGRRKAFSTCSSHLCMVGLFFGSAIVMYMAPKSRHPEEQQKVLSLFYSLFNP

SEQ ID NO: 81 - protein, 217 aa, Homo sapiens
IIDISYASNKVPKMLTNLGLNKRKTISFVPCTMQTFLYMAFAHTECLILVMMSYDRYMAICHPLQYSVIMRWGVCTVLAVTSWACGSLLALVHVVLILRLPFCGPHEINHFFCEILSVLKLACADTWLNQVVIFAASVFILVGPLCLVLVSYSRILAAILGIQSGEGRRKAFSTCSSHLCMVGLFFGSAIVMYMAPKSRHPEEQQKVLSLFYSLFNP

SEQ ID NO: 82 - protein, 194 aa, Artificial Sequence
  Description of Artificial Sequence 7tm_1, 7 transmembrane receptor domain sequence
GNLLVILVILRTKKLRTPTNIFLLNLAVADLLFLLTLPPWALYYLVGGDWVFGDALCKLVGALFVVNGYASILLLTAISIDRYLAIVHPLRYRRIRTPRRAKVLILLVWVLALLLSLPPLLFSWLRTVEEGNTTVCLIDFPEESVKRSYVLLSTLVGFVLPLLVILVCYTRLPTALLITLWLAYVNSCLNPIIY

SEQ ID NO: 83 - protein, 321 aa, Homo sapiens
MERKNQTAITEFIILGFSNLNELQFLLFTIFFLTYFCTLGGNILIILTTVTDPHLHTPMYYFLGNLAFIDICYTTSNVPQMMVHLLSKKKSISYVGCVVQLFAFVFFVGSECLLLAAMAYDRYIAICNPLRYSVILSKVLCNQLAASCWAAGFLNSVVHTVLTFCLPFCGNNQINYFFCDIPPLLILSCGNTSVNELALLSTGVFIGWTPFLCIVLSYICIISTILRIQSSEGRRKAFSTCASHLAIVFLFYGSAIFTYVRPISTYSLKKDRLVSVLYSVVTPMLNPIIYTLRNKDIKEAVKTIGSKWQPPISSLDSKLTY

SEQ ID NO: 84 - protein, 327 aa, Mus musculus; identical to SEQ ID NO: 68

SEQ ID NO: 85 - protein, 327 aa, Rattus norvegicus; identical to SEQ ID NO: 69
  VARIANT (35): Where Xaa is Asp, Val, Ser or Gly

SEQ ID NO: 86 - protein, 314 aa, Mus musculus
MLDMNITLVSEFILVGFPTAPWLQILLFFIFLVVYMLIIAENLVIIFTVWSTGSLHKPMYYFLSSMSFLEIWYVSVTVPKMLDGFLLQRRHISFTGCMTQLYFFISLACTECVLLAAMAYDRYVAICHPLRYPVIMTTVYCMQLMALSYFSGFMVSVVKVYFISHVAFCGSNVMNHFFCDISPILKLACKDMSTAELVDFALAIVILVFPLITTVLSYVYIVSTILRIPSTQGRKKAFSTCASHLTVVIIYYTAMIFMYVRPRAIASFNSNKLISAVYAVLTPMLNPFIYCLRNREVKDAIKKTLGGGQCFLLC

SEQ ID NO: 87 - protein, 317 aa, Mus musculus
MEGKNQTAPSEFIILGFDHLNELQYLLFTIFFLTYICTLGGNVFIIVVTIADSHLHTPMYYFLGNLALIDICYTTTNVPQMMVHLLSEKKIISYGGCVTQLFAFIFFVGSECLLLAAMAYDRYIAICKPLRYSFIMNKALCSWLAASCWTCGFLNSVLHTVLTFHLPFCGNNQINYFFCDIPPLLILSCGDTSLNELALLSIGILIGWTPFLCIILSYLYIISTILRIRSSEGRQKAFSTCASHLLIVILYYGSAIFTYVRPISSYSLEKDRLISVLYSVFTPMLNPIIYALRNKDIKEAVKAIGRKWQPPVFSSDM

SEQ ID NO: 88 - protein, 253 aa, Artificial Sequence
  Description of Artificial Sequence 7tm_1, 7 transmembrane receptor domain sequence
NLLVILVILRTKKLRTPTNIFLLNLAVADLLFLLTLPPWALYYLVGGDWVFGDALCKLVGALFVVNGYASILLLTAISIDRYLAIVHPLRYRRIRTPRRAKVLILLVWVLALLLSLPPLLFSWLRTVEEGNTTVCLIDFPEESVKRSYVLLSTLVGFVLPLLVILVCYTRILRTLRKRARSQRSLKRRSSSERKAAKMLLVVVVVFVLCWLPYHIVLLLDSLCLLSIWRVLPTALLITLWLAYVNSCLNPIIY

SEQ ID NO: 89 - protein, 213 aa, Homo sapiens
FIDACYSSVNTPKLITDSLYENKTILFNGCMTQVFGEHFFRGVEVILLTVMAYDHYVAICKPLHYTTIMKQHVCSLLVGVSWVGGFLHATIQILFICQLPFCGPNVIDHFMCDLYTLINLACTNTHTLGLFIAANSGFICLLNCLLLLVSCVVILYSLKTHSLEARHEALSTCVSHITVVILSFIPCIFVYMRPPATLPIDKAVAVFYTMITS

SEQ ID NO: 90 - protein, 308 aa, Mus musculus
MEIPHNITEFFMLGLSQRPEIQRLLFVVFLVIYAVTVCGNMLIVVTVTFSSSLASPMYFFLSNLSFIDTCYSSSLAPKLIADSLYEGTTLSYEGCMAQLFGAHFLGGVEIILLTVMAYDRYVAICKPLHYTTTMTRHLCVVLVAVAWLGGFLHSLVQILLIFQLPFCGPNVINHFVCDLYPLLELACTNTYVIGLLVVANSGVICLLNFLMLAASYIVILHSLRSHSAEGRRKALSTCGAHFTVVTMFFVPCIFSYMRPSTTLPIDKNMAVFYGILTPMLNPLIYTLRNEEVKDAMRKLFTRSEVVGA

SEQ ID NO: 91 - protein, 302 aa, Mus musculus
MDSPRNVTEFFMLGLSQNPQVQRMLFGLFLLVFLVSVGGNMLIIITITFSPTLGSPMYFFLSYLSFIDTCYSSCMTPKLIADSLHEGRAISFEGCLAQFFVAHLLGGTEIILLTVMAYDRYVAICKPLHYTTTMTRHVCIVLVAVAWLGGILHSTAQLFLVLQLPFCGPNVINHFVCDLYPLLELACTDTYVIGLLVVANSGVICLLNFLMLAASYIVILRTLRSHSAEGRRKALSTCGAHFTVVALFFVPCIFIYMRPSSTLSIDKIVAVFYCILTPMFNPLIYTLRNAEVKNAMKNLWRK

SEQ ID NO: 92 - protein, 307 aa, Rattus norvegicus
MGENNNITEFILLGLTQDPDGRKALFVIFFLIYIVTMMGNLLIVVTVIASPSLGSPMYFFLASLSLLDALFSTAISPKLIADLLYDQKTISFRACMSQLFIEHLFGGVDIVILVAMAYDRYVAICKPLHYLAIMNRRVCITLLIFAWTGGFTHSLIQIVFVYNLPFCGPNVIDHFICDMSPLLVLACTDTYFIGLTVIANGGVNCIVIFTLLLGSYGIILRSLKTQSQEGRRKALSTCSSHILVVILFFVPCIFMYARPVYNFPIDKCITVFYTIITPMLNPLIYTLRNSEIKSCMKKLWCKMLHAD

SEQ ID NO: 93 - protein, 189 aa, Mus musculus
SFLSLIDGCCSSSMTPKMLADSLSVRKTISFSGCMTQVFAEHFFGAAEIILLTVMAYDRYVAICKPLRYTIIMNRFVCGLLVGVAWAGGFIHATIQILFTVWLPFCGPNVIDHFMCDLTPLLKLVCMDTHNLGLFVAANSGFICLLNFLLLMISYIVILDALKSHSKEGRRKALSTCVSHITVVILFFV

SEQ ID NO: 94 - protein, 254 aa, Artificial Sequence; identical to SEQ ID NO: 40
  Description of Artificial Sequence7tm_1, 7 transmembrane receptor domain sequence

SEQ ID NO: 95 - protein, 312 aa, Mus musculus
MEVDSNSSSGTFILMGVSDHPHLEIIFFAVILASYLLTLVGNLTIILLSRLDARLHTPMYFFLSNLSSLDLAFTTSSVPQMLKNLWGPDKTISYGGCVTQLYVFLWLGATECILLVVMAFDRYVAVCRPLHYMTVMNPRLCWGLAAISWLGGLGNSVIQSTFTLQLPFCGHRKVDNFLCEVPAMIKLACGDTSLNEAVLNGVCTFFTVVPVSVILVSYCFIAQAVMKIRSVEGRRKAFNTCVSHLVVVFLFYGSAIYGYLLPAKSSNQSQGKFISLFYSVVTPMVNPLIYTLRNKEVKGALGRLLGKGRGAS

SEQ ID NO: 96 - protein, 320 aa, Homo sapiens
MDQSNYSSLHGFILLGFSNHPKMEMILSGVVAIFYLITLVGNTAIILASLLDSQLHTPMYFFLRNLSFLDLCFTTSIIPQMLVNLWGPDKTISYVGCIIQLYVYMWLGSVECLLLAVMSYDRFTAICKPLHYFVVMNPHLCLKMIIMIWSISLANSVVLCTLTLNLPTCGNNILDHFLCELPALVKIACVDTTTVEMSVFALGIIIVLTPLILILISYGYIAKAVLRTKSKASQRKAMNTCGSHLTVVSMFYGTIIYMYLQPGNRASKDQGKFLTLFYTVITPSLNPLIYTLRNKDMKDALKKLMRFHHKSTKIKRNCKS

SEQ ID NO: 97 - protein, 320 aa, Homo sapiens [sequence omitted]

SEQ ID NO: 98 - protein, 312 aa, Homo sapiens
MLMKKNASFEDFFILLGFSNWPHLEVVLFVVILIFYLITLIGNLFIIILSYLDSHLHTPMYFFLSNLSFLDLCYTTSSIPQLLVNLWGPEKTISYAGCTVQLYFVLALGTAECVLLVVMSYDRYAAVCRPLHYTVLMHPRFCRLLAAASWVSGFTTSALHSSFTFWIPLCRHRLVDHFFCEVPALLRLSCVDTQANELTLMVMSSIFVLIPLILILTSYGAIARAVLSMQSTTGLQKVLRTCGAHLMVVSLFFIPVMCMYLQPPSENSQDQGKFIALFYTVVTPSLNPLIYTFRNKDVRGAVKRLMGWEWGM

SEQ ID NO: 99 - protein, 320 aa, Homo sapiens [sequence omitted]

SEQ ID NO: 100 - protein, 254 aa, Artificial Sequence; identical to SEQ ID NO: 40
  Description of Artificial Sequence7tm_1, 7 transmembrane receptor domain sequence

SEQ ID NO: 101 - protein, 311 aa, Homo sapiens
MEEYNTSSTDFTFMGLFNRKETSGLIFAIISIIFFTALMANGVMIFLIQTDLRLHTPMYFLLSHLSLIDMMYISTIVPKMLVNYLLDQRTISFVGCTAQHFLYLTLVGAEFFLLGLMAYDRYVAICNPLRYPVLMSRRVCWMIIAGSWFGGSLDGFLLTPITMSFPFCNSREINHFFCEAPAVLKLACADTALYETVMYVCCVLMLLIPFSVVLASYARILTTVQCMSSVEGRKKAFATCSSHMTVVSLFYGAAMYTYMLPHSYHKPAQDKVLSVFYTILTPMLNPLIYSLRNKDVTGALKRALGRFKGPQ

SEQ ID NO: 102 - protein, 223 aa, Mus musculus
SHLSFIDMMYISTIVPKMLVDYLLGQRTISFVGCTAQHFLYLTLVGAEFFLLGLMAYDRYVAICNPLRYPVLMSRRICWIIIAGSWFGGSLDGFLLTPITMSFPFCRSREINHFFCEAPAVLKLACADTALYETVMYVCCVLMLLIPFSVVISSYARILATVYHMSSVEGRKKAFATCSSHMTVVTLFYGAAIYTYMVPHSYHSPSQDKIFSVFYTILTPMLN

SEQ ID NO: 103 - protein, 216 aa, Homo sapiens
LIDMMYISTIVPKMLVNYLLDQRTISFVGCTAQHFLYLTLVGAEFFLLGLMAYDRYVAICNPLRYPVLMSRRVCWMIIAGSWFGGSLDGFLLTPITMSFPFCNSREINHFFCEAPAVLKLACADTALYETVMYVCCVLMLLIPFSVVLASYARILTTVQCMSSVEGRKKAFATCSSHMTVVSLFYGAAMYTYMLPHSYHKPAQDKVLSVFYTILTP

SEQ ID NO: 104 - protein, 316 aa, Mus musculus
MEPWNSTLGTDFNLVGILDDSGSPELLCATFTALYMLALISNGLLILVITMDARLHVPMYFLLGQLSLMDLLFTSVVTPKAVIDFLLRDNTISFEGCSLQMFLALTLGGAEDLLLAFMAYDRYVAICHPLNYMIFMRPSICWLMVATSWVLASLMALGYTTYTMQYSYCKSRKIRHLLCEIPPLLKLACADTSKYELMVYVMGVTFLIPPLAAILASYSLILFTVLHMPSNEGRKKALVTCSSHLTVVGMFYGAATFMYVLPNSFHSPRQDNIISVFYTIVTPALNPLIYSLRNKEVTGALIRVLGRYIVPAHPTL

SEQ ID NO: 105 - protein, 315 aa, Mus musculus
MEVCNSTLRSGFILMGILDDNDFPELLCATITALYLLALTSNGLLLLVITMDTRLHVPMYLLLWQLSLMDLLLTSVITPKAILDYLLKDNTISFGGCALQMFLALTLGTAEDLLLSFMAYDRYVAICHPLNYTILMSQKVCCLMIATSWSLASLSALGYSMYTMQYPFCKSRQIRHLFCEIPPLLKLACADTSTYELMVYLMGVTLLFPALAAILASYSLILFTVLHMPSNEGRRKALVTCSSHLTVVGMWYGGAIVMYVLPSSFHSPKQDNISSVFYTIFTPALNPLIYSLRNKEVTGALRRVLGKRLSVQSTF

SEQ ID NO: 106 - protein, 253 aa, Artificial Sequence; identical to SEQ ID NO: 88
  Description of Artificial Sequence7tm_1, 7 transmembrane receptor domain sequence

SEQ ID NO: 107 - protein, 312 aa, Mus musculus; identical to SEQ ID NO: 95

SEQ ID NO: 108 - protein, 313 aa, Rattus norvegicus; identical to SEQ ID NO: 50

SEQ ID NO: 109 - protein, 357 aa, Homo sapiens; identical to SEQ ID NO: 48

SEQ ID NO: 110 - protein, 316 aa, Homo sapiens
MDNQSSTPGFLLLGFSEHPGLERTLFVVVFTSYLLTLVGNTLIILLSALDPKLHSPMYFFLSNLSFLDLCFTTSCVPQMLVNLWGPKKTISFLDCSVQIFIFLSLGTTECILLTVMAFDRYVAVCQPLHYATIIHPRLCWQLASVAWVIGLVESVVQTPSTLHLPFCPDRQVDDFVCEVPALIRLSCEDTSYNEIQVAVASVFILVVPLSLILVSYGAITWAVLRINSAKGRRKAFGTCSSHLTVVTLFYSSVIAVYLQPKNPYAQERGKFFGLFYAVGTPSLNPLIYTLRNKEVTRAFRRLLGKERDSRESWRAA

SEQ ID NO: 111 - protein, 357 aa, Homo sapiens; identical to SEQ ID NO: 12

SEQ ID NO: 112 - protein, 241 aa, Artificial Sequence; identical to SEQ ID NO: 58
  Description of Artificial Sequence7tm_1, 7 transmembrane receptor domain sequence

SEQ ID NO: 113 - protein, 321 aa, Homo sapiens; identical to SEQ ID NO: 83

SEQ ID NO: 114 - protein, 327 aa, Mus musculus; identical to SEQ ID NO: 68

SEQ ID NO: 115 - protein, 314 aa, Mus musculus; identical to SEQ ID NO: 86

SEQ ID NO: 116 - protein, 327 aa, Rattus norvegicus; identical to SEQ ID NO: 69
  VARIANT (35): Where Xaa is Asp, Val, Ser or Gly

SEQ ID NO: 117 - protein, 317 aa, Mus musculus; identical to SEQ ID NO: 87

SEQ ID NO: 118 - protein, 253 aa, Artificial Sequence; identical to SEQ ID NO: 88
  Description of Artificial Sequence7tm_1, 7 transmembrane receptor domain sequence

SEQ ID NO: 119 - DNA, 22 bp, Artificial Sequence
  Description of Artificial Sequence  PCR Primer Sequence
accatcatcaagagtgctatgg

SEQ ID NO: 120 - DNA, 26 bp, Artificial Sequence
  Description of Artificial Sequence  PCR Primer Sequence
tcctttcgaagcttctgcatcatcct

SEQ ID NO: 121 - DNA, 22 bp, Artificial Sequence
  Description of Artificial Sequence  PCR Primer Sequence
aggcatgtcagcaagaatacat

SEQ ID NO: 122 - DNA, 22 bp, Artificial Sequence
  Description of Artificial Sequence  PCR Primer Sequence
atggcatttgatcgctatgtag

SEQ ID NO: 123 - DNA, 27 bp, Artificial Sequence
  Description of Artificial Sequence  PCR Primer Sequence
tgagatataccaccatcttgactccca

SEQ ID NO: 124 - DNA, 22 bp, Artificial Sequence
  Description of Artificial Sequence  PCR Primer Sequence
ccatagcactcttgatgatggt

SEQ ID NO: 125 - DNA, 22 bp, Artificial Sequence
  Description of Artificial Sequence  PCR Primer Sequence
accatcatcaagagtgctatgg

SEQ ID NO: 126 - DNA, 26 bp, Artificial Sequence
  Description of Artificial Sequence  PCR Primer Sequence
tcctttcgaagcttctgcatcatcct

SEQ ID NO: 127 - DNA, 22 bp, Artificial Sequence
  Description of Artificial Sequence  PCR Primer Sequence
aggcatgtcagcaagaatacat

SEQ ID NO: 128 - DNA, 22 bp, Artificial Sequence
  Description of Artificial Sequence  PCR Primer Sequence
ccatggcatttgatcactatgt

SEQ ID NO: 129 - DNA, 27 bp, Artificial Sequence
  Description of Artificial Sequence  PCR Primer Sequence
tgagatataccaccatcttgactccca

SEQ ID NO: 130 - DNA, 22 bp, Artificial Sequence
  Description of Artificial Sequence  PCR Primer Sequence
ccatagcactcttgatgatggt

SEQ ID NO: 131 - DNA, 22 bp, Artificial Sequence
  Description of Artificial Sequence  PCR Primer Sequence
ccatggcatttgatcactatgt

SEQ ID NO: 132 - DNA, 27 bp, Artificial Sequence
  Description of Artificial Sequence  PCR Primer Sequence
tgagatataccaccatcttgactccca

SEQ ID NO: 133 - DNA, 22 bp, Artificial Sequence
  Description of Artificial Sequence  PCR Primer Sequence
ccatagcactcttgatgatggt

SEQ ID NO: 134 - DNA, 22 bp, Artificial Sequence
  Description of Artificial Sequence  PCR Primer Sequence
atcccactgtgcttcatgtatc

SEQ ID NO: 135 - DNA, 26 bp, Artificial Sequence
  Description of Artificial Sequence  PCR Primer Sequence
atcccgggcaactgcacaattctttt

SEQ ID NO: 136 - DNA, 22 bp, Artificial Sequence
  Description of Artificial Sequence  PCR Primer Sequence
agtgagcgctctgttttaatga

SEQ ID NO: 137 - DNA, 22 bp, Artificial Sequence
  Description of Artificial Sequence  PCR Primer Sequence
atcccactgtgcttcatgtatc

SEQ ID NO: 138 - DNA, 26 bp, Artificial Sequence
  Description of Artificial Sequence  PCR Primer Sequence
atcccgggcaactgcacaattctttt

SEQ ID NO: 139 - DNA, 22 bp, Artificial Sequence
  Description of Artificial Sequence  PCR Primer Sequence
agtgagcgctctgttttaatga

SEQ ID NO: 140 - DNA, 22 bp, Artificial Sequence
  Description of Artificial Sequence  PCR Primer Sequence
atcccactgtgcttcatgtatc

SEQ ID NO: 141 - DNA, 26 bp, Artificial Sequence
  Description of Artificial Sequence  PCR Primer Sequence
atcccgggcaactgcacaattctttt

SEQ ID NO: 142 - DNA, 22 bp, Artificial Sequence
  Description of Artificial Sequence  PCR Primer Sequence
agtgagcgctctgttttaatga

SEQ ID NO: 143 - DNA, 22 bp, Artificial Sequence
  Description of Artificial Sequence  PCR Primer Sequence
ctctcttctgtggaatcattgc

SEQ ID NO: 144 - DNA, 27 bp, Artificial Sequence
  Description of Artificial Sequence  PCR Primer Sequence
cccatgctgaatccccttatatataca

SEQ ID NO: 145 - DNA, 22 bp, Artificial Sequence
  Description of Artificial Sequence  PCR Primer Sequence
ggcttcctttacctctttgttc

SEQ ID NO: 146 - DNA, 22 bp, Artificial Sequence
  Description of Artificial Sequence  PCR Primer Sequence
agattccaccctttgtgatgtt

SEQ ID NO: 147 - DNA, 26 bp, Artificial Sequence
  Description of Artificial Sequence  PCR Primer Sequence
tcttgacaatctttggcaatctgaca

SEQ ID NO: 148 - DNA, 22 bp, Artificial Sequence
  Description of Artificial Sequence  PCR Primer Sequence
tgaaatccacatgtgacacaag

SEQ ID NO: 149 - DNA, 22 bp, Artificial Sequence
  Description of Artificial Sequence  PCR Primer Sequence
agattccaccctttgtgatgtt

SEQ ID NO: 150 - DNA, 26 bp, Artificial Sequence
  Description of Artificial Sequence  PCR Primer Sequence
tcttgacaatctttggcaatctgaca

SEQ ID NO: 151 - DNA, 22 bp, Artificial Sequence
  Description of Artificial Sequence  PCR Primer Sequence
tgaaatccacatgtgacacaag

SEQ ID NO: 152 - DNA, 22 bp, Artificial Sequence
  Description of Artificial Sequence  PCR Primer Sequence
ggaaagtgtcctccctgttcta

SEQ ID NO: 153 - DNA, 26 bp, Artificial Sequence
  Description of Artificial Sequence  PCR Primer Sequence
ccataatagtccccgtgttaaaccca

SEQ ID NO: 154 - DNA, 22 bp, Artificial Sequence
  Description of Artificial Sequence  PCR Primer Sequence
ctttgacatccttgttcctcaa

SEQ ID NO: 155 - DNA, 22 bp, Artificial Sequence
  Description of Artificial Sequence  PCR Primer Sequence
ggaaagtgtcctccctgttcta

SEQ ID NO: 156 - DNA, 26 bp, Artificial Sequence
  Description of Artificial Sequence  PCR Primer Sequence
ccataatagtccccgtgttaaaccca

SEQ ID NO: 157 - DNA, 22 bp, Artificial Sequence
  Description of Artificial Sequence  PCR Primer Sequence
ctttgacatccttgttcctcaa

SEQ ID NO: 158 - DNA, 22 bp, Artificial Sequence
  Description of Artificial Sequence  PCR Primer Sequence
cctctccagcattctacacaac

SEQ ID NO: 159 - DNA, 26 bp, Artificial Sequence
  Description of Artificial Sequence  PCR Primer Sequence
tctacagaaggcaggtccaaagcctt

SEQ ID NO: 160 - DNA, 22 bp, Artificial Sequence
  Description of Artificial Sequence  PCR Primer Sequence
caattatgtgggaactgcaagt

SEQ ID NO: 161 - DNA, 22 bp, Artificial Sequence
  Description of Artificial Sequence  PCR Primer Sequence
attccacacaccttttgtgaac

SEQ ID NO: 162 - DNA, 26 bp, Artificial Sequence
  Description of Artificial Sequence  PCR Primer Sequence
acattggcctagccaaatatgcatgt

SEQ ID NO: 163 - DNA, 22 bp, Artificial Sequence
  Description of Artificial Sequence  PCR Primer Sequence
ggaaaacccataccaaatgttt

SEQ ID NO: 164 - DNA, 22 bp, Artificial Sequence
  Description of Artificial Sequence  PCR Primer Sequence
attccacacaccttttgtgaac

SEQ ID NO: 165 - DNA, 26 bp, Artificial Sequence
  Description of Artificial Sequence  PCR Primer Sequence
acattggcctagccaaatatgcatgt

SEQ ID NO: 166 - DNA, 22 bp, Artificial Sequence
  Description of Artificial Sequence  PCR Primer Sequence
ggaaaacccataccaaatgttt

SEQ ID NO: 167 - DNA, 22 bp, Artificial Sequence
  Description of Artificial Sequence  PCR Primer Sequence
caaccagccacagagatagttg

SEQ ID NO: 168 - DNA, 26 bp, Artificial Sequence
  Description of Artificial Sequence  PCR Primer Sequence
cttcttgcaggaagccctccagcatt

SEQ ID NO: 169 - DNA, 21 bp, Artificial Sequence
  Description of Artificial Sequence  PCR Primer Sequence
tctctacacctccgcagtgat

SEQ ID NO: 170 - DNA, 22 bp, Artificial Sequence
  Description of Artificial Sequence  PCR Primer Sequence
gctcaggtgacaactctcattc

SEQ ID NO: 171 - DNA, 28 bp, Artificial Sequence
  Description of Artificial Sequence  PCR Primer Sequence
tgtgttctgcctcactattccttttgga

SEQ ID NO: 172 - DNA, 22 bp, Artificial Sequence
  Description of Artificial Sequence  PCR Primer Sequence
caccacaattctggcataagat

SEQ ID NO: 173 - DNA, 22 bp, Artificial Sequence
  Description of Artificial Sequence  PCR Primer Sequence
gcgcaatagaatacgtcaattt

SEQ ID NO: 174 - DNA, 26 bp, Artificial Sequence
  Description of Artificial Sequence  PCR Primer Sequence
cagctcctaagcctcttagaccccaa

SEQ ID NO: 175 - DNA, 22 bp, Artificial Sequence
  Description of Artificial Sequence  PCR Primer Sequence
aaccatgactccaatctcaatg

SEQ ID NO: 176 - DNA, 22 bp, Artificial Sequence
  Description of Artificial Sequence  PCR Primer Sequence
taccgatcatagcacatcatca

SEQ ID NO: 177 - DNA, 27 bp, Artificial Sequence
  Description of Artificial Sequence  PCR Primer Sequence
tcagacactctgtaatagcaaacgcca

SEQ ID NO: 178 - DNA, 22 bp, Artificial Sequence
  Description of Artificial Sequence  PCR Primer Sequence
tgctccttgcatacttcagact

SEQ ID NO: 179 - DNA, 22 bp, Artificial Sequence
  Description of Artificial Sequence  PCR Primer Sequence
attctcaagaacggaggaagat

SEQ ID NO: 180 - DNA, 26 bp, Artificial Sequence
  Description of Artificial Sequence  PCR Primer Sequence
tttacagccttttcaacccgatcctg

SEQ ID NO: 181 - DNA, 22 bp, Artificial Sequence
  Description of Artificial Sequence  PCR Primer Sequence
tctgcattcctaaggctgtaga

SEQ ID NO: 182 - DNA, 22 bp, Artificial Sequence
  Description of Artificial Sequence  PCR Primer Sequence
attctcaagaacggaggaagat

SEQ ID NO: 183 - DNA, 26 bp, Artificial Sequence
  Description of Artificial Sequence  PCR Primer Sequence
tttacagccttttcaacccgatcctg

SEQ ID NO: 184 - DNA, 22 bp, Artificial Sequence
  Description of Artificial Sequence  PCR Primer Sequence
tctgcattcctaaggctgtaga

SEQ ID NO: 185 - DNA, 21 bp, Artificial Sequence
  Description of Artificial Sequence  PCR Primer Sequence
aggaagatcctttccctgttt

SEQ ID NO: 186 - DNA, 26 bp, Artificial Sequence
  Description of Artificial Sequence  PCR Primer Sequence
tacagccttttcaacccgatcctgaa

SEQ ID NO: 187 - DNA, 22 bp, Artificial Sequence
  Description of Artificial Sequence  PCR Primer Sequence
ctctctttagagcccctttcac

SEQ ID NO: 188 - DNA, 22 bp, Artificial Sequence
  Description of Artificial Sequence  PCR Primer Sequence
catgctgtgaccatcatcatta

SEQ ID NO: 189 - DNA, 27 bp, Artificial Sequence
  Description of Artificial Sequence  PCR Primer Sequence
ccttcctaatcattgccctgtcctatg

SEQ ID NO: 190 - DNA, 22 bp, Artificial Sequence
  Description of Artificial Sequence  PCR Primer Sequence
cttcagaagagggaatcctcaa

SEQ ID NO: 191 - DNA, 22 bp, Artificial Sequence
  Description of Artificial Sequence  PCR Primer Sequence
gaagaggatctgtatggttgca

SEQ ID NO: 192 - DNA, 26 bp, Artificial Sequence
  Description of Artificial Sequence  PCR Primer Sequence
tcctacccatgacactcccactagca

SEQ ID NO: 193 - DNA, 22 bp, Artificial Sequence
  Description of Artificial Sequence  PCR Primer Sequence
tgcactataccaccatcatgaa

SEQ ID NO: 194 - DNA, 22 bp, Artificial Sequence
  Description of Artificial Sequence  PCR Primer Sequence
ggctgtggtgtctctgttttac

SEQ ID NO: 195 - DNA, 26 bp, Artificial Sequence
  Description of Artificial Sequence  PCR Primer Sequence
catcttcatgtatctccagccagcca

SEQ ID NO: 196 - DNA, 21 bp, Artificial Sequence
  Description of Artificial Sequence  PCR Primer Sequence
ctatgaacttgccctgctcat

SEQ ID NO: 197 - DNA, 22 bp, Artificial Sequence
  Description of Artificial Sequence  PCR Primer Sequence
acctcccaacaaccttctgtag

SEQ ID NO: 198 - DNA, 26 bp, Artificial Sequence
  Description of Artificial Sequence  PCR Primer Sequence
ccgtgacatccttgttcctaaggctg

SEQ ID NO: 199 - DNA, 22 bp, Artificial Sequence
  Description of Artificial Sequence  PCR Primer Sequence
ccatgctcaatccactcattta

SEQ ID NO: 200 - DNA, 22 bp, Artificial Sequence
  Description of Artificial Sequence  PCR Primer Sequence
ctattttggggaataccaccat

SEQ ID NO: 201 - DNA, 26 bp, Artificial Sequence
  Description of Artificial Sequence  PCR Primer Sequence
tctcgtctggaacccaagcctcatat

SEQ ID NO: 202 - DNA, 22 bp, Artificial Sequence
  Description of Artificial Sequence  PCR Primer Sequence
ggaaggagagatgagaaaggaa

SEQ ID NO: 203 - DNA, 22 bp, Artificial Sequence
  Description of Artificial Sequence  PCR Primer Sequence
acgcagtgttgaggattaagtc

SEQ ID NO: 204 - DNA, 26 bp, Artificial Sequence
  Description of Artificial Sequence  PCR Primer Sequence
acagaaagcattcgggacctgcttct

SEQ ID NO: 205 - DNA, 22 bp, Artificial Sequence
  Description of Artificial Sequence  PCR Primer Sequence
tgatggttccataaaagatggt

(19) United States
(12) Patent Application Publication
(10) Pub. No.: US 2003/0232331 A1
(43) Pub. Date: Dec. 18, 2003

(54) Novel proteins and nucleic acids encoding same

(76) Inventors: Stacie J. Casman, North Haven, CT (US); Muralidhara Padigaru, Branford, CT (US); Catherine E. Burgess, Wethersfield, CT (US); Richard A. Shimkets, West Haven, CT (US); Kimberly A. Spytek, New Haven, CT (US); Jennifer A. Gilbert, Madison, CT (US); Jane E. Mayotte, Middlefield, CT (US); Jason C. Baumgartner, New Haven, CT (US); Vishnu Mishra, Gainesville, FL (US); Corine A.M. Vernet, Branford, CT (US); Kevin S. Dickinson, Meriden, CT (US); Robert A. Ballinger, Newington, CT (US); Adam R. Wolenc, New Haven, CT (US); Shlomit R. Edinger, New Haven, CT (US); John R. MacDougall, Hamden, CT (US); Glennda Smithson, Guilford, CT (US); Karen Ellerman, Branford, CT (US); David J. Stone, Guilford, CT (US); Erik Gunther, Branford, CT (US); Valerie Gerlach, Branford, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/005,041

(22) Filed: Dec. 4, 2001

Related U.S. Application Data
(60) Provisional application No. 60/251,459, filed on Dec. 5, 2000.
(60) Provisional application No. 60/259,007, filed on Dec. 29, 2000.

(51) Int. Cl. C12Q 1/68; G01N 33/574; C07H 21/04; C12P 21/02; C12N 5/06; C07K 14/47; A61K 48/00; C12N 15/00

(52) U.S. Cl. 435/6; 435/7.23; 435/69.1; 435/183; 435/320.1; 435/325; 530/350; 536/23.2; 514/12; 514/44

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. Nos. 60/251,459 filed Dec. 5, 2000; and 60/259,007 filed Dec. 29, 2000, each of which is incorporated by reference in its entirety.

### BACKGROUND OF THE INVENTION

The invention generally relates to nucleic acids and polypeptides. More particularly, the invention relates to nucleic acids encoding novel G-protein coupled receptor (GPCR) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as GPCR1, GPCR2, GPCR3, GPCR4, GPCR5, GPCR6, GPCR7, GPCR8, GPCR9, GPCR10, GPCR11, GPCR12, GPCR13 and GPCR14 nucleic acids and polypeptides. These nucleic acids and polypeptides, as well as derivatives, homologs, analogs and fragments thereof, will hereinafter be collectively designated as “GPCRX” nucleic acid or polypeptide sequences.

In one aspect, the invention provides an isolated GPCRX nucleic acid molecule encoding a GPCRX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31 and 33. In some embodiments, the GPCRX nucleic acid molecule will hybridize under stringent conditions to a nucleic acid sequence complementary to a nucleic acid molecule that includes a protein-coding sequence of a GPCRX nucleic acid sequence. The invention also includes an isolated nucleic acid that encodes a GPCRX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, 32 and 34. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29, 31 and 33. Also included in the invention is an oligonucleotide, e.g., an oligonucleotide which includes at least 6 contiguous nucleotides of a GPCRX nucleic acid (e.g., SEQ ID NOS: 1, 3, 5, 7, 9, 11, 13, 15, 17, 19, 21, 23, 25, 27, 29, 31 and 33) or a complement of said oligonucleotide.

Also included in the invention are substantially purified GPCRX polypeptides (e.g., SEQ ID NOS: 2, 4, 6, 8, 10, 12, 14, 16, 18, 20, 22, 24, 26, 28, 30, 32 and 34). In certain embodiments, the GPCRX polypeptides include an amino acid sequence that is substantially identical to the amino acid sequence of a human GPCRX polypeptide.

The invention also features antibodies that immunoselectively bind to GPCRX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a GPCRX nucleic acid, a GPCRX polypeptide, or an antibody specific for a GPCRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a GPCRX nucleic acid, under conditions allowing for expression of the GPCRX polypeptide encoded by the DNA. If desired, the GPCRX polypeptide can then be recovered.

In another aspect, the invention includes a method of detecting the presence of a GPCRX polypeptide in a sample. In the method, a sample is contacted with a compound that selectively binds to the polypeptide under conditions allowing for formation of a complex between the polypeptide and the compound. The complex is detected, if present, thereby identifying the GPCRX polypeptide within the sample.

The invention also includes methods to identify specific cell or tissue types based on their expression of a GPCRX.

Also included in the invention is a method of detecting the presence of a GPCRX nucleic acid molecule in a sample by contacting the sample with a GPCRX nucleic acid probe or primer, and detecting whether the nucleic acid probe or primer bound to a GPCRX nucleic acid molecule in the sample.

In a further aspect, the invention provides a method for modulating the activity of a GPCRX polypeptide by contacting a cell sample that includes the GPCRX polypeptide with a compound that binds to the GPCRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., developmental diseases; MHCII and III diseases (immune diseases); taste and scent detectability disorders; Burkitt's lymphoma; corticoneurogenic disease; signal transduction pathway disorders; metabolic pathway disorders; retinal diseases including those involving photoreception; cell growth rate disorders; cell shape disorders; metabolic disorders; feeding disorders; control of feeding; the metabolic syndrome X; wasting disorders associated with chronic diseases; obesity; potential obesity due to over-eating or metabolic disturbances; potential disorders due to starvation (lack of appetite); diabetes; noninsulin-dependent diabetes mellitus (NIDDM1); infectious disease; bacterial, fungal, protozoal and viral infections (particularly infections caused by HIV-1 or HIV-2); pain; cancer (including but not limited to neoplasm; adenocarcinoma; lymphoma; prostate cancer; uterus cancer); cancer-associated cachexia; anorexia; bulimia; asthma; Parkinson's disease; acute heart failure; hypotension; hypertension; urinary retention; osteoporosis; Crohn's disease; multiple sclerosis; Albright Hereditary Ostoeodystrophy; angina pectoris; myocardial infarction; ulcers; allergies; benign prostatic hypertrophy; and psychotic and neurological disorders; including anxiety; schizophrenia; manic depression; delirium; dementia; neurodegenerative disorders; Alzheimer's disease; severe mental retardation; Dentatorubro-pallidoluysian atrophy (DRPLA); Hypophosphatemic rickets; autosomal dominant (2) acrocallosal syndrome and dyskinesias, such as Huntington's disease or Gilles de la Tourette syndrome; immune disorders; adrenoleukodystrophy; congenital adrenal hyperplasia; hemophilia; hypercoagulation; idiopathic thrombocytopenic purpura; autoimmume disease; immunodeficiencies; transplantation; Von Hippel-Lindau (VHL) syndrome; stroke; tuberous sclerosis; hypercalceimia; cerebral palsy; epilepsy; Lesch-Nyhan syndrome; ataxia-telangiectasia; Leukodystrophies; Behavioral disorders; Addiction; Neuroprotection; cirrhosis; transplantation; systemic lupus erythematosus; emphysema; scleroderma; ARDS; renal artery stenosis; interstitial nephritis; glomerulonephritis; polycystic kidney disease; systemic lupus erythematosus; renal tubular acidosis; IgA nephropathy; cardiomyopathy; atherosclerosis; congenital heart defects; aortic stenosis; atrial septal defect (ASD); atrioventricular (A-V) canal defect; ductus arteriosus; pulmonary stenosis; subaortic stenosis; ventricular septal defect (VSD); valve diseases; scleroderma; fertility; pancreatitis; endocrine dysfunctions; growth and reproductive disorders; inflammatory bowel disease; diverticular disease; leukodystrophies; graft vesus host; hyperthyroidism; endometriosis; hematopoietic disorders and/or other pathologies and disorders of the like. The therapeutic can be, e.g., a GPCRX nucleic acid, a GPCRX polypeptide, or a GPCRX-specific antibody, or biologically-active derivatives or fragments thereof.

For example, the compositions of the present invention will have efficacy for treatment of patients suffering from the diseases and disorders listed above and/or other pathologies and disorders.

The polypeptides can be used as immunogens to produce antibodies specific for the invention, and as vaccines. They can also be used to screen for potential agonist and antagonist compounds. For example, a cDNA encoding GPCRX may be useful in gene therapy, and GPCRX may be useful when administered to a subject in need thereof. By way of nonlimiting example, the compositions of the present invention will have efficacy for treatment of patients suffering the diseases and disorders listed above and/or other pathologies and disorders.

The invention further includes a method for screening for a modulator of disorders or syndromes including, e.g., diseases and disorders listed above and/or other pathologies and disorders and those disorders related to cell signal processing and metabolic pathway modulation. The method includes contacting a test compound with a GPCRX polypeptide and determining if the test compound binds to said GPCRX polypeptide. Binding of the test compound to the GPCRX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including the diseases and disorders listed above and/or other pathologies and disorders or other disorders related to cell signal processing and metabolic pathway modulation by administering a test compound to a test animal at increased risk for the aforementioned disorders or syndromes. The test animal expresses a recombinant polypeptide encoded by a GPCRX nucleic acid. Expression or activity of GPCRX polypeptide is then measured in the test animal, as is expression or activity of the protein in a control animal which recombinantly-expresses GPCRX polypeptide and is not at increased risk for the disorder or syndrome. Next, the expression of GPCRX polypeptide in both the test animal and the control animal is compared. A change in the activity of GPCRX polypeptide in the test animal relative to the control animal indicates the test compound is a modulator of latency of the disorder or syndrome.

In yet another aspect, the invention includes a method for determining the presence of or predisposition to a disease associated with altered levels of a GPCRX polypeptide, a GPCRX nucleic acid, or both, in a subject (e.g., a human subject). The method includes measuring the amount of the GPCRX polypeptide in a test sample from the subject and comparing the amount of the polypeptide in the test sample to the amount of the GPCRX polypeptide present in a control sample. An alteration in the level of the GPCRX polypeptide in the test sample as compared to the control sample indicates the presence of or predisposition to a disease in the subject. Preferably, the predisposition includes diseases and disorders listed above and/or other pathologies and disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a GPCRX polypeptide, a GPCRX nucleic acid, or a GPCRX-specific antibody to a subject (e.g., a human subject), in an amount sufficient to alleviate or prevent the pathological condition. In preferred embodiments, the disorder, includes the diseases and disorders listed above and/or other pathologies and disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based, in part, upon the discovery of novel nucleic acid sequences that encode novel polypeptides. The novel nucleic acids and their encoded polypeptides are referred to individually as GPCR1, GPCR2, GPCR3, GPCR4, GPCR5, GPCR6, GPCR7, GPCR8, GPCR9, GPCR10, GPCR11, GPCR12, GPCR13 and GPCR14. The nucleic acids, and their encoded polypeptides, are collectively designated herein as “GPCRX”.

The novel GPCRX nucleic acids of the invention include the nucleic acids whose sequences are provided in Tables 1A, 1C, 1E, 2A, 2C, 3A, 4A, 5A, 6A, 7A, 8A, 9A, 10A, 11A, 12A, 13A, 14A, inclusive, or a fragment, derivative, analog or homolog thereof. The novel GPCRX proteins of the invention include the protein fragments whose sequences are provided in Tables 1B, 1D, 1F, 2B, 2D, 3B, 4B, 5B, 6B, 7B, 8B, 9B, 10B, 11B, 12B, 13B, 14B inclusive. The individual GPCRX nucleic acids and proteins are described below. Within the scope of this invention is a method of using these nucleic acids and peptides in the treatment or prevention of a disorder related to cell signaling or metabolic pathway modulation.

G-Protein Coupled Receptor proteins (“GPCRs”) have been identified as a large family of G protein-coupled receptors in a number of species. These receptors share a seven transmembrane domain structure with many neurotransmitter and hormone receptors, and are likely to underlie the recognition and G-protein-mediated transduction of various signals. Human GPCR generally do not contain introns and belong to four different gene subfamilies, displaying great sequence variability. These genes are dominantly expressed in olfactory epithelium. See, e.g., Ben-Arie et al., Hum. Mol. Genet. 1994 3:229-235; and, Online Mendelian Inheritance in Man (“OMIM”) entry # 164342(http://www.ncbi.nlm.nih.gov/entrez/dispomim.cgi?).

The olfactory receptor (“OR”) gene family constitutes one of the largest GPCR multigene families and is distributed among many chromosomal sites in the human genome. See Rouquier et al., Hum. Mol. Genet. 7(9):1337-45 (1998); Malnic et al., Cell 96:713-23 (1999). Olfactory receptors constitute the largest family among G protein-coupled receptors, with up to 1000 members expected. See Vanderhaeghen et al., Genomics 39(3):239-46 (1997); Xie et al., Mamm. Genome 11(12):1070-78 (2000); Issel-Tarver et al., Proc. Natl. Acad. Sci. USA 93(20):10897-902 (1996). The recognition of odorants by olfactory receptors is the first stage in odor discrimination. See Krautwurst et al., Cell 95(7):917-26 (1998); Buck et al., Cell 65(1):175-87 (1991). Many ORs share some characteristic sequence motifs and have a central variable region corresponding to a putative ligand binding site. See Issel-Tarver et al., Proc. Natl. Acad. Sci. USA 93:10897-902 (1996).

Other examples of seven membrane spanning proteins that are related to GPCRs are chemoreceptors. See Thomas et al., Gene 178(1-2):1-5 (1996). Chemoreceptors have been identified in taste, olfactory, and male reproductive tissues. See id.; Walensky et al., J. Biol. Chem. 273(16):9378-87 (1998); Parmentier et al., Nature 355(6359):453-55 (1992); Asai et al., Biochem. Biophys. Res. Commun. 221(2):240-47 (1996).

The GPCRX nucleic acids of the invention encoding GPCR-like proteins include the nucleic acids whose sequences are provided herein, or fragments thereof. The invention also includes mutant or variant nucleic acids any of whose bases may be changed from the corresponding base shown herein while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The GPCRX proteins of the invention include the GPCR-like proteins whose sequences are provided herein. The invention also includes mutant or variant proteins any of whose residues may be changed from the corresponding residue shown herein while still encoding a protein that maintains its GPCR-like activities and physiological functions, or a functional fragment thereof. The invention further encompasses antibodies and antibody fragments, such as F_{ab} or (F_{ab})₂, that bind immunospecifically to any of the proteins of the invention.

The GPCRX nucleic acids and proteins are useful in potential therapeutic applications implicated in various GPCR-related pathological disorders and/or OR-related pathological disorders, described further below. For example, a cDNA encoding the GPCR (or olfactory-receptor) like protein may be useful in gene therapy, and the receptor -like protein may be useful when administered to a subject in need thereof. The nucleic acids and proteins of the invention are also useful in potential therapeutic applications used in the treatment of developmental diseases; MHCII and III diseases (immune diseases); taste and scent detectability disorders; Burkitt's lymphoma; corticoneurogenic disease; signal transduction pathway disorders; metabolic pathway disorders; retinal diseases including those involving photoreception; cell growth rate disorders; cell shape disorders; metabolic disorders; feeding disorders; control of feeding; the metabolic syndrome X; wasting disorders associated with chronic diseases; obesity; potential obesity due to over-eating or metabolic disturbances; potential disorders due to starvation (lack of appetite); diabetes; noninsulin-dependent diabetes mellitus (NIDDM1); infectious disease; bacterial, fungal, protozoal and viral infections (particularly infections caused by HIV-1 or HIV-2); pain; cancer (including but not limited to neoplasm; adenocarcinoma; lymphoma; prostate cancer; uterus cancer); cancer-associated cachexia; anorexia; bulimia; asthma; Parkinson's disease; acute heart failure; hypotension; hypertension; urinary retention; osteoporosis; Crohn's disease; multiple sclerosis; Albright hereditary ostoeodystrophy; angina pectoris; myocardial infarction; ulcers; allergies; benign prostatic hypertrophy; and psychotic and neurological disorders; including anxiety; schizophrenia; manic depression; delirium; dementia; neurodegenerative disorders; Alzheimer's disease; severe mental retardation; dentatorubro-pallidoluysian atrophy (DRPLA); hypophosphatemic rickets; autosomal dominant (2) acrocallosal syndrome and dyskinesias, such as Huntington's disease or Gilles de la Tourette syndrome; immune disorders; adrenoleukodystrophy; congenital adrenal hyperplasia; hemophilia; hypercoagulation; idiopathic thrombocytopenic purpura; autoimmume disease; immunodeficiencies; transplantation; Von Hippel-Lindau (VHL) syndrome; stroke; tuberous sclerosis; hypercalceimia; cerebral palsy; epilepsy; Lesch-Nyhan syndrome; ataxia-telangiectasia; leukodystrophies; behavioral disorders; addiction; neuroprotection; cirrhosis; transplantation; systemic lupus erythematosus; emphysema; scleroderma; ARDS; renal artery stenosis; interstitial nephritis; glomerulonephritis; polycystic kidney disease; systemic lupus erythematosus; renal tubular acidosis; IgA nephropathy; cardiomyopathy; atherosclerosis; congenital heart defects; aortic stenosis; atrial septal defect (ASD); atrioventricular (A-V) canal defect; ductus arteriosus; pulmonary stenosis; subaortic stenosis; ventricular septal defect (VSD); valve diseases; scleroderma; fertility; pancreatitis; endocrine dysfunctions; growth and reproductive disorders; inflammatory bowel disease; diverticular disease; leukodystrophies; graft vesus host; hyperthyroidism; endometriosis; hematopoietic disorders and/or other pathologies and disorders. Other GPCR-related diseases and disorders are contemplated.

The protein similarity information, expression pattern, and map location for the olfactory receptor-like GPCR proteins and nucleic acids disclosed herein suggest that these olfactory receptors may have important structural and/or physiological functions characteristic of the olfactory receptor family. Therefore, the GPCR nucleic acids and proteins are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

GPCR polypeptides are useful in the generation of antibodies that bind immunospecifically to the GPCR polypeptides of the invention, and as vaccines. The antibodies are for use in therapeutic or diagnostic methods. These antibodies may be generated according to methods known in the art, using prediction from hydrophobicity charts, as described in the “Anti-GPCRX Antibodies” section below.

GPCR polypeptides can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the compositions of the present invention will have efficacy for treatment of patients suffering from the diseases and disorders disclosed above and/or other pathologies and disorders. The novel nucleic acid encoding GPCR-like protein, and the GPCR-like protein of the invention, or fragments thereof, may further be useful in diagnostic applications, wherein the presence or amount of the nucleic acid or the protein are to be assessed.

GPCR1

GPCR1 includes three novel G-protein coupled receptor (“GPCR”) proteins disclosed below. The disclosed proteins have been named GPCR1a, GPCR1b and GPCR1c, and are related to olfactory receptors.

GPCR1a

The disclosed GPCR1a nucleic acid of 953 nucleotides (also referred to as SC35113271_A_da1) is shown in Table 1A. The disclosed GPCR1a open reading frame (“ORF”) begins with a TCT at nucleotides 2-4 which encodes a serine (the first amino acid of the mature protein) and ending with a TGA codon at nucleotides 950-952. The start and stop codons of the open reading frame are highlighted in bold type. Putative untranslated regions, if any, are found upstream from the initiation codon and downstream from the termination codon.

TABLE 1A

GPCR1a nucleotide sequence.

(SEQ ID NO:1)

CTCTGCCATGATCATTTTCAACCTGAGCAGTTACAATCCAGCACCCTTCATTCTGGTAGGGATCCCAGG
CCTGGAGCAATTCCATGTGTGGATTGGAATTCCCTTCTGTATCATCTACATTGTAGCTGTTGTGGGAAA
CTGCATCCTTCTCTACCTCATTGTGGTGGAGCATAGTCTTCATGAACCCATGTTCTTCTTTCTCTCCAT
GCTGGCCATGACTGACCTCATCTTGTCCACAGCTGGTGTGCCTAAAACACTCAGTATCTTTTGGCTAGG
GGCTCGCGAAATCACATTCCCAGGATGCCTTACACAAATGTTCTTCCTTCACTATAACTTTGTCCTGGA
TTCAGCCATTCTGATGGCCATGGCATTTGATCGCTATGTAGCTATCTGTTCTCCCTTGAGATATACCAC
CATCTTGACTCCCAAGACCATCATCAAGAGTGCTATGGGCATCTCCTTTCGAAGCTTCTGCATCATCCT
GCCAGATGTATTCTTGCTGACATGCCTGCCTTTCTGCAGGACACGCATCATACCCCACACATACTGTGA
GCATATAGGTGTTGCCCAGCTCGCCTGTCCTGATATCTCCATCAACTTCTGGTATGGCTTTTGTCTTCC
CATCATGACAGTCATCTCAGATGTGATTCTCATTGCTGTTTCCTACCCACACATCCTCTGTGCTGTCTT
TTGCCTTCCCTCCCAAGATGCCCGCCAGAAAGCCCTCGGCACTTGTGGTTCTCATGTCTGTGTCATCCT
CATGTTTTATACACCTGCCTTTTTCTCCATCCTCGCCCATCGCTTTGGACACAATGTCTCTCGCACCTT
CCACATCATGTTTGCCAATCTCTACATTGTTATCCCACCTGCACTCAACCCCATGGTTTACGGAGTGAA
GACCAAGCAGATCAGAGATAAGGTTATACTTTTGTTTTCTAAGGGTACAGGATGAT

The GPCR1a the nucleic acid sequence of this invention has 604 of 904 bases (66%) identical to a gb:GENBANK-ID:AF121975|acc:AF121975.1 mRNA from Mus musculus (Mus musculus odorant receptor S18 gene, complete cds) (E=2.4e−⁶⁸).

The disclosed GPCR1a polypeptide (SEQ ID NO: 2) encoded protein having 316 amino acid residues is presented in Table 1B using the one-letter amino acid code. The Signal P, Psort and/or Hydropathy results predict that GPCR1 a has a signal peptide and is likely to be localized at the endoplasmic reticulum (membrane) with a certainty of 0.6850. In other embodiments, it is localized at the plasma membrane with a certainty of 0.6400, at the Golgi body with a certainty of 0.4600 or at the endoplasmic reticulum (lumen) with a certainty of 0.1000. The most likely cleavage site for a GPCR1 a peptide is between amino acids 45 and 46, at: VVG-NC.

TABLE 1B

Encoded GPCR1a protein sequence.

(SEQ ID NO:2)

SAMIIFNLSSYNPGPFILVGIPGLEQFHVWIGIPFCIIYIVAVVGNCILLYLIVVEHSLHEPMFFFLSMLANTDLIL
STAGVPKTLSIFWLGAREITFPGCLTQMFFLHYNFVLDSAILMAMAFDRYVAICSPLRYTTILTPKTIIKSAMGISF
RSFCIILPDVFLLTCLPFCRTRIIPHTYCEHIGVAQLACADISINFWYGFCVPIMTVISDVILIAVSYAHILCAVFC
LPSQDARQKALGTCGSHVCVILMFYTPAFFSILAHRFGHNVSRTFHIMFANLYIVIPPALNPMVYGVKTKQIRDKVI
LLFSKGTG

The disclosed GPCR1 a amino acid sequence of the protein of the invention was found to have 183 of 312 amino acid residues (58%) identical to, and 236 of 312 amino acid residues (75%) similar to, the 321 amino acid residue ptnr:SPTREMBL-ACC:Q9WU89 protein from Mus musculus (Mouse) (ODORANT RECEPTOR S18) (E=3.3e⁻¹⁰¹).

GPCR1b

The disclosed GPCR1b nucleic acid of 953 nucleotides (also referred to as CG55798-03) is shown in Table 1C. The disclosed GPCR1b open reading frame (“ORF”) begins with an ATG at nucleotides 8-10 and ending with a TGA codon at nucleotides 950-952. The start and stop codons of the open reading frame are highlighted in bold type. Putative untranslated regions, if any, are found upstream from the initiation codon and downstream from the termination codon.

TABLE 1C

GPCR1b nucleotide sequence.

(SEQ ID NO:3)

CTCTGCCATGATCATTTTCAACCTGAGCAGTTACAATCCAGGACCCTTCATTCTGGTAGGGATCCCAGGCCTGG
AGCAATTCCATGTGTGGATTGGAATTCCCTTCTGTATCATCTACATTGTAGCTGTTGTGGGAAACTGCATCCTT
CTCTACCTCATTGTGGTGGAGCATAGTCTTCATGAACCCATGTTCTTCTTTCTCTCCATGCTGGCCATGACTGA
CCTCATCTTGTCCACAGCTGGTGTGCCTAAAACACTCAGTATCTTTTGGCTAGGGGCTCGCGAAATCACATTCC
CAGGATGCCTTACACAAATGTTCTTCCTTCACTATAACTTTGTCCTGGATTCAGCCATTCTGATGGCCATGGCA
TTTGATCGCTATGTAGCTATCTGTTCTCCCTTGAGATATACCACCATCTTGACTCCCAAGACCATCATCAAGAG
TGCTATGGGCATCTCCTTTCGAAGCTTCTGCATCATCCTGCCAGATGTATTCTTGCTGACATGCCTGCCTTTCT
GCAGGACACGCATCATACCCCACACATACTGTGAGCATATAGGTGTTGCCCGGCTCGCCTGTGCTGATATCTCC
ATCAACTTCTGGTATGGCTTTTGTGTTCCCATCATGACAGTCATCTCAGATGTGATTCTCATTGCTGTTTCCTA
CGCACACATCCTCTGTGCTGTCTTTTGCCTTCCCTCCCAAGATGCCCGCCAGAAAGCCCTCGGCACTTGTGGTT
CTCATGTCTGTGTCATCCTCATGTTTTATACACCTGCCTTTTTCTCCATCCTCGCCCATCGCTTTGGACACAAT
GTCTCTCGCACCTTCCACATCATGTTTGCCAATCTCTACATTGTTATCCCACCTGCACTCAACCCCATGGTTTA
CGGAGTGAAGACCAAGCAGATCAGAGATAAGGTTATACTTTTGTTTTCTAAGGGTACAGGATGAT

The GPCR1b the nucleic acid sequence of this invention has bases (66%) identical to a gb:GENBANK-ID:AF121975|acc:AF121975.1 mRNA from Mus musculus (Mus musculus odorant receptor S18 gene, complete cds) (E=1.2e−⁶⁸).

The disclosed GPCR1b polypeptide (SEQ ID NO: 4) encoded protein having 314 amino acid residues is presented in Table 1D using the one-letter amino acid code. The Signal P, Psort and/or Hydropathy results predict that GPCR1b has a signal peptide and is likely to be localized at the endoplasmic reticulum (membrane) with a certainty of 0.6850. In other embodiments, it is localized at the plasma membrane with a certainty of 0.6400, at the Golgi body with a certainty of 0.4600 or at the endoplasmic reticulum (lumen) with a certainty of 0.1000. The most likely cleavage site for a GPCR1b peptide is between amino acids 43 and 44, at: VVG-NC.

TABLE 1D

Encoded GPCR1b protein sequence.

(SEQ ID NO:4)

MIIFNLSSYNPGPFILVGIPGLEQFHVWIGIPFCIIYIVAVVGNCILLYLIVVEHSLHEPMFFFLSMLAMTDLILST
AGVPKTLSIFWLGAREITFPGCLTQMFFLHYNFVLDSAILMAMAFDRYVAICSPLRYTTILTPKTIIKSAMGISFRS
FCIILPDVFLLTCLPFCRTRIIPHTYCEHIGVARLACADISINFWYGFCVPIMTVISDVILIAVSYAHILCAVFCLP
SQDARQKALGTCGSHVCVILMFYTPAFFSILAHRFGHNVSRTFHIMFANLYIVIPPALNPMVYGVKTKQ
IRDKVILLFSKGTG

The disclosed GPCR1b amino acid sequence of the protein of the invention was found to have 184 of 310 amino acid residues (59%) identical to, and 234 of 310 amino acid residues (75%) similar to, the 321 amino acid residue ptnr:SPTREMBL-ACC:Q9WU89 protein from Mus musculus (Mouse) (ODORANT RECEPTOR S18) (E=2.3e⁻¹⁰¹).

GPCR1c

The disclosed GPCR1c nucleic acid of 953 nucleotides (also referred to as CG55798-04) is shown in Table 1E. The disclosed GPCR1c open reading frame (“ORF”) begins with an ATG at nucleotides 8-10 and ending with a TGA codon at nucleotides 950-952. The start and stop codons of the open reading frame are highlighted in bold type. Putative untranslated regions (underlined), if any, are found upstream from the initiation codon and downstream from the termination codon.

TABLE 1E

GPCR1c nucleotide sequence.

(SEQ ID NO:5)

CTCTGCCATGATCATTTTCAACCTGAGCAGTTACAATCCAGGACCCTTCATTCTGGTAGGGATCCCAGGCCTGG
AGCAATTCCATGTGTGGATTGGAATTCCCTTCTGTATCATCTACATTGTAGCTGTTGTGGGAAACTGCATCCTT
CTCTACCTCATTGTGGTGGAGCATAGTCTTCATGAACCCATGTTCTTCTTTCTCTCCATGCTGGCCATGACTGA
CCTCATCTTGTCCACAGCTGGTGTGCCTAAAACACTCAGTATCTTTTGGCTAGGGGCTCGCGAAATCACATTCC
CAGGATGCCTTACACAAATGTTCTTCCTTCACTATAACTTTGTCCTGGATTCAGCCATTCTGATGGCCATGGCA
TTTGATCACTATGTAGCTATCTGTTCTCCCTTGAGATATACCACCATCTTGACTCCCAAGACCATCATCAAGAG
TGCTATGGGCATCTCCTTTCGAAGCTTCTCCATCATCCTGCCAGATGTATTCTTGCTGACATGCCTGCCTTTCT
GCAGCACACGCATCATACCCCACACATACTGTGAGCATATGGGTGTTGCCCAGCTCGCCTGTGCTGATATCTCC
ATCAACTTCTGGTATGGCTTTTGTGTTCCCATCATGACAGTCATCTCAGATGTGATTCTCATTGCTGTTTCCTA
CGCACACATCCTCTGTGCTGTCTTTTGCCTTCCCTCCCAAGATGCCCGCCAGAAGGCCCTCGGCACTTGTGGTT
CTCATGTCTGTGTCATCCTCATGTTTTATACACCTGCCTTTTTCTCCATCCTCGCCCATCGCTTTGGACACAAT
GTCTCTCGCACCTTCCACATCATGTTTGCCAATCTCTACATTGTTATCCCACCTGCACTCAACCCCATGGTTTA
CCGAGTGAAGACCAAGCAGATCAGAGATAAGGTTATACTTTTGTTTTCTAAGGGTACAGGATGAT

The GPCR1c the nucleic acid sequence of this invention has 604 of 904 bases (66%) identical to a gb:GENBANK-ID:AF121975|acc:AF121975.1 mRNA from Mus musculus (Mus musculus odorant receptor S18 gene, complete cds) (E=2.8e⁻⁶⁸).

The disclosed GPCR1 c polypeptide (SEQ ID NO: 6) encoded protein having 314 amino acid residues is presented in Table 1F using the one-letter amino acid code. The Signal P, Psort and/or Hydropathy results predict that GPCR1c has a signal peptide and is likely to be localized at the endoplasmic reticulum (membrane) with a certainty of 0.6850. In other embodiments, it is localized at the plasma membrane with a certainty of 0.6400, at the Golgi body with a certainty of 0.4600 or at the endoplasmic reticulum (lumen) with a certainty of 0.1000. The most likely cleavage site for a GPCR1c peptide is between amino acids 43 and 44, at: VVG-NC.

TABLE 1F

Encoded GPCR1c protein sequence.

(SEQ ID NO:6)

MIIFNLSSYNPGPFILVGIPGLEQFHVWIGIPFCIIYIVAVVGNCILLYLIVVEHSLHEPMFFFLSMLAMTDLILST
AGVPKTLSIFWLCAREITFPGCLTQMFFLHYNFVLDSAILMAMAFDHYVAICSPLRYTTILTPKTIIKSAMGISFRS
FCIILPDVFLLTCLPFCRTRIIPHTYCEHMGVAQLACADISINFWYGFCVPIMTVISDVILIAVSYAHILCAVFCLP
SQDARQKALGTCGSHVCVILMFYTPAFFSILAHRFGHNVSRTFHIMFANLYIVIPPALNPMVYGVKTKQIRDKVILL
FSKGTG

The disclosed GPCR1c amino acid sequence of the protein of the invention was found to have 181 of 310 amino residues (58%) identical to, and 233 of310 amino acid residues (75%) similar to, the 321 amino acid residue ptnr:SPTREMBL-ACC:Q9WU89 protein from Mus musculus (Mouse) (ODORANT RECEPTOR S18) (E=4.9e⁻¹⁰⁰).

Possible small nucleotide polymorphisms (SNPs) found for NOV1b are listed in Tables 1G and 1H. Depth represents the number of clones covering the region of the SNP. The putative allele frequency (PAF) is the fraction of these clones containing the SNP. Silent indicates that the SNP results in no amino acid sequence change. A dash, when shown, means that a base is not present. The sign “>” means “is changed to.”

TABLE 1G
SNPs

| Consensus | | Base | |
|---|---|---|---|
| Position | Depth | Change | PAF |
| 621 | 18 | T > C | 0.278 |
| 680 | 18 | T > C | 0.389 |

TABLE 1H
SNPs

| | | Amino | |
|---|---|---|---|
| Nucleotide | Base | Acid | Base |
| Position | Change | Position | Change |
| 97 | A > G | Silent | N/A |
| 254 | A > G | 83 | Thr > Ala |
| 313 | A > G | Silent | N/A |
| 378 | G > A | 124 | Arg > His |
| 385 | A > G | Silent | N/A |
| 631 | A > G | Silent | N/A |

Possible SNPs found for GPCR1c are listed in table 1I.

TABLE 1I
SNPs

| Consensus | | Base | |
|---|---|---|---|
| Position | Depth | Change | PAF |
| 254 | 17 | A > G | 0.294 |
| 378 | 18 | G > A | 0.278 |
| 559 | 12 | A > G | 0.250 |
| 631 | 11 | G > A | 0.455 |
| 692 | 11 | G > T | 0.455 |
| 713 | 11 | T > C | 0.455 |
| 721 | 11 | A > G | 0.455 |

GPCR1 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. Expression information was derived from the tissue sources of the sequences that were included in the derivation of the sequence.

The term GPCR1 is used to refer to all GPCR1 variants or members of the GPCR1 family disclosed herein unless we identify a specific family member or variant.

Nucleotide sequence homologies between the GPCR1 variants is shown in a Clustal W in Table 1J.

[image]

[image]

Amino acid sequence homologies between the GPCR1 variants is shown in a Clustal W in Table 1K.

[image]

[image]

The amino acid sequence of GPCR1 a has high homology to other proteins as shown in Table 1L.

TABLE 1L
BLASTX results for GPCR1a

| | | | Smallest |
|---|---|---|---|
| | | | Sum |
| | | High | Prob |
| Sequences producing High-scoring Segment Pairs: | Reading | Score | P (N) |
| ptnr: SPTREMBL-ACC:Q9WU89 ODOR RECP S18- Mus musculus, 321 | aa | 1005 | 3.3e−101 |

The disclosed GPCR1a has homology to the amino acid sequences shown in the BLASTP data listed in Table 1M.

TABLE 1M
BLASTP results for GPCR1a

| | | Length | Identity | Positives | |
|---|---|---|---|---|---|
| Gene Index/Identifier | Protein/Organism | (aa) | (%) | (%) | Expect |
| gi\|9938014 ref\|NP_064 | Odorant receptor | 321 | 183/312 | 236/312 | 1e−93 |
| 686.1\| | S18 gene [Mus musculus] | | (58%) | (74%) | |
| gi\|7305349 ref\|NP_038 | olfactory | 326 | 178/298 | 226/298 | 2e−86 |
| 647.1\| | receptor 67 [Mus musculus] | | (59%) | (75%) | |
| gi\|9935442 ref\|NP_064 | odorant receptor | 318 | 169/310 | 229/310 | 3e−82 |
| 688.1\| | S46 gene [Mus musculus] | | (54%) | (73%) | |
| gi\|14423837\|sp\|Q9H346 | OLFACTORY | 318 | 170/310 | 226/310 | 9e−81 |
| \|OYD1_HUMAN | RECEPTOR 52D1 | | (54%) | (72%) | |
| | (HOR5′BETA14) | | | | |
| gi\|6532001 gb AAD2759 | odorant receptor | 339 | 158/290 | 206/290 | 2e−77 |
| 6.2 AF121976_1 | S19 [Mus musculus] | | (54%) | (70%) | |

The homology of these sequences is shown graphically in the ClustalW analysis shown in Table 1N.

[image]

[image]

The homologies shown above are shared by GPCR1a-GPCR1c insofar as GPCR1a, GPCR1b and GPCR1c are homologous as shown in Table 1K.

Table 1O lists the domain description from DOMAIN analysis results against GPCR2a. This indicates that the GPCR2a sequence has properties similar to those of other proteins known to contain this domain as well as to the 254 amino acid 7tm domain itself.

TABLE 10

Domain Analysis of GPCR1a

gnl|Pfam|pfam00001, 7tm_1, 7 transmembrane receptor (rhodopsin
family). (SEQ ID NO:40)
Length = 254 residues, 100% aligned
Score = 81.6 bits (200), Expect = 6e−17

| | | | |
|---|---|---|---|
| GPCR1a: | 45 | GNCILLYLIVVEHSLHEPMFFFLSMLAMTDLILSTAGVPKTLSIFWLGAREITFPGCLTQ | 104 |
| | | \|\| +++ +\|+ \| \| \|\| \|\|+ \|\|+ \| \| \| \| | |
| Gnl\|Pfam\|pfam00001: | 1 | GNLLVILVILRTKKLRTPTNIGLLNLAVADLLFLLTLPPWALYYLVGGDWVFGDALCKLV | 60 |
| GPCR1a: | 105 | MFFLHYNFVLDSAILMAMAFDRYVAICSPLRYTTILTPKTIIKSAMGISFRSFCIILPDV | 164 |
| | | \| +\| \|++ \|\|\|+\|\| \|\|\|\| \| \|\|+ + + + + \|\| + | |
| Gnl\|Pfam\|pfam00001: | 61 | GALFVVNGYASILLLTAISIDRYLAIVHPLRYRRIRTPRRAKVLILLVWVLALLLSLPPL | 120 |
| GPCR1a: | 165 | FLLTCLPFCRTRIIPHTYCEHIGVAQLACADISINFWYGFCVPIMTVISDILIAVSYAH | 224 |
| | | + + +\| + + ++ \| \|\| + | |
| Gnl\|Pfam\|pfam00001: | 121 | LFSWLRTVEEGNTTVCLIDFPEESVKRSYVLLSTLVGFVLPLLVILVCYTRILRTLRKRA | 180 |
| GPCR1a: | 225 | ILCAVGCLPSQDARQKALGTCGSHVCVILMFYTPAFFSILA--HRFGHNVSRTGHIMFAN | 282 |
| | | \| \|+ \| \| +\| + +\| ++ | |
| Gnl\|Pfam\|pfam00001: | 181 | RSQRSLKRRSSSERKAAKMLLVVVVVFVLCWLPYHIVLLLDSLCLLSIWRVLPTALLITL | 240 |
| GPCR1a: | 283 | LYIVIPPALNPMVY | 296 |
| | | + \|\|\|++\| | |
| Gnl\|Pfam\|pfam00001: | 241 | WLAYVNSCLNPIIY | 254 |

The protein similarity information, expression pattern, and map location for the olfactory receptor-like GPCR1 proteins and nucleic acids disclosed herein suggest that these olfactory receptors may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for the treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders, cell shape disorders, feeding disorders, potential obesity due to over-eating, potential disorders due to starvation (lack of appetite), noninsulin-dependent diabetes mellitus ((NIDDM1)), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, allergies, Parkinson's disease, acute heart failure, hypotension, hypertension, urinary retention, osteoporosis, Crohn's disease, multiple sclerosis, Albright hereditary ostoeodystrophy, angina pectoris, myocardial infarction, ulcers, benign prostatic hypertrophy, psychotic and neurological disorders (including anxiety, schizophrenia, manic depression, delirium, dementia, and severe mental retardation), dentatorubro-pallidoluysian atrophy (DRPLA), hypophosphatemic rickets, autosomal dominant (2) acrocallosal syndrome and dyskinesias, such as Huntington's disease or Gilles de la Tourette syndrome, as well as other diseases, disorders and conditions.

The disclosed GPCR1 protein has multiple hydrophilic regions, each of which can be used as an immunogen. In one embodiment, a contemplated GPCR1 epitope is from about amino acids 160 to 170. In additional embodiments, a GPCR1 epitope is from about amino acids 240 to 249, from about amino acids 260 to 270, from about amino acids 290 to 300 and from 305 to 310. The GPCR1 protein also have value in the development of powerful assay system for functional analysis of various human disorders, which will help in understanding of pathology of the disease and development of new drug targets for various disorders.

GPCR2

GPCR2 includes two GPCR proteins disclosed below. The disclosed proteins have been named GPCR2a and GPCR2b, and are related to olfactory receptors.

GPCR2a

The disclosed GPCR2a nucleic acid of 966 nucleotides (also referred to as AC011711_da2) is shown in Table 2A. The disclosed GPCR2a open reading frame (“ORF”) begins with a ATG at nucleotides 20-22 which encodes a serine (the first amino acid of the mature protein) and ending with a TAA codon at nucleotides 962-964. The start and stop codons of the open reading frame are highlighted in bold type. Putative untranslated regions, if any, are found upstream from the initiation codon and downstream from the termination codon.

TABLE 2A

GPCR2a nucleotide sequence.

(SEQ ID NO:7)

CCCCTTGTCTCCTCACACAATGACCCTGGGATCCCTCGGAAACACCAGCAGCAGCGTTTCTGCTACCTTCCTGCTGA
GGGCATCCCTGGGCTGGAGCGCATGCACATCTGGATCTCCATCCCACTGTGCTTCATGTATCTGGTTTCCATCCCGG
GCAACTGCACAATTCTTTTTATCATTAAAACAGAGCGCTCACTTCATGAACCTATGTATCTCTTCCTGTCCATGCTG
GCTCTGATTGACCTGGGTCTCTCCCTTTGCACTCTCCCTACAGTCCTGGGCATCTTTTGGGTTGGAGCACGAGAAAT
TAGCCATGATGCCTGCTTTGCTCAGCTCTTTTTCATTCACTGCTTCTCCTTCCTCGAGTCCTCTGTGCTACTGTCTA
TGGCCTTTGACCGCTTTGTGGCTATCTGCCACCCCTTGCACTATGTTTCCATTCTCACCAACACAGTCATTGGCAGG
ATTGGCCTCCTCTCTCTGGGTCGTAGTGTAGCACTCATTTTTCCATTACCTTTTATGCTCAAAAGATTCCCCTATTG
TGGCTCCCCAGTTCTCTCACATTCTTATTGTCTCCACCAAGAAGTGATGAAATTGGCCTGTGCCGACATGAAGGCCA
ACAGCATCTACGGCATGTTTGTCATCCTCTCTACAGTGGGTATAGACTCACTGCTCATCCTCTTCTCTTATGCTCTG
ATCCTGCGCACCGTGCTGTCCATCGCCTCCAGGGCTGAGAGATTCAAGGCCCTTAACACCTGTGTTTCCCACATCTG
TGCTGTGCTGCTCTTCTACACTCCCATGATTGGCCTCTCTGTCATCCATCGCTTTGGAAAGCACGCACCCCACCTGG
TCCAGGTGGTCATGGGTTTCATGTATCTTCTCTTTCCTCCTGTGATGAATCCCATTGTCTACAGTGTGAAGACCAAA
CAGATCCGGGATCGAGTGACGCATGCCTTTTGTTACTAACT

The GPCR2 nucleic acid sequence of this invention has 586 of 883 bases (66%) identical to a gb:GENBANK-ID:AR009514|acc:AR009514.1 mRNA from Unknown. (Sequence 1 from patent U.S. Pat. No. 5,756,309) (E=4.1e⁻⁶⁷).

The disclosed GPCR2a polypeptide (SEQ ID NO: 8) encoded protein having 314 amino acid residues is presented in Table 2B using the one-letter amino acid code. The Signal P, Psort and/or Hydropathy results predict that GPCR2a has a signal peptide and is likely to be localized at the plasma membrane with a certainty of 0.6400. In other embodiments, it is localized at the Golgi body with a certainty of 0.4600, at the endoplasmic reticulum (membrane) with a certainty of 0.3700 or at the endoplasmic reticulum (lumen) with a certainty of 0.1000. The most likely cleavage site for a GPCR2a peptide is between amino acids 49 and 50, at: NCT-IL.

TABLE 2B

Encoded GPCR2a protein sequence.

(SEQ ID NO:8)

MTLGSLGNSSSSVSATFLLSGIPGLERMHIWISIPLCFMYLVSIPGNCTILFIIKTERSLHEPMYLFLSMLALIDLG
LSLCTLPTVLGIFWVGAREISHDACFAQLFFIHCFSFLESSVLLSMAFDRFVAICHPLHYVSILTNTVIGRIGLVSL
GRSVALIFPLPFMLKRFPYCHSPVLSHSYCLHQEVMKLACADMKANSIYGMFVIVSTVGIDSLLILFSYALILRTVL
SIASRAERFKALNTCBSHICAVLLFYTPMIGLSVIHRFGKQAPHLVQVVMGFMYLLFPPVMNPIVYSVKTKQIRDRV
THAFCY

The disclosed GPCR2a amino acid sequence of the protein of the invention was found to have 171 of 305 amino acid residues (56%) identical to, and 219 of 305 amino acid residues (71%) similar to, the 320 amino acid residue ptnr:SPTREMBL-ACC:O88628 protein from Rattus norvegicus (Rat) (PUTATIVE G-PROTEIN COUPLED RECEPTOR RA1C) (E=3.7e⁻⁹²).

GPCR2b

The disclosed GPCR2b nucleic acid of 966 nucleotides (also referred to as CG50147-01) is shown in Table 2C. The disclosed GPCR2b open reading frame (“ORF”) begins with an ATG at nucleotides 20-22 and ending with a TAA codon at nucleotides 962-964. The start and stop codons of the open reading frame are highlighted in bold type. Putative untranslated regions, if any, are found upstream from the initiation codon and downstream from the termination codon.

TABLE 2C

GPCR2b nucleotide sequence.

(SEQ ID NO:9)

CCCCTTGTCTCCTCACACAATGACCCTGGGATCCCTGCGAAACAGCAGCAGCAGCGTTTCTGCTACCTTCCTGC
TGAGTGGCATCCCTGGGCTCGAGCGCATGCACATCTGGATCTCCATCCCACTGTGCTTCATGTATCTGGTTTCC
ATCCCGGGCAACTGCACAATTCTTTTTATCATTAAAACAGAGCGCTCACTTCATGAACCTATGTATCTCTTCCT
GTCCATGCTGGCTCTGATTGACCTGGGTCTCTCCCTTTGCACTCTCCCTACAGTCCTGGGCATCTTTTGGGTTG
GAGcACGACAAATTAGCCATGATGCCTGCTTTGCTCAGCTCTTTTTCATTCACTGCTTCTCCTTCCTCGAGTCC
TCTGTGCTACTGTCTATGGCCTTTGACCGCTTTGTCGCTATCTGCCACCCCTTGCACTATGTTTCCATTCTCAC
CAACACAGTCATTGGCAGGATTGGCCTCGTCTCTCTCGGTCGTAGTGTAGCACTCATTTTTCCATTACCTTTTA
TGCTCAAAAGATTCCCCTATTGTCGCTCCCCAGTTCTCTCACATTCTTATTGTCTCCACCAAGAAGTGATGAAA
TTGGCCTGTGCCGACATGAAGGCCAACAGCATCTACCGCATGTTTGTCATCGTCTCTACAGTGGGTATAGACTC
ACTGCTCATCCTCTTCTCTTATGCTCTGATCCTGCGCACCGTGCTGTCCATCCCCTCCAGGGCTGAGACATTCA
AGGCCCTTAACACCTGTGTTTCCCACATCTGTGCTGTGCTGCTCTTCTACACTCCCATGATTGGCCTCTCTGTC
ATCCATCGCTTTGGAAAGCAGGCACCCCACCTGGTCCAGGTGGTCATGGGTTTCATGTATCTTCTCTTTCCTCC
TGTGATGAATCCCATTGTCTACAGTGTGAAGACCAAACAGATCCGGGATCGAGTGACGCATGCCTTTTGTTACT
AACT

The disclosed GPCR2b of this invention maps to chromosome 9 p13.1-13.3 and the GPCR2B the nucleic acid sequence of this invention has 585 of 883 bases (66%) identical to a gb:GENBANK-ID:AR009514|acc:AR009514.1 mRNA from Unknown. (Sequence 1 from patent U.S. Pat. No. 5756309) (E=1.1e⁻⁶⁶).

The disclosed GPCR2b polypeptide (SEQ ID NO: 10) encoded protein having 314 amino acid residues is presented in Table 2D using the one-letter amino acid code. The Signal P, Psort and/or Hydropathy results predict that GPCR2b has a signal peptide and is likely to be localized at the plasma membrane with a certainty of 0.6400. In other embodiments, it is localized at the Golgi body with a certainty of 0.4600, at the endoplasmic reticulum (membrane) with a certainty of 0.3700 or at the endoplasmic reticulum (lumen) with a certainty of 0.1000. The most likely cleavage site for a GPCR2b peptide is between amino acids 49 and 50, at: NCT-IL.

TABLE 2D
Encoded GPCR2b protein sequence

(SEQ ID NO:10)

MTLGSLGNSSSSVSATFLLSGIPGLERMHIWISIPLCFMYLVSIPGNCTILFIIKTERSLHEPMYLFLSMLALIDLG
LSLCTLPTVLGIFNVGARQISHDACFAQLFFIHCFSFLESSVLLSMAFDRFVAICHPLHYVSILTNTVIGRIGLVSL
GTSVALIFPLPFMLKRFPYCGSPVLSHSYCLHQEVMKLACADMKANSIYGMFVIVSTVGIDSLLILFSYALILRTVL
SIASRABRFKALNTCVSHICAVLLFYTPNIGLSVIHRFGKQAPHLVQVVMGFMYLLFPPVMNPIVYSVKTKQIRDRV
THAFCY

The disclosed GPCR2b amino acid sequence of the protein of the invention was found to have 170 of 305 amino acid residues (55%) identical to, and 219 of 305 amino acid residues (71%) similar to, the 320 amino acid residue ptnr:SPTREMBL-ACC:O88628 protein from Rattus norvegicus (Rat) (PUTATIVE G-PROTEIN COUPLED RECEPTOR RA1C) (E=7.7e⁻⁹²).

Possible SNPs found for GPCR2b are listed in Tables 2E and 2F.

TABLE 2E
SNPs

| Consensus | | Base | |
|---|---|---|---|
| Position | Depth | Change | PAF |
| 313 | 21 | C > G | 0.429 |
| 543 | 28 | T > — | 0.071 |

TABLE 2F
SNPs

| | | Amino | |
|---|---|---|---|
| Nucleotide | Base | Acid | Base |
| Position | Change | Position | Change |
| 124 | A > G | Silent | N/A |
| 145 | T > C | Silent | N/A |
| 789 | A > G | 257 | Tyr > Cys |
| 841 | C > T | Silent | N/A |

GPCR2 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypithalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putament, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

The term GPCR2 is used to refer to all GPCR2 variants or members of the GPCR2 family disclosed herein unless we identify a specific family member or variant.

Nucleotide sequence homologies between the GPCR2 variants is shown in a Clustal W in Table 2G.

[image]

[image]

Amino acid sequence homologies between the GPCR2 variants is shown in a Clustal W in Table 2H.

[image]

The amino acid sequence of GPCR2a has high homology to other proteins as shown in Table 2I.

TABLE 2I
BLASTX results for GPCR2a

| | | | Smallest |
|---|---|---|---|
| | | | Sum |
| | | High | Prob |
| Sequences producing High-scoring Segment Pairs: | Reading | Score | P (N) |
| ptnr:SPTREMBL-ACC:088628 GPCRECP - Rattus norv, 320 | aa . . . | 920 | 3.7e−92 |

The disclosed GPCR2a has homology to the amino acid sequences shown in the BLASTP data listed in Table 2J.

TABLE 2J
BLASTP results for GPCR2a

| | | Length | Identity | Positives | |
|---|---|---|---|---|---|
| Gene Index/Identifier | Protein/Organism | (aa) | (%) | (%) | Expect |
| gi 13540539 ref NP_1104 | prostate specific G- | 320 | 172/305 | 224/305 | 2e−85 |
| 01.1 | protein coupled | | (56%) | (73%) | |
| | receptor | | | | |
| | [Homo sapiens] | | | | |
| gi 17063576 gb AAL35109 | prostate-specific G | 320 | 173/305 | 220/305 | 4e−85 |
| .1\|AF378854 1 | protein-coupled | | (56%) | (71%) | |
| | receptor RA1c | | | | |
| | [Mus musculus] | | | | |
| gi 11908220 gb AAG41684 | MOR 3′Beta4 | 319 | 179/306 | 225/306 | 9e−85 |
| .1\| | [Mus musculus] | | (58%) | (73%) | |
| gi 3420759 gb AAD12761. | putative G-protein | 320 | 171/305 | 219/305 | 2e−84 |
| 1 | coupled receptor | | (56%) | (71%) | |
| | RA1c | | | | |
| | [Rattus norvegicus] | | | | |
| gi 14423836 sp Q9H344 O | OLFACTORY RECEPTOR | 312 | 173/298 | 218/298 | 2e−83 |
| XI2 HUMAN | 51I2 (HOR5′BETA12) | | (58%) | (73%) | |

The homology of these sequences is shown graphically in the ClustalW analysis shown in Table 2K.

[image]

[image]

The homologies shown above are shared by GPCR2a and GPCR2b insofar as GPCR2a and GPCR2b are homologous as shown in Table 2H.

Table 2L lists the domain description from DOMAIN analysis results against GPCR2a. This indicates that the GPCR2a sequence has properties similar to those of other proteins known to contain this domain as well as to the 254 amino acid 7tm domain itself.

TABLE 2L
Domain Analysis of GPCR2a

gnl|Pfam|pfam00001, 7tm_1, 7 transmembrane receptor
(rhodopsin family). (SEQ ID NO:46)
Length = 254 residues, 100% aligned
Score = 70.9 bites (172), Expect = 1e−13

| | | | |
|---|---|---|---|
| GPCR2a: | 46 | GNCTILFIIKTERSLHEPMYLFLSMLALIDLGLSLCTLPTVLGIFWVGAREISHDACGAQ | 105 |
| | | \|\| ++ +\| + \| \| +\|\| \|\|+ \|\| \| \| \| \| \| | |
| Gnl\|Pfam\|pfam00001: | 1 | GNLLVILVILRTKKLRTPTNIFLLNLAVADLLFLLTLPPWALYYLVGGDWVFGDALCKLV | 60 |
| GPCR2a: | 106 | LFFIHCFSFLESSVLLSMAFDRFVAICHPLHYVSILTNTVIGRIGLVSLGRSVALIFPLP | 165 |
| | | + +\| +++ \|\|++\|\| \|\|\| \| \| \| + \|+ ++ \| \| | |
| Gnl\|Pfam\|pfam00001: | 61 | GALFVVNGYASILLLTAISIDRYLAIVHPLRYRRIRTPRRAKVLILLVWVLALLLSLPPL | 120 |
| GPCR2a: | 166 | FM--LKRFPYCGSPVLSHSYCLHQEVMKLACADMKANSIYGMFVIVSTVGIDSLLILFSY | 223 |
| | | \|+ + \| + \| + + + \|+\|\| \| | |
| Gnl\|Pfam\|pfam00001: | 121 | LFSWLRTVEEGNTTVCLIDFPEE-----------SYKRSYVLLSTLVGFVLPLLVILVCY | 169 |
| GPCR2a: | 224 | ALILRTVLSIASRAERF--------KALNTCVSHICAVLLFYTPMIGLSVIHRFGKQAPH | 275 |
| | | \|\|\|\|+ \| \|\| + + +\| + \| + ++ + | |
| Gnl\|Pfam\|pfam00001: | 170 | TRILRTLRKRARSQRSLKRRSSSERKAAKMLLVVVVVFVLCWLPYHIVLLLDSLCLLSIW | 229 |
| GPCR2a: | 276 | LVQVVMGFMYLLFP---PVMNPIVY 297 | |
| | | \| + \| +\|\|\|+\| | |
| Gnl\|Pfam\|pfam00001: | 230 | PVLPTALLITLWLAYVNSCLNPIIY 254 | |

The protein similarity information, expression pattern, and map location for the olfactory receptor-like protein and nucleic acid GPCR2 suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) Acrocallosal syndrome and dyskinesias, such as Huntington's disease or Gilles de la Tourette syndrome and/or other pathologies and disorders of the like.

The disclosed GPCR2 protein has multiple hydrophilic regions, each of which can be used as an immunogen. In one embodiment, a contemplated GPCR2 epitope is from about amino acids 170 to 180. In additional embodiments, a GPCR2 epitope is from about amino acids 185 to 190, from about amino acids 195 to 200, and from 280 to 315. The GPCR2 protein also have value in the development of powerful assay system for functional analysis of various human disorders, which will help in understanding of pathology of the disease and development of new drug targets for various disorders.

GPCR3

The disclosed GPCR3 nucleic acid of 1251 nucleotides (also referred to as GMAC024428_A_) is shown in Table 3. The disclosed GPCR3 open reading frame begins with a ATG at nucleotides 91-93 and ending with a TGA codon at nucleotides 1162-1164. The start and stop codons of the open reading frame are highlighted in bold type. Putative untranslated regions, if any, are found upstream from the initiation codon and downstream from the termination codon.

TABLE 3A
GPCR3 nucleotide sequence.

(SEQ ID NO:11)

CAAAATGATTATAGCTGACAAATCAGGAAGTGTGTTGTTAACTTCCTGACTTCTTATATTTCAGAGAACGAAGAGT
TGAACCATTTAAATGAATTGGGTAAATAAGAGTGTCCCACAGGAGTTCATTCTGTTAGTTTTCTCAGATCAACCAT
GGCTAGAATTCCACCCTTTGTGATGTTTCTGTTTTCCTATATCTTGACAATCTTTGGCAATCTGACAATAATTCTT
GTGTCACATGTGGATTCAAACTCCACACCCCTATGTACTTTTTTCTTAGCAATCTCTCACTCCTGGACCTTTGCTA
TACCACAAGTACAGTTCCACAAATGCTGGTAAACATATGCAACACCAGGAAAGTAATCAGTTATGGTGGCTGTGTGG
CCCAGCTTTTCATTTTCCTGGCCTTGGGTTCCACAGAATGTCTTCTCCTGGCCGTCATGTGCTTTGATAGGTTGTA
GCTATTTGTCGGCCTCTCCATTACTCAATTATCATGCACCAGAGGCTCTGCTTCCAGTTGGCAGCTGCATCCTGGAT
TAGTGGCTTTAGCAATTCAGTATTACAGTCCACCTGGACACTTAAGATGCCACTGTGTGGTCACAAAGAAGTGGATC
ACTTCTTCTGTGAAGTCCCTGCTCTGCTCAAGTTGTCCTGTGTTGACACAACAGCAAATGAGGCTGAACTATTCTTC
ATCAGTGTGCTATTCCTTCTAATACCCGTGACACTCATCCTTATATCGTATGCTTTTATTGTCCAAGCAGTGTTGAG
AATCCAGTCTGCTGAAGGTCAACGAAAGGCATTTGGGACATGTGGCTCCCATCTAATTGTCGTGTCACTTTTTTATG
GTACAGCTATCTCCATGTACCTGCAACCACCTTCACCCAGCTCCAAAGACCGGGGAAAGATGGTTTCTCTCTTCTGT
GGAATCATTGCACCCATGCTGAATCCCCTTATATATACACTTAGGAACAAAGAGGTAAAGGAAGCCTTTAAAAGGTT
GGTTGCAAAGAGTCTTCTTAATCAAGAAATAAGAAATATGCAAATGATAAGCTTTGCTAAAGACACAGTGCTTACTT
ACCTTACTAACTTCTCCGCAAGTTGTCCTATTTTTGTCATTACTATAGAAAACTATTGTAATCTCCCTCAAAGAAAA
TTTCCTTGACAAAAAGCTATATTTGTTTCTGTTGCCTAAACATTTTCATTGAACAAGCCCCCAGAATTGGCCTTCCA
ATGCACCAAAAACTGTAAT

The disclosed GPCR3 of this invention maps to chromosome 3 the nucleic acid sequence of this invention has 881 of 1119 bases (78%) identical to a gb:GENBANK-ID:RATOL1RECE|acc:L34074.1 mRNA from Rattus norvegicus (Rat OL1 receptor gene, complete cds (E=2.1e⁻¹⁴²). Chromosome localization information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

The disclosed GPCR3 polypeptide (SEQ ID NO: 12) encoded protein having 357 amino acid residues is presented in Table 3B using the one-letter amino acid code. The Signal P, Psort and/or Hydropathy results predict that GPCR3 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the microbody (peroxisome) with a certainty of 0.3000. The most likely cleavage site for a GPCR3 peptide is between amino acids 41 and 42, at: IFG-NL.

TABLE 3B
Encoded GPCR3 protein sequence.

(SEQ ID NO:12)

MNWVNKSVPQEFILLVFSDQPWLEIPPFVMFLFSYILTIFGNLTIILVSHVDFKLHTPMYFFLSNLSLLDLCYTTST
VPQMLVNICNTRKVISYGGVAQLFIFLALGFSTECLLLAVMCFDRFVAICRPLHYSIIMHQRLCFQLAAASWISGFS
NSVIQSTWTLKMPLCGHKEVDEFFCEVPALLKLSCVDTTANEAELFFISVLFLLIPVTLILISYAFIVQAVLRIQSA
EGQRKAFGTCGSHLIVVSLFYGTAISMYLQPPSPSSKDRGKMVSLFCGIIAPMLNPLIYTLRNKEVKFAFK2LVAKS
LLNQEIRNMQMISFAlGJTVLTYLTNFSASCPIFVITIENYCNLPQRKFP

The disclosed GPCR3 full amino acid sequence of the protein of the invention was found to have 310 of 310 amino acid residues (100%) identical to, and 310 of 310 amino acid residues (100%) similar to, the 310 amino acid residue ptnr:SPTREMBL-ACC:Q9Y299 protein from Homo sapiens (Human) (OLFACTORY RECEPTOR (E=8.6e⁻¹⁶⁵).

GPCR3 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

Possible SNPs found for GPCR3 are listed in Tables 3C and 3D. The SNP at bp 1122 found in Table 3D is also referred to as Accession Number CG92194-01.

TABLE 3C
SNPs

| Consensus | | Base | |
|---|---|---|---|
| Position | Depth | Change | PAF |
| 124 | 12 | A > C | 0.167 |
| 319 | 11 | A > G | 0.273 |

TABLE 3D
SNPs

| Nucleotide | Base | Amino | Base |
|---|---|---|---|
| Position | Change | Acid | Change |
| | | Position | |
| 129 | T > C | Silent | N/A |
| 318 | T > C | Silent | N/A |
| 1122 | T > C | Silent | N/A |

The amino acid sequence of GPCR3 has high homology to other proteins as shown in Table 3E.

TABLE 3E
BLASTX results for GPCR3

| | | | Smallest |
|---|---|---|---|
| | | | Sum |
| | | High | Prob |
| Sequences producing High-scoring Segment Pairs: | Reading | Score | P (N) |
| ptnr:SPTREMBL-ACC:Q9Y299 OLF RECP - Homo Sapiens, 310 | aa . . . | 1605 | 8.6e−165 |

The disclosed GPCR3 has homology to the amino acid sequences shown in the BLASTP data listed in Table 3F.

TABLE 3F
BLASTP results for GPCR3

| | | Length | Identity | Positives | |
|---|---|---|---|---|---|
| Gene Index/Identifier | Protein/Organism | (aa) | (%) | (%) | Expect |
| gi\|15304846\|ref XP_05 | olfactory | 357 | 357/357 | 357/357 | 0.0 |
| 3609.1 | receptor, family | | (100%) | (100%) | |
| | 2, subfamily B, | | | | |
| | member 2 | | | | |
| | [Homo sapiens] | | | | |
| gi\|14780900\|ref NP_14 | olfactory | 357 | 356/357 | 357/357 | 0.0 |
| 9046.1 | receptor, family | | (99%) | (99%) | |
| | 2, subfamily B, | | | | |
| | member 2 | | | | |
| | [Homo sapiens] | | | | |
| gi\|3080467 emb\|CAB114 | olfactory | 310 | 310/310 | 310/310 | e−159 |
| 27.1 | receptor | | (100%) | (100%) | |
| | [Homo sapiens] | | | | |
| gi\|11177906\|ref NP_06 | Olfactory | 313 | 261/310 | 284/310 | e−134 |
| 8632.1 | receptor | | (84%) | (91%) | |
| | [Rattus norvegicus] | | | | |
| gi 14423785 sp P58173 O | OLFACTORY RECEPTOR | 313 | 253/310 | 278/310 | e−128 |
| 236 HUMAN | 2B6 (HS6M1-32) | | (81%) | (89%) | |
| | (OLFACTORY RECEPTOR | | | | |
| | 6-31) | | | | |
| | (OR6-31) | | | | |

The homology of these sequences is shown graphically in the ClustalW analysis shown in Table 3G.

[image]

[image]

Table 3H lists the domain description from DOMAIN analysis results against GPCR3. This indicates that the GPCR3asequence has properties similar to those of other proteins known to contain this domain as well as to the 254 amino acid 7tm domain itself.

TABLE 3H
Domain Analysis of GPCR3

gnl|Pfam|pfam00001, 7tm_1, 7 transmembrane receptor
(rhodopsin family). (SEQ ID NO:52)
Length = 254 residues, 94.5% aligned
Score = 110 bits (275), Expect = 1e−25

| | | | |
|---|---|---|---|
| GPCR3a: | 41 | GNLTIILVSHVDFKLHTPMYFFLSNLSLLDLCYTTSTVPQMLVNICNTRKVISYGGCVAQ | 100 |
| | | \|\|\| +\|\|\| \|\| \|\| \|\| \|\|++ \|\| + + \| \| + \| \| | |
| Gnl\|Pfam\|pfam00001: | 1 | GNLLVILVILRTKKLRTPTNIFLLNLAVADLLFLLTLPPWALYYLVGGDWVFGDALCKLV | 60 |
| GPCR3a: | 101 | LFIFLALGSTECLLLAVMCFDRFVAICRPLHYSIIMHQRLCFQLAAASWISGFSNSVLQS | 160 |
| | | +\|+ \| \|\|\| + \|\|++\|\| \|\| \| \| \| \| \|+ \|+ | |
| Gnl\|Pfam\|pfam00001: | 61 | GALFVVNGYASILLLTAISIDRYLAIVHPLRYRRIRTPRRAKVLILLVWVLALLLSLPPL | 120 |
| GPCR3a: | 161 | T--WTLKMPLCGHKEVDHFFCEVPALLKLSCVDTTANEAELFFISVLFLLIPVTLILISY | 218 |
| | | \| + \| \| + +++ ++\|+ +\|\|+ \| | |
| Gnl\|Pfam\|fam00001: | 121 | LFSWLRTVEEGNTTVCLIDFPEESVKRSY-----------VLLSTLVGFVLPLLVILVCY | |
| CPCR3a: | 219 | AFIV---------QAVLRIQSAEGQRKAFGTCGSHLIVVSLFYGTAISMYL----QPPSP | 265 |
| | | \|+ \| \|+ +\|+ ++ \| ++ \| + \| + \| | |
| Gnl\|Pfam\|pfam00001: | 170 | TRILRTLRKRARSQRSLKRRSSSERKAAKMLLVVVVVFVLCWLPYHIVLLLDSLCLLSIW | 229 |
| GPCR3a: | 266 | SSKDRGKMVSLFCGIIAPMLNPLIY 290 | |
| | | +++\|+ + \|\|\|+\|\| | |
| Gnl\|Pfam\|pfam00001: | 230 | RVLPTALLITLWLAYVNSCLNPIIY 254 | |

The protein similarity information, expression pattern, and map location for the olfactory receptor-like GPCR3 protein and nucleic acid disclosed herein suggest that GPCR3 may have important structural and/or physiological functions characteristic of the GPCR family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) acrocallosal syndrome and dyskinesias, such as Huntington's disease or Gilles de la Tourette syndrome and/or other pathologies and disorders of the like.

The disclosed GPCR3 protein has multiple hydrophilic regions, each of which can be used as an immunogen. In one embodiment, a contemplated GPCR3 epitope is from about amino acids 140 to 160. In additional embodiments, a GPCR3 epitope is from about amino acids 235 to 245, from about amino acids 260 to 275, from 295 to 310, from 320 to 325 and from 330 to 335. The GPCR3 protein also have value in the development of powerful assay system for functional analysis of various human disorders, which will help in understanding of pathology of the disease and development of new drug targets for various disorders.

GPCR4

The disclosed GPCR4 nucleic acid of 953 nucleotides (also referred to as CG50169-01) is shown in Table 4A. The disclosed GPCR4 open reading frame (“ORF”) begins with a ATG at nucleotides 16-18 and ending with a TAA codon at nucleotides 946-948. The start and stop codons of the open reading frame are highlighted in bold type. Putative untranslated regions, if any, are found upstream from the initiation codon and downstream from the termination codon.

TABLE 4A
GPCR4 nucleotide sequence.

(SEQ ID NO:13)

GCCTGACCAAAAAGAATGGCAGCCAAAAACTCTTCTGTGACAGAGTTTATCCTCGAAGGCTTAACCCACCAGCCGGG
ACTGCGGATCCCCCTCTTCTTCCTGTTTCTGGGTTTCTACACGGTCACCGTGGTGGGGAACCTGGGCTTGATAACCC
TGATTGGGCTGAACTCTCACCTGCACACTCCCATGTACTTCTTCCTTTTTAACCTCTCTTTAATAGATTTCTGTTTC
TCCACTACCATCACTCCCAAAATGCTGATGAGTTTTGTCTCAAGGAAGAACATCATTTCCTTCACAGGGTGTATGAC
TCAGCTCTTCTTCTTCTGCTTCTTTGTCGTCTCTGAGTCCTTCATCCTGTCAGCGATGGCGTATGACCGCTACGTGG
CCATCTGTAACCCACTGTTGTACACAGTCACCATGTCTTGCCAGGTGTGTTTGCTCCTTTTGTTGGGTGCCTATGGG
ATGGGGTTTGCTGGGGCCATGGCCCACACAGGAAGCATAATGAACCTGACCTTCTGTGCTGACAACCTTGTCAATCA
TTTCATGTGTGACATCCTTCCTCTCCTTGAGCTCTCCTGCAACAGCTCTTACATGAATGAGCCGGTGGTCTTTATTG
TGGTGGCTGTTGACGTTGGAATGCCCATTGTCACTGTCTTTATTTCTTATGCCCTCATCCTCTCCAGCATTCTACAC
TTCTGGTGCTTTCATGTATCTCAAACCCCTTTCCATCCTGCCCCTCGAGCAAGGGAAAGTGTCCTCCCTGTTCTATA
CCATAATAGTCCCCCGTGTTAAACCCATTAATCTATAGCTTGAGGAACAAGGATGTCAAAGTTGCCCTGAGAGAACT
TTGGGCAGAAAAAATCTTTTCTTAAGAAAG

The GPCR4 the nucleic acid sequence of this invention has 527 of 660 bases (79%) identical to a gb:GENBANK-ID:SCO233799|acc:AJ233799.1 mRNA from Stenella coeruleoalba (Stenella coeruleoalba olfactory receptor pseudogene, partial, clone SCor3) (E=7.5e⁻⁸⁹).

The disclosed GPCR4 polypeptide (SEQ ID NO: 14) encoded protein having 310 amino acid residues is presented in Table 4B using the one-letter amino acid code. The Signal P, Psort and/or Hydropathy results predict that GPCR4 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the microbody (peroxisome) with a certainty of 0.3000. The most likely cleavage site for a GPCR4 peptide is between amino acids 40 and 41, at: VVG-NL.

TABLE 4B
Encoded GPCR4 protein seqnence.

(SEQ ID NO:14)

MAAKNSSVTEFILEGLTHQPGLRIPLEFFLFLGFYTVTVVGNLGLITLIGLNSHLHTPMYFFLFNLSLIDFCFSTTIT
PKMLMSFVSRKNIISFTGCMTQLFFFCFFVVSESFILSAMAYDRYVAICNPLLYTVTMSCQVCLLLLLLGAYGMGFAG
AAMAHTGSIMNLTFCADNLVNHFMCDILPLLELSCNSSYNMEPVVFIVVAVDVGMPIVTVFISYALILSSILHNSSTE
GGRSKAFSTCSSHIIVVSLFFGSGAFMYLKPLSILPLEQGKVSSLFYTIIVPVLNPLIYSLRNKDVKVALRRTLGRKI
FS

The disclosed GPCR4 amino acid sequence of the protein of the invention was found to have 255 of 304 amino acid residues (83%) identical to, and 282 of 304 amino acid residues (92%) similar to, the 304 amino acid residue ptnr:SPTREMBL-ACC:Q9QW36 protein from Rattus sp (OR14=ODORANT RECEPTOR) (E=3.2e⁻¹³⁴).

GPCR4 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

The amino acid sequence of GPCR4 has high homology to other proteins as shown in

TABLE 4C
BLASTX results for GPCR4

| | | | Smallest |
|---|---|---|---|
| | | | Sum |
| Sequences producing High-scoring | | High | Prob |
| Segment Pairs: | Reading | Score | P(N) |
| ptnr:SPTREMBL-ACC:QPQW36 ODOR | 304 aa. | 1317 | 3.2e−134 |
| RECP - Rattus sp, | | | |

The disclosed GPCR4 has homology to the amino acid sequences shown in the BLASTP data listed in Table 4D.

TABLE 4D
BLASTP results for GPCR4

| | | Length | Identity | Positives | |
|---|---|---|---|---|---|
| Gene Index/Identifier | Protein/Organism | (aa) | (%) | (%) | Expect |
| gi\|11692541\|gb\|AAG398 | odorant receptor | 310 | 256/310 | 283/310 | e−120 |
| 67.1\|AF282282_1 | K23 [Mus musculus] | | (82%) | (90%) | |
| | Length = 310 | | | | |
| gi\|11692535\|gb\|AAG398 | odorant receptor | 310 | 259/310 | 283/310 | e−117 |
| 64.1\|AF282279_1 | K21 [Mus musculus] | | (83%) | (90%) | |
| gi\|11692539\|gb\|AAG398 | odorant receptor | 309 | 258/310 | 284/310 | e−117 |
| 66.1\|AF282281_1 | K22 [Mus musculus] | | (83%) | (91%) | |
| gi\|423701\|pir \|S29709 | olfactory | 304 | 255/304 | 282/304 | e−117 |
| | receptor OR14 - | | (83%) | (91%) | |
| | rat | | | | |
| gi\|14423794\|sp\|Q15620 | OLFACTORY | 311 | 248/311 | 276/311 | e−110 |
| O8B8_HUMAN | RECEPTOR 8B8 | | (79%) | (88%) | |
| | (OLFACTORY | | | | |
| | RECEPTOR TPCR85) | | | | |

The homology of these sequences is shown graphically in the ClustalW analysis shown in Table 4E.

[image]

[image]

Table 4F lists the domain description from DOMAIN analysis results against GPCR4. This indicates that the GPCR4 sequence has properties similar to those of other proteins known to contain this domain as well as to the 254 amino acid 7tm domain itself.

TABLE 4F
Domain Analysis of GPCR4

gnl|Pfam|pfam00001, 7tm_1, 7 transmembrane receptor
(rhodospin family). (SEQ ID NO:58)
Length = 254 residues, 94.9% aligned
Score = 62.0 bits (149), Expect = 5e−11

| | | | |
|---|---|---|---|
| GPCR4: | 53 | HLHTPMYFFLFNLSLIDFCFSTTITPKMLMSFVSRKNIISFTGCMTQLFFFCFFVVSESF | 112 |
| | | \| \|\| \|\| \|\|++ \| \| \|+ \| \| \| + \| \| + | |
| Gnl\|Pfam\|pfam00001: | 14 | KLRTPTNIFLLNLAVADLLFLLTLPPWALYYLVGGDWVFGDALCKLVGALFVVNGYASIL | 73 |
| GPCR4: | 113 | ILSAMAYDRYVAICNPLLYTVTMSCQVCLLLLLGAYGMGFAGAMAHTGSIMNLTFCADNL | 172 |
| | | +\|+\|++ \|\|\|+\|\| +\|\| \| + + +\|+\| + + ++ ++ | |
| Gnl\|Pfam\|pfam00001: | 74 | LLTAISIDRYLAIVHPLRYRRIRTPRRAKVLILLVWVLALLLSLPP---LLFSWLRTVEE | 130 |
| GPCR4: | 173 | VNHFMCDILPLLELSCNSSYMNEPVVFIVVAVDVGMPIVTVFISYALIL---------SS | 223 |
| | | \| +\| \| \| \| + +\| \|+ \|++ + + \| \|\| | |
| Gnl\|Pfam\|pfam00001: | 131 | GNTTVCLIDFPEESVKRSYVLLSTLVGFVL------PLLVILVCYTRILRTLRKRARSQR | 184 |
| GPCR4: | 224 | ILHNSSTEGRSKAFSTCSSHIIVVSLFFGSGAFMYLKPLSILP----LEQGKVSSLFYTI | 279 |
| | | \| \|+ \| \| ++ \| + + \| \| +\| \| + +\|+ | |
| Gnl\|Pfam\|pfam00001: | 185 | SLKRRSSSERKAAKMLLVVVVVFVLCWLPYHIVLLLDSLCLLSIWRVLPTALLITLWLAY | 244 |
| GPCR4: | 280 | IVPVLNPLIY 289 | |
| | + \|\|\|+\|\| | | |
| Gnl\|Pfam\|pfam00001: | 245 | VNSCLNPIIY 254 | |

The protein similarity information, expression pattern, and map location for the olfactory receptor-like GPCR 4 protein and nucleic acid disclosed herein suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of appetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) Acrocallosal syndrome and dyskinesias, such as Huntington's disease or Gilles de la Tourette syndrome and/or other pathologies and disorders of the like.

The disclosed GPCR4 protein has multiple hydrophilic regions, each of which can be used as an immunogen. In one embodiment, a contemplated GPCR4 epitope is from about amino acids 220 to 240. In additional embodiments, a GPCR4 epitope is from about amino acids 280 to 310. The GPCR4 protein also have value in the development of powerful assay system for functional analysis of various human disorders, which will help in understanding of pathology of the disease and development of new drug targets for various disorders.

GPCR5

The disclosed GPCR5 nucleic acid of 1103 nucleotides (also referred to as AC009758_da1) is shown in Table 5A. The disclosed GPCR5 open reading frame (“ORF”) begins with a ATG at nucleotides 66-68 which encodes a serine (the first amino acid of the mature protein) and ending with a TAA codon at nucleotides 1020-1022. The start and stop codons of the open reading frame are highlighted in bold type. Putative untranslated regions, if any, are found upstream from the initiation codon and downstream from the termination codon.

TABLE 5A
GPCRS nucleotide sequence.

(SEQ ID NO:15)

TGCAGATTTTATCTTCTTTCTACCTCTGTGAGTAGAAGGTGAGGTTCTGANAGTTCTCCCCAGCTATGCCTACTGTA
AACCACAGTGGCACTAGCCACACAGTCTTCCACTTGCTGGGCATCCCTGGCCTACAGGACCAGCACATGTGGATTTC
TATCCCATTCTTCATTTCCTATGTCACCGCCCTTCTTGGGAACAGCCTGCTCATCTTCATTATCCTCACAAAGCGCA
GCCTCCATGAACCCATGTACCTCTTCCTCTGCATGCTGGCTGGAGCAGACATTGTCCTCTCCACGTGCACCATTCCT
CAGCCCTTAGCTATCTTCTGGTTCCGTGCTGGGGACATCTCCCTGGATCGTTGCATCACTCAGCTCTTCTTCATCCA
TTCCACCTTCATCTCTGAGTCAGGGATCTTGCTGGTGATGGCCTTTGACCACTATATTGCCATATGCTACCCACTGA
GGTACACCACCATTCTTACAAATGCTCTGATCAAGAAAATTTGTGTGACTGTCTCTCTGAGAAGTTATGGTACAATT
TTCCCTATCATATTTCTTTTAAAAAGATTGACTTTCTGCCAGAATAATATTATTCCACACACCTTTTGTGAACACAT
TGGCCTAGCCAAATATGCATGTAATGACATTCGAATAAACATTTGGTATGGGTTTTCCATTCTAATGTCGACGGTCC
TCTTAAATGTTGTACTAATTTTTATTTCCTATATGCTGATTCTCCATGCTGTCTTCCACATACCTTCTCCAGATGCT
TGCCACAAAGCTCTCAACACATTTGGCTCCCATGTCTGCATCATCATCCTCTTTTATGGGTCTGGCATCTTCACAAT
CCTTACCCAGAGGTTTGGACGCCACATTCCACCTTGTATCCACATCCCGTTGGCTAATGTCTGCATTCTGGCTCCAC
CTATGCTGAATCCCATTATTTATGGGATCAAAACCAAGCAAATCCAGGAACAGGTGGTTCAGTTTTTGTTTATAAAA
CAGAAAATAACTTTGGTTTAAGAACTGAGTTTTCAGAATCTCTAGCTATCTGGTAAGTGGGTATGAAAGTGGTAGAT
GGGAGAGGTCAGCTGATACCGTAGA

The disclosed GPCR5 of this invention maps to chromosome 11 and the GPCR5 the nucleic acid sequence of this invention has 687 of 1030 bases (66%) identical to a gb:GENBANK-ID:AF121975|acc:AF121975.1 mRNA from Mus musculus (Mus musculus odorant receptor S18 gene, complete cds) (E=6.7e⁻⁷⁴). Chromosome localization information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

The disclosed GPCR5 polypeptide (SEQ ID NO: 16) encoded protein having 318 amino acid residues is presented in Table 5B using the one-letter amino acid code. The Signal P, Psort and/or Hydropathy results predict that GPCR5 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the mitochondrial inner membrane with a certainty of 0.3000. The most likely cleavage site for a GPCR5 peptide is between amino acids 53 and 54, at: ILT-KR.

TABLE 5B
Encoded GPCR5 protein sequence.

(SEQ ID NO:16)

MPTVNESGTSETVFHLLGIPGLQDQHMWISIPFFISYVTALLGNSLLIFIILTKRSLNEPNYLFLCMLAGADIVLST
CTIPQALAIFWFRAGDISLDRCITQLFFIHSTFISESGILLVMAFDHYIAICYPLRYTTILTNALIKKICVTVSLRS
YGTIFPIIFLLKRLTFCQNNIIPHTFCEHIGLAKYACNDIRINIWYGFSILMSTVVLDVVLIFISYMLILHAVFHMP
SPDACHKALNTFGSHVCIIILFYGSGIFTILTQRFGRHIPPCIHIPLANVCILAPPMLNPIIYGIKTKQIQEQVVQF
LFIKQKITLV

The disclosed GPCR5 amino acid sequence of the protein of the invention was found to have 184 of 303 amino acid residues (60%) identical to, and 237 of 303 amino acid residues (78%) similar to, the 321 amino acid residue ptnr:SPTREMBL-ACC:Q9WU89 protein from Mus musculus (Mouse) (ODORANT RECEPTOR S18) (E=1.8e⁻¹⁰¹).

GPCR5 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

The amino acid sequence of GPCR5 has high homology to other proteins as shown in Table 5C.

TABLE 5C
BLASTX results for GPCR5

| | | | Smallest |
|---|---|---|---|
| | | | Sum |
| Sequences producing High-scoring | | High | Prob |
| Segment Pairs: | Reading | Score | P (N) |
| Ptnr:SPTREMBL-ACC:Q9WU89 | 321 aa . . . | 1008 | 1.8e−101 |
| ODOR RECP - Mus musculus, | | | |

The disclosed GPCR5 has homology to the amino acid sequences shown in the BLASTP data listed in Table 5D.

TABLE 5D
BLASTP results for GPCR5

| | | Length | Identity | Positives | |
|---|---|---|---|---|---|
| Gene Index/Identifier | Protein/Organism | (aa) | (%) | (%) | Expect |
| gi\|9938014 ref\|NP_064 | odorant receptor | 321 | 184/303 | 237/303 | 6e−93 |
| 686.1\| | S18 gene | | (60%) | (77%) | |
| | [Mus musculus] | | | | |
| gi\|7305349\|ref\|NP_038 | MOR 3′Beta1 | 326 | 168/307 | 227/307 | 2e−79 |
| 647.1\| | [Mus musculus] | | (54%) | (73%) | |
| gi\|6532001\|gb AAD2759 | odorant receptor | 339 | 149/293 | 214/293 | 6e−75 |
| 6.2 AF121976_1 | S19 [Mus musculus] | | (50%) | (72%) | |
| gi\|14423837\|sp\|Q9H346 | OLFACTORY | 318 | 153/299 | 212/299 | 9e−74 |
| \|OYD1_HUMAN | RECEPTOR 52D1 | | (51%) | (70%) | |
| | (HOR5′BETA14) | | | | |
| gi\|9935442\|ref\|NP_064 | odorant receptor | 318 | 154/305 | 212/305 | 4e−69 |
| 688.1\| | S46 gene | | (50%) | (69%) | |
| | [Mus musculus] | | | | |

The homology of these sequences is shown graphically in the ClustalW analysis shown in Table 5E.

[image]

[image]

Table 5F lists the domain description from DOMAIN analysis results against GPCR2a. This indicates that the GPCR2a sequence has properties similar to those of other proteins known to contain this domain as well as to the 254 amino acid 7tm domain itself.

TABLE 5F

Domain Analysis of GPCR5

gnl|Pfam|pfam0001, 7tm_1, 7 transmembrane receptor (rhodopsin
family). (SEQ ID NO: 64)
Length = 254 residues, 100.0% aligned
Score = 87.4 bits (215), Expect = 1e−18

| | | | |
|---|---|---|---|
| GPCRS: | 43 | GNSLLIFTILTKRSLHEPMYLBLCMLAGADIVLSTCTIPQALAIFWF2AODISLDRCITQ | 102 |
| | | \|\| \|+\| +\|\|+ + \| \| +\|\| \|\| \|\|++ \| \| \| | |
| Gnl\|Pfam\|pfam0001: | 1 | GNLLVILVILRTKKLRTPTNIFLLNLAVADLLFLLTLPPWALYYLVGGDWVFGDALCKLV | 60 |
| GPCR5: | 103 | LFFIHSTFISESGILLVMAFDHYTAICYPLRYFTILTNALIKKICVTVSLRSYGTIFPII | 162 |
| | | + +\| ++ \| \|+\|\| +\|\|\|\| \| \| \| + + \| + + \| + | |
| Gnl\|pFAM\|PFAM0001: | 61 | GALFVVNOYASILLLTAISIDRYLAIVUPLRYRRIRTPRRAKVLILLVWVLALLLSLPPL | 120 |
| GPCR5: | 163 | FLLKRLTFCQNNIIPHTFCEHIGLAKYACNDIRINIWYGFSILMSTVVLDVVLIFISYML | 222 |
| | | \| + \| \| + + + + +\|+ \| +\|+ + | |
| Gnl\|Pfamp\|fam0001: | 121 | LFSWLRTVEEGNTTVCLIFFPEESVKRSYVLLSTLVGFVLPLLVILVCYTRILRTLRKRA | 180 |
| GPCR5: | 223 | ILHAVFHNPSPDACHKALNTFGSHVCIITLFYGSGIFTILTQRFOREIPPCIHIP--LAN | 280 |
| | | \| \| \| ++ + \| +\| \| + + | |
| Gnl\|Pfam\|pfam0001: | 181 | RSQRSLKRRSSSERKAAKMLLVVVVVFVLCWLPYHIVLLLDSLCLLSIWRVLPTALLITL | 240 |
| GPCR5: | 281 | VCILAPPMLNPIIY 294 | |
| | | \|\|\|\|\|\| | |
| Gnl\|Pfam\|pfam0001: | 241 | WLAYVNSCLNPIIY 254 | |

The protein similarity information, expression pattern, and map location for the olfactory receptor-like GPCR5 protein and nucleic acid disclosed herein suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders; control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) acrocallosal syndrome and dyskinesias, such as Huntington's disease or Gilles de la Tourette syndrome and/or other pathologies and disorders of the like.

The disclosed GPCR5 protein has multiple hydrophilic regions, each of which can be used as an immunogen. In one embodiment, a contemplated GPCR5 epitope is from about 170 to 175 amino acids. In additional embodiments, a GPCR5 epitope is from about amino acids 180 to 185, from about amino acids 190 to 192, from about amino acids 195 to 198, from 240 to 245, from about 290 to 295, from 298 to 300 and from 310 to 320. The GPCR5 protein also have value in the development of powerful assay system for functional analysis of various human disorders, which will help in understanding of pathology of the disease and development of new drug targets for various disorders.

GPCR6

The disclosed GPCR6 nucleic acid of 972 nucleotides (also referred to as CG50149-01) is shown in Table 6A. The disclosed GPCR6 open reading frame (“ORF”) begins with a ATG at nucleotides 18-20 and ending with a TGA codon at nucleotides 963-965. The start and stop codons of the open reading frame are highlighted in bold type. Putative untranslated regions, if any, are found upstream from the initiation codon and downstream from the termination codon.

TABLE 6A
GPCR6 nucleotide sequence.

(SEQ ID NO:17)

AAACTTGGACGATCGACATQGAAATTGTCTCCACAGGAAACGAAACTATTACTGAATTTGTCCTCCTTGGCTTCTAT
GACATCCCTGAACTGCATTTCTTGTTTTTTATTGTATTCACTGCTGTCTATGTCTTCATCATCATAGCGAATATGCT
GATTATTGTAGCAGTGGTTAGCTCCCAGAGGCTCCACAAACCCATGTATATTTTCTTGGCGAATCTGTCCTTCCTGG
ATATTCTCTACACCTCCGCAGTGATGCCAAAAATGCTGGAGGGCTTCCTGCAAGAAGCAACTATCTCTGTGGCTGGT
TGCTTGCTCCAGTTCTTTATCTTCQGCTCTCTAGCCACAGCTGAATGCTTACTGCTGGCTGTCATGGCATATGACCG
CTACCTGGCAATTTGCTACCCACTCCACTACCCACTCCTGATGGGGCCCAGACGGTACATGGGGCTGGTGGTCACAA
CCTGGCTCTCTGGATITGTGGTAGATGGACTGGTTGTGGCCCTGGTGGCCCAGCTGACGTTCTGTGGCCCCAACCAC
ATTGACCAGTTTTACTGTGACTTTATGCTTTTCGTGGGCCTGGCTTGCTCGGATCCCAGAGTGGCTCAGGTGACAAC
TCTCATTCTGTCTGTGTTCTGCCTCACTATTCCTTTTGGACTGATTCTGACATCTTATGCCAGAATTGTGGTGGCAG
TGCTGAGAGTTCCTGCTGGGGCAAGCAAGAGAAGGGCTTTCTCCACATGCTCCTCCCACCTAGCTGTAGTGACCACA
TTCTATGGAACGCTCATGATCTTTTATGTTGCACCCTCTGCTGTCCATTCCCAGCTCCTCTCCAAGGTCTTCTCCCT
GCTCTACACTGTGGTCACCCCTCTCTTCAATCCTGTGATCTATACCATGAGGAACAAGGAGGTGCATCAGGCACTTC
GGAAGATTCTCTGTATCAAACAAACTGAAACACTTGATTGAAGGAGAG

The GPCR6 the nucleic acid sequence of this invention has 595 of 707 bases (84%) identical to a gb:GENBANK-ID:AF044038|acc:AF044038.1 mRNA from Marmota marmota (Marmota marmota clone AMOR 6 olfactory receptor mRNA, partial cds) (E=5.0e⁻¹⁰⁸).

The disclosed GPCR6 polypeptide (SEQ ID NO: 18) encoded protein having 315 amino acid residues is presented in Table 6B using the one-letter amino acid code. The Signal P, Psort and/or Hydropathy results predict that GPCR6 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the microbody (peroxisome) with a certainty of 0.3000. The most likely cleavage site for a GPCR6 peptide is between amino acids 54 and 55, at: VSS-QR.

TABLE 6B
Encoded GPCR6 protein sequence

(SEQ ID NO:18)

MEIVSTGNETITEFVLLGFYDIPELHFLFFIVFTAVYVFIIIGNMLIIVAVVSSQRLHKPMYIFLANLSFLDILYTS
AVMPKMLEGFLQEATISVAGCLLQFFIFGSLATAECLLLAVMAYDRYLAICYPLHYPLLMGPRRYMGLVVTTWLSGF
VVDGLVVALVAQLRFCGPNHIDQFYCDFMLFVGLACSDPRVAQVTTLILSVFCLTIPFGLILTSYARIVVAVLRVPA
GASRRRAFSTCSSHLAVVTTFYGTLMIFYVAPSAVHSQLLSKVFSLLYTVVTPLFNPVIYTMRNKEVHQALRKILCI
KQTETLD

The disclosed GPCR6 amino acid sequence of the protein of the invention was found to have 140 of 297 amino acid residues (47%) identical to, and 188 of 297 amino acid residues (63%) similar to, the 324 amino acid residue ptnr:SPTREMBL-ACC:Q9WU86 protein from Mus musculus (Mouse) (ODORANT RECEPTOR S1) (E=1.2e⁻⁶⁸).

GPCR6 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

Possible SNPs found for GPCR6 are listed in Tables 6C.

TABLE 6C
SNPs

| | | Amino | |
|---|---|---|---|
| Nucleotide | Base | Acid | Base |
| Position | Change | Position | Change |
| 376 | C > T | 120 | Ala > Val |
| 810 | G > A | 265 | Ala > Thr |

The amino acid sequence of GPCR6 has high homology to other proteins as shown in Table 6D.

TABLE 6D
BLASTX results for GPCR6

| | | | Smallest |
|---|---|---|---|
| | | | Sum |
| Sequences producing High-scoring | | High | Prob |
| Segment Pairs: | Reading | Score | P (N) |
| Ptnr:SPTREMBL-ACC:Q9WU86 ODOR | 324 aa. | 698 | 1.2e−68 |
| RECP S1 - Mus musculus, | | | |

The disclosed GPCR6 has homology to the amino acid sequences shown in the BLASTP data listed Table 6E.

TABLE 6E
BLASTP results for GPCR6

| | | Length | Identity | Positives | |
|---|---|---|---|---|---|
| Gene Index/Identifier | Protein/Organism | (aa) | (%) | (%) | Expect |
| gi\|7363441\|ref\|NP_039 | olfactory | 315 | 315/315 | 315/315 | e−148 |
| 225.1\| | receptor, family | | (100%) | (100%) | |
| | 11, subfamily A, | | | | |
| | member 1 | | | | |
| | [Homo sapiens] | | | | |
| gi\|12054471\|emb CAC20 | olfactory | 315 | 314/315 | 314/315 | e−147 |
| 543.1\| | receptor | | (99%) | (99%) | |
| | [Homo sapiens] | | | | |
| gi\|5901488 gb AAD5530 | olfactory | 236 | 194/236 | 210/236 | 1e−88 |
| 9.1 AFC44038_1 | receptor | | (82%) | (88%) | |
| | [Marmota marmota] | | | | |
| gi\|7242165\|ref\|NP_035 | olfactory | 327 | 133/305 | 191/305 | 1e−61 |
| 113.1\| | receptor 41 | | (43%) | (62%) | |
| | [Mus musculus] | | | | |
| gi\|13928994\|ref NP_11 | olfactory | 327 | 133/307 | 194/307 | 6e−61 |
| 3898.1 | receptor 41 | | (43%) | (62%) | |
| | [Rattus norvegicus] | | | | |

The homology of these sequences is shown graphically in the ClustalW analysis shown in Table 6F.

[image]

[image]

Table 6G lists the domain description from DOMAIN analysis results against GPCR6. This indicates that the GPCR6 sequence has properties similar to those of other proteins known to contain this domain as well as to the 254 amino acid 7tm domain itself.

TABLE 6G

Domain Analysis of GPCR6

gnl|Pfam|pfam0001, 7tm_1, 7 transmembrane receptor (rhodopsin
family). (SEQ ID NO: 70)
Length = 254 residues, 100.0% aligned
Score = 99.8 bits (247), Expect = 2e−22

| | | | |
|---|---|---|---|
| GPCR6: | 43 | GNMLIIVAVVSSQRLHKPMYIFLANLSFLDILYTSAVMPKMLEGFLQEA-TISVAGCLLQ | 101 |
| | | \|\|+\|+\|+ ++ +++\| \| \|\|\| \|\|+ \|+\|+ + \| \| + \| \| \| | |
| Gnl\|Pfam\|pfam0001: | 1 | GNLLVILVILRTKKLRTPTNIFLLNLAVADLLFLLTLPPWALYYLVGGDNVFGOALCKLV | 60 |
| GPCR6: | 102 | FFIFGSLATAECLLLAVMAYDRYLAICYPLHYPLLMGPRRYMGLVVTTWLSGFVVDGLVV | 161 |
| | | +\| \| \|\|\| ++ \|\|\|\|\|\| +\|\| \| + \|\|\| \|++ \|+ + + | |
| Gnl\|Pfam\|pfam0001 | 61 | GALFVVNGYASILLLTAISIDRYLAIVEPLRYRRIRTPRRAKVLILLVNVLALLLSLPPL | 120 |
| GPCR6: | 162 | ALVAQLRFCGPNHIDQFYCDFMLFVGLACSDPRVAQVTTLILSVFCLTIPFGLILTSYAR | 221 |
| | | \| \| + \| + \|+ ++ +\| +\|\| \| \| | |
| Gnl\|Pfam\|pfam00001: | 121 | LFSWLRTVEEGN---TTVCLIDF------PEESVKRSYVLLSTLVGFVLPLLVILVCYTR | 171 |
| GPCR6: | 222 | IVVAVLRV---------PAGASRRRAFSTCSSHLAVVTTFYGTLMIFYVAP----SAVHS | 268 |
| | | \|+ + + + + \|+ \| + \| + ++ + \| | |
| Gnl\|Pfam\|pfam00001: | 172 | ILRTLRKRARSQRSLKRRSSSERKAAKMLLVVVVVFVLCWLPYHIVLLLDSLCLLSIWRV | 231 |
| GPCR6: | 269 | QLLSKVFSLLYTVVTPLFNPVIY 291 | |
| | | + + +\| \| \|\|+\|\| | |
| Gnl\|Pfam\|pfam00001: | 232 | LPTALLITLWLAYVNSCLNPIIY 254 | |

The protein similarity information, expression pattern, and map location for the olfactory receptor-like GPCR6 protein and nucleic acid disclosed herein suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) acrocallosal syndrome and dyskinesias, such as Huntington's disease or Gilles de la Tourette syndrome and/or other pathologies and disorders of the like.

The disclosed GPCR6 protein has multiple hydrophilic regions, each of which can be used as an immunogen. In one embodiment, a contemplated GPCR6 epitope is from about amino acids 160 to 165. In additional embodiments, a GPCR6 epitope is from about amino acids 230 to 235, from about amino acids 285 to 295, and from 305 to 310. The GPCR6 protein also have value in the development of powerful assay system for functional analysis of various human disorders, which will help in understanding of pathology of the disease and development of new drug targets for various disorders.

GPCR7

The disclosed GPCR7 nucleic acid of 1383 nucleotides (also referred to as GM_{—}33202597_A_da1) is shown in Table 7A. The disclosed GPCR7 open reading frame (“ORF”) begins with a ATG at nucleotides 6-8 and ending with a TGA codon at nucleotides 939-941. The start and stop codons of the open reading frame are highlighted in bold type. Putative untranslated regions, if any, are found upstream from the initiation codon and downstream from the termination codon.

TABLE 7A
GPCR7 nucleotide sequence.

(SEQ ID NO: 19)

TAGAAATGGAACGACCACAAAGTGATTTTAACCAAACTGAAGTTGCTCAATTTTTCCTCATGGGATTTTCGAATTCC
TGGGATATTCAGATTGTACATGCTGCTCTATTCTTCCTAGTTTACCTCGCAGCTGTCATAGGAAATCTCCTAATCAT
CATACTTACCACTCTGGATGTTCACCTCCAAACCCCAATGTATTTCTTTTTGAGAAACTTGTCTTTCTTAGATTTTT
GTTACATCTCTGTCACAATTCCAAAATCTATTGTTAGTTCCTTGACTCATGATACTTCCATTTCTTTCTTTGGGTGT
GCTCTGCAAGCCTTCTTTTTCATGGACTTGGCAACTACQGAQGTAQCCATCCTTACAGTGATGTCCTATGACCGCTA
TATGCCCATCTGCCGGCCTTTACATTATGAGGTCATCATAAACCAAGGTGTCTGTCTGACGATGATGGCCATGTCGT
GGCTCAGTGGGGTGATCTGTCGATTCATGCATGTGATAGCAACATTCTCATTACCATTCTGTGGGCGCAATAGAATA
CGTCAATTTTTCTGTAATATTCCACAGCTCCTAAGCCTCTTAGACCCCAAAGTAATTACCATTGAGATTGGAGTCAT
GGTTTTTGGTACAAGTCTTGTGATAATCTCCTTTGTTGTAATTACTCTCTCCTACATGTACATTTTTTCTGTCATCA
TGACGATTCCTTCTAAGGAGGGTAGATCAAAAACATTTTCTACCTGCATTCCACATCTTGTGGTTGTAACACTCTTT
ATGATATCTGGCAGCATTGCCTATGTGAAGCCAATTTCAAATTCTCCCCCCGTTCTGGATGTTTTCCTGTCTGCGTT
CTACACAGTCGTGCCCCCGACCCTGAACCCCGTCATCTATAGTCTGAGGAATAGGGACATGAAGGCAGCCCTGAGAA
GGCAGTGTGGTCCCTGAGAAGGCAGTGTGGTATCCTAGATGAACAATTTGATTACGGACCAGACTCTTGAACTCTTG
CTCTAATCAGGCAATTTQTAAACTCTCTGGGCTTATATTTTCAATTGATTGCTGAGCTCTTTCTTGATTTTTAAATT
AAATTCACCTATAGTTTACCATACTAACCATATTTGGGTGAATTCAACCTTAGAATGCACTAAAAGGTACTTCGGGG
TGTTCAAAACATTAGAACTTGTGCTTTTATTCATTTTTATGAGTTGTAGATCTCAAAATTTTATAAATTATATTGTA
CTCAGTAATACAACATATTAATTAATATAAAAGTGAATTACTAATATAAATGAATTACTACCGTATGTGTATATTAA
TTACAGCAGAACAAGATCATATGGATTAAAAACAACCAAATGGAAAATAAACATAAGGTAACAGTGTGTGCTCA

The disclosed GPCR7 of this invention maps to chromosome 1 the GPCR7 the nucleic acid sequence of this invention has 583 of 916 bases (63%) identical to a gb:GENBANK-ID:RATOLFPROQ|acc:M64391.1 mRNA from Rattus norvegicus (Rat olfactory protein mRNA, complete cds) (E=1.1e⁻⁴⁰). Chromosome localization information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

The disclosed GPCR7 polypeptide (SEQ ID NO: 20) encoded protein having 311 amino acid residues is presented in Table 7B using the one-letter amino acid code. The Signal P, Psort and/or Hydropathy results predict that GPCR7 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the mitochondrial inner membrane with a certainty of 0.0300. The most likely cleavage site for a GPCR7 peptide is between amino acids 59 and 60, at: VHL-QT.

TABLE 7B
Encoded GPCR7 protein sequence.

(SEQ ID NO: 20)

MERPQSDFNQTEVAEFFLMGFSNSWDIQIVHAALFFLVYLAAVIGNLLIIILTTLDVHLQTPMYFFLRNLSFLDFCY
ISVTIPKSIVSSLTHDTSISFFGCALQAFFFMDLATTEVAILTVMSYDRYMAICRPLHYEVIINQGVCLRMMAMSWL
SGVICGFMHVIATFSLPFCGRNRIRQFFCNIPQLLSLLDPKVITIETGVMVFGTSLVIISFVVITLSYMYIFSVIMR
IPSKEGRSKTFSTCIPHLVVVTLFMISGSIAYVKPISNSPPVLDVFLSAFYTVVPPTLNPVIYSLRNRDMKAALRRQ
CGP

The disclosed GPCR7 amino acid sequence of the protein of the invention was found to have 154 of 299 amino acid residues (51%) identical to, and 205 of 299 amino acid residues (68%) similar to, the 321 amino acid residue ptnr:SPTREMBL-ACC:Q9UGF5 protein from Homo sapiens (Human) (BA150A6.4 (NOVEL 7 TRANSMEMBRANE RECEPTOR (RHODOPSIN FAMYLY) (OLFACTORY RECEPTOR LIKE) PROTEIN (HS6M1-28) (E=2.7e⁻⁸⁰).

GPCR7 disclosed in this invention is expressed in at least the following tissues: hematopoietic tissues. This information was derived by determining the tissue sources of the sequences that were included in the invention including but not limited to SeqCalling sources.

Possible SNPs found for GPCR7 are listed in Tables 7C and 7D.

TABLE 7C
SNPs

| Consensus | | Base | |
|---|---|---|---|
| Position | Depth | Change | PAF |
| 419 | 16 | T > C | 0.250 |
| 699 | 37 | A > T | 0.054 |
| 821 | 54 | C > T | 0.074 |
| 929 | 58 | A > G | 0.052 |
| 961 | 51 | T > — | 0.059 |
| 972 | 51 | C > — | 0.039 |
| 1368 | 7 | A > — | 0.286 |

TABLE 7D
SNPs

| | | Amino | |
|---|---|---|---|
| Nucleotide | Base | Acid | Base |
| Position | Change | Position | Change |
| 459 | T > C | 152 | Ser > Pro |
| 566 | G > A | Silent | N/A |
| 967 | A > G | Silent | N/A |

The amino acid sequence of GPCR7 has high homology to other proteins as shown in Table 7E.

TABLE 7E
BLASTX results for GPCR7

| | | | Smallest |
|---|---|---|---|
| | | | Sum |
| Sequences producing High-scoring | | High | Prob |
| Segment Pairs: | Reading | Score | P (N) |
| ptnr:SPTREMBL-ACC:Q9UGF5 OLF | 321 aa. | 808 | 2.7e−80 |
| RECP - Homo sapiens, | | | |

The disclosed acid sequence of GPCR7 hs homology to the amino acid sequences shown in the BLAST data listed Table 7F

TABLE 7F
BLASTP results for GPCR7

| | | Length | Identity | Positives | |
|---|---|---|---|---|---|
| Gene Index/Identifier | Protein/Organism | (aa) | (%) | (%) | Expect |
| sp\|Q9UGF5 O5U1_HUMAN | OLFACTORY | 321 | 154/299 | 205/299 | 3e−85 |
| | RECEPTOR 5U1 | | (51%) | (68%) | |
| | (HS6M1-28) | | | | |
| emb\|CAC43449.1 | dM538M10.6 (novel | 321 | 152/299 | 205/299 | 1e−80 |
| | 7 transmembrane | | (50%) | (67%) | |
| | receptor | | | | |
| | (rhodopsin | | | | |
| | family) | | | | |
| | (olfactory | | | | |
| | receptor like) | | | | |
| | protein similar | | | | |
| | to | | | | |
| | human HS6M1-28) | | | | |
| | [Mus musculus] | | | | |
| emb\|CAC43446.1 | dM538M10.3 (novel | 304 | 146/292 | 198/292 | 1e−79 |
| | 7 transmembrane | | (50%) | (67%) | |
| | receptor | | | | |
| | (rhodopsin | | | | |
| | family) | | | | |
| | (olfactory | | | | |
| | receptor like) | | | | |
| | protein) | | | | |
| | [Mus musculus] | | | | |
| emb\|CAC43445.1 | dM538M10.2 (novel | 320 | 148/299 | 203/299 | 2e−78 |
| | 7 transmembrane | | (49%) | (67%) | |
| | receptor | | | | |
| | (rhodopsin | | | | |
| | family) | | | | |
| | (olfactory | | | | |
| | receptor like) | | | | |
| | protein) | | | | |
| | [Mus musculus] | | | | |
| emb\|CAC43448.1 | dM538M10.5 (novel | 312 | 142/299 | 202/299 | 9e−78 |
| | 7 transmembrane | | (47%) | (67%) | |
| | receptor | | | | |
| | (rhodopsin | | | | |
| | family) | | | | |
| | (olfactory | | | | |
| | receptor like) | | | | |
| | protein similar | | | | |
| | to | | | | |
| | human HS6M1-28) | | | | |
| | [Mus musculus] | | | | |

The homology of these sequences is shown graphically in the ClustalW analysis shown in Table 7G.

[image]

[image]

Table 7H lists the domain description from DOMAIN analysis results against GPCR7. This indicates that the GPCR7 sequence has properties similar to those of other proteins known to contain this domain as well as to the 254 amino acid 7tm domain itself.

TABLE 7H
Domain Analysis of GPCR7

gnl|Pfam|pfam00001, 7tm_1, 7 transmembrane receptor (rhodopsin
family). (SEQ ID NO: 76)
Length = 254 residues, 100.0% aligned
Score = 99.0 bits (245), Expect = 4e−22

| | | | |
|---|---|---|---|
| GPCR7: | 45 | GNLLIIILTTLDVHLQTPMYFFLRNLSPLDFCYISVTIPKSIVSSLTHDTSISFFGCALQ | 104 |
| | | \|\|\|\|+\|++ \|+\|\| \|\| \|\|+ \| ++ \| ++ + \| \| \| | |
| Gnl\|Pfam\|pfam00001: | 1 | GNLLVILVILRTKKLRTPTNIFLLNLAVADLLFLLTLPPWALYYLVGGDWVFGDALCKLV | 60 |
| GPCR7: | 105 | AFFFMDLATTEVAILTVMSYDRYMAICRPLHYEVIINQGVCLRMMAMSWLSGVICGFMHV | 164 |
| | | \|+ + +\|\| +\| \|\|\|+\|\| \|\| \| \| ++ + \|+ ++ + | |
| Gnl\|Pfam\|pfam00001: | 61 | GALFVVNGYASILLLTAISIDRYLAIVHPLRYRRIRTPRRRAKVLILLVWVLALLLSLPPL | 120 |
| GPCR7: | 165 | IATFSLPFCCGRNRIRQFFCNIPQLLSLLDPKVITIEIGVMVFGTSLVIISFVVITLSYMY | 224 |
| | | + ++ \| + + + \|+ \|+ \|\| + ++ \|\| | |
| Gnl\|Pfam\|pfam00001: | 121 | LFSWLRTVEEGNTTVCLIDFPEESVKRSYVLLSTLVGFVLPLLVILVCYTRILRTLRKRA | 180 |
| GPCR7: | 225 | IFSVIMRIPSKEGRSKTFSTCIPHLVVVTLFMISGSIAYVKPISNSPPVLDV----FLSA | 280 |
| | | ++ \| \| + +\| \| ++ + + + + ++ | |
| Fnl\|Pfam\|pfam00001: | 181 | RSQRSLKRRSSSERKAAKMLLVVVVVFVLCWLPYHIVLLLDSLCLLSIWRVLPTALLITL | 240 |
| GPCR7: | 281 | FYTVVPPTLNPVIY 294 | |
| | | + \| \|\|\|+\|\| | |
| Gnl\|Pfam\|pfam00001: | 241 | WLAYVNSCLNPIIY 254 | |

The protein similarity information, expression pattern, and map location for the olfactory receptor-like GPCR7 protein and nucleic acid disclosed herein suggest that this olfactory receptor may have important structural and/or physiological functions characteristic of the rhodopsin-like GPCR family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: disorders of the olfactory system reproductive system, dendritic cells, NK cells and mast cell/basophils (based on the expression of olfactory receptors like genes in these organs) and other diseases, disorders and conditions of the like and other diseases, disorders and conditions of the like.

Based on the Taqman results, the presence of the novel GPCR7 on NK cells implies some role for GPCR7 and its ligand in either the trafficking or the effector action of NK cells to sites of inflammation, transplantation, tumor sites or infection. As NK cells are of importance in the eradication of tumors as well as virally infected cells, GPCR7 may play a role in NK cell action in tumor rejection, where the ligand could be used to augment trafficking or effector action of NK cells. Alternatively a mAb could be used to block GPCR7 which could prove useful in transplantation of organs or bone marrow.

The presence of the orphan chemokine receptor on dendritic cells suggests that GPCR7 and its ligand could be involved in trafficking and localization of dendritic cells in lymphoid organs at sites of antigen presentation. Also ligand action on the dendritic cell may alter the antigen presenting properties of the dendritic cell and direct T cell activation to either Th1, Th2 or a Tr1 cell response. Therefore blockade of this orphan chemokine receptor with a mAb may alter the immune response in terms of magnitude and also the type of response generated. Alternatively the ligand could be used to modulate immune response. Due to this, mAb to the GPCR or its ligand may be of useful on redirecting the immune response in chronic inflammatory diseases such as Rheumatoid Arthritis, Inflammatory Bowel disease, Multiple Sclerosis, atopic dermatitis, allergic rhinitis and asthma between Th1 and Th2 responses.

The presence of GPCR7 on a cell line KU-812 which has properties of mast cell and basophils suggests that GPCR7 and its ligand may modulate mast cell and basophil trafficking and localization as well as their effector action. Therefore the ligand or a mAb to GPCR7 may prove useful in blocking mast cell action and therefore be of use in diseases/conditions such as asthma, allergy, inflammation and infections.

The disclosed GPCR7 protein has multiple hydrophilic regions, each of which can be used as an immunogen. In one embodiment, a contemplated GPCR7 epitope is from about amino acids 5 to 10. In additional embodiments, a GPCR7 epitope is from about amino acids 140 to 145, from about amino acids 170 to 175, from 240 to 250, and from 280 to 310. The GPCR7 protein also have value in the development of powerful assay system for functional analysis of various human disorders, which will help in understanding of pathology of the disease and development of new drug targets for various disorders.

GPCR8

The disclosed GPCR8 nucleic acid of 958 nucleotides (also referred to AC076959_da2) is shown in Table 8A. The disclosed GPCR8 open reading frame (“ORF”) begins with a ATG at nucleotides 4-7 and ending with a TGA codon at nucleotides 934-936. The start and stop codons of the open reading frame are highlighted in bold type. Putative untranslated regions, if any, are found upstream from the initiation codon and downstream from the termination codon.

TABLE 8A
GPCR8 nucleotide sequence.

(SEQ ID NO: 21)

AACATGGAAAGCAATCAGACCTGGATCACAGAAGTCATCCTGTTGGGATTCCAGGTGGACCCAGCTCTGGAGTTGTT
CCTCTTTGGGTTTTTCTTGCTATTCTACAGCTTAACCCTGATGGGAAATGGGATTATCCTGGGGCTCATCTACTTGG
ACTCTAGACTGCACACACCCATGTATGTCTTCCTGTCACACCTGGCCATTGTGGACATGTCCTATGCCTCGAGTACT
GTCCCTAAGATGCTAGCAAATCTTGTGATGCACAAAAAAGTCATCTCCTTTGCTCCTTGCATACTTCAGACTTTTTT
GTATTTGGCGTTTGCTATTACAGAGTGTCTGATTTTGGTGATGATGTGCTATGATCGGTATGTGGCAATCTGTCACC
CCTTGCAATACACCCTCATTATGAACTGGAGAQTGTCCACTGTCCTGGCCTCAACTTGCTCGATATTTAGCTTTCTC
TTGGCTCTGGTCCATATTACTCTTATTCTGAGGCTGCCTTTTTGTGGCCCACAAAAGATCAACCACTTTTTCTGTCA
AATCATGTCCGTATTCAAATTCCCCTGTGCTGACACTAGGCTCAACCAGGTGGTCCTATTTGCGCGTTCTGCGTTCA
TCTTAGTGGGGCCGCTCTGCCTGGTGCTGGTCTCCTACTTGCACATCCTGGTGGCCATCTTGAGGATCCAGTCTGGG
GAGGGCCGCAGAAAGGCCTTCTCTACCTGCTCCTCCCACCTCTGCGTGGTGGGGCTTTTCTTTGGCAGCGCCATTGT
CATGTACATGGCCCCCAGGTCAAACCATTCTCAAGAACGGAGGAAGATCCTTTCCCTGTTTTACAGCCTTTTCAACC
CGATCCTGAACCCCCTCATCTACAGCCTTAGGAATGCAGAGGTGAAAGGGGCTCTAAAGAGAGTCCTTTGGAAACAG
AGATCAATGTGAACAATCATTTGAGATATCCTGA

The GPCR8 the nucleic acid sequence of this invention has 716 of 944 bases (75%) identical to a gb:GENBANK-ID:AR092423|acc:AR092423.1 mRNA from Unknown (Sequence 1 from patent U.S. Pat. No. 5,998,164) (E=1.7e⁻¹⁰⁸).

The disclosed GPCR8 polypeptide (SEQ ID NO: 22) encoded protein having 310 amino acid residues is presented in Table 8B using the one-letter amino acid code. The Signal P, Psort and/or Hydropathy results predict that GPCR8 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the microbody (peroxisome) with a certainty of 0.3000. The most likely cleavage site for a GPCR8 peptide is between amino acids 46 and 47, at: ILG-LI.

TABLE 8B
Encoded GPCR8 protein sequence.

(SEQ ID NO: 22)

MESNQTWITEVILLGFQVDPALELFLFGFFLLFYSLTLMGNGIILGLIYLDSRLHTPMYVFLSHLAIVDMSYASSTV
PKMLANLVMHKKVISFAPCILQTFLYLAFAITECLILVMMCYDRYVAICHPLQYTLIMNWRVCTVLASTCWIFSFLL
ALVHITLILRLPFCCPQKINHFFCQIMSVFKLACALTRLNQVVLFAGSAFILVGPLCLVLVSYLHILVAILRIQSGE
GRRKAFSTCSSHLCVVGLFFGSAIVMYMAPRSNHSQERRKILSLFYSLFMPILNPLIYSLRNAEVKCALKRVLWKQR
SM

The disclosed GPCR8 amino acid sequence of the protein of the invention was found to have 188 of 310 amino acid residues (60%) identical to, and 238 of 310 amino acid residues (76%) similar to, the 310 amino acid residue ptnr:SPTREMBL-ACC:O95047 protein from Homo sapiens (Human) (WUGSC:H_DJ0988G15.2 PROTEIN) (E=1.1e⁻⁹⁸).

GPCR8 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

Possible SNPs found for GPCR8 are listed in Tables 8C and 8D

TABLE 8C
SNPs

| Consensus | | Base | |
|---|---|---|---|
| Position | Depth | Change | PAF |
| 147 | 11 | C > T | 0.182 |

TABLE 8D
SNPs

| | | Amino | |
|---|---|---|---|
| Nucleotide | Base | Acid | Base |
| Position | Change | Position | Change |
| 143 | T > C | 47 | Leu > Pro |
| 257 | T > C | 85 | Val > Ala |
| 374 | C > T | 124 | Ala > Val |
| 770 | T > C | 256 | Val > Ala |
| 788 | G > A | 262 | Arg > Lys |
| 794 | A > G | 264 | Asn > Ser |

The amino acid sequence of GPCR8 has high homology to other proteins as shown in Table 8E.

TABLE 8E
BLASTX results for GPCR8

| | | | Smallest |
|---|---|---|---|
| | | | Sum |
| Sequences producing High-scoring | | High | Prob |
| Segment Pairs: | | Score | P (N) |
| Ptnr:SPTREMBL-ACC:095407 Protein - | 310 aa. | 981 | 1.1e−98 |
| Homo sapiens, | | | |

The disclosed GPCR8 has homology to the amino acid sequences shown in the BLASTP data listed in Table 8F.

TABLE 8F
BLASTP results for GPCR8

| | | Length | Identity | Positives | |
|---|---|---|---|---|---|
| Gene Index/Identifier | Protein/Organism | (aa) | (%) | (%) | Expect |
| gi\|14747795\|ref XP_04 | hypothetical | 310 | 190/310 | 240/310 | 2e−86 |
| 2034.1 | protein XP_042034 | | (61%) | (77%) | |
| | [Homo sapiens] | | | | |
| gi\|13929212\|ref NP_11 | olfactory | 310 | 188/310 | 238/310 | 1e−85 |
| 2170.1 | receptor, family | | (60%) | (76%) | |
| | 2 , subfamily A, | | | | |
| | member 4 | | | | |
| | [Homo sapiens] | | | | |
| gi\|2921716\|gb\|AAC3963 | olfactory | 217 | 168/217 | 189/217 | 2e−80 |
| 3.1 | receptor | | (77%) | (86%) | |
| | [Homo sapiens] | | | | |
| gi\|2921710 gb AAC3963 | olfactory | 217 | 167/217 | 188/217 | 7e−80 |
| 0.1 | receptor | | (76%) | (85%) | |
| | [Homo sapiens] | | | | |
| gi\|15293775\|gb\|AAK950 | olfactory | 217 | 167/217 | 188/217 | 1e−79 |
| 80.1 | receptor | | (76%) | (85%) | |
| | [Homo sapiens] | | | | |

The homology of these sequences is shown graphically in the ClustalW analysis shown in Table 8G.

[image]

[image]

Table 8H lists the domain description from DOMAIN analysis results against GPCR8. This indicates that the GPCR8 sequence has properties similar to those of other proteins known to contain this domain as well as to the 254 amino acid 7tm domain itself.

TABLE 8H

Domain Analysis of GPCR8

gnl|Pfam|pfam00001, 7tm_1, 7 transmembrane receptor (rhodopsin
family). (SEQ ID NO: 82)
Length = 254 residues, 100.0% aligned
Score = 115 bits (288), Expect = 4e−27

| | | | |
|---|---|---|---|
| GPCR8: | 40 | GNGIILGLTYLDSRLETPMYVFLSHLAIVDMSYASSTVPKMLANLVMHKKVISPAPCILQ | 99 |
| | | \|\| +++ +\| +\| \|\| +\|\| +\|\|+ \|+ + + \| \| \|\| \| \| \| \| | |
| Gnl\|Pfam\|pfam00001: | 1 | GNLLVTLVILRTKKLRTPTNIFLLNLAVADLLFLLTLPPWALYYLVGGDWVFGDALCKLV | 60 |
| GPCR8: | 100 | TFLYLAFAITECLILVMMCYDRYVAICHPLQYTLIMNWRVCTVLASTCWIFSFLLALVHI | 159 |
| | | \|++ \|+\| + \|\|\|+\|\| \|\|\|+\| \| \| \|\| \|+ + \|\|+\| + | |
| Gnl\|Pfam\|pfam00001: | 61 | GALFVVNGYASILLLTAISIDRYLAIVHPLRYRRIRTPRRAKVLILLVWVLALLLSLPPL | 120 |
| GPCR8: | 160 | TLILRLPFCGPQKINHFFCQIMSVFKLACADTRLNQVVLFAGSAFILVGPLCLVLVSYLH | 219 |
| | | ++ \| \| \| + + + + \| \|\| ++\|\| \| | |
| Gnl\|Pfam\|pfam00001: | 121 | LFSWLRT---VEEGNTTVCLIDFPE------ESVKRSYVLLSTLVGFVLPLLVILVCYTR | 171 |
| GPCR8: | 220 | ILVA--------ILRIQSGEGRRKAFSTCSSHLCVVGLFFGSAIVMYMAP----RSNHS | 266 |
| | | \|\| \|+ +\| \|+ \| + \| + \|\|+ + | |
| Gnl\|Pfam\|pfam00001: | 172 | ILRTLRKRARSQRSLKRRSSSERKAAKMLLVVVVVFVLCWLPYHIVLLDSLCLLSIWRV | 231 |
| GPCR8: | 267 | QERRKILSLFYSLFNPILNPLIY 289 | |
| | | +++\|+ + \| \|\|\|+\|\| | |
| Gnl\|Pfam\|pfam00001: | 232 | LPTALLITLWLAYVNSCLNPIIY 254 | |

The protein similarity information, expression pattern, and map location for the olfactory receptor-like GPCR8 protein and nucleic acid disclosed herein suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: familial Mediterranian fever, developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) acrocallosal syndrome and dyskinesias, such as Huntington's disease or Gilles de la Tourette syndrome and/or other pathologies and disorders of the like.

The disclosed GPCR8 protein has multiple hydrophilic regions, each of which can be used as an immunogen. In one embodiment, a contemplated GPCR8 epitope is from about amino acids 230 to 240. In additional embodiments, a GPCR8 epitope is from about amino acids 260 to 270, from about amino acids 290 to 300. The GPCR8 protein also have value in the development of powerful assay system for functional analysis of various human disorders, which will help in understanding of pathology of the disease and development of new drug targets for various disorders.

GPCR9

The disclosed GPCR9 nucleic acid of 968 nucleotides (also referred to as AC073364_da1) is shown in Table 9A. The disclosed GPCR9 open reading frame (“ORF”) begins with a ATG at nucleotides 16-18 and ending with a TAA codon at nucleotides 961-963. The start and stop codons of the open reading frame are highlighted in bold type. Putative untranslated regions, if any, are found upstream from the initiation codon and downstream from the termination codon.

TABLE 9A
GPCR9 nucleotide sequence.

(SEQ ID NO:23)

AGCTCTACTAGAAATATGGAGAGCAGAAACCAATCAACAGTGACTGAATTTATCTTCACTGGATTCCCTCAGCTTCA
GGATGGTAGTCTCCTGTACTTCTTTCCTTTACTTTTCATCTATACTTTTATTATCATTGATAACTTATTAATCTTCT
CTGCTGTAAGGCTGGACACCCATCTCCACAACCCCATGTATAATTTTATCAGTATATTTTCCTTTCTGGAGATCTGG
TACACCACAGCCACCATTCCCAAGATGCTCTCCAACCTCATCAGTGAAAGAAGGCCATCTCAATGACTGGCTGCAT
CTTGCAGATGTATTTCTTCCACTCACTTGAAAACTCAGAGGGGATCTTGCTGACCACCATGGCCATTGACAGATACG
TTGCCATCTGCAACCCTCTTCGCTATCAAATGATCATGACCCCCCGGCTCTGTGCTCAACTCTCTGCAGGTTCCTGC
CTCTTCGGTTTCCTTATCCTGCTTCCCGAGATTGTGATGATTTCCACACTGCCTTTCTGTGGGCCCAACCAAATCCA
TCAGATCTTCTGTGACTTGGTCCCTGTGCTAAGCCTGGCCTGTACAGACACGTCCATGATTCTGATTGAGGATGTGA
TTCATGCTGTGACCATCATCATTACCTTCCTAATCATTGCCCTGTCCTATGTAAGAATTGTCACTGTGATATTGAGG
ATTCCCTCTTCTGAAGGGAGGCAAAGGCTTTTTCTACCTGTGCAGGCCACCCCATGGTCTTCCCGATATTCTTTGG
CAGTGTATCACTCATGTACTTGCGTTTCAGCGACACTTATCCACCAGTTTTGGACACACCCATTGCACTGATGTTTA
CTGTACTTGCTCCATTCTTCAATCCCATCATTTATAGCCTGAGAAACAAGGACATGAACAATGCGATTAAAAAACTG
TTCTGTCTTCAAAAAGTGTTGAACAAGCCTGGAGGTTAATACAG

The GPCR9 the nucleic acid sequence of this invention has 670 of 954 bases (70%) identical to a gb:GENBANK-ID:AP002533|acc:AP002533.1 mRNA from Homo sapiens (Homo sapiens genomic DNA, chromosome 1q22-q23, CD1 region, section 2/4 (E=1.5e⁻⁸⁷).

The disclosed GPCR9 polypeptide (SEQ ID NO: 24) encoded protein having 315 amino acid residues is presented in Table 3B using the one-letter amino acid code. The Signal P, Psort and/or Hydropathy results predict that GPCR9 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the mitochondrial inner membrane with a certainty of 0.3000. The most likely cleavage site for a GPCR9 peptide is between amino acids 50 and 51, at: AVR-LD.

TABLE 9B
Encoded GPCR9 protein sequence.

(SEQ ID NO:24)

MESRNQSTVTEFIFTGFPQLQDGSLLYFFPLLFIYTFIIIDNLLIFSAVRLDTHLHNPMYNFISIFSFLEIWYTTAT
IPKMLSNLISEKKAISMTGCILQMYFFHSLENSEGILLTTMAIDRYVAICNPLRYQMIMTPRLCAQLSAGSCLFGFL
ILLPEIVMISTLPFCGPNQIHQIFCDLVPVLSLACTDTSMILIEDVIHAVTIIITFLIIALSYVRIVTVILRIPSSE
GRQKAFSTCAGHPMVFPIFFGSVSLMYLRFSDTYPPVLDTAIALMFTVLAPFFNPIIYSLRNKDMNNAIKKLFCLQK
VLNKPGG

The disclosed GPCR9 amino acid sequence of the protein of the invention was found to have 135 of 304 amino acid residues (44%) identical to, and 192 of 304 amino acid residues (63%) similar to, the 321 amino acid residue ptnr:SPTREMBL-ACC:Q9UGF6 protein from Homo sapiens (Human) (BA150A6.2 (NOVEL 7 TRANSMEMBRANE RECEPTOR (RHODOPSIN FAMILY) (OLFACTORY RECEPTOR LIKE) PROTEIN (HS6M1-21) (E=1.7e⁻⁶⁵).

GPCR9 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue. Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

Possible SNPs found for GPCR9 are listed in Tables 9C. The SNP located at 758 bp is also referred to as variant 13374101.

TABLE 9C
SNPs

| | | Amino | |
|---|---|---|---|
| Nucleotide | Base | Acid | Base |
| Position | Change | Position | Change |
| 25 | A > G | 4 | Arg > Gly |
| 186 | C > T | Silent | N/A |
| 468 | C > T | Silent | N/A |
| 697 | C > T | 228 | Pro > Ser |
| 713 | G > A | 233 | Arg > Lys |
| 758 | C > T | 248 | Pro > Leu |

The amino acid sequence of GPCR9 has high homology to other proteins as shown in Table 9D.

TABLE 9D
BLASTX results for GPCR9

| | | | Smallest |
|---|---|---|---|
| | | | Sum |
| Sequences producing High-scoring | | High | Prob |
| Segment Pairs: | | Score | P (N) |
| ptnr:SPTREMBL-ACC:Q9UGF6 OLF | 321 aa. | 668 | 1.7e−65 |
| RECP - Homo sapiens, | | | |

The disclosured GPCR9 has homology to the amino acid sequences shown in the BLASTP data listed in Table 9E.

TABLE 9E
BLASTP results for GPCR9

| | | Length | Identity | Positives | |
|---|---|---|---|---|---|
| Gene Index/Identifier | Protein/Organism | (aa) | (%) | (%) | Expect |
| gi\|13562084\|ref\|NP_11 | olfactory | 321 | 135/304 | 192/304 | 9e−59 |
| 0503.1 | receptor, family | | (44%) | (62%) | |
| | 5, subfamily V | | | | |
| | member 1 | | | | |
| | [Homo sapiens] | | | | |
| gi\|7242165\|ref\|NP_035 | olfactory | 327 | 131/308 | 190/308 | 8e−58 |
| 113.1\| | receptor 41 | | (42%) | (61%) | |
| | [Mus musculus] | | | | |
| gi\|13928994\|ref NP_11 | olfactory | 327 | 134/308 | 191/308 | 2e−57 |
| 3898.1\| | receptor 41 | | (43%) | (61%) | |
| | [Rattus norvegicus] | | | | |
| gi\|12007416\|gb\|AAG451 | m51 olfactory | 314 | 139/303 | 189/303 | 2e−57 |
| 89.1 | receptor | | (45%) | (61%) | |
| | [Mus musculus] | | | | |
| gi\|14596246\|emb\|CAC43 | dM538M10.1 (novel | 317 | 134/306 | 186/306 | 3e−57 |
| 444.1\| | 7 transmembrane | | (43%) | (59%) | |
| | receptor | | | | |
| | (rhodopsin | | | | |
| | family) | | | | |
| | (olfactory | | | | |
| | receptor like) | | | | |
| | protein similar | | | | |
| | to | | | | |
| | human HS6M1-21) | | | | |
| | [Mus musculus] | | | | |

The homology of these sequences is shown graphically in the ClustalW analysis shown in Table 9F.

[image]

[image]

Table 9G lists the domain description from DOMAIN analysis results against GPCR9. This indicates that the GPCR9 sequence has properties similar to those of other proteins known to contain this domain as well as to the 254 amino acid 7tm domain itself.

TABLE 9G
Domain Analysis of GPCR9

gnl|Pfam|pfam00001, 7tm_1, 7 transmembrane receptor (rhodopsin
family). (SEQ ID NO:88)
Length = 254 residues, 99.6% aligned
Score = 95.5 bits (236), Expect = 4e−21

| | | | |
|---|---|---|---|
| GPCR9: | 42 | NLLIFSAVRLDTHLHNPMYNFISTFSFLEIWYTTATIPKMLSNLISEKKAISMTGCILQM | 101 |
| | | \|\|\|+ + \| \| \|+ + ++ + \| \| \|+ \| \| | |
| Gnl\|Pfam\|pfam00001: | 2 | NLLVILVILRTKKLRTPTNFLLNAVADLLFLLTLPPWALYYLVGGDWVFGDALCKLVG | 61 |
| GPCR9: | 102 | YFFHSLENSEGILLTTMAIDRYVAICNPLRYQMIMTPRLCAQLSAGSCLFGFLILLPEIV | 161 |
| | | \| + +\|\|\| ++\|\|\|\|+\|\| +\|\|\|\|+ \| \|\|\| \| + \|+ \|\| ++ | |
| Gnl\|Pfam\|pfam00001: | 62 | ALFVVNGYASILLLTAISIDRYLAIVHPLRYRRIRTPRRAKVLILLVWVLALLLSLPPLL | 121 |
| GPCR9 | 162 | MISTLPFCGPNQIHQIFCDLVPVLSLACTDTSMILIEDVIHAVTIIITFLI-IALSYVRI | 220 |
| | | \| + + + \| ++ + \| ++ \| \| | |
| Gnl\|Pfam\|pfam00001: | 122 | FSWLRTVEEGNTTVCLIDFPEESVKRSYVLLSTLVGFVLPLLVILVCYTRILRTLRKRAR | 181 |
| GPCR9: | 221 | VTVILRIPSSEGRQKAFSTCAGHPMVF----PIFFGSVSLMYLRFSDTYPPVLDTAIALM | 276 |
| | | \|+ \|\| \|+ \| + \| + \| \| \| | |
| Gnl\|Pfam\|pfam00001: | 182 | SQRSLKRRSSSERKAAKMLLVVVVVFVLCWLPYHIVLLLDSLCLLSIWRVLPTALLITLW | 241 |
| GPCR9: | 277 | FTVLAPFFNPIIY | 289 |
| | | + \|\|\|\|\| | |
| Gnl\|Pfam\|pfam00001: | 242 | LAYVNSCLNPIIY | 264 |

The protein similarity information, expression pattern, and map location for the olfactory receptor-like GPCR9 protein and nucleic acid disclosed herein suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: familial Mediterranian fever, developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy (DRPLA) hypophosphatemic rickets, autosomal dominant (2) acrocallosal syndrome and dyskinesias, such as Huntington's disease or Gilles de la Tourette syndrome and/or other pathologies and disorders of the like.

The disclosed GPCR9 protein has multiple hydrophilic regions, each of which can be used as an immunogen. In one embodiment, a contemplated GPCR9 epitope is from about amino acids 50 to 10. In additional embodiments, a GPCR9 epitope is from about amino acids 60 to 65, from about amino acids 140 to 145, from 240 to 242, and from 290 to 310. The GPCR9 protein also have value in the development of powerful assay system for functional analysis of various human disorders, which will help in understanding of pathology of the disease and development of new drug targets for various disorders.

GPCR10

The disclosed GPCR10 nucleic acid of 928 nucleotides (also referred to as CG55732-01) is shown in Table 10A. The disclosed GPCR10 open reading frame (“ORF”) begins with a TTT codon which codes for the amino acid phenylalanine at nucleotides 24 and ending with a TAA codon at nucleotides 845-847. The start and stop codons of the open reading frame are highlighted in bold type. Putative untranslated regions, if any, are found upstream from the initiation codon and downstream from the termination codon.

TABLE 10A
GPCR10 nucleotide sequence.

(SEQ ID NO:25)

GTTTTCTGTCATCTACATCAACGCCATGATAGGAAATGTGCTCATTGTGGTCACCATCACTGCCAGCCCATCACTGA
GATCCCCCATGTACTTTTTCCTGGCCTATCTCTCCTTTATTGATGCCTGCTATTCCTCTGTCAATGCCCCTAAGCTG
ATCACAGATTCACTCTATGAAAACAAGACTATCTTATTCAATGGATGTATGACTCAAGTCTTTGGAGAACATTTTTT
CAGAGGTGTTGAGGTCATCCTACTTACTGTAATGGCCTATGACCACTATGTGGCCATCTGCAAGCCCTTGCACTATA
CCACCATCATGAAGAAGCATGTTTGTAGCCTGCTAGTGGGAGTGTCATGGGTAGGAGGCTTTCTTCATGCAACCATA
CAGATCCTCTTCATCTGTCAATTACCTTTCTGTGGTCCTAATGTCATAGATCACTTTATGTGTGATCTCTACACTTT
GATCAATCTTGCCTGCACTAATACCCACACTCTAGGACTCTTCATTGCTGCCAACAGTGGGTTCATATGCCTGTTAA
ACTGTCTCTTGCTCCTGGTCTCCTGCGTGCTCATACTGTACTCCTTAAAGACCCACAGCTTAGAGGCAAGCCACGAA
GCCCTCTCTACCTGTGTCTCCCACATCACAGTTGTCATCTTATCCTTTATACCCTGCATATTTGTGTACATGACACC
TCCAGCTACTTTACCCATTGATAAAGCAGTTGCTGTATTCTACACTATGATAACTTCTATGTTAAACCCCTTAATCT
ACACCTTGAGGAATGCTCAAATGAAAAATGCCATTAGGAAATTGTGTAGTAGGAAAGCTATTTCAAGTGTCAAATAA
ATGTGACTGGAGCCCAACATGATTCAACTGAGGCAAGGGTCAAAAGGACATTTTGGGTAATGCCAGCAAAGGAATAC
TTAT

The GPCR10 the nucleic acid sequence of this invention has 575 of 821 bases (70%) identical to a gb:GENBANK-ID:AB030896|acc:AB030896.1 mRNA from Mus musculus (Mus musculus gene for odorant receptor A16, complete cds) (E=3.0e⁻⁷⁶).

The disclosed GPCR10 polypeptide (SEQ ID NO: 26) encoded protein having 281 amino acid residues is presented in Table 10B using the one-letter amino acid code. The Signal P, Psort and/or Hydropathy results predict that GPCR10 has a signal peptide and is likely to be localized at the plasma membrane with a certainty of 0.6400. In other embodiments, it is localized at the Golgi body with a certainty of 0.4600, at the endoplasmic reticulum (membrane) with a certainty of 0.3700 or at the endoplasmic reticulum (lumen) with a certainty of 0.1000. The most likely cleavage site for a GPCR1 0 peptide is between amino acids 44 and 45, at: CYS-SV.

TABLE 10B
Encoded GPCR10 protein sequence.

(SEQ ID NO:26)

FSVIYINAMIGNVLIVVTITASPSLRSPMYFFLAYLSFIDACYSSVNAPKLITDSLYENKTILFNGCMTQVFGEHFF
RGVEVILLTVMAYDHYVAICKPLHYTTIMKKHVCSLLVGVSWVGGFLHATIQILFICQLPFCGPNVIDHPMCDLYTL
INLACTNTHTLGLFIAANSGFICLLNCLLLLVSCVVILYSLKTHSLEAREEALSTCVSHITVVILSFIPCIFVYMRP
PATLPIDKAVAVFYTMITSMLNPLIYTLRNAQMKNAIRKLCSRKAISSVK

The disclosed GPCR10 amino acid sequence of the protein of the invention was found to have 182 of 277 amino acid residues (65%) identical to, and 221 of 277 amino acid residues (79%) similar to, the 308 amino acid residue ptnr:SPTREMBL-ACC:Q9R0K2 protein from Mus musculus (Mouse) (ODORANT RECEPTOR MOR18) (E=7.9e⁻⁹⁹).

GPCR10 disclosed in this invention is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources.

Possible SNPs found for GPCR10b are listed in Table 10C.

TABLE 10C
SNPs

| Consensus | | Base | |
|---|---|---|---|
| Position | Depth | Change | PAF |
| 611 | 7 | T > G | 0.429 |

The amino acid sequence of GPCR10 has high homology to other proteins as shown in Table 10D.

TABLE 10D
BLASTX results for GPCR10

| | | | Smallest |
|---|---|---|---|
| | | | Sum |
| Sequences producing High-scoring | | High | Prob |
| Segment Pairs: | | Score | P (N) |
| ptnr:SPTREMBL-ACC:Q9R0K2 | 308 aa. | 983 | 7.9e−99 |
| ODOR RECP - Mus Musculus, | | | |

The disclosed GPCR 10 has homology to the amino acid sequences shown in the BLASTP data listed in Table 10E.

TABLE 10E
BLASTP results for GPCR10

| | | Length | Identity | Positives | |
|---|---|---|---|---|---|
| Gene Index/Identifier | Protein/Organism | (aa) | (%) | (%) | Expect |
| gi\|15293735\|gb\|AAK950 | olfactory | 213 | 211/213 | 212/213 | 1e−95 |
| 60.1 | receptor | | (99%) | (99%) | |
| | [Homo sapiens] | | | | |
| gi\|11496249\|ref NP_06 | odorant receptor | 308 | 182/277 | 221/277 | 3e−89 |
| 7343.1 | 16 [Mus musculus] | | (65%) | (79%) | |
| gi\|11464995\|ref NP_06 | gene for odorant | 302 | 169/271 | 214/271 | 1e−84 |
| 5261.1 | receptor A16 | | (62%) | (78%) | |
| | [Mus musculus] | | | | |
| gi\|423702\|pir \|S29710 | olfactory | 307 | 162/271 | 204/271 | 1e−73 |
| | receptor OR18 - | | (59%) | (74%) | |
| | rat | | | | |
| gi\|3983372 gb AAD1331 | olfactory | 220 | 157/220 | 177/220 | 6e−73 |
| 4.1 | receptor C3 | | (71%) | (80%) | |
| | [Mus musculus] | | | | |

The homology of these sequences is shown graphically in the ClustalW analysis shown in Table 10F.

[image]

[image]

Table 10G lists the domain description from DOMAIN analysis results against GPCR10. This indicates that the GPCR10 sequence has properties similar to those of other proteins known to contain this domain as well as to the 254 amino acid 7tm domain itself.

TABLE 10G
Domain Analysis of GPCR10

gnl|Pfam|pfam00001:, 7tm_1, 7 transmembrane receptor (rhodopsin
family). (SEQ ID NO:94)
Length = 254 residues, 100.0% aligned
Score = 157/220 (71%), Expect= 1e−18

| | | | |
|---|---|---|---|
| GPCR10: | 11 | GNVLIVVTITASPSLRSPMYFFLAYLSFIDACYSSVNAPKLITDSLYENKTILFNGCMTQ | 70 |
| | | \|\|+\|+++ \| + \|\|+\| \|\| \|+ \| + \| + + + \| | |
| Gnl\|Pfam\|pfam00001: | 1 | GNLLVILVILRTKKLRTPTNIFLLNLAVADLLFLLTLPPWALYYLVGGDWVFGDALCKLV | 60 |
| GPCR10: | 71 | VFGEHFFRGVEVILLTVMAYDHYVAICKPLHYTTIMKKHVCSLLVGVSWVGGFLHATIQI | 130 |
| | | ++\|\|\| ++ \| \|+\|\| \|\| \| \| +\|+ + \|\| \| + + | |
| Gnl\|Pfam\|pfarm00001: | 61 | GALFVVNGYASILLLTAISIDRYLAIVHPLRYRRIRTPRRAKVLILLVWVLALLLSLPPL | 120 |
| GPCR10: | 131 | LFICQLPFCGPNVTDEFMCDLYTLTNACTNTHTLGLFIAANSGFICLLNCLLLLVSCVV | 190 |
| | | \|\| \| + + + \|\| \|+ + +\| + | |
| Gnl\|Pfam\|pfam00001: | 121 | LFSWLRTVEEGNTTVCLIDFPEESVKRSYVLLSTLVGFVLPLLVILVCYTRILRTLRKRA | 180 |
| GPCR10: | 191 | ILYSLKTHSLEARHEALSTCVSHITVVILSFIPC-IFVYMRP------PATLPIDKAVAV | 243 |
| | | + +\| + + \| +\| ++\| \| + + \|\| + + | |
| Gnl\|Pfam\|pfam00001: | 181 | RSQRSLKRRSSSERKAAKMLLVVVVVFVLCWLPYHIVLLLDSLCLLSIWRVLPTALLITL | 240 |
| GPCR10: | 244 | FYTMITSMLNPLIY | 257 |
| | | + + \| \|\|\|+\|\| | |
| Gnl\|Pfam\|pfam00001: | 241 | WLAYVNSCLNPIIY | 254 |

The protein similarity information, expression pattern, and map location for the olfactory receptor-like GPCR10 protein and nucleic acid disclosed herein suggest that this olfactory receptor may have important structural and/or physiological functions characteristic of the olfactory receptor family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) acrocallosal syndrome and dyskinesias, such as Huntington's disease or Gilles de la Tourette syndrome and/or other pathologies and disorders of the like.

The disclosed GPCR10 protein has multiple hydrophilic regions, each of which can be used as an immunogen. In one embodiment, a contemplated GPCR10 epitope is from about amino acids 50 to 55. In additional embodiments, a GPCR10 epitope is from about amino acids 57 to 60, from about amino acids 60 to 62, from 67 to 70, from 95 to 105, from 195 to 205 and from 260 to 275. The GPCR10 protein also have value in the development of powerful assay system for functional analysis of various human disorders, which will help in understanding of pathology of the disease and development of new drug targets for various disorders.

GPCR11

The disclosed GPCR11 nucleic acid of 971 nucleotides (also referred to as sggc_draft_ba656o22_20000731_da1) is shown in Table 11A. The disclosed GPCR11 open reading frame (“ORF”) begins with a ATG at nucleotides 3-5 and ending with a TAG codon at nucleotides 963-965. The start and stop codons of the open reading frame are highlighted in bold type. Putative untranslated regions, if any, are found upstream from the initiation codon and downstream from the termination codon.

TABLE 11A
GPCR11 nucleotide sequence.

(SEQ ID NO:27)

CAATGATGGAAATAGCCAATGTGAGTTCTCCAGAAGTCTTTGTCCTCCTGGGCTTCTCCGCACGACCCTCACTAGAA
ACTGTCCTCTTCATAGTTGTCTTGAGTTTTTACATGGTATCGATCTTGGGCAATGGCATCATCATTCTGGTCTCCCA
TACAGATGTGCACCTCCACACACCTATGTACTTCTTTCTTGCCACCTCTCCTTCCTGGACATGAGCTTCACCACGA
GCATTGTCCCACAGCTCCTGGCTAACCTCTGGGGACCACAGAAAACCATAAGCTATGGAGGGTGTGTGGTCCAGTTC
TATATCTCCCATTGGCTGGGGGCAACCGAGTGTGTCCTGCTGGCCACCATGTCCTATGACCGCTACGCTGCCATCTG
CAGGCCACTCCATTACACTGTCATTATGCATCCACAGCTTTGCCTTCGGCTAGCTTTGGCCTCCTGGCTGGGGGGTC
TGACCACCAGCATGGTGGGCTCCACGCTCACCATGCTCCTACCGCTGTGTGGGAACAATTGCATCGACCACTTCTTT
TGCGAGATGCCCCTCATTATGCAACTGGCTTGTGTGGATACCAGCCTCAATGAGATGGAGATGTACCTGGCCAGCTT
TGTCTTTGTTGTCCTGCCTCTGGGGCTCATCCTGGTCTCTTACGGCCACATTGCCCGGGCCGTGTTGAAGATCAGGT
CAGCAGAAGGGCGGAGAAAGGCATTCAACACCTGTTCTTCCCACGTGGCTGTGGTGTCTCTGTTTTACGGGAGCATC
ATCTTCATGTATCTCCAGCCAGCCAAGAGCACCTCCCATGAGCAGGGCAAGTTCATAGCTCTGTTCTACACCGTAGT
CACTCCTGCGCTGAACCCAGTTATTTACAACCTGAGGAACACGGAGGTGAAGAGCGCCCTCCGGCACATGGTATTAG
AGAACTGCTGTGGCTCTGCAGGCAAGCTGGCGCAAATTTAGAGACTC

The GPCR11 the nucleic acid sequence has 601 of 906 bases (66%) identical to a Homo sapiens olfactory receptor-like protein (OR2C1) gene mRNA (GENBANK-ID: AF098664|acc:AF098664) (E=1.9e⁻⁶⁷).

The disclosed GPCR11 polypeptide (SEQ ID NO: 28) encoded protein having 320 amino acid residues is presented in Table 3B using the one-letter amino acid code.

TABLE 11B
Encoded GPCR11 protein sequence.

(SEQ ID NO:28)

MMEIANVSSPEVFVLLGFSARPSLETVLFIVVLSFYMVSILGNGIIILVSHTDVHLHTPMYFFLANLSFLDMSFTTS
IVPQLLANLWGPQKTISYGGCVVQFYISHWLGATECVLLATMSYDRYAAICRPLHYTVIMHPQLCLGLALASWLGGL
TTSMVGSTLTMLLPLCGNNCIDHFFCEMPLIMQLACVDTSLNEMEMYLASFVFVVLPLGLILVSYGHIARAVLKIRS
AEGRRKAFNTCSSHVAVVSLFYGSIIFMYLQPAKSTSHEQGKFIALFYTVVTPALNPVIYNLRNTEVKSALRHMVLE
NCCGSAGKLAQI

The disclosed GPCR11 amino acid sequence of the protein of the invention was found to have 181 of 305 amino acid residues (59%) identical to, and 232 of 305 residues (76%) positive with, the 312 amino acid residue OLFACTORY RECEPTOR 15 (OR3) protein from Mus musculus (ptnr: SWISSPROT-ACC:P23275) (E=1.8e⁻⁹⁷).

Possible SNPs found for GPCR11 are listed in Tables 11C.

TABLE 11C
SNPs

| | | Amino | |
|---|---|---|---|
| Nucleotide | Base | Acid | Base |
| Position | Change | Position | Change |
| 71 | A > G | Silent | N/A |
| 185 | C > T | Silent | N/A |
| 389 | G > C | 129 | Arg > Ser |
| 541 | G > A | 180 | Cys > Tyr |
| 780 | T > C | 260 | Tyr > His |

The amino acid sequence of GPCR11 has high homology to other proteins as shown in Table 11D.

TABLE 11D
BLASTX results for GPCR11

| | | | Smallest |
|---|---|---|---|
| | | | Sum |
| Sequences producing High-scoring | | High | Prob |
| Segment Pairs: | | Score | P (N) |
| ptnr:SWISSPROT-ACC:P23725 OLF | 312 aa. | 977 | 1.8e−97 |
| RECP - Mus Musculus, | | | |

The disclosed GPCR11 has homology to the amino acid sequences shown in the BLASTP data listed in Table 11E.

TABLE 11E
BLASTP results for GPCR11

| | | Length | Identity | Positives | |
|---|---|---|---|---|---|
| Gene Index/Identifier | Protein/Organism | (aa) | (%) | (%) | Expect |
| gi\|6679170 ref\|NP_032 | olfactory | 312 | 181/305 | 232/305 | 3e−96 |
| 788.1\| | receptor 15 | | (59%) | (75%) | |
| | [Mus musculus] | | | | |
| gi\|13624329\|ref\|NP_11 | olfactory | 320 | 172/305 | 229/305 | 2e−92 |
| 2165.1 | receptor, family | | (56%) | (74%) | |
| | 2, subfamily W, | | | | |
| | member 1 | | | | |
| | [Homo sapiens] | | | | |
| gi\|12054431\|emb CAC20 | olfactory | 320 | 172/305 | 229/305 | 3e−92 |
| 523.1\| | receptor | | (56%) | (74%) | |
| | [Homo sapiens] | | | | |
| gi\|12054355\|emb CAC20 | olfactory | 312 | 175/306 | 230/306 | 4e−92 |
| 485.1\| | receptor | | (57%) | (74%) | |
| | [Homo sapiens] | | | | |
| gi\|12054429\|emb CAC20 | olfactory | 320 | 172/305 | 228/305 | 5e−92 |
| 522.1\| | receptor [Homo sapiens] | | (56%) | (74%) | |

The homology of these sequences is shown graphically in the ClustalW analysis shown in Table 11F.

[image]

[image]

Table 11G lists the domain description from DOMAIN analysis results against GPCR11. This indicates that the GPCR11 sequence has properties similar to those of other proteins known to contain this domain as well as to the 254 amino acid 7tm domain itself.

TABLE 11G
Domain Analysis of GPCR11

gnl|Pfam|pfam00001, 7tm_1, 7 transmembrane receptor (rhodopsin
family). (SEQ ID NO:100)
Length = 254 residues, 100.0% aligned
Score = 102 bits (254), Expect = 3e−23

| | | | |
|---|---|---|---|
| GPCR11: | 42 | GNGIIILVSHTDVHLHTPMYFFLANLSFLDMSFTTSIVPQLLANLWGPQKTISYGGCVVQ | 101 |
| | | \|\| ++\|\|\| \| \|\| \|\| \|\|+ \|+ \| ++ \| \| \| \| \| + | |
| Gnl\|Pfam\|pfam00001: | 1 | GNLLVILVILRTKKLRTPTNIFLLNLAVADLLFLLTLPPWALYYLVGGDWVFCDALCKLV | 60 |
| GPCR11: | 102 | FYISHWLGATECVLIATMSYDRYAAICRPLHYTVIMHPQLCLGLALASWLGGLTTSMVGS | 161 |
| | | + \| +\|\| +\| \|\|\| \|\| \|\| \| \|+ \| \| \|+ \| \|+ | |
| Gnl\|Pfam\|pfam00001: | 61 | GALFVVNGYASILLLTAISIDRYLAIVHPLRYRRIRTPRRAKVLILLVWVLALLLSLPPL | 120 |
| GPCR11: | 162 | TLTMLLPLCGNNCIDHFFCEMPLIMQLACVDTSLNEMEMYLASFVFVVLPLGLILVSYGH | 221 |
| | | + \| + \| + + \| \|++ \| \|\|\|\| +\|\|\| \| | |
| Gnl\|Pfam\|pfam00001: | 121 | LFSWLRT---VEEGNTTVCLIDFPEESVKRSYVL------LSTLVGFVLPLLVILVCYTR | 171 |
| GPCR11: | 222 | IARAV---------LKIRSAEGRRKAFNTCSSHVAVVSLFYGSIIFMYLQPAKSTSHEQ- | 271 |
| | | \| \| + \|\| \|\|+ \|+ \| \| \| + \| + \| \| + | |
| Gnl\|Pfam\|pfam00001: | 172 | ILRTLRKRARSQRSLKRRSSSERKAAKMLLVVVVVFVLCWLPYHIVLLLDSLCLLSIWRV | 231 |
| GPCR11: | 272 | ---GKFIALFYTVVTPALNPVIY | 291 |
| | | \| \|+ \| \|\|\|+\|\| | |
| Gnl\|Pfam\|pfam00001: | 232 | LPTALLITLWLAYVNSCLNPIIY | 254 |

GPCR12

The disclosed GPCR12 nucleic acid of 958 nucleotides (also referred to as ba407h12_da1) is shown in Table 12A. The disclosed GPCR12 open reading frame (“ORF”) begins with a ATG at nucleotides 3-5 and ending with a TAA codon at nucleotides 1162-1164. The start and stop codons of the open reading frame are highlighted in bold type. Putative untranslated regions, if any, are found upstream from the initiation codon and downstream from the termination codon.

TABLE 12A
GPCR12 nucleotide sequence.

(SEQ ID NO:29)

CTATGGAGCAGAGCAATTATTCCGTGTATGCCGACTTTATCCTTCTGGGTTTGTTCAGCAACGCCCGTTTCCCCTGG
CTTCTCTTTGCCCTCATTCTCCTGGTCTTTGTGACCTCCATAGCCAGCAACGTGGTCAAGATCATTCTCATCCACAT
AGACTCCCGCCTCCACACCCCCATGTACTTCCTGCTCAGCCAGCTCTCCCTCAGGGACATCTTGTATATTTCCACCA
TTGTGCCCAAAATGCTGGTCGACCAGGTGATGAGCCAGAGAGCCATTTCCTTTGCAGGATGCACTGCCCAACACTTC
CTCTACTTGACCTTAGCAGGGGCTGAGTTCTTCCTCCTAGGACTCATGTCCTGTGATCGCTACGTAGCCATCTGCAA
CCCTCTGCACTATCCTGACCTCATGAGCCGCAAGATCTGCTGGTTGATTGTGGCOGCAGCCTGGCTGGGAGGGTCTA
TCGATGGTTTCTTGCTCACCCCCGTCACCATGCAGTTCCCCTTCTGTGCCTCTCGGGAGATCAACCACTTCTTCTCC
GAGGTGCCTGCCCTTCTGAAGCTCTCCTGCACGGACACATCAGCCTACGAGACAGCCATGTATGTCTGCTGTATTAT
GATGCTCCTCATCCCTTTCTCTGTGATCTCGGGCTCTTACACAAGAATTCTCATTACTGTTTATAGGATGAGCGAGG
CAGAGGGGAGGCGAAAGGCTGTGGCCACCTGCTCCTCACACATGGTCCTTGTCAGCCTCTTCTATGCGGCTGCCATG
TACACATACGTGCTGCCTCATTCTTACCACACCCCTGAGCAGGACAAAGCTGTATCTGCCTTCTACACCATCCTCAC
TCCCATGCTCAATCCACTCATTTACAGCCTTAGGAACAAGGATGTCACGGGGGCCCTACAGAAGGTTGTTGGGAGGT
GTGTGTCCTCAGGAAAGGTAACCACTTTCTAAAC

The disclosured GPCR12 polypeptide (SEQ ID NO: 30) encoded protein having 317 amino acid residues is presented in Table 3B using the one-letter amino acid code.

TABLE 12B
Encoded GPCR12 protein sequence.

(SEQ ID NO:30)

MEQSNYSVYADFILLGLFSNARFPWLLFALILLVFVTSIASNVVKIILIHIDSRLHTPMYFLLSQLSLRDILYISTI
VPMLVDQVMSQRAISFAGCTAQHFLYLTLAGAEFFLLGLMSCDRYVAICNPLHYPDLMSRKICWLTVAAAWLGGSI
DGFLLTPVTMQFPFCASREINHFFCEVPALLKLSCTDTSAYETANYVCCIMMLLIPFSVISGSYTRILITVYRMSEA
EGRRKAVATCSSHMVVVSLFYGAAMYTYVLPHSYHTPEQDKAVSAFYTILTPMLNPLIYSLRNKDVTGALQKVVGRC
VSSGKVTTF

The disclosured GPCR12 amino acid sequence has 146 of 313 amino acid residues (46%) identical to, and 215 of 313 residues (68%) positive with, the Rattus norvegicus (rat) 313 amono acid residue olfactory receptor protein (ptnr: SPTREMBL-ACC:Q63394)(E=1.0e⁻⁷⁴).

possible SNPs found for GPCR12 are listed in Tables 12C.

TABLE 12C
SNPs

| | | Amino | |
|---|---|---|---|
| Nucleotide | Base | Acid | Base |
| Position | Change | Position | Change |
| 62 | C > T | Silent | N/A |
| 136 | A > T | 45 | Lys > Met |
| 216 | T > C | Silent | N/A |
| 287 | A > T | Silent | N/A |
| 361 | G > A | 120 | Cys > Tyr |
| 403 | A > T | 134 | Asp > Val |
| 896 | G > A | Silent | N/A |

The amino acid sequence of GPCR12 has high homology to other proteins as shown in Table 12D.

TABLE 12D
BLASTX results for GPCR12

| | | | Smallest |
|---|---|---|---|
| | | | Sum |
| Sequences producing High-scoring | | High | Prob |
| Segment Pairs: | | Score | P (N) |
| Ptnr:SPTREMBL-ACC:Q63394 OL1 | 313 aa . . . | 755 | 1.0e−74 |
| RECP - Rattus norv, | | | |

The disclosed GPCR12 has homology to the amino acid sequences shown in the BLASTP data listed in Table 12E.

TABLE 12E
BLASTP results for GPCR12

| | | Length | Identity | Positives | |
|---|---|---|---|---|---|
| Gene Index/Identifier | Protein/Organism | (aa) | (%) | (%) | Expect |
| gi\|14423768\|sp\|O43869 | OLFACTORY | 311 | 217/307 | 252/307 | e−110 |
| \|O2T1_HUMAN | RECEPTOR 2T1 | | (70%) | (81%) | |
| | (OLFACTORY | | | | |
| | RECEPTOR 1-25) | | | | |
| | (OR1-25) | | | | |
| gi\|3983382 gb AAD1331 | olfactory | 223 | 168/223 | 191/223 | 2e−87 |
| 9.1 | receptor E3 | | (75%) | (85%) | |
| | [Mus musculus] | | | | |
| gi\|2921628 gb AAC3961 | olfactory | 216 | 163/216 | 185/216 | 2e−84 |
| 1.1 | receptor | | (75%) | (85%) | |
| | [Homo sapiens] | | | | |
| gi\|12007423\|gb\|AAG451 | T2 olfactory | 316 | 157/309 | 212/309 | 1e−76 |
| 96.1 | receptor | | (50%) | (67%) | |
| | [Mus musculus] | | | | |
| gi\|12007424\|gb\|AAG451 | T3 olfactory | 315 | 156/310 | 212/310 | 4e−75 |
| 97.1 | receptor | | (50%) | (68%) | |
| | [Mus musculus] | | | | |

The homology of these sequences is shown graphically in the ClustalW analysis shown in Table 12F.

[image]

[image]

Table 12G lists the domain description from DOMAIN analysis results against GPCR12. This indicates that the GPCR12 sequence has properties similar to those of other proteins known to contain this domain as well as to the 254 amino acid 7tm domain itself.

TABLE 12G
Domain Analysis of GPCR12

gn1|Pfam|pfam00001, 7tm_1, 7 transmembrane receptor (rhodopsin
family). (SEQ ID NO:106)
Length = 254 residues, 99.6%aligned
Score = 105 bits (262), Expect = 4e−24

| | | | |
|---|---|---|---|
| GPCR12: | 42 | NVVKIILIHTDSRLHTPMYFLLSQLSLRDILYISTIVPKMLVDQVMSQRAISFAGCTAQH | 101 |
| | | \|++ \|++\| +\| \|\| \| \|++ \|+\|++ \|+ \| \| \| \| \| | |
| Gn1\|Pfam\|pfam00001: | 2 | NLLVILVILRTKKLRTPTNIFLLNLAVADLLFLLTLPPWALYYLVGGDWVFGDALCKLVG | 61 |
| GPCR12: | 102 | FLYLTLAGAEFFLLGLMSCDRYVAICNPLHYPDLMSRKICWLIVAAAWLGGSIDGFLLTP | 101 |
| | | \|++ \| \|\| +\| \|\|\|+\|\| +\|\| \| + + + +++ \|+ \|+ + \| \| | |
| Gn1\|Pfam\|pfam00001: | 62 | ALFVVNGYASILLLTAISIDRYLAIVHPLRYRRIRTPRRAKVLILLVWVLALL---LSLP | 118 |
| GPCR12: | 162 | VTMQFPFCASREINHFFCEVPALLKLSCTDTSAYETAMYVC-CIMMLLIPFSVISGSYTR | 220 |
| | | + \| \| \| \| + + + + ++ ++\| \|\| \|\|\| | |
| Gn1\|Pfam\|pfam00001: | 119 | PLLFSWLRTVEEGNTTVC-------LIDFPEESVKRSYVLLSTLVGFVLPLLVILVCYTR | 171 |
| GPCR12: | 221 | ILITV---------YRMSEAEGRRKAVATCSSHMVVVSLFYG----AAMYTYVLPHSYHT | 267 |
| | | \|\| \|+ + + \|+ \| +\| \| + + + \| + | |
| Gn1\|Pfam\|pfam00001: | 172 | ILRTLTKRARSQRSLKRRSSSERKAAKMLLVVVVVFVLCWLPYHIVLLLDSLCLLSIWRV | 231 |
| GPCR12: | 268 | PEQDKAVSAFYTILTPMLNPLIY | 290 |
| | | ++ + + \|\|\|+\|\| | |
| Gn1\|Pfam\|pfam00001: | 232 | LPTALLITLWLAYVNSCLNPIIY | 254 |

GPCR13

The disclosed GPCR13 nucleic acid of 1013 nucleotides (also referred to as AC074365_da1) is shown in Table 13A. The disclosed GPCR13 open reading frame (“ORF”) begins with a ATG at nucleotides 9-11 and ending with a TGA codon at nucleotides 1011-1013. The start and stop codons of the open reading frame are highlighted in bold type. Putative untranslated regions, if any, are found upstream from the initiation codon and downstream from the termination codon.

TABLE 13A
GPCR13 nucleotide sequence.

(SEQ ID NO:31)

CCTATGTGATGTGTTATCTTTCTCAGCTATGCCTCAGCCTTGGGGAACACACTTTACATATGGGGATGGTGAGACAT
ACCAATGAGAGCACCTAGCAGGTTTCATCCTTTTAGGGTTTTCTGATTATCCTCAGTTACAGAAGGTTCTATTTGT
GCTCATATTGATTCTGTATTTACTAACTATTTTGGGGATACCACCATCATTCTGGTTTCTCGTCTGGAACCCAAGC
CTCATATGCCGATGTATTTCTTCCTTTCTCATCTCTCCTTCCTGTACCGCTGCTTCACCAGCAGTGTTATTCCCCG
CTCCTGGTAAACCTGTGGGAACCCATGAAAACTATCGCCTATGGTGGCTGTTTGGTTCACCTTTACAACTCCCATGC
CCTGGGATCCACTGAGTGCGTCCTCCCGGCTCTGATGTCCTGTGACCGCTATGTGGCTGTCTGCCGTCCTCTCCATT
ACACTGTCTTAATGCATATCCATCTCTGCATGGCCTTGGCATCTATGGCATGGCTCAGTGGAATAGCCACCACCCTG
GTACAGTCCACCCTCACCCTGCAGCTGCCCTTCTGTGGGCATCGCCAAGTGGATCATTTCATCTGCGAGGTCCCTGT
GCTCATCAAGCTGGCTTGTGTGGGCACCACGTTTAACGAGGCTGAGCTTTTTGTGGCTAGTATCCTTTTCCTTATAG
TGCCTGTCTCATTCATCCTGGTCTCCTCTGGCTACATTGCCCACGAGTGTTGAGGATTAAGTCAGCTACCGGGAGA
CAGAAAGCATTCGGGACCTGCTTCTCCCACCTGACAGTGGTCACCATCTTTTATGGAACCATCATCTTCATGTATCT
GCAGCCAGCCAAGACTAGATCCAGGGACCAGGGCAAGTTTGTTTCTCTCTTCTACACTGTGGTAACCCGCATGCTTA
ACCCTCTTATTTATACCTTGAGGATCAAGGAGGTGAAAGGGGCATTAAGAAAGTTCTAGCAAAGGCTCTGGGAGTA
AATATTTTATGA

The disclosed GPCR13 polypeptide (SEQ ID NO: 32) encoded protein having 334 amino acid residues is presented in Table 13B using the one-letter amino acid code. The Signal P, Psort and/or Hydropathy results predict that GPCR13 has a signal peptide and is likely to be localized at the plasma membrane with a certainty of 0.6000. The most likely cleavage site for a GPCR13 peptide is between amino acids 61 and 62 at: ILG-NT.

TABLE 13B
Encoded GPCR13 protein sequence.

(SEQ ID NO:32)

MCYLSQLCLSLGEHTLHMGMVRHTNESNLAGFILLGFSDYPQLQKVLFVLILILYLLTILGNTTIILVSRLEPKPHM
PMYFFLSHLSFLYRCFTSSVIPQLLVNLWEPMKTIAYGGCLVHLYNSHALGSTECVLPALMSCDRYVAVCRPLHYTV
LMHIHLCMALASMAWLSGIATTLVQSTLTLQLPFCGHRQVDHFICEVPVLIKLACVGTTFNEAELFVASILFLIVPV
SFILVSSGYIAAVLRIKSATGRQKAFGTCFSHLTVVTIFYGTIIFMYLQPAKSRSRDQGKFVSLFYTVVTRMLNPL
IYTLRIKEVKGALKKVLAKALGVNIL

The disclosed GPCR13 amino acid sequence has 178 of 305 amino acid residues (58%) identical to, and 234 of 305 residues (76%) positive with, the Rattus norvegicus (rat) 313 amino acid residue olfatory receptor protein (ptnr: SPTREMBL-ACC:Q63394)(E=2.6e⁻⁹⁶).

and a T to C SNP at 802 bp of the nucleotide sequence that results in a Leu to Pro change at amino acid 265 of protein sequence.

Possible SNPs found for GPCR13 are listed in Tables 13C.

TABLE 13C
SNPs

| | | Amino | |
|---|---|---|---|
| Nucleotide | Base | Acid | Base |
| Position | Change | Position | Change |
| 129 | C > G | 41 | Pro > Ala |
| 309 | C > T | 101 | Leu > Phe |
| 324 | T > C | 106 | Trp > Arg |
| 417 | C > G | 137 | Leu > Val |
| 361 | G > A | 120 | Cys > Tyr |
| 458 | C > T | Silent | N/A |
| 495 | G > A | 163 | Ala > Thr |
| 507 | A > G | 167 | Met > Val |
| 510 | G > A | 168 | Ala > Thr |
| 512 | A > G | Silent | N/A |
| 559 | T > C | 184 | Leu > Pro |
| 765 | G > A | 253 | Gly > Arg |
| 802 | T > C | 265 | Leu > Pro |

The amino acid sequence of GPCR13 has high homology to other proteins as shown in Table 13D.

TABLE 13D
BLASTX results for GPCR13

| | | | Smallest |
|---|---|---|---|
| | | | Sum |
| Sequences producing High-scoring | | High | Prob |
| Segment Pairs: | | Score | P (N) |
| Ptnr:SPTREMBL-ACC:Q63394 | 313 aa . . . | 959 | 2.6e−96 |
| OL1 RECP - Rattus norv, | | | |

The disclosed GPCR13 has homology to the amino acid sequences shown in the BLASTP data listed in Table 13E.

TABLE 13E
BLASTP results for GPCR13

| | | Length | Identity | Positives | |
|---|---|---|---|---|---|
| Gene Index/Identifier | Protein/Organism | (aa) | (%) | (%) | Expect |
| gi\|6679170\|ref\|NP_032 | olfactory | 312 | 179/308 | 232/308 | 8e−91 |
| 788.1\| | receptor 15 | | (58%) | (75%) | |
| | [Mus musculus] | | | | |
| gi\|11177906\|ref NP_06 | Olfactory | 313 | 178/305 | 234/305 | 4e−90 |
| 8632.1\| | receptor | | (58%) | (76%) | |
| | [Rattus norvegicus] | | | | |
| gi\|14780900\|ref NP_14 | olfactory | 357 | 179/305 | 232/305 | 2e−89 |
| 9046.1\| | receptor, family | | (58%) | (75%) | |
| | 2, subfamily B, | | | | |
| | member 2 | | | | |
| | [Homo sapiens] | | | | |
| gi\|12231029\|sp\|Q15062 | OLFACTORY | 316 | 173/303 | 230/303 | 4e−89 |
| O2H3 HUMAN | RECEPTOR 2H3 | | (57%) | (75%) | |
| | (OLFACTORY | | | | |
| | RECEPTOR-LIKE | | | | |
| | PROTEIN FAT11) | | | | |
| gi\|15304846\|ref XP_05 | olfactory | 357 | 178/305 | 232/305 | 7e−89 |
| 3609.1 | receptor, family | | (58%) | (75%) | |
| | 2, subfamily B, | | | | |
| | member 2 | | | | |
| | [Homo sapiens] | | | | |

The homology of these sequences is shown graphically in the ClustalW analysis shown in Table 13F.

[image]

[image]

Table 13G lists the domain description from DOMAIN analysis results against GPCR13. This indicates that the GPCR13 sequence has properties similar to those of other proteins known to contain this domain as well as to the 377 amino acid 7tm domain itself.

TABLE 13G
Domain Analysis of GPCR13

gnl|Pfam|pfam00001, 7tm_1, 7 transmembrane receptor (rhodopsin
family). (SEQ ID NO:112)
Length = 254 residues, 94.9% aligned
Score = 70.9 bits (172), Expect = 1e−13

| | | | |
|---|---|---|---|
| GPCR13: | 74 | KPHMPMYFFLSHLSFLYRCFTSSVIPQLLVNLWEPMKTIAYGGCLVHLYNSHALGSTECV | 133 |
| | | \| \|\| +\|+ \| ++ \| \| \| \| + \| + | |
| Gn1\|Pfam\|pfam00001: | 14 | KLRTPTNIFLLNLAVADLLFLLTLPPWALYYLVGGDWVFGDALCKLVGALFVVNGYASIL | 73 |
| GPCR13: | 134 | LPALMSCDRYVAVCRPLHYTVLMHIHLCMALASMAWLSGIATTLVQSTLTLQLPFCGERQ | 193 |
| | | \| +\| \|\|\|+\|+ \|\| \| + \| + + + \| \|\| | |
| Gnl\|Pfam\|pfam00001: | 74 | LLTAISIDRYLAIVHPLRYRRIRTPRRAKVLILLVWVLALL---------LSLPPLLFSW | 124 |
| GPCR13: | 194 | VDHFIVEVPVLIKLACVGTTFNEAELFVASILFLIVPVSFILVSSGYTAHAV | 245 |
| | | + + + + + + +++++ ++\|+ \|\|\| \| + | |
| Gnl\|Pfam\|pfam00001: | 125 | LRTVEEGNTTVCLIDFPEESVKRSYVLLSTLVGFVLPLLVILVCYTRILRTLRKRARSQR | 184 |
| GPCR13: | 246 | -LRIKSATGRQKAFGTCFSHLTVVTIFYGTIIFMYL----QPAKSRSRDQGKFVSLFYTV | 300 |
| | | \|+ +\|++ \|+ \| + + + \| + \| + \| ++\|+ | |
| Gnl\|Pfam\|pfam00001: | 185 | SLKRSSSERKAAKMLLVVVVVFVLCWLPYHIVLLLDSLCLLSIWRVLPTALLITLWLAY | 244 |
| GPCR13: | 201 | VTRMLNPLIY | 310 |
| | | \| \|\|\|+\|\| | |
| Gnl\|Pfam\|pfam00001: | 245 | VNSCLNPIIY | 254 |

GPCR14

The disclosed GPCR14 nucleic acid of 996 nucleotides (also referred to as CG50161-03) is shown in Table 13A. The disclosed GPCR14 open reading frame (“ORF”) begins with at nucleotides 12-14 and ending with a TAA codon at nucleotides 993-995. The start and stop codons of the open reading frame are highlighted in bold type. Putative untranslated regions, if any, are found upstream from the initiation codon and downstream from the termination codon.

TABLE 14A
GPCR14 nucleotide sequence.

(SEQ ID NO:33)

CATGCTGGACTATGCCCTCTCCATTTACAGGTAGCTCTACTAGAAATATGGAGAGCAGAAACCAATCAACAGTGACT
GAATTTATCTTCACTGGATTCCCTCAGCTTCAGGATGGTAGTCTCCTGTACTTCTTTCCTTTACTTTTCATCTATAC
TTTTATTATCATTGATAACTTATTAATCTTCTCTGCTGTAAGGCTGGACACCCATCTCCACAACCCCATGTATAATT
TTATCAGTATATTTTCCTTTCTGGAGATCTGGTACACCACAGCCACCATTCCCAAGATGCTCTCCAACCTCATCAGT
GAAAAGAAGGCCATCTCAATGACTGGCTGCATCTTGCAGATGTATTTCTTCCACTCACTTGAAAACTCAGAGGGGAT
CTTGCTGACCACCATGGCCATTGACAGATACGTTGCCATCTGCAACCCTCTTCGCTATCAAATGATCATGACCCCCC
GGCTCTGTGCTCAACTCTCTGCAGGTTCCTGCCTCTTCGGTTTCCTTATCCTGCTTCCCGAGATTGTGATGATTTCC
ACACTGCCTTTCTGTCGGCCCAACCAAATCCATCAGATCTTCTGTGACTTGGTCCCTGTGCTAAGCCTCGCCTGTAC
AGACACGTCCATGATTCTGATTGAGGATGTGATTCATGCTGTGACCATCATCATTACCTTCCTAATCATTGCCCTGT
CCTATGTAAGAATTGTCACTGTGATATTGAGGATTCCCTCTTCTGAAGGGAGGCAAAAGGCTTTTTCTACCTGTGCA
GGCCACCTCATGGTCTTCCCGATATTCTTTCGCAGTGTATCACTCATGTACTTGCGTTTCAGCGACACTTATCCACC
AGTTTTGGACACAGCCATTGCACTGATGTTTACTGTACTTGCTCCATTCTTCAATCCCATCATTTATAGCCTGAGAA
ACAAGGACATGAACAATGCGATTAAAAAACTGTTCTGTCTTCAAAAAGTGTTGAACAAGCCTGGAGGTTAAT

The disclosed GPCR14 of this invention maps to chromosome 1 and the GPCR14 the nucleic acid sequence of this invention has 943 of 945 bases (99%) identical to a gb:GENBANK-ID:AX241804|acc:AX241804.1 mRNA from synthetic construct (Sequence 552 from Patent WO0127158) (E=1.8e⁻²⁰⁶). Chromosome localization information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

The disclosed GPCR14 polypeptide (SEQ ID NO: 34) encoded protein having 327 amino acid residues is presented in Table 14B using the one-letter amino acid code. The Signal P, Psort and/or Hydropathy results predict that GPCR14 has a signal peptide and is likely to be localized at the plasma membrane with a certainty of 0.6000. In other embodiments, it is localized at the Golgi body with a certainty of 0.4000, at the endoplasmic reticulum (membrane) with a certainty of 0.3000 or at the endoplasmic reticulum with a certainty of 0.3000. The most likely cleavage site for a GPCR14 peptide is between amino acids 62 and 63, at: AVR-LD.

TABLE 14B
Encoded GPCR14 protein sequence.

(SEQ ID NO:34)

MPSPFTGSSTRNMESRNQSTVTEFIFTGFPQLQDGSLLYFFPLLFIYTFIIIDNLLIFSAVRLDTHLHNPMYNFISI
FSFLEIWYTTATIPKMLSNLISEKKAISMTGCILQMYFFHSLENSEGILLTTMAIDRYVAICNPLRYQMIMTPRLCA
QLSAGSCLFGFLILLPEIVMISTLPFCGPNQIHQIFCDLVPVLSLACTDTSMILIEDVIHAVTIIITFLIIALSYVR
IVTVILRIPSSEGRQKAFSTCAGHLMVFPIFFGSVSLMYLRFSDTYPPVLDTAIALMFTVLAPFFNPIIYSLRNKDM
NNAIKKLFCLQKVLNKPGG

The disclosed GPCR14 amino acid sequence of the protein of the invention was found to have 136 of 304 amino acid residues (44%) identical to, and 193 of 304 amino acid residues (63%) similar to, the 321 amino acid residue ptnr:SWISSPROT-ACC:Q9UGF6 protein from Homo sapiens (Human) (Olfactory receptor 5V1 (Hs6M1-21) (E=4.4e⁻⁶⁶).

GPCR14 is expressed in at least the following tissues: apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. Expression information was derived from the tissue sources of the sequences that were included in the derivation of the sequence.

The amino acid sequence of GPCR14 has high homology to other proteins as shown in Table 14C.

TABLE 14C
BLASTX results for GPCR14

| | | Smallest |
|---|---|---|
| | | Sum |
| | High | Prob |
| Sequences producing High-scoring Segment Pairs: | Score | P(N) |
| ptnr:SWISSPROT-ACC:Q9UGF6 OLF RECP - Homo sapiens, 321 aa . . . | 675 | 4.4e−66 |

The disclosed GPCR14 has homology to the amino acid sequences shown in the BLASTP data listed in Table 14D.

TABLE 14D
BLASTP results for GPCR14

| | | Length | Identity | Positives | |
|---|---|---|---|---|---|
| Gene Index/Identifier | Protein/Organism | (aa) | (%) | (%) | Expect |
| gi\|13562084\|ref\|NP_11 | olfactory | 321 | 136/304 | 193/304 | 8e−60 |
| 0503.1\| | receptor, family | | (44%) | (62%) | |
| | 5, subfamily V | | | | |
| | member 1 | | | | |
| | [Homo sapiens] | | | | |
| gi\|7242165\|ref\|NP_035 | olfactory | 327 | 132/308 | 191/308 | 9e−59 |
| 113.1\| | receptor 41 | | (42%) | (61%) | |
| | [Mus musculus] | | | | |
| gi\|12007416\|gb\|AAG451 | m51 olfactory | 314 | 140/303 | 190/303 | 1e−58 |
| 89.1 | receptor | | (46%) | (62%) | |
| | [Mus musculus] | | | | |
| gi\|13928994\|ref NP_11 | olfactory | 327 | 135/308 | 192/308 | 2e−58 |
| 3898.1 | receptor 41 | | (43%) | (61%) | |
| | [Rattus norvegicus] | | | | |
| gi\|14596246\|emb CAC43 | dM538M10.1 (novel | 317 | 135/306 | 187/306 | 3e−58 |
| 444.1\| | 7 transmembrane | | (44%) | (60%) | |
| | receptor | | | | |
| | (rhodopsin | | | | |
| | family) | | | | |
| | (olfactory | | | | |
| | receptor like) | | | | |
| | protein similar | | | | |
| | to | | | | |
| | human HS6M1-21) | | | | |
| | [Mus musculus] | | | | |

The homology of these sequences is shown graphically in the ClustalW analysis shown in Table 14E.

[image]

[image]

Table 14F lists the domain description from DOMAIN analysis results against GPCR14. This indicates that the GPCR14 sequence has properties similar to those of other proteins known to contain this domain as well as to the 377 amino acid 7tm domain itself.

TABLE 14F
Domain Analysis of GPCR14

gnl, Pfam|pfam00001, 7tm_1, 7 transmembrane receptor (rhodopsin
family). (SEQ ID NO:118)
Length = 254 residues, 99.6% aligned
Score = 97.1 bits (240), Expect = 1e−21

| | | | |
|---|---|---|---|
| GPCR14: | 54 | NLLIFSAVLDTHLHNPMYNFISIFSFLEIWYTTATIPKMLSNLISEKKAISMTGCILQM | 113 |
| | | \|\|\|+ + \| \| \|+ + ++ + \| \| \|+ \| \| | |
| Gnl\|Pfam\|pfam00001: | 2 | NLLVILVILRTKKLRTPTNIFLLNLAVADLLFLLTLPPWALYYLVGGDWVFGDALCKLVG | 61 |
| GPCR14: | 114 | YFFHSLENSEGILLTTMAIDRYVAICNPLRYQMIMTPRLCAQLSAGSCLFGFLILLPEIV | 173 |
| | | \| + +\|\|\| ++\|\|\|\|\|+\|\| +\|\|\|\|+ \| \|\|\| \| + \|+ \|\| ++ | |
| Gnl\|Pfam\|pfam00001: | 62 | ALFVNGYASILLLTAISIDRYLAIVHPLRYRRIRTPRRAKVLILLVWVLALLLSLPPLL | 121 |
| GPCR14: | 174 | MISTLPFCGPNQIHQIFCDLVPVLSLACTDTSMILIEDVIHAVTIIITFLI-IALSYVRI | 232 |
| | | \| + + + \| ++ + \| ++ \| \| | |
| Gnl\|Pfam\|pfam00001: | 122 | FSWLRTVEEGNTTVCLIDFPEESVKRSYVLLSTLVGFVLPLLVILVCYTRILRTLRKRAR | 181 |
| GPCR14: | 233 | VTVILRIPSSEGRQKAFSTCAGHLMVF----PIFFGSVSLMYLRFSDTYPPVLDTAIALM | 288 |
| | | \|+ \|\| \|+ \| ++ \| + \| \| \| | |
| Gnl\|Pfam\|pfam00001: | 182 | SQRSLKRSSSERKAAKMLLVVVVVFVLCWLPYHIVLLLDSLCLLSIWRVLPTALLITLW | 241 |
| GPCR14: | 289 | FTVLAPFFNPIIY | 301 |
| | | + \|\|\|\|\| | |
| Gnl\|Pfam\|pfam00001: | 242 | LAYVNSCLNPIIY | 254 |

The protein similarity information, expression pattern, cellular localization, and map location for the protein and nucleic acid disclosed herein suggest that GPCR 14 may have important structural and/or physiological functions characteristic of the GPCR family. The nucleic acids and proteins of the invention are useful in potential diagnostic and therapeutic applications implicated in various GPCR- or olfactory receptor (OR)-related pathologies and/or disorders. For example, the compositions of the present invention will have efficacy for the treatment of patients suffering from: developmental diseases, MHCII and III diseases (immune diseases), taste and scent detectability disorders, Burkitt's lymphoma, corticoneurogenic disease, signal transduction pathway disorders, retinal diseases including those involving photoreception, cell growth rate disorders; cell shape disorders, feeding disorders; control of feeding; potential obesity due to over-eating; potential disorders due to starvation (lack of appetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and treatment of Albright hereditary ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation, dentatorubro-pallidoluysian atrophy(DRPLA) hypophosphatemic rickets, autosomal dominant (2) acrocallosal syndrome and dyskinesias, such as Huntington's disease.

The disclosed GPCR14 protein has multiple hydrophilic regions, each of which can be used as an immunogen. In one embodiment, a contemplated GPCR14 epitope is from about amino acids 5 to 10. In additional embodiments, a GPCR14 epitope is from about amino acids 90 to 95, from about amino acids 130 to 132, from 145 to 150, from 240 to 250, from 310 to 320 and from 330 to 340. The GPCR14 protein also have value in the development of powerful assay system for functional analysis of various human disorders, which will help in understanding of pathology of the disease and development of new drug targets for various disorders.

A summary of the GPCRX nucleic acids and proteins of the invention is provided in Table 15.

TABLE 15
GPCR Summary

| | | | Nucle- | |
|---|---|---|---|---|
| | | | ic | Amino |
| | | | Acid | Acid |
| | | | SEQ | SEQ |
| | | Clone:Description | ID | ID |
| Name | Tables | of Homolog | NO | NO |
| GPCR1 | 1A-1O | GPCR1a:SC35113271_{—} | 1 | 2 |
| | | A_da1 | | |
| | | GPCR1b:CG55798-03 | 3 | 4 |
| | | GPCR1c:CG55798-04 | 5 | 6 |
| GPCR2 | 2A-2L | GPCR2a:AC011711_da2 | 7 | 8 |
| | | GPCR2b:CG50147-01 | 9 | 10 |
| GPCR3 | 3A-3H | GPCR3:GMAC024428_A_{—} | 11 | 12 |
| | | (also known as | | |
| | | CG92194-01) | | |
| GPCR4 | 4A-4F | GPCR4:CG50169-01 | 13 | 14 |
| GPCR5 | 5A-5F | GPCR5:AC009758_da1 | 15 | 16 |
| GPCR6 | 6A-6G | GPCR6:CG50149-01 | 17 | 18 |
| GPCR7 | 7A-7H | GPCR7:GM_33202597_{—} | 19 | 20 |
| | | A_da1 | | |
| GPCR8 | 8A-8H | GPCR8:AC076959_da2 | 21 | 22 |
| GPCR9 | 9A-9G | GPCR9:AC073364_da1 | 23 | 24 |
| GPCR10 | 10A-10G | GPCR10:CG55732-01 | 25 | 26 |
| GPCR11 | 11A-11G | GPCR11:sggc_draft_{—} | 27 | 28 |
| | | ba656o22_20000731_da1 | | |
| GPCR12 | 12A-12G | GPCR12:ba407h12_da1 | 29 | 30 |
| GPCR13 | 13A-13G | GPCR13:AC074365_da1 | 31 | 32 |
| GPCR14 | 14A-14F | GPCR14:CG50161-03 | 33 | 34 |

GPCRX Nucleic Acids and Polypeptides

One aspect of the invention pertains to isolated nucleic acid molecules that encode GPCRX polypeptides or biologically active portions thereof. Also included in the invention are nucleic acid fragments sufficient for use as hybridization probes to identify GPCRX-encoding nucleic acids (e.g., GPCRX mRNAs) and fragments for use as PCR primers for the amplification and/or mutation of GPCRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An GPCRX nucleic acid can encode a mature GPCRX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated GPCRX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS: 1, 3, 5, 7, 9, 11, 13, 15, 17, 19, 21, 23, 25, 27, 29, 31 and 33 or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, 31 and 33 as a hybridization probe, GPCRX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GPCRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 1, 3, 5, 7, 9, 11, 13, 15, 17, 19, 21, 23, 25, 27, 29, 31 and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29, 31 and 33, or a portion of this nucleotide sequence (e.g., a fragment that can be used as a probe or primer or a fragment encoding a biologically-active portion of an GPCRX polypeptide). A nucleic acid molecule that is complementary to the nucleotide sequence shown in SEQ ID NOS: 1, 3, 5, 7, 9, 11, 13, 15, 17, 19, 21, 23, 25, 27, 29, 31 and 33 is one that is sufficiently complementary to the nucleotide sequence shown in SEQ ID NOS: 1, 3, 5, 7, 9, 11, 13, 15, 17, 19, 21, 23, 25, 27, 29, 31 and 33 that it can hydrogen bond with little or no mismatches to the nucleotide sequence shown SEQ ID NOS: 1, 3, 5, 7, 9, 11, 13, 15, 17, 19, 21, 23, 25, 27, 29, 31 and 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence, ” or variations thereof, refer to sequences characterized by a homology at the nucleotide level or amino acid level as discussed above. Homologous nucleotide sequences encode those sequences coding for isoforms of GPCRX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an GPCRX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human GPCRX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31 and 33, as well as a polypeptide possessing GPCRX biological activity. Various biological activities of the GPCRX proteins are described below.

An GPCRX polypeptide is encoded by the open reading frame (“ORF”) of an GPCRX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human GPCRX genes allows for the generation of probes and primers designed for use in identifying and/or cloning GPCRX homologues in other cell types, e.g. from other tissues, as well as GPCRX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS: 1, 3, 5, 7, 9, 11, 13, 15, 17, 19, 21, 23, 25, 27, 29, 31 and 33; or an anti-sense strand nucleotide sequence of SEQ ID NOS: 1, 3, 5, 7, 9, 11, 13, 15, 17, 19, 21, 23, 25, 27, 29, 31 and 33; or of a naturally occurring mutant of SEQ ID NOS: 1, 3, 5, 7, 9, 11, 13, 15, 17, 19, 21, 23, 25, 27, 29, 31 and 33.

Probes based on the human GPCRX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an GPCRX protein, such as by measuring a level of an GPCRX-encoding nucleic acid in a sample of cells from a subject e.g., detecting GPCRX mRNA levels or determining whether a genomic GPCRX gene has been mutated or deleted.

“A polypeptide having a biologically-active portion of an GPCRX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of GPCRX” can be prepared by isolating a portion SEQ ID NOS: 1, 3, 5, 7, 9, 11, 13, 15, 17, 19, 21, 23, 25, 27, 29, 31 and 33 that encodes a polypeptide having an GPCRX biological activity (the biological activities of the GPCRX proteins are described below), expressing the encoded portion of GPCRX protein (e.g., by recombinant expression in vitro) and assessing the activity of the encoded portion of GPCRX.

GPCRX Nucleic Acid and Polypeptide Variants

The invention further encompasses nucleic acid molecules that differ from the nucleotide sequences shown SEQ ID NOS: 1, 3, 5, 7, 9, 11, 13, 15, 17, 19, 21, 23, 25, 27, 29, 31 and 33 due to degeneracy of the genetic code and thus encode the same GPCRX proteins as that encoded by the nucleotide sequences shown in SEQ ID NOS: 1, 3, 5, 7, 9, 11, 13, 15, 17, 19, 21, 23, 25, 27, 29, 31 and 33. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32 and 34. In addition to the human GPCRX nucleotide sequences shown in SEQ ID NOS: 1, 3, 5, 7, 9, 11, 13, 15, 17, 19, 21, 23, 25, 27, 29, 31 and 33 it will be appreciated by those skilled in the art that DNA sequence polymorphisms that lead to changes in the amino acid sequences of the GPCRX polypeptides may exist within a population (e.g., the human population). Such genetic polymorphism in the GPCRX genes may exist among individuals within a population due to natural allelic variation. As used herein, the terms “gene” and “recombinant gene” refer to nucleic acid molecules comprising an open reading frame (ORF) encoding an GPCRX protein, preferably a vertebrate GPCRX protein. Such natural allelic variations can typically result in 1-5% variance in the nucleotide sequence of the GPCRX genes. Any and all such nucleotide variations and resulting amino acid polymorphisms in the GPCRX polypeptides, which are the result of natural allelic variation and that do not alter the functional activity of the GPCRX polypeptides, are intended to be within the scope of the invention.

Moreover, nucleic acid molecules encoding GPCRX proteins from other species, and thus that have a nucleotide sequence that differs from the human sequence SEQ ID NOS: 1, 3, 5, 7, 9, 11, 13, 15, 17, 19, 21, 23, 25, 27, 29, 31 and 33 are intended to be within the scope of the invention. Nucleic acid molecules corresponding to natural allelic variants and homologues of the GPCRX cDNAs of the invention can be isolated based on their homology to the human GPCRX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31 and 33.

In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding GPCRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOS: 1, 3, 5, 7, 9, 11, 13, 15, 17, 19, 21, 23, 25, 27, 29, 31 and 33 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31 and 33 or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that maybe used are well-known within the art. See, e.g., Ausubel, et al. (eds.), 1993, CURRENT PROTOCOLS IN MOLECULAR BIOLOGY, John Wiley & Sons, NY, and Knegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS: 1, 3, 5, 7, 9, 11, 13, 15, 17, 19, 21, 23, 25, 27, 29, 31 and 33 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of GPCRX sequences that may exist in the population, the skilled artisan will further appreciate that changes can be introduced by mutation into the nucleotide sequences of SEQ ID NOS: 1, 3, 5, 7, 9, 11, 13, 15, 17, 19, 21, 23, 25, 27, 29, 31 and 33 thereby leading to changes in the amino acid sequences of the encoded GPCRX proteins, without altering the functional ability of said GPCRX proteins. For example, nucleotide substitutions leading to amino acid substitutions at “non-essential” amino acid residues can be made in the sequence of SEQ ID NOS: 2, 4, 6, 8, 10, 12, 14, 16, 18, 20, 22, 24, 26, 28, 30, 32 and 34. A “non-essential” amino acid residue is a residue that can be altered from the wild-type sequences of the GPCRX proteins without altering their biological activity, whereas an “essential” amino acid residue is required for such biological activity. For example, amino acid residues that are conserved among the GPCRX proteins of the invention are predicted to be particularly non-amenable to alteration. Amino acids for which conservative substitutions can be made are well-known within the art.

Another aspect of the invention pertains to nucleic acid molecules encoding GPCRX proteins that contain changes in amino acid residues that are not essential for activity. Such GPCRX proteins differ in amino acid sequence from SEQ ID NOS: 2, 4, 6, 8, 10, 12, 14, 16, 18, 20, 22, 24, 26, 28, 30, 32 and 34 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS: 2, 4, 6, 8, 10, 12, 14, 16, 18, 20, 22, 24, 26, 28, 30, 32 and 34. Preferably, the protein encoded by the nucleic acid molecule is at least about 60% homologous to SEQ ID NOS: SEQ ID NOS: 2, 4, 6, 8, 10, 12, 14, 16, 18, 20, 22, 24, 26, 28, 30, 32 and 34; more preferably at least about 70% homologous to SEQ ID NOS: 2, 4, 6, 8, 10, 12, 14, 16, 18, 20, 22, 24, 26, 28, 30, 32 and 34; still more preferably at least about 80% homologous to SEQ ID NOS: SEQ ID NOS: 2, 4, 6, 8, 10, 12, 14, 16, 18, 20, 22, 24, 26, 28, 30, 32 and 34; even more preferably at least about 90% homologous to SEQ ID NOS: 2, 4, 6, 8, 10, 12, 14, 16, 18, 20, 22, 24, 26, 28, 30, 32 and 34; and most preferably at least about 95% homologous to SEQ ID NOS: 2, 4, 6, 8, 10, 12, 14, 16, 18, 20, 22, 24, 26, 28, 30, 32 and 34.

An isolated nucleic acid molecule encoding an GPCRX protein homologous to the protein of SEQ ID NOS: 2, 4, 6, 8, 10, 12, 14, 16, 18, 20, 22, 24, 26, 28, 30, 32 and 34 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31 and 33 such that one or more amino acid substitutions, additions or deletions are introduced into the encoded protein.

Mutations can be introduced into SEQ ID NOS: 2, 4, 6, 8, 10, 12, 14, 16, 18, 20, 22, 24, 26, 28, 30, 32 and 34 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the GPCRX protein is replaced with another amino acid residue from the same side chain family. Alternatively, in another embodiment, mutations can be introduced randomly along all or part of an GPCRX coding sequence, such as by saturation mutagenesis, and the resultant mutants can be screened for GPCRX biological activity to identify mutants that retain activity. Following mutagenesis of SEQ ID NOS: 1, 3, 5, 7, 9, 11, 13, 15, 17, 19, 21, 23, 25, 27, 29, 31 and 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant GPCRX protein can be assayed for (i) the ability to form protein:protein interactions with other GPCRX proteins, other cell-surface proteins, or biologically-active portions thereof, (ii) complex formation between a mutant GPCRX protein and an GPCRX ligand; or (iii) the ability of a mutant GPCRX protein to bind to an intracellular target protein or biologically-active portion thereof, (e.g. avidin proteins).

In yet another embodiment, a mutant GPCRX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31 and 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire GPCRX coding strand, or to only a portion thereof. Nucleic acid molecules encoding fragments, homologs, derivatives and analogs of an GPCRX protein of SEQ ID NOS: 1, 3, 5, 7, 9, 11, 13, 15, 17, 19, 21, 23, 25, 27, 29, 31 and 33, or antisense nucleic acids complementary to an GPCRX nucleic acid sequence of SEQ ID NOS: 1, 3, 5, 7, 9, 11, 13, 15, 17, 19, 21, 23, 25, 27, 29, 31 and 33, are additionally provided.

In one embodiment, an antisense nucleic acid molecule is antisense to a “coding region” of the coding strand of a nucleotide sequence encoding an GPCRX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the GPCRX protein. The term “noncoding region” refers to 5′ and 3′ sequences which flank the coding region that are not translated into amino acids (i.e., also referred to as 5′ and 3′ untranslated regions).

Given the coding strand sequences encoding the GPCRX protein disclosed herein, antisense nucleic acids of the invention can be designed according to the rules of Watson and Crick or Hoogsteen base pairing. The antisense nucleic acid molecule can be complementary to the entire coding region of GPCRX mRNA, but more preferably is an oligonucleotide that is antisense to only a portion of the coding or noncoding region of GPCRX mRNA. For example, the antisense oligonucleotide can be complementary to the region surrounding the translation start site of GPCRX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g. an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an GPCRX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave GPCRX mRNA transcripts to thereby inhibit translation of GPCRX mRNA. A ribozyme having specificity for an GPCRX-encoding nucleic acid can be designed based upon the nucleotide sequence of an GPCRX cDNA disclosed herein (i.e., SEQ ID NOS: 1, 3, 5, 7, 9, 11, 13, 15, 17, 19, 21, 23, 25, 27, 29, 31 and 33). For example, a derivative of a Tetrahymena L-19 IVS RNA can be constructed in which the nucleotide sequence of the active site is complementary to the nucleotide sequence to be cleaved in an GPCRX-encoding mRNA. See, e.g., U.S. Pat. No. 4,987,071 to Cech, et al. and U.S. Pat. No. 5,116,742 to Cech, et al. GPCRX mRNA can also be used to select a catalytic RNA having a specific ribonuclease activity from a pool of RNA molecules. See, e.g., Bartel et al., (1993) Science 261:1411-1418.

Alternatively, GPCRX gene expression can be inhibited by targeting nucleotide sequences complementary to the regulatory region of the GPCRX nucleic acid (e.g., the GPCRX promoter and/or enhancers) to form triple helical structures that prevent transcription of the GPCRX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the GPCRX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of GPCRX can be used in therapeutic and diagnostic applications. For example, PNAs can be used as antisense or antigene agents for sequence-specific modulation of gene expression by, e.g., inducing transcription or translation arrest or inhibiting replication. PNAs of GPCRX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of GPCRX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of GPCRX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a bybridization-triggered cleavage agent, and the like.

GPCRX Polypeptides

A polypeptide according to the invention includes a polypeptide including the amino acid sequence of GPCRX polypeptides whose sequences are provided in SEQ ID NOS: 2, 4, 6, 8, 10, 12, 14, 16, 18, 20, 22, 24, 26, 28, 30, 32 and 34. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32 and 34 while still encoding a protein that maintains its GPCRX activities and physiological functions, or a functional fragment thereof.

In general, an GPCRX variant that preserves GPCRX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated GPCRX proteins, and biologically-active portions thereof, or derivatives, fragments, analogs or homologs thereof. Also provided are polypeptide fragments suitable for use as immunogens to raise anti-GPCRX antibodies. In one embodiment, native GPCRX proteins can be isolated from cells or tissue sources by an appropriate purification scheme using standard protein purification techniques. In another embodiment, GPCRX proteins are produced by recombinant DNA techniques. Alternative to recombinant expression, an GPCRX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the GPCRX protein is derived, or substantially free from chemical precursors or other chemicals when chemically synthesized. The language “substantially free of cellular material” includes preparations of GPCRX proteins in which the protein is separated from cellular components of the cells from which it is isolated or recombinantly-produced. In one embodiment, the language “substantially free of cellular material” includes preparations of GPCRX proteins having less than about 30% (by dry weight) of non-GPCRX proteins (also referred to herein as a “contaminating protein”), more preferably less than about 20% of non-GPCRX proteins, still more preferably less than about 10% of non-GPCRX proteins, and most preferably less than about 5% of non-GPCRX proteins. When the GPCRX protein or biologically-active portion thereof is recombinantly-produced, it is also preferably substantially free of culture medium, i.e., culture medium represents less than about 20%, more preferably less than about 10%, and most preferably less than about 5% of the volume of the GPCRX protein preparation.

The language “substantially free of chemical precursors or other chemicals” includes preparations of GPCRX proteins in which the protein is separated from chemical precursors or other chemicals that are involved in the synthesis of the protein. In one embodiment, the language “substantially free of chemical precursors or other chemicals” includes preparations of GPCRX proteins having less than about 30% (by dry weight) of chemical precursors or non-GPCRX chemicals, more preferably less than about 20% chemical precursors or non-GPCRX chemicals, still more preferably less than about 10% chemical precursors or non-GPCRX chemicals, and most preferably less than about 5% chemical precursors or non-GPCRX chemicals.

Biologically-active portions of GPCRX proteins include peptides comprising amino acid sequences sufficiently homologous to or derived from the amino acid sequences of the GPCRX proteins (e.g., the amino acid sequence shown in SEQ ID NOS: 2, 4, 6, 8, 10, 12, 14, 16, 18, 20, 22, 24, 26, 28, 30, 32 and 34) that include fewer amino acids than the fall-length GPCRX proteins, and exhibit at least one activity of an GPCRX protein. Typically, biologically-active portions comprise a domain or motif with at least one activity of the GPCRX protein. A biologically-active portion of an GPCRX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native GPCRX protein.

In an embodiment, the GPCRX protein has an amino acid sequence shown in SEQ ID NOS: 2, 4, 6, 8, 10, 12, 14, 16, 18, 20, 22, 24, 26, 28, 30, 32 and 34. In other embodiments, the GPCRX protein is substantially homologous to SEQ ID NOS: 2, 4, 6, 8, 10, 12, 14, 16, 18, 20, 22, 24, 26, 28, 30, 32 and 34, and retains the functional activity of the protein of SEQ ID NOS: 2, 4, 6, 8, 10, 12, 14, 16, 18, 20, 22, 24, 26, 28, 30, 32 and 34, yet differs in amino acid sequence due to natural allelic variation or mutagenesis, as described in detail, below. Accordingly, in another embodiment, the GPCRX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32 and 34, and retains the functional activity of the GPCRX proteins of SEQ ID NOS: 2, 4, 6, 8, 10, 12, 14, 16, 18, 20, 22, 24, 26, 28, 30, 32 and 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31 and 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides GPCRX chimeric or fusion proteins. As used herein, an GPCRX “chimeric protein” or “fusion protein” comprises an GPCRX polypeptide operatively-linked to a non-GPCRX polypeptide. An “GPCRX polypeptide” refers to a polypeptide having an amino acid sequence corresponding to an GPCRX protein (SEQ ID NOS: 2, 4, 6, 8, 10, 12, 14, 16, 18, 20, 22, 24, 26, 28, 30, 32 and 34), whereas a “non-GPCRX polypeptide” refers to a polypeptide having an amino acid sequence corresponding to a protein that is not substantially homologous to the GPCRX protein, e.g., a protein that is different from the GPCRX protein and that is derived from the same or a different organism. Within an GPCRX fusion protein the GPCRX polypeptide can correspond to all or a portion of an GPCRX protein. In one embodiment, an GPCRX fusion protein comprises at least one biologically-active portion of an GPCRX protein. In another embodiment, an GPCRX fusion protein comprises at least two biologically-active portions of an GPCRX protein. In yet another embodiment, an GPCRX fusion protein comprises at least three biologically-active portions of an GPCRX protein. Within the fusion protein, the term “operatively-linked” is intended to indicate that the GPCRX polypeptide and the non-GPCRX polypeptide are fused in-frame with one another. The non-GPCRX polypeptide can be fused to the N-terminus or C-terminus of the GPCRX polypeptide.

In one embodiment, the fusion protein is a GST-GPCRX fusion protein in which the GPCRX sequences are fused to the C-terminus of the GST (glutathione S-transferase) sequences. Such fusion proteins can facilitate the purification of recombinant GPCRX polypeptides.

In another embodiment, the fusion protein is an GPCRX protein containing a heterologous signal sequence at its N-terminus. In certain host cells (e.g., mammalian host cells), expression and/or secretion of GPCRX can be increased through use of a heterologous signal sequence.

In yet another embodiment, the fusion protein is an GPCRX-immunoglobulin fusion protein in which the GPCRX sequences are fused to sequences derived from a member of the immunoglobulin protein family. The GPCRX-immunoglobulin fusion proteins of the invention can be incorporated into pharmaceutical compositions and administered to a subject to inhibit an interaction between an GPCRX ligand and an GPCRX protein on the surface of a cell, to thereby suppress GPCRX-mediated signal transduction in vivo. The GPCRX-immunoglobulin fusion proteins can be used to affect the bioavailability of an GPCRX cognate ligand. Inhibition of the GPCRX ligand/GPCRX interaction may be useful therapeutically for both the treatment of proliferative and differentiative disorders, as well as modulating (e.g. promoting or inhibiting) cell survival. Moreover, the GPCRX-immunoglobulin fusion proteins of the invention can be used as immunogens to produce anti-GPCRX antibodies in a subject, to purify GPCRX ligands, and in screening assays to identify molecules that inhibit the interaction of GPCRX with an GPCRX ligand.

An GPCRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An GPCRX-encoding nucleic acid can be cloned into such an expression vector such that the fusion moiety is linked in-frame to the GPCRX protein.

GPCRX Agonists and Antagonists

The invention also pertains to variants of the GPCRX proteins that function as either GPCRX agonists (i.e., mimetics) or as GPCRX antagonists. Variants of the GPCRX protein can be generated by mutagenesis (e.g., discrete point mutation or truncation of the GPCRX protein). An agonist of the GPCRX protein can retain substantially the same, or a subset of, the biological activities of the naturally occurring form of the GPCRX protein. An antagonist of the GPCRX protein can inhibit one or more of the activities of the naturally occurring form of the GPCRX protein by, for example, competitively binding to a downstream or upstream member of a cellular signaling cascade which includes the GPCRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the GPCRX proteins.

Variants of the GPCRX proteins that function as either GPCRX agonists (i.e., mimetics) or as GPCRX antagonists can be identified by screening combinatorial libraries of mutants (e.g., truncation mutants) of the GPCRX proteins for GPCRX protein agonist or antagonist activity. In one embodiment, a variegated library of GPCRX variants is generated by combinatorial mutagenesis at the nucleic acid level and is encoded by a variegated gene library. A variegated library of GPCRX variants can be produced by, for example, enzymatically ligating a mixture of synthetic oligonucleotides into gene sequences such that a degenerate set of potential GPCRX sequences is expressible as individual polypeptides, or alternatively, as a set of larger fusion proteins (e.g., for phage display) containing the set of GPCRX sequences therein. There are a variety of methods which can be used to produce libraries of potential GPCRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCRX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the GPCRX protein coding sequences can be used to generate a variegated population of GPCRX fragments for screening and subsequent selection of variants of an GPCRX protein. In one embodiment, a library of coding sequence fragments can be generated by treating a double stranded PCR fragment of an GPCRX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the GPCRX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GPCRX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify GPCRX variants. See, e.g., Arkin and Yourvan, 1992. Proc. Natl. Acad. Sci. USA 89: 7811-7815; Delgrave, et al., 1993. Protein Engineering 6:327-331.

Anti-GPCRX Antibodies

Also included in the invention are antibodies to GPCRX proteins, or fragments of GPCRX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated GPCRX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of GPCRX-related protein that is located on the surface of the protein, e.g., a hydrophilic region. A hydrophobicity analysis of the human GPCRX-related protein sequence will indicate which regions of a GPCRX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, NY, incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Strect. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96). In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers. Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fᵥ (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, a pha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an GPCRX protein is facilitated by generation of hybridomas that bind to the fragment of an GPCRX protein possessing such a domain. Thus, antibodies that are specific for a desired domain within an GPCRX protein, or derivatives, fragments, analogs or homologs thereof, are also provided herein.

Anti-GPCRX antibodies may be used in methods known within the art relating to the localization and/or quantitation of an GPCRX protein (e.g., for use in measuring levels of the GPCRX protein within appropriate physiological samples, for use in diagnostic methods, for use in imaging the protein, and the like). In a given embodiment, antibodies for GPCRX proteins, or derivatives, fragments, analogs or homologs thereof, that contain the antibody derived binding domain, are utilized as pharmacologically-active compounds (hereinafter “Therapeutics”).

An anti-GPCRX antibody (e.g., monoclonal antibody) can be used to isolate an GPCRX polypeptide by standard techniques, such as affinity chromatography or immunoprecipitation. An anti-GPCRX antibody can facilitate the purification of natural GPCRX polypeptide from cells and of recombinantly-produced GPCRX polypeptide expressed in host cells. Moreover, an anti-GPCRX antibody can be used to detect GPCRX protein (e.g., in a cellular lysate or cell supernatant) in order to evaluate the abundance and pattern of expression of the GPCRX protein. Anti-GPCRX antibodies can be used diagnostically to monitor protein levels in tissue as part of a clinical testing procedure, e.g. to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

GPCRX Recombinant Expression Vectors and Host Cells

Another aspect of the invention pertains to vectors, preferably expression vectors, containing a nucleic acid encoding an GPCRX protein, or derivatives, fragments, analogs or bomologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GPCRX proteins, mutant forms of GPCRX proteins, fusion proteins, etc.).

The recombinant expression vectors of the invention can be designed for expression of GPCRX proteins in prokaryotic or eukaryotic cells. For example, GPCRX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the GPCRX expression vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). Alternatively, GPCRX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264, 166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to GPCRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis, ” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, GPCRX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding GPCRX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GPCRX protein. Accordingly, the invention further provides methods for producing GPCRX protein using the host cells of the invention. In one embodiment, the method comprises culturing the host cell of invention (into which a recombinant expression vector encoding GPCRX protein has been introduced) in a suitable medium such that GPCRX protein is produced. In another embodiment, the method further comprises isolating GPCRX protein from the medium or the host cell.

Transgenic GPCRX Animals

The host cells of the invention can also be used to produce non-human transgenic animals. For example, in one embodiment, a host cell of the invention is a fertilized oocyte or an embryonic stem cell into which GPCRX protein-coding sequences have been introduced. Such host cells can then be used to create non-human transgenic animals in which exogenous GPCRX sequences have been introduced into their genome or homologous recombinant animals in which endogenous GPCRX sequences have been altered. Such animals are useful for studying the function and/or activity of GPCRX protein and for identifying and/or evaluating modulators of GPCRX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GPCRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GPCRX-encoding nucleic acid into the male pronuclei of a fertilized oocyte (e.g., by microinjection, retroviral infection) and allowing the oocyte to develop in a pseudopregnant female foster animal. The human GPCRX cDNA sequences of SEQ ID NOS: 1, 3, 5, 7, 9, 11, 13, 15, 17, 19, 21, 23, 25, 27, 29, 31 and 33 can be introduced as a transgene into the genome of a non-human animal. Alternatively, a non-human homologue of the human GPCRX gene, such as a mouse GPCRX gene, can be isolated based on hybridization to the human GPCRX cDNA (described further supra) and used as a transgene. Intronic sequences and polyadenylation signals can also be included in the transgene to increase the efficiency of expression of the transgene. A tissue-specific regulatory sequence(s) can be operably-linked to the GPCRX transgene to direct expression of GPCRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the GPCRX transgene in its genome and/or expression of GPCRX mRNA in tissues or cells of the animals. A transgenic founder animal can then be used to breed additional animals carrying the transgene. Moreover, transgenic animals carrying a transgene-encoding GPCRX protein can further be bred to other transgenic animals carrying other transgenes.

To create a homologous recombinant animal, a vector is prepared which contains at least a portion of an GPCRX gene into which a deletion, addition or substitution has been introduced to thereby alter, e.g., functionally disrupt, the GPCRX gene. The GPCRX gene can be a human gene (e.g., the cDNA of SEQ ID NOS: 1, 3, 5, 7, 9, 11, 13, 15, 17, 19, 21, 23, 25, 27, 29, 31 and 33), but more preferably, is a non-human homologue of a human GPCRX gene. For example, a mouse homologue of human GPCRX gene of SEQ ID NOS: 1, 3, 5, 7, 9, 11, 13, 15, 17, 19, 21, 23, 25, 27, 29, 31 and 33 can be used to construct a homologous recombination vector suitable for altering an endogenous GPCRX gene in the mouse genome. In one embodiment, the vector is designed such that, upon homologous recombination, the endogenous GPCRX gene is functionally disrupted (i.e., no longer encodes a functional protein; also referred to as a “knock out” vector).

Alternatively, the vector can be designed such that, upon homologous recombination, the endogenous GPCRX gene is mutated or otherwise altered but still encodes functional protein (e.g., the upstream regulatory region can be altered to thereby alter the expression of the endogenous GPCRX protein). In the homologous recombination vector, the altered portion of the GPCRX gene is flanked at its 5′- and 3′-termini by additional nucleic acid of the GPCRX gene to allow for homologous recombination to occur between the exogenous GPCRX gene carried by the vector and an endogenous GPCRX gene in an embryonic stem cell. The additional flanking GPCRX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced GPCRX gene has homologously-recombined with the endogenous GPCRX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The GPCRX nucleic acid molecules, GPCRX proteins, and anti-GPCRX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an GPCRX protein or anti-GPCRX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express GPCRX protein (e.g., via a recombinant expression vector in a host cell in gene therapy applications), to detect GPCRX mRNA (e.g., in a biological sample) or a genetic lesion in an GPCRX gene, and to modulate GPCRX activity, as described further, below. In addition, the GPCRX proteins can be used to screen drugs or compounds that modulate the GPCRX protein activity or expression as well as to treat disorders characterized by insufficient or excessive production of GPCRX protein or production of GPCRX protein forms that have decreased or aberrant activity compared to GPCRX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-GPCRX antibodies of the invention can be used to detect and isolate GPCRX proteins and modulate GPCRX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i. e., candidate or test compounds or agents (e.g., peptides, peptidomimetics, small molecules or other drugs) that bind to GPCRX proteins or have a stimulatory or inhibitory effect on, e.g., GPCRX protein expression or GPCRX protein activity. The invention also includes compounds identified in the screening assays described herein.

In one embodiment, the invention provides assays for screening candidate or test compounds which bind to or modulate the activity of the membrane-bound form of an GPCRX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of GPCRX protein, or a biologically-active portion thereof, on the cell surface is contacted with a test compound and the ability of the test compound to bind to an GPCRX protein determined. The cell, for example, can of mammalian origin or a yeast cell. Determining the ability of the test compound to bind to the GPCRX protein can be accomplished, for example, by coupling the test compound with a radioisotope or enzymatic label such that binding of the test compound to the GPCRX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of GPCRX protein, or a biologically-active portion thereof, on the cell surface with a known compound which binds GPCRX to form an assay mixture, contacting the assay mixture with a test recompound, and determining the ability of the test compound to interact with an GPCRX protein, wherein determining the ability of the test compound to interact with an GPCRX protein comprises determining the ability of the test compound to preferentially bind to GPCRX protein or a biologically-active portion thereof as compared to the known compound.

In another embodiment, an assay is a cell-based assay comprising contacting a cell expressing a membrane-bound form of GPCRX protein, or a biologically-active portion thereof, on the cell surface with a test compound and determining the ability of the test compound to modulate (e.g., stimulate or inhibit) the activity of the GPCRX protein or biologically-active portion thereof. Determining the ability of the test compound to modulate the activity of GPCRX or a biologically-active portion thereof can be accomplished, for example, by determining the ability of the GPCRX protein to bind to or interact with an GPCRX target molecule. As used herein, a “target molecule” is a molecule with which an GPCRX protein binds or interacts in nature, for example, a molecule on the surface of a cell which expresses an GPCRX interacting protein, a molecule on the surface of a second cell, a molecule in the extracellular milieu, a molecule associated with the internal surface of a cell membrane or a cytoplasmic molecule. An GPCRX target molecule can be a non-GPCRX molecule or an GPCRX protein or polypeptide of the invention. In one embodiment, an GPCRX target molecule is a component of a signal transduction pathway that facilitates transduction of an extracellular signal (e.g. a signal generated by binding of a compound to a membrane-bound GPCRX molecule) through the cell membrane and into the cell. The target, for example, can be a second intercellular protein that has catalytic activity or a protein that facilitates the association of downstream signaling molecules with GPCRX.

Determining the ability of the GPCRX protein to bind to or interact with an GPCRX target molecule can be accomplished by one of the methods described above for determining direct binding. In one embodiment, determining the ability of the GPCRX protein to bind to or interact with an GPCRX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an GPCRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an GPCRX protein or biologically-active portion thereof with a test compound and determining the ability of the test compound to bind to the GPCRX protein or biologically-active portion thereof. Binding of the test compound to the GPCRX protein can be determined either directly or indirectly as described above. In one such embodiment, the assay comprises contacting the GPCRX protein or biologically-active portion thereof with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX or biologically-active portion thereof as compared to the known compound.

In still another embodiment, an assay is a cell-free assay comprising contacting GPCRX protein or biologically-active portion thereof with a test compound and determining the ability of the test compound to modulate (e.g. stimulate or inhibit) the activity of the GPCRX protein or biologically-active portion thereof. Determining the ability of the test compound to modulate the activity of GPCRX can be accomplished, for example, by determining the ability of the GPCRX protein to bind to an GPCRX target molecule by one of the methods described above for determining direct binding. In an alternative embodiment, determining the ability of the test compound to modulate the activity of GPCRX protein can be accomplished by determining the ability of the GPCRX protein further modulate an GPCRX target molecule. For example, the catalytic/enzymatic activity of the target molecule on an appropriate substrate can be determined as described, supra.

In yet another embodiment, the cell-free assay comprises contacting the GPCRX protein or biologically-active portion thereof with a known compound which binds GPCRX protein to form an assay mixture, contacting the assay mixture with a test compound, and determining the ability of the test compound to interact with an GPCRX protein, wherein determining the ability of the test compound to interact with an GPCRX protein comprises determining the ability of the GPCRX protein to preferentially bind to or modulate the activity of an GPCRX target molecule.

The cell-free assays of the invention are amenable to use of both the soluble form or the membrane-bound form of GPCRX protein. In the case of cell-free assays comprising the membrane-bound form of GPCRX protein, it may be desirable to utilize a solubilizing agent such that the membrane-bound form of GPCRX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N, N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either GPCRX protein or its target molecule to facilitate separation of complexed from uncomplexed forms of one or both of the proteins, as well as to accommodate automation of the assay. Binding of a test compound to GPCRX protein, or interaction of GPCRX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-GPCRX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or GPCRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of GPCRX protein binding or activity determined using standard techniques.

Other techniques for immobilizing proteins on matrices can also be used in the screening assays of the invention. For example, either the GPCRX protein or its target molecule can be immobilized utilizing conjugation of biotin and streptavidin. Biotinylated GPCRX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with GPCRX protein or target molecules, but which do not interfere with binding of the GPCRX protein to its target molecule, can be derivatized to the wells of the plate, and unbound target or GPCRX protein trapped in the wells by antibody conjugation. Methods for detecting such complexes, in addition to those described above for the GST-immobilized complexes, include immunodetection of complexes using antibodies reactive with the GPCRX protein or target molecule, as well as enzyme-linked assays that rely on detecting an enzymatic activity associated with the GPCRX protein or target molecule.

In another embodiment, modulators of GPCRX protein expression are identified in a method wherein a cell is contacted with a candidate compound and the expression of GPCRX mRNA or protein in the cell is determined. The level of expression of GPCRX mRNA or protein in the presence of the candidate compound is compared to the level of expression of GPCRX mRNA or protein in the absence of the candidate compound. The candidate compound can then be identified as a modulator of GPCRX mRNA or protein expression based upon this comparison. For example, when expression of GPCRX mRNA or protein is greater (i.e., statistically significantly greater) in the presence of the candidate compound than in its absence, the candidate compound is identified as a stimulator of GPCRX mRNA or protein expression. Alternatively, when expression of GPCRX mRNA or protein is less (statistically significantly less) in the presence of the candidate compound than in its absence, the candidate compound is identified as an inhibitor of GPCRX mRNA or protein expression. The level of GPCRX mRNA or protein expression in the cells can be determined by methods described herein for detecting GPCRX mRNA or protein.

In yet another aspect of the invention, the GPCRX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with GPCRX (“GPCRX-binding proteins” or “GPCRX-bp”) and modulate GPCRX activity. Such GPCRX-binding proteins are also likely to be involved in the propagation of signals by the GPCRX proteins as, for example, upstream or downstream elements of the GPCRX pathway.

The two-hybrid system is based on the modular nature of most transcription factors, which consist of separable DNA-binding and activation domains. Briefly, the assay utilizes two different DNA constructs. In one construct, the gene that codes for GPCRX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an GPCRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with GPCRX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the GPCRX sequences, SEQ ID NOS: 1, 3, 5, 7, 9, 11, 13, 15, 17, 19, 21, 23, 25, 27, 29, 31 and 33, or fragments or derivatives thereof, can be used to map the location of the GPCRX genes, respectively, on a chromosome. The mapping of the GPCRX sequences to chromosomes is an important first step in correlating these sequences with genes associated with disease.

Briefly, GPCRX genes can be mapped to chromosomes by preparing PCR primers (preferably 15-25 bp in length) from the GPCRX sequences. Computer analysis of the GPCRX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the GPCRX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GPCRX sequences to design oligonucleotide primers, sub-localization can be achieved with panels of fragments from specific chromosomes. Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the GPCRX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The GPCRX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms, ” described in U.S. Pat. No. 5,272,057).

Furthermore, the sequences of the invention can be used to provide an alternative technique that determines the actual base-by-base DNA sequence of selected portions of an individual's genome. Thus, the GPCRX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The GPCRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31 and 33 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining GPCRX protein and/or nucleic acid expression as well as GPCRX activity, in the context of a biological sample (e.g., blood, serum, cells, tissue) to thereby determine whether an individual is afflicted with a disease or disorder, or is at risk of developing a disorder, associated with aberrant GPCRX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with GPCRX protein, nucleic acid expression or activity. For example, mutations in an GPCRX gene can be assayed in a biological sample. Such assays can be used for prognostic or predictive purpose to thereby prophylactically treat an individual prior to the onset of a disorder characterized by or associated with GPCRX protein, nucleic acid expression, or biological activity.

Another aspect of the invention provides methods for determining GPCRX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of GPCRX in clinical trials.

These and other agents are described in further detail in the following sections.

Diagnostic Assays

An exemplary method for detecting the presence or absence of GPCRX in a biological sample involves obtaining a biological sample from a test subject and contacting the biological sample with a compound or an agent capable of detecting GPCRX protein or nucleic acid (e.g., mRNA, genomic DNA) that encodes GPCRX protein such that the presence of GPCRX is detected in the biological sample. An agent for detecting GPCRX mRNA or genomic DNA is a labeled nucleic acid probe capable of hybridizing to GPCRX mRNA or genomic DNA. The nucleic acid probe can be, for example, a full-length GPCRX nucleic acid, such as the nucleic acid of SEQ ID NOS: 1, 3, 5, 7, 9, 11, 13, 15, 17, 19, 21, 23, 25, 27, 29, 31 and 33, or a portion thereof, such as an oligonucleotide of at least 15, 30, 50, 100, 250 or 500 nucleotides in length and sufficient to specifically hybridize under stringent conditions to GPCRX mRNA or genomic DNA. Other suitable probes for use in the diagnostic assays of the invention are described herein.

An agent for detecting GPCRX protein is an antibody capable of binding to GPCRX protein, preferably an antibody with a detectable label. Antibodies can be polyclonal, or more preferably, monoclonal. An intact antibody, or a fragment thereof (e.g. Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect GPCRX mRNA, protein, or genomic DNA in a biological sample in vitro as well as in vivo. For example, in vitro techniques for detection of GPCRX mRNA include Northern hybridizations and in situ hybridizations. In vitro techniques for detection of GPCRX protein include enzyme linked immunosorbent assays (ELISAs), Western blots, immunoprecipitations, and immunofluorescence. In vitro techniques for detection of GPCRX genomic DNA include Southern hybridizations. Furthermore, in vivo techniques for detection of GPCRX protein include introducing into a subject a labeled anti-GPCRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GPCRX protein, mRNA, or genomic DNA, such that the presence of GPCRX protein, mRNA or genomic DNA is detected in the biological sample, and comparing the presence of GPCRX protein, mRNA or genomic DNA in the control sample with the presence of GPCRX protein, mRNA or genomic DNA in the test sample.

The invention also encompasses kits for detecting the presence of GPCRX in a biological sample. For example, the kit can comprise: a labeled compound or agent capable of detecting GPCRX protein or mRNA in a biological sample; means for determining the amount of GPCRX in the sample; and means for comparing the amount of GPCRX in the sample with a standard. The compound or agent can be packaged in a suitable container. The kit can further comprise instructions for using the kit to detect GPCRX protein or nucleic acid.

Prognostic Assays

The diagnostic methods described herein can furthermore be utilized to identify subjects having or at risk of developing a disease or disorder associated with aberrant GPCRX expression or activity. For example, the assays described herein, such as the preceding diagnostic assays or the following assays, can be utilized to identify a subject having or at risk of developing a disorder associated with GPCRX protein, nucleic acid expression or activity. Alternatively, the prognostic assays can be utilized to identify a subject having or at risk for developing a disease or disorder. Thus, the invention provides a method for identifying a disease or disorder associated with aberrant GPCRX expression or activity in which a test sample is obtained from a subject and GPCRX protein or nucleic acid (e.g., mRNA, genomic DNA) is detected, wherein the presence of GPCRX protein or nucleic acid is diagnostic for a subject having or at risk of developing a disease or disorder associated with aberrant GPCRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant GPCRX expression or activity. For example, such methods can be used to determine whether a subject can be effectively treated with an agent for a disorder. Thus, the invention provides methods for determining whether a subject can be effectively treated with an agent for a disorder associated with aberrant GPCRX expression or activity in which a test sample is obtained and GPCRX protein or nucleic acid is detected (e.g., wherein the presence of GPCRX protein or nucleic acid is diagnostic for a subject that can be administered the agent to treat a disorder associated with aberrant GPCRX expression or activity).

The methods of the invention can also be used to detect genetic lesions in an GPCRX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an GPCRX-protein, or the misexpression of the GPCRX gene. For example, such genetic lesions can be detected by ascertaining the existence of at least one of: (i) a deletion of one or more nucleotides from an GPCRX gene; (ii) an addition of one or more nucleotides to an GPCRX gene; (iii) a substitution of one or more nucleotides of an GPCRX gene, (iv) a chromosomal rearrangement of an GPCRX gene; (v) an alteration in the level of a messenger RNA transcript of an GPCRX gene, (vi) aberrant modification of an GPCRX gene, such as of the methylation pattern of the genomic DNA, (vii) the presence of a non-wild-type splicing pattern of a messenger RNA transcript of an GPCRX gene, (viii) a non-wild-type level of an GPCRX protein, (ix) allelic loss of an GPCRX gene, and (x) inappropriate post-translational modification of an GPCRX protein. As described herein, there are a large number of assay techniques known in the art which can be used for detecting lesions in an GPCRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4, 683, 202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the GPCRX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an GPCRX gene under conditions such that hybridization and amplification of the GPCRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an GPCRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in GPCRX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in GPCRX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GPCRX gene and detect mutations by comparing the sequence of the sample GPCRX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g. PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the GPCRX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type GPCRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. NatL Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GPCRX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an GPCRX sequence, e.g., a wild-type GPCRX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039. In other embodiments, alterations in electrophoretic mobility will be used to identify mutations in GPCRX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control GPCRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an GPCRX gene.

Furthermore, any cell type or tissue, preferably peripheral blood leukocytes, in which GPCRX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on GPCRX activity (e.g., GPCRX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of GPCRX protein, expression of GPCRX nucleic acid, or mutation content of GPCRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of GPCRX protein, expression of GPCRX nucleic acid, or mutation content of GPCRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an GPCRX modulator, such as a modulator identified by one of the exemplary screening assays described herein.

Monitoring of Effects During Clinical Trials

Monitoring the influence of agents (e.g., drugs, compounds) on the expression or activity of GPCRX (e.g., the ability to modulate aberrant cell proliferation and/or differentiation) can be applied not only in basic drug screening, but also in clinical trials. For example, the effectiveness of an agent determined by a screening assay as described herein to increase GPCRX gene expression, protein levels, or upregulate GPCRX activity, can be monitored in clinical trails of subjects exhibiting decreased GPCRX gene expression, protein levels, or downregulated GPCRX activity. Alternatively, the effectiveness of an agent determined by a screening assay to decrease GPCRX gene expression, protein levels, or downregulate GPCRX activity, can be monitored in clinical trails of subjects exhibiting increased GPCRX gene expression, protein levels, or upregulated GPCRX activity. In such clinical trials, the expression or activity of GPCRX and, preferably, other genes that have been implicated in, for example, a cellular proliferation or immune disorder can be used as a “read out” or markers of the immune responsiveness of a particular cell.

By way of example, and not of limitation, genes, including GPCRX, that are modulated in cells by treatment with an agent (e.g., compound, drug or small molecule) that modulates GPCRX activity (e.g., identified in a screening assay as described herein) can be identified. Thus, to study the effect of agents on cellular proliferation disorders, for example, in a clinical trial, cells can be isolated and RNA prepared and analyzed for the levels of expression of GPCRX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of GPCRX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an GPCRX protein, mRNA, or genomic DNA in the preadministration sample; (iii) obtaining one or more post-administration samples from the subject; (iv) detecting the level of expression or activity of the GPCRX protein, mRNA, or genomic DNA in the post-administration samples; (v) comparing the level of expression or activity of the GPCRX protein, mRNA, or genomic DNA in the pre-administration sample with the GPCRX protein, mRNA, or genomic DNA in the post administration sample or samples; and (vi) altering the administration of the agent to the subject accordingly. For example, increased administration of the agent may be desirable to increase the expression or activity of GPCRX to higher levels than detected, ie., to increase the effectiveness of the agent. Alternatively, decreased administration of the agent may be desirable to decrease expression or activity of GPCRX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant GPCRX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright hereditary ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant GPCRX expression or activity, by administering to the subject an agent that modulates GPCRX expression or at least one GPCRX activity. Subjects at risk for a disease that is caused or contributed to by aberrant GPCRX expression or activity can be identified by, for example, any or a combination of diagnostic or prognostic assays as described herein. Administration of a prophylactic agent can occur prior to the manifestation of symptoms characteristic of the GPCRX aberrancy, such that a disease or disorder is prevented or, alternatively, delayed in its progression. Depending upon the type of GPCRX aberrancy, for example, an GPCRX agonist or GPCRX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating GPCRX expression or activity for therapeutic purposes. The modulatory method of the invention involves contacting a cell with an agent that modulates one or more of the activities of GPCRX protein activity associated with the cell. An agent that modulates GPCRX protein activity can be an agent as described herein, such as a nucleic acid or a protein, a naturally-occurring cognate ligand of an GPCRX protein, a peptide, an GPCRX peptidomimetic, or other small molecule. In one embodiment, the agent stimulates one or more GPCRX protein activity. Examples of such stimulatory agents include active GPCRX protein and a nucleic acid molecule encoding GPCRX that has been introduced into the cell. In another embodiment, the agent inhibits one or more GPCRX protein activity. Examples of such inhibitory agents include antisense GPCRX nucleic acid molecules and anti-GPCRX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an GPCRX protein or nucleic acid molecule. In one embodiment, the method involves administering an agent (e.g., an agent identified by a screening assay described herein), or combination of agents that modulates (e.g., up-regulates or down-regulates) GPCRX expression or activity. In another embodiment, the method involves administering an GPCRX protein or nucleic acid molecule as therapy to compensate for reduced or aberrant GPCRX expression or activity.

Stimulation of GPCRX activity is desirable in situations in which GPCRX is abnormally downregulated and/or in which increased GPCRX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The GPCRX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's disease, Parkinson's disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the GPCRX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's disease, Parkinson's disorder, immune disorders, hematopoietic disorders, and the various dyslipidemias.

Both the novel nucleic acid encoding the GPCRX protein, and the GPCRX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

The invention will be further described in the following examples, which do not limit the scope of the invention described in the claims.

### EXAMPLES

### Example 1

Identification of GPCRX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel GPCRX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence.

### Example 3

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1X TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, MD) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 1 00 M non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 gM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 gM non essential amino acids (Gibco), 1 mM sodium pyruvate (Gibco), mercaptoethanol (5.5×10⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, UT), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 100 μM non essential amino acids (Gibco), 1 mM sodium pyruvate (Gibco), mercaptoethanol 5.5×10⁻⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁻⁵ cells/ml for 8 days, changing the media every 3 days and adjusting the cell concentration to 5×10⁻⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-i anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 51

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4 =Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; patient not demented but showing sever AD-like

pathology

SupTemporal Ctx=Superior Temporal Cortex

Inf Temporal Ctx=Inferior Temporal Cortex

GPCR1a and GPCR1b

Expression of these GPCR1 genes (also referred to as SC35113271_A_da1, CG55798-03) was assessed using the primer-probe sets Ag1533 and Ag2269, described in Tables 16 and 17. Results of the RTQ-PCR runs are shown in Tables 18, 19, 20, 21, 22, 23 and 24.

TABLE 16
Probe Name Ag1533

| | | | Start | SEQ ID |
|---|---|---|---|---|
| Primers | Sequences | Length | Position | NO: |
| Forward | 5′-accatcatcaagagtgctatgg-3′ | 22 | 431 | 119 |
| Probe | TET-5′-tcctttcgaagcttctgcatcatcct-3′-TAMRA | 26 | 458 | 120 |
| Reverse | 5′-aggcatgtcagcaagaatacat-3′ | 22 | 489 | 121 |

TABLE 17
Probe Name Ag2269

| | | | Start | SEQ ID |
|---|---|---|---|---|
| Primers | Sequences | Length | Positon | NO: |
| Forward | 5′-atggcatttgatcgctatgtag-3′ | 22 | 365 | 122 |
| Probe | TET-5′-tgagatataccaccatcttgactccca-3′-TAMRA | 27 | 402 | 123 |
| Reverse | 5′-ccatagcactcttgatgatggt-3′ | 22 | 431 | 124 |

TABLE 18
CNS_neurodegeneration_v1.0

| | Rel. | | Rel. |
|---|---|---|---|
| | Exp. (%) | | Exp. (%) |
| | Ag1533, | | Ag1533, |
| | Run | | Run |
| Tissue Name | 225432469 | Tissue Name | 225432469 |
| AD 1 Hippo | 36.6 | Control (Path) 3 | 16.5 |
| | | Temporal Ctx | |
| AD 2 Hippo | 11.6 | Control (Path) 4 | 42.9 |
| | | Temporal Ctx | |
| AD 3 Hippo | 50.7 | AD 1 Occipital Ctx | 39.2 |
| AD 4 Hippo | 75.3 | AD 2 Occipital Ctx | 0.0 |
| | | (Missing) | |
| AD 5 Hippo | 20.4 | AD 3 Occipital Ctx | 25.9 |
| AD 6 Hippo | 69.3 | AD 4 Occipital Ctx | 82.9 |
| Control 2 Hippo | 22.5 | AD 5 Occipital Ctx | 11.0 |
| Control 4 Hippo | 53.2 | AD 5 Occipital Ctx | 7.1 |
| Control (Path) 3 | 29.7 | Control 1 Occipital | 11.9 |
| Hippo | | Ctx | |
| AD 1 Temporal Ctx | 96.6 | Control 2 Occipital | 29.1 |
| | | Ctx | |
| AD 2 Temporal Ctx | 34.6 | Control 3 Occipital | 41.2 |
| | | Ctx | |
| AD 3 Temporal Ctx | 54.3 | Control 4 Occipital | 11.9 |
| | | Ctx | |
| AD 4 Temporal Ctx | 68.3 | Control (Path) 1 | 23.3 |
| | | Occipital Ctx | |
| AD 5 Inf Temporal | 90.8 | Control (Path) 2 | 14.3 |
| Ctx | | Occipital Ctx | |
| AD 5 Sup Temporal | 53.2 | Control (Path) 3 | 35.1 |
| Ctx | | Occipital Ctx | |
| AD 6 Inf Temporal | 62.0 | Control (Path) 4 | 30.8 |
| Ctx | | Occipital Ctx | |
| AD 6 Sup Temporal | 55.5 | Control 1 Parietal | 18.2 |
| Ctx | | Ctx | |
| Control 1 Temporal | 5.3 | Control 2 Parietal | 69.3 |
| Ctx | | Ctx | |
| Control 2 Temporal | 19.6 | Control 3 Parietal | 6.3 |
| Ctx | | Ctx | |
| Control 3 Temporal | 38.7 | Control (Path) 1 | 33.9 |
| Ctx | | Parietal Ctx | |
| Control 3 Temporal | 17.0 | Control (Path) 2 | 18.4 |
| Ctx | | Parietal Ctx | |
| Control (Path) 1 | 29.1 | Control (Path) 3 | 26.8 |
| Temporal Ctx | | Parietal Ctx | |
| Control (Path) 2 | 23.7 | Control (Path) 4 | 100.0 |
| Temporal Ctx | | Parietal Ctx | |

TABLE 19
General_screening_panel_v1.4

| | Rel. | | Rel. |
|---|---|---|---|
| | Exp. (%) | | Exp. (%) |
| | Ag2269, | | Ag2269, |
| | Run | | Run |
| Tissue Name | 216861070 | Tissue Name | 216861070 |
| Adipose | 4.2 | Renal ca. TK-10 | 0.7 |
| Melanoma* | 0.3 | Bladder | 16.0 |
| Hs688(A).T | | | |
| Melanoma* | 1.2 | Gastric ca. (liver | 8.7 |
| Hs688(B).T | | met.) NCI-N87 | |
| Melanoma* M14 | 0.1 | Gastric ca. KATO | 0.0 |
| | | III | |
| Melanoma* | 0.0 | Colon ca. SW-948 | 0.3 |
| LOXIMVI | | | |
| Melanoma* SK | 0.2 | Colon ca. SW480 | 0.0 |
| MEL-5 | | | |
| Squamous cell | 100.0 | Colon ca.* (SW480 | 0.1 |
| carcinoma SCC-4 | | met) SW620 | |
| Testis Pool | 3.2 | Colon ca. HT29 | 0.0 |
| Prostate ca.* | 1.3 | Colon ca. HCT-116 | 0.7 |
| (bone met) PC-3 | | | |
| Prostate Pool | 8.1 | Colon ca. CaCo-2 | 0.2 |
| Placenta | 1.4 | Colon cancer | 0.5 |
| | | tissue | |
| Uterus Pool | 1.6 | Colon ca. SW1116 | 0.1 |
| Ovarian ca. | 10.4 | Colon ca. Colo-205 | 0.0 |
| OVCAR-3 | | | |
| Ovarian ca. SK- | 38.2 | Colon ca. SW-48 | 0.0 |
| OV-3 | | | |
| Ovarian ca. | 0.0 | Colon Pool | 6.5 |
| OVCAR-4 | | | |
| Ovarian ca. | 1.3 | Small Intestine | 17.7 |
| OVCAR-5 | | Pool | |
| Ovarian ca. | 0.1 | Stomach Pool | 11.0 |
| IGROV-1 | | | |
| Ovarian ca. | 0.1 | Bone Marrow Pool | 3.5 |
| OVCAR-8 | | | |
| Ovary | 4.6 | Fetal Heart | 6.5 |
| Breast ca. MCF-7 | 0.1 | Heart Pool | 4.1 |
| Breast ca. MDA- | 0.1 | Lymph Node Pool | 13.6 |
| MB-231 | | | |
| Breast ca. BT 549 | 7.7 | Fetal Skeletal | 3.3 |
| | | Muscle | |
| Breast ca. T47D | 9.1 | Skeletal Muscle | 0.5 |
| | | Pool | |
| Breast ca. MDA-N | 8.4 | Spleen Pool | 8.9 |
| Breast Pool | 17.4 | Thymus Pool | 8.2 |
| Trachea | 9.0 | CNS cancer (glio/ | 0.3 |
| | | astro) U87-MG | |
| Lung | 5.1 | CNS cancer (glio/ | 1.0 |
| | | astro) U-118-MG | |
| Fetal Lung | 25.3 | CNS cancer | 0.0 |
| | | (neuro;met) SK-N- | |
| | | AS | |
| Lung ca. NCI-N417 | 0.0 | CNS cancer (astro) | 0.7 |
| | | SF-539 | |
| Lung ca. LX-1 | 0.0 | CNS cancer (astro) | 2.1 |
| | | SNB-75 | |
| Lung ca. NCI-H146 | 0.7 | CNS cancer (glio) | 0.1 |
| | | SNB-19 | |
| Lung ca. SHP-77 | 0.0 | CNS cancer (glio) | 6.8 |
| | | SF-295 | |
| Lung ca. A549 | 0.4 | Brain (Amygdala) | 0.7 |
| | | Pool | |
| Lung ca. NCI-H526 | 0.0 | Brain (cerebellum) | 0.1 |
| Lung ca. NCI-H23 | 1.6 | Brain (fetal) | 2.1 |
| Lung ca. NCI-H460 | 51.8 | Brain (Hippo- | 1.1 |
| | | campus) Pool | |
| Lung ca. HOP-62 | 3.4 | Cerebral Cortex | 0.4 |
| | | Pool | |
| Lung ca. NCI-H522 | 0.1 | Brain (Substantia | 0.2 |
| | | nigra) Pool | |
| Liver | 0.2 | Brain (Thalamus) | 0.9 |
| | | Pool | |
| Fetal Liver | 2.6 | Brain (whole) | 0.7 |
| Liver ca. HepG2 | 0.0 | Spinal Cord Pool | 8.4 |
| Kidney Pool | 20.0 | Adrenal Gland | 3.9 |
| Fetal Kidney | 25.0 | Pituitary gland | 0.2 |
| | | Pool | |
| Renal ca. 786-0 | 0.3 | Salivary Gland | 0.4 |
| Renal ca. A498 | 1.1 | Thyroid (female) | 0.5 |
| Renal ca. ACHN | 0.9 | Pancreatic ca. | 0.4 |
| | | CAPAN2 | |
| Renal ca. UO-31 | 1.1 | Pancreas Pool | 10.2 |

TABLE 20
General_screening_panel_v1.5

| | Rel. Exp. (%) | Rel. Exp. (%) | | Rel. Exp. (%) | Rel. Exp. (%) |
|---|---|---|---|---|---|
| | Ag1533, Run | Ag2269, Run | | Ag1533, Run | Ag2269, Run |
| Tissue Name | 228632846 | 229393891 | Tissue Name | 228632846 | 229393891 |
| Adipose | 12.9 | 7.7 | Renal ca. TK-10 | 2.4 | 1.2 |
| Melanoma* | 1.5 | 1.7 | Bladder | 43.8 | 24.7 |
| Hs688(A).T | | | | | |
| Melanoma* | 0.4 | 2.8 | Gastric ca. (liver | 54.7 | 46.3 |
| Hs688(B).T | | | met.) NCI-N87 | | |
| Melanoma* | 0.0 | 0.0 | Gastric ca. | 0.4 | 1.1 |
| M14 | | | KATO III | | |
| Melanoma* | 0.5 | 0.8 | Colon ca. SW- | 0.0 | 0.0 |
| LOXIMVI | | | 948 | | |
| Melanoma* | 0.3 | 1.2 | Colon ca. SW480 | 0.0 | 0.0 |
| SK-MEL-5 | | | | | |
| Squamous cell | 0.0 | 1.3 | Colon ca.* | 0.0 | 0.0 |
| carcinoma | | | (SW480 met) | | |
| SCC-4 | | | SW620 | | |
| Testis Pool | 15.1 | 17.6 | Colon ca. HT29 | 0.0 | 1.0 |
| Prostate ca.* | 1.9 | 2.0 | Colon ca. HCT- | 2.8 | 2.4 |
| (bone met) | | | 116 | | |
| PC-3 | | | | | |
| Prostate Pool | 23.7 | 17.9 | Colon ca. CaCo-2 | 0.7 | 1.3 |
| Placenta | 2.9 | 5.1 | Colon cancer | 1.5 | 4.5 |
| | | | tissue | | |
| Uterus Pool | 11.5 | 15.5 | Colon ca. | 0.3 | 0.0 |
| | | | SW1116 | | |
| Ovarian ca. | 25.5 | 20.2 | Colon ca. Colo- | 0.0 | 0.5 |
| OVCAR-3 | | | 205 | | |
| Ovarian ca. | 87.7 | 91.4 | Colon ca. SW-48 | 0.0 | 0.0 |
| SK-OV-3 | | | | | |
| Ovarian ca. | 0.0 | 0.0 | Colon Pool | 37.4 | 28.7 |
| OVCAR-4 | | | | | |
| Ovarian ca. | 10.1 | 3.9 | Small Intestine | 43.5 | 33.4 |
| OVCAR-5 | | | Pool | | |
| Ovarian ca. | 0.0 | 0.7 | Stomach Pool | 27.4 | 21.3 |
| IGROV-1 | | | | | |
| Ovarian ca. | 2.4 | 1.4 | Bone Marrow | 22.1 | 20.6 |
| OVCAR-8 | | | Pool | | |
| Ovary | 22.2 | 27.2 | Fetal Heart | 57.0 | 36.9 |
| Breast ca. | 0.0 | 0.5 | Heart Pool | 12.2 | 9.2 |
| MCF-7 | | | | | |
| Breast ca. | 1.3 | 0.4 | Lymph Node | 66.9 | 47.6 |
| MDA-MB- | | | Pool | | |
| 231 | | | | | |
| Breast ca. BT | 1.4 | 3.3 | Fetal Skeletal | 11.4 | 10.5 |
| 549 | | | Muscle | | |
| Breast ca. | 0.8 | 0.0 | Skeletal Muscle | 7.5 | 4.1 |
| T47D | | | Pool | | |
| Breast ca. | 0.7 | 0.4 | Spleen Pool | 21.3 | 13.2 |
| MDA-N | | | | | |
| Breast Pool | 59.5 | 62.0 | Thymus Pool | 38.7 | 31.9 |
| Trachea | 19.1 | 12.4 | CNS cancer | 1.6 | 4.0 |
| | | | (glio/astro) U87- | | |
| | | | MG | | |
| Lung | 27.2 | 17.1 | CNS cancer | 1.5 | 2.4 |
| | | | (glio/astro) U- | | |
| | | | 118-MG | | |
| Fetal Lung | 100.0 | 75.8 | CNS cancer | 0.0 | 0.0 |
| | | | (neuro;met) SK- | | |
| | | | N-AS | | |
| Lung ca. NCI- | 0.0 | 0.0 | CNS cancer | 1.1 | 0.5 |
| N417 | | | (astro) SF-539 | | |
| Lung ca. LX- | 0.4 | 0.0 | CNS cancer | 4.5 | 5.7 |
| 1 | | | (astro) SNB-75 | | |
| Lung ca. NCI- | 4.7 | 3.7 | CNS cancer | 1.1 | 0.0 |
| H146 | | | (glio) SNB-19 | | |
| Lung ca. | 0.0 | 0.0 | CNS cancer | 20.6 | 29.1 |
| SHP-77 | | | (glio) SF-295 | | |
| Lung ca. | 2.9 | 1.0 | Brain | 2.1 | 1.3 |
| A549 | | | (Amygdala) Pool | | |
| Lung ca. NCI- | 0.0 | 0.0 | Brain | 0.9 | 0.6 |
| H526 | | | (cerebellum) | | |
| Lung ca. NCI- | 6.7 | 10.3 | Brain (fetal) | 7.9 | 7.3 |
| H23 | | | | | |
| Lung ca. NCI- | 9.2 | 1.9 | Brain | 1.6 | 2.6 |
| H460 | | | (Hippocampus) | | |
| | | | Pool | | |
| Lung ca. | 8.4 | 3.3 | Cerebral Cortex | 2.6 | 2.5 |
| HOP-62 | | | Pool | | |
| Lung ca. NCI- | 0.0 | 0.3 | Brain (Substantia | 2.0 | 0.8 |
| H522 | | | nigra) Pool | | |
| Liver | 0.4 | 0.2 | Brain (Thalamus) | 2.5 | 5.1 |
| | | | Pool | | |
| Fetal Liver | 8.7 | 6.5 | Brain (whole) | 3.8 | 2.6 |
| Liver ca. | 0.7 | 0.0 | Spinal Cord Pool | 5.0 | 3.6 |
| HepG2 | | | | | |
| Kidney Pool | 67.8 | 72.2 | Adrenal Gland | 11.0 | 11.3 |
| Fetal Kidney | 94.6 | 100.0 | Pituitary gland | 5.3 | 4.0 |
| | | | Pool | | |
| Renal ca. 786- | 1.2 | 4.2 | Salivary Gland | 1.7 | 1.4 |
| 0 | | | | | |
| Renal ca. | 1.8 | 5.7 | Thyroid (female) | 2.5 | 5.5 |
| A498 | | | | | |
| Renal ca. | 3.6 | 2.8 | Pancreatic ca. | 4.7 | 6.3 |
| ACHN | | | CAPAN2 | | |
| Renal ca. UO- | 5.8 | 3.1 | Pancreas Pool | 39.8 | 24.1 |
| 31 | | | | | |

TABLE 21
Panel 1.2

| | Rel. | | Rel. |
|---|---|---|---|
| | Exp. (%) | | Exp. (%) |
| | Ag1533, | | Ag1533, |
| | Run | | Run |
| Tissue Name | 142223910 | Tissue Name | 142223910 |
| Endothelial cells | 4.4 | Renal ca. 786-0 | 1.8 |
| Heart (Fetal) | 3.9 | Renal ca. A498 | 7.5 |
| Pancreas | 4.8 | Renal ca. RXF 393 | 1.8 |
| Pancreatic ca. | 1.1 | Renal ca. ACHN | 5.0 |
| CAPAN 2 | | | |
| Adrenal Gland | 20.9 | Renal ca. UO-31 | 9.0 |
| Thyroid | 1.1 | Renal ca. TK-10 | 2.3 |
| Salivary gland | 52.1 | Liver | 9.8 |
| Pituitary gland | 0.7 | Liver (fetal) | 6.0 |
| Brain (fetal) | 0.7 | Liver ca. | 1.0 |
| | | (hepatoblast) | |
| | | HepG2 | |
| Brain (whole) | 0.0 | Lung | 1.5 |
| Brain (amygdala) | 1.0 | Lung (fetal) | 2.9 |
| Brain (cere- | 0.3 | Lung ca. (small | 0.2 |
| bellum) | | cell) LX-1 | |
| Brain (hippo- | 6.3 | Lung ca. (small | 5.2 |
| campus) | | cell) NCI-H69 | |
| Brain (thalamus) | 1.9 | Lung ca. (s. cell | 0.0 |
| | | var.) SHP-77 | |
| Cerebral Cortex | 9.2 | Lung ca. (large | 2.8 |
| | | cell)NCI-H460 | |
| Spinal cord | 2.9 | Lung ca. (non-sm. | 5.1 |
| | | cell) A549 | |
| glio/astro U87-MG | 1.6 | Lung ca. (non- | 10.2 |
| | | s. cell) NCI-H23 | |
| glio/astro U- | 1.1 | Lung ca. (non- | 28.3 |
| 118-MG | | s. cell) HOP-62 | |
| astrocytoma | 1.2 | Lung ca. (non- | 2.0 |
| SW1783 | | s. cl) NCI-H522 | |
| neuro*; met SK- | 0.2 | Lung ca. (squam.) | 3.6 |
| N-AS | | SW 900 | |
| astrocytoma SF- | 7.4 | Lung ca. (squam.) | 0.7 |
| 539 | | NCI-H596 | |
| astrocytoma SNB- | 1.6 | Mammary gland | 3.4 |
| 75 | | | |
| glioma SNB-19 | 8.1 | Breast ca.* | 1.3 |
| | | (pl. ef) MCF-7 | |
| glioma U251 | 5.9 | Breast ca.* | 0.3 |
| | | (pl. ef) | |
| | | MDA-MB-231 | |
| glioma SF-295 | 12.2 | Breast ca.* | 12.8 |
| | | (pl. ef) T47D | |
| Heart | 15.3 | Breast ca. BT-549 | 2.9 |
| Skeletal Muscle | 4.9 | Breast ca. MDA-N | 0.8 |
| Bone marrow | 4.4 | Ovary | 9.3 |
| Thymus | 0.8 | Ovarian ca. OVCAR-3 | 42.0 |
| Spleen | 5.6 | Ovarian ca. OVCAR-4 | 1.4 |
| Lymph node | 1.6 | Ovarian ca. OVCAR-5 | 18.2 |
| Colorectal | 10.9 | Ovarian ca. OVCAR-8 | 0.0 |
| Stomach | 3.2 | Ovarian ca. IGROV-1 | 0.0 |
| Small intestine | 7.8 | Ovarian ca. | 100.0 |
| | | (ascites) | |
| | | SK-OV-3 | |
| Colon ca. SW480 | 0.0 | Uterus | 6.5 |
| Colon ca.* SW620 | 0.0 | Placenta | 1.8 |
| (SW480 met) | | | |
| Colon ca. HT29 | 0.9 | Prostate | 23.7 |
| Colon ca. HCT-116 | 1.8 | Prostate ca.* | 4.5 |
| | | (bone met) PC-3 | |
| Colon ca. CaCo-2 | 1.5 | Testis | 2.7 |
| CC Well to Mod | 2.9 | Melanoma | 0.2 |
| Diff (ODO3866) | | Hs688(A).T | |
| Colon ca. HCC- | 11.6 | Melanoma* (met) | 0.9 |
| 2998 | | Hs688(B).T | |
| Gastric ca. | 44.4 | Melanoma UACC-62 | 4.5 |
| (liver met) NCI- | | | |
| N87 | | | |
| Bladder | 98.6 | Melanoma M14 | 4.2 |
| Trachea | 1.5 | Melanoma LOX | 0.4 |
| | | IMVI | |
| Kidney | 40.1 | Melanoma* (met) | 0.0 |
| | | SK-MEL-5 | |
| Kidney (fetal) | 25.5 | | |

TABLE 22
Panel 2D

| | Rel. | | Rel. |
|---|---|---|---|
| | Exp. (%) | | Exp. (%) |
| | Ag1533, | | Ag1533, |
| | Run | | Run |
| Tissue Name | 145165498 | Tissue Name | 145165498 |
| Normal Colon | 48.0 | Kidney Margin | 6.1 |
| | | 8120608 | |
| CC Well to Mod | 5.6 | Kidney Cancer | 0.0 |
| Diff (ODO3866) | | 8120613 | |
| CC Margin | 4.6 | Kidney Margin | 6.9 |
| (ODO3866) | | 8120614 | |
| CC Gr.2 recto- | 3.5 | Kidney Cancer | 10.2 |
| sigmoid (ODO3868) | | 9010320 | |
| CC Margin | 1.0 | Kidney Margin | 15.7 |
| (ODO3868) | | 9010321 | |
| CC Mod Diff | 4.4 | Normal Uterus | 36.3 |
| (ODO3920) | | | |
| CC Margin | 6.6 | Uterine Cancer | 45.7 |
| (ODO3920) | | 064011 | |
| CC Gr.2 ascend | 1.6 | Normal Thyroid | 8.1 |
| colon (ODO3921) | | | |
| CC Margin | 6.1 | Thyroid Cancer | 7.7 |
| (ODO3921) | | | |
| CC from Partial | 0.0 | Thyroid Cancer | 27.0 |
| Hepatectomy | | A302152 | |
| (ODO4309) Mets | | | |
| Liver Margin | 4.8 | Thyroid Margin | 29.5 |
| (ODO4309) | | A302153 | |
| Colon mets to | 4.3 | Normal Breast | 40.9 |
| lung | | | |
| (OD04451-01) | | | |
| Lung Margin | 1.2 | Breast Cancer | 3.5 |
| (OD04451-02) | | | |
| Normal Prostate | 26.1 | Breast Cancer | 18.9 |
| 6546-1 | | (OD04590-01) | |
| Prostate Cancer | 64.2 | Breast Cancer | 34.6 |
| (OD04410) | | Mets | |
| | | (OD04590-03) | |
| Prostate Margin | 43.8 | Breast Cancer | 19.3 |
| (OD04410) | | Metastasis | |
| Prostate Cancer | 42.0 | Breast Cancer | 21.2 |
| (OD04720-01) | | | |
| Prostate Margin | 34.2 | Breast Cancer | 35.1 |
| (OD04720-02) | | | |
| Normal Lung | 31.9 | Breast Cancer | 7.7 |
| | | 9100266 | |
| Lung Met to Muscle | 0.0 | Breast Margin | 2.9 |
| (ODO4286) | | 9100265 | |
| Muscle Margin | 1.9 | Breast Cancer | 14.8 |
| (ODO4286) | | A209073 | |
| Lung Malignant | 13.1 | Breast Margin | 23.3 |
| Cancer (OD03126) | | A2090734 | |
| Lung Margin | 26.8 | Normal Liver | 21.9 |
| (OD03126) | | | |
| Lung Cancer | 5.4 | Liver Cancer | 22.4 |
| (OD04404) | | | |
| Lung Margin | 37.9 | Liver Cancer 1025 | 0.0 |
| (OD04404) | | | |
| Lung Cancer | 2.9 | Liver Cancer 1026 | 2.1 |
| (OD04565) | | | |
| Lung Margin | 9.3 | Liver Cancer | 13.5 |
| (OD04565) | | 6004-T | |
| Lung Cancer | 24.8 | Liver Tissue | 5.9 |
| (OD04237-01) | | 6004-N | |
| Lung Margin | 10.5 | Liver Cancer | 0.0 |
| (OD04237-02) | | 6005-T | |
| Ocular Mel Met | 10.1 | Liver Tissue | 0.0 |
| to Liver | | 6005-N | |
| (ODO4310) | | | |
| Liver Margin | 8.7 | Normal Bladder | 42.0 |
| (ODO4310) | | | |
| Melanoma | 0.0 | Bladder Cancer | 5.8 |
| Metastasis | | | |
| Lung Margin | 25.0 | Bladder Cancer | 65.1 |
| (OD04321) | | | |
| Normal Kidney | 100.0 | Bladder Cancer | 5.6 |
| | | (OD04718-01) | |
| Kidney Ca, | 52.1 | Bladder Normal | 32.8 |
| Nuclear grade | | Adjacent | |
| 2 (OD04338) | | (OD04718-03) | |
| Kidney Margin | 22.1 | Normal Ovary | 5.7 |
| (OD04338) | | | |
| Kidney Ca | 41.5 | Ovarian Cancer | 9.4 |
| Nuclear grade | | | |
| 1/2 (OD04339) | | | |
| Kidney Margin | 46.0 | Ovarian Cancer | 8.8 |
| (OD04339) | | (OD04768-07) | |
| Kidney Ca, Clear | 27.0 | Ovary Margin | 3.3 |
| cell | | (OD04768-08) | |
| type (OD04340) | | | |
| Kidney Margin | 24.7 | Normal Stomach | 30.6 |
| (OD04340) | | | |
| Kidney Ca, | 9.1 | Gastric Cancer | 0.0 |
| Nuclear grade | | 9060358 | |
| 3 (OD04348) | | | |
| Kidney Margin | 92.7 | Stomach Margin | 0.0 |
| (OD04348) | | 9060359 | |
| Kidney Cancer | 5.0 | Gastric Cancer | 5.3 |
| (OD04622-01) | | 9060395 | |
| Kidney Margin | 2.0 | Stomach Margin | 3.1 |
| (OD04622-03) | | 9060394 | |
| Kidney Cancer | 10.9 | Gastric Cancer | 4.7 |
| (OD04450-01) | | 9060397 | |
| Kidney Margin | 17.6 | Stomach Margin | 0.0 |
| (OD04450-03) | | 9060396 | |
| Kidney Cancer | 0.8 | Gastric Cancer | 11.3 |
| 8120607 | | 064005 | |

TABLE 23
Panel 4.1D

| | Rel. | | Rel. |
|---|---|---|---|
| | Exp. (%) | | Exp. (%) |
| | Ag1533, | | Ag1533, |
| | Run | | Run |
| Tissue Name | 223794696 | Tissue Name | 223794696 |
| Secondary Th1 | 0.0 | HUVEC IL-1beta | 11.8 |
| act | | | |
| Secondary Th2 | 20.4 | HUVEC IFN gamma | 11.5 |
| act | | | |
| Secondary Tr1 | 13.1 | HUVEC TNF | 4.0 |
| act | | alpha + IFN | |
| | | gamma | |
| Secondary Th1 | 25.3 | HUVEC TNF | 18.7 |
| rest | | alpha + IL4 | |
| Secondary Th2 | 8.0 | HUVEC IL-11 | 29.1 |
| rest | | | |
| Secondary Tr1 | 33.9 | Lung Micro- | 83.5 |
| rest | | vascular EC | |
| | | none | |
| Primary Th1 act | 0.0 | Lung Micro- | 14.0 |
| | | vascular EC | |
| | | TNFalpha | |
| | | + IL-1beta | |
| Primary Th2 act | 25.0 | Microvascular | 8.5 |
| | | Dermal EC none | |
| Primary Tr1 act | 7.9 | Microsvasular | 0.0 |
| | | Dermal EC | |
| | | TNFalpha + | |
| | | IL-1beta | |
| Primary Th1 rest | 14.5 | Bronchial | 15.2 |
| | | epithelium | |
| | | TNFalpha + | |
| | | IL1beta | |
| Primary Th2 rest | 4.3 | Small airway | 0.0 |
| | | epithelium | |
| | | none | |
| Primary Tr1 rest | 20.7 | Small airway | 20.9 |
| | | epithelium | |
| | | TNFalpha + | |
| | | IL-1beta | |
| CD45RA CD4 | 21.2 | Coronery artery | 0.0 |
| lymphocyte act | | SMC rest | |
| CD45RO CD4 | 65.5 | Coronery artery | 0.0 |
| lymphocyte act | | SMC | |
| | | TNFalpha + | |
| | | IL-1beta | |
| CD8 lymphocyte | 25.9 | Astrocytes rest | 20.3 |
| act | | | |
| Secondary CD8 | 19.6 | Astrocytes | 10.3 |
| lymphocyte rest | | TNFalpha + | |
| | | IL-1beta | |
| Secondary CD8 | 4.5 | KU-812 (Baso- | 10.9 |
| lymphocyte act | | phil) rest | |
| CD4 lymphocyte | 53.2 | KU-812 (Baso- | 34.2 |
| none | | phil) | |
| | | PMA/ionomycin | |
| 2ry Th1/Th2/ | 19.2 | CCD1106 | 0.0 |
| Tr1_anti- | | (Keratinocytes) | |
| CD95 CH11 | | none | |
| LAK cells rest | 22.1 | CCD1106 | 8.5 |
| | | (Keratinocytes) | |
| | | TNFalpha + | |
| | | IL-1beta | |
| LAK cells IL-2 | 51.1 | Liver cirrhosis | 26.6 |
| LAK cells | 3.1 | NCI-H292 none | 19.9 |
| IL-2 + IL-12 | | | |
| LAK cells | 24.0 | NCI-H292 IL-4 | 27.5 |
| IL-2 + IFN | | | |
| gamma | | | |
| LAK cells | 14.6 | NCI-H292 IL-9 | 29.3 |
| IL-2 + IL-18 | | | |
| LAK cells | 0.0 | NCI-H292 IL-13 | 16.2 |
| PMA/ionomycin | | | |
| NK Cells IL-2 | 38.7 | NCI-H292 IFN | 44.1 |
| rest | | gamma | |
| Two Way MLR 3 | 61.6 | HPAEC none | 8.5 |
| day | | | |
| Two Way MLR 5 | 35.6 | HPAEC TNF | 10.7 |
| day | | alpha + IL-1 | |
| | | beta | |
| Two Way MLR 7 | 9.9 | Lung fibroblast | 60.7 |
| day | | none | |
| PBMC rest | 26.2 | Lung fibroblast | 40.6 |
| | | TNF alpha + | |
| | | IL-1 beta | |
| PBMC PWM | 3.6 | Lung fibroblast | 8.8 |
| | | IL-4 | |
| PBMC PHA-L | 4.5 | Lung fibroblast | 21.3 |
| | | IL-9 | |
| Ramos (B cell) | 0.0 | Lung fibroblast | 4.8 |
| none | | IL-13 | |
| Ramos (B cell) | 0.0 | Lung fibroblast | 4.6 |
| ionomycin | | IFN gamma | |
| B lymphocytes | 5.1 | Dermal fibro- | 0.0 |
| PWM | | blast | |
| | | CCD1070 rest | |
| B lymphocytes | 17.0 | Dermal fibro- | 20.0 |
| CD40L and IL-4 | | blast | |
| | | CCD1070 TNF | |
| | | alpha | |
| EOL-1 dbcAMP | 10.9 | Dermal fibro- | 4.0 |
| | | blast CCD1070 | |
| | | IL-1 beta | |
| EOL-1 dbcAMP | 0.0 | Dermal fibro- | 10.7 |
| PMA/ionomycin | | blast IFN gamma | |
| Dendritic cells | 4.1 | Dermal fibro- | 26.6 |
| none | | blast IL-4 | |
| Dendritic cells | 17.7 | Dermal Fibro- | 10.4 |
| LPS | | blasts rest | |
| Dendritic cells | 14.8 | Neutrophils | 10.0 |
| anti-CD40 | | TNFa + LPS | |
| Monocytes rest | 10.7 | Neutrophils | 21.3 |
| | | rest | |
| Monocytes LPS | 16.4 | Colon | 0.0 |
| Macrophages rest | 18.6 | Lung | 4.2 |
| Macrophages LPS | 8.0 | Thymus | 100.0 |
| HUVEC none | 5.4 | Kidney | 81.8 |
| HUVEC starved | 2.6 | | |

TABLE 24
Panel 4D

| | Rel. | | Rel. |
|---|---|---|---|
| | Exp. (%) | | Exp. (%) |
| | Ag2269, | | Ag2269, |
| | Run | | Run |
| Tissue Name | 151751515 | Tissue Name | 151751515 |
| Secondary Th1 act | 5.0 | HUVEC IL-1beta | 1.2 |
| Secondary Th2 act | 6.5 | HUVEC IFN gamma | 10.2 |
| Secondary Tr1 act | 2.3 | HUVEC TNF alpha + IFN | 1.9 |
| | | gamma | |
| Secondary Th1 rest | 3.5 | HUVEC TNF alpha + IL4 | 1.2 |
| Secondary Th2 rest | 12.1 | HUVEC IL-11 | 9.7 |
| Secondary Tr1 rest | 12.3 | Lung Microvascular EC | 17.7 |
| | | none | |
| Primary Th1 act | 3.8 | Lung Microvascular EC | 9.9 |
| | | TNFalpha + IL-1beta | |
| Primary Th2 act | 6.7 | Microvascular Dermal EC | 1.3 |
| | | none | |
| Primary Tr1 act | 4.9 | Microsvasular Dermal EC | 0.0 |
| | | TNFalpha + IL-1beta | |
| Primary Th1 rest | 56.6 | Bronchial epithelium | 0.0 |
| | | TNFalpha + IL1beta | |
| Primary Th2 rest | 11.0 | Small airway epithelium | 1.2 |
| | | none | |
| Primary Tr1 rest | 8.9 | Small airway epithelium | 27.5 |
| | | TNFalpha + IL-1beta | |
| CD45RA CD4 | 5.3 | Coronery artery SMC rest | 3.9 |
| lymphocyte act | | | |
| CD45RO CD4 | 21.8 | Coronery artery SMC | 0.0 |
| lymphocyte act | | TNFalpha + IL-1beta | |
| CD8 lymphocyte act | 5.3 | Astrocytes rest | 3.3 |
| Secondary CD8 | 7.6 | Astrocytes TNFalpha + | 5.4 |
| lymphocyte rest | | IL-1beta | |
| Secondary CD8 | 0.8 | KU-812 (Basophil) rest | 14.1 |
| lymphocyte act | | | |
| CD4 lymphocyte none | 22.5 | KU-812 (Basophil) | 25.7 |
| | | PMA/ionomycin | |
| 2ry Th1/Th2/Tr1_anti- | 4.8 | CCD1106 (Keratinocytes) | 0.0 |
| CD95 CH11 | | none | |
| LAK cells rest | 25.3 | CCD1106 (Keratinocytes) | 1.3 |
| | | TNFalpha + IL-1beta | |
| LAK cells IL-2 | 17.9 | Liver cirrhosis | 10.6 |
| LAK cells IL-2 + IL-12 | 9.9 | Lupus kidney | 11.0 |
| LAK cells IL-2 + IFN | 40.9 | NCI-H292 none | 18.3 |
| gamma | | | |
| LAK cells IL-2 + IL-18 | 21.2 | NCI-H292 IL-4 | 28.1 |
| LAK cells | 4.0 | NCI-H292 IL-9 | 32.3 |
| PMA/ionomycin | | | |
| NK Cells IL-2 rest | 15.8 | NCI-H292 IL-13 | 12.4 |
| Two Way MLR 3 day | 35.6 | NCI-H292 IFN gamma | 23.3 |
| Two Way MLR 5 day | 3.6 | HPAEC none | 6.0 |
| Two Way MLR 7 day | 5.5 | HPAEC TNF alpha + IL-1 | 1.5 |
| | | beta | |
| PBMC rest | 7.1 | Lung fibroblast none | 15.9 |
| PBMC PWM | 13.1 | Lung fibroblast TNF alpha + | 4.9 |
| | | IL-1 beta | |
| PBMC PHA-L | 1.6 | Lung fibroblast IL-4 | 17.0 |
| Ramos (B cell) none | 2.8 | Lung fibroblast IL-9 | 7.3 |
| Ramos (B cell) | 2.8 | Lung fibroblast IL-13 | 15.9 |
| ionomycin | | | |
| B lymphocytes PWM | 7.9 | Lung fibroblast IFN | 10.0 |
| | | gamma | |
| B lymphocytes CD40L | 7.1 | Dermal fibroblast | 2.6 |
| and IL-4 | | CCD1070 rest | |
| EOL-1 dbcAMP | 5.1 | Dermal fibroblast | 6.0 |
| | | CCD1070 TNF alpha | |
| EOL-1 dbcAMP | 1.4 | Dermal fibroblast | 2.6 |
| PMA/ionomycin | | CCD1070 IL-1 beta | |
| Dendritic cells none | 1.9 | Dermal fibroblast IFN | 3.5 |
| | | gamma | |
| Dendritic cells LPS | 9.1 | Dermal fibroblast IL-4 | 6.7 |
| Dendritic cells anti- | 0.0 | IBD Colitis 2 | 0.0 |
| CD40 | | | |
| Monocytes rest | 5.5 | IBD Crohn's | 0.0 |
| Monocytes LPS | 5.3 | Colon | 33.2 |
| Macrophages rest | 1.7 | Lung | 19.1 |
| Macrophages LPS | 1.2 | Thymus | 52.1 |
| HUVEC none | 10.1 | Kidney | 100.0 |
| HUVEC starved | 5.0 | | |

CNS_neurodegeneration_v1.0 Summary: Ag1533 The GPCR1a and GPCR1b genes show widespread expression across all regions of the brain, with highest expression in the parietal cortex of a control patient (CT=33.5). The GPCR1a and GPCR1 genes appear to be upregulated in the temporal cortex of patients with Alzheimer's disease. The temporal cortex is a region which shows severe degeneration in Alzheimer's disease, suggesting the expression of these genes may play a role in the pathogenesis of this disease. Therapeutic modulation of the GPCR1a and GPCR1b genes or treatment with an antagonist to the receptor may be of benefit in treating Alzheimer's disease or dementia.

General_screening_panel_v1.4 Summary: Ag2269 Highest expression of the GPCR1 a and GPCR1b genes is detected in the squamous cell carcinoma cell line SCC4 (CT=28.3). Significant expression is also seen in cell lines derived from lung and ovarian cancer. Thus, expression of these genes could be used to differentiate between these cell lines and other samples in this panel. Furthermore, therapeutic modulation of the expression or function of the protein encoded by the GPCR1 a and GPCR1b genes could be useful in the treatment of lung, ovarian or squamous cell carcinoma.

Among tissues with metabolic function, the GPCR1a and GPCR1b genes are moderately expressed (CT values=32-33) in adipose, adult and fetal heart, fetal skeletal muscle, adrenal and pancreas. Thus, these gene products may be a small molecule drug target z z for the treatment of metabolic disease, including obesity and Types 1 and 2 diabetes.

The GPCR1a and GPCR1b genes are expressed at low levels in the fetal brain and moderate levels in the spinal cord. The encoded proteins are homologous to the GPCR family of receptors. Several neurotransmitter receptors are GPCRs, including the dopamine receptor family, the serotonin receptor family, the GABAB receptor, muscarinic acetylcholine receptors, and others; thus this GPCR may represent a novel neurotransmitter receptor. Targeting various neurotransmitter receptors (dopamine, serotonin) has proven to be an effective therapy in psychiatric illnesses such as schizophrenia, bipolar disorder and depression. Therapeutic modulation of these genes or their protein products may be beneficial in one or more of these diseases, as may blockade of the receptor coded for by these genes. Levels of the GPCR1a and GPCR1b genes are considerably higher, however, in areas outside of the central nervous system (such as kidney and fetal lung), suggesting the possibility of a wider role in intercellular signaling.

General_screening_panel_v1.5 Summary: Ag1533/Ag2269 Two runs with two different probes and primers show highest expression in the fetal lung and kidney (CTs=30). Significant levels of expression are also detected in adult kidney and lung. This expression profile suggests that the GPCR1a and GPCR1b gene products may be involved in the normal homeostasis of these organs. Therefore, therapeutic modulation of the expression or function of these genes may be effective in the treatment of diseases of these organs including diabetes, polycystic kidney disease, systemic lupus erythematosus, asthma, emphysema, and acute respiratory distress syndrome (ARDS).

The GPCR1a and GPCR1b genes are also moderately expressed in a a variety of metabolic tissues including adipose, adult and fetal heart, adult and fetal skeletal muscle, adrenal, pituitary, thyroid and pancreas. Thus, these gene products may be a small molecule drug target for the treatment of metabolic disease, including obesity and Types 1 and 2 diabetes. Furthermore, this gene is differentially expressed in adult (CT value=38) versus fetal liver (CT values=33-34), and may be used to differentiate between the adult and fetal phenotype in this tissue.

There is moderate expression in some tissues of the central nervous system, including the fetal brain and the spinal cord. Please see Panel General_screening_panel_v1.4 Summary for discussion of potential utility in the central nervous system.

There is also significant expression of the GPCR1a and GPCR1b genes in a cell lines derived from ovarian cancer and gastric cancer. Please see Panel 1.2 for discussion of utility of this gene in the context of this expression profile.

Panel 1.2 Summary: Ag1533 The expression of the GPCR1a and GPCR1b genes is highest in a sample derived from an ovarian cancer cell line (CT=29.1). Interestingly, this cell line was derived from a unique form of ovarian cancer, ascites. In addition, there appears to be substantial expression of this gene in samples derived from other ovarian cancer cell lines as well as normal bladder tissue, normal kidney tissue and a cell lined derived from a gastric cancer. Thus, the expression of these genes in these tissues could be used to distinguish these samples from other samples in the panel. Additionally, the expression of the GPCR1a and GPCR1b genes could be used to distingush ascites derived samples from other samples in the panel. Furthermore, therapeutic modulation of this gene, through the use of small molecule drugs, antibodies or protein therapeutics might be of benefit in the treatment of ovarian cancer.

The GPCR1a and GPCR1b genes are also expressed at moderate levels in a variety of metabolic tissues including adult and fetal liver, adult and fetal heart, skeletal muscle, adrenal and pancreas. This widespread expression suggests that the protein encoded by these genes could be useful in the treatment of metabolic diseases, including obesity and diabetes.

Panel 2D Summary: Ag1533 The expression of the GPCR1a and GPCR1b genes appears to be highest in a sample derived from normal kidney tissue (CT=31.9). In addition, there is substantial expression in samples derived from other samples of normal kidney tissue adjacent to malignant kidney. Moreover, there also appears to be expression associated with tissues, normal or malignant, derived from uterus, prostate, breast, bladder and thyroid. Thus, the expression of these genes could be used to distinguish samples derived from these tissue types when compared to other samples in the panel. Further, therapeutic modulation of these genes, or gene products, through the use of small molecule drugs, antibodies or protein therapeutics might be of benefit in the treatment of cancers of the above listed tissues.

Panel 4.1D Summary: Ag1533: These transcripts is expressed on most tissues in panel 4.1D regardless of treatment, with highest expression in the thymus (CT=32.8). These transcripts encodes a GPCR-like molecule with potential signaling activity and may important in maintaining normal cellular functions in a number of tissues. Therapies designed with the protein encoded for by these transcripts could be important in regulating cellular viability or function.

Panel 4D Summary: Ag2269 These transcripts are expressed on most tissues in panel 4D regardless of treatment, with highest expression in the kidney (CT31.3). These transcripts encode a GPCR-like molecule with potential signaling activity and may important in maintaining normal cellular functions in a number of tissues. Therapies designed with the proteins encoded for by these transcripts could be important in regulating cellular viability or function

GPCR1 c

Expression of the GPCR1c gene (also referred to as CG55798-04) was assessed using the primer-probe sets Ag1533, Ag2617 and Ag2862, described in Tables 25, 26 and 27. Results of the RTQ-PCR runs are shown in Tables 28, 29, 30, 31, 32, 33 and 34.

TABLE 25

Probe Name Ag 1533

| | | | | SEQ ID |
|---|---|---|---|---|
| Primers | Sequences | Length | Start Position | NO: |
| Forward | 5′-accatcatcaagagtgctatgg-3′ | 22 | 431 | 125 |
| Probe | TET-5′-tcctttcgaagcttctgcatcatcct-3′-TAMRA | 26 | 458 | 126 |
| Reverse | 5′-aggcatgtcagcaagaatacat-3′ | 22 | 489 | 127 |

TABLE 26

Probe Name Ag2617

| | | | | SEQ ID |
|---|---|---|---|---|
| Primers | Sequences | Length | Start Position | NO: |
| Forward | 5′-ccatggcatttgatcactatgt-3′ | 22 | 363 | 128 |
| Probe | TET-5′-tgagatataccaccatcttgactccca-3′-TAMRA | 27 | 402 | 129 |
| Reverse | 5′-ccatagcactcttgatgatggt-3′ | 22 | 431 | 130 |

TABLE 27

Probe Name Ag2862

| | | | | SEQ ID |
|---|---|---|---|---|
| Primers | Sequences | Length | Start Positon | NO: |
| Forward | 5′-ccatggcatttgatcactatgt-3′ | 22 | 363 | 131 |
| Probe | TET 5′-tgagatataccaccatcttgactccca-3′-TAMRA | 27 | 402 | 129 |
| Reverse | 5′-ccatagcactcttgatgatggt-3′ | 22 | 431 | 130 |

TABLE 28
CNS_neurodegeneration_v1.0

| | Rel. | | Rel. |
|---|---|---|---|
| | Exp. (%) | | Exp. (%) |
| | Ag1533, | | Ag1533, |
| | Run | | Run |
| Tissue Name | 225432469 | Tissue Name | 225432469 |
| AD 1 Hippo | 36.6 | Control (Path) 3 | 16.5 |
| | | Temporal Ctx | |
| AD 2 Hippo | 11.6 | Control (Path) 4 | 42.9 |
| | | Temporal Ctx | |
| AD 3 Hippo | 50.7 | AD 1 Occipital Ctx | 39.2 |
| AD 4 Hippo | 75.3 | AD 2 Occipital Ctx | 0.0 |
| | | (Missing) | |
| AD 5 Hippo | 20.4 | AD 3 Occipital Ctx | 25.9 |
| AD 6 Hippo | 69.3 | AD 4 Occipital Ctx | 82.9 |
| Control 2 Hippo | 22.5 | AD 5 Occipital Ctx | 11.0 |
| Control 4 Hippo | 53.2 | AD 5 Occipital Ctx | 7.1 |
| Control (Path) 3 | 29.7 | Control 1 Occipital | 11.9 |
| Hippo | | Ctx | |
| AD 1 Temporal Ctx | 96.6 | Control 2 Occipital | 29.1 |
| | | Ctx | |
| AD 2 Temporal Ctx | 34.6 | Control 3 Occipital | 41.2 |
| | | Ctx | |
| AD 3 Temporal Ctx | 54.3 | Control 4 Occipital | 11.9 |
| | | Ctx | |
| AD 4 Temporal Ctx | 68.3 | Control (Path) 1 | 23.3 |
| | | Occipital Ctx | |
| AD 5 Inf Temporal | 90.8 | Control (Path) 2 | 14.3 |
| Ctx | | Occipital Ctx | |
| AD 5 Sup Temporal | 53.2 | Control (Path) 3 | 35.1 |
| Ctx | | Occipital Ctx | |
| AD 6 Inf Temporal | 62.0 | Control (Path) 4 | 30.8 |
| Ctx | | Occipital Ctx | |
| AD 6 Sup Temporal | 55.5 | Control 1 Parietal | 18.2 |
| Ctx | | Ctx | |
| Control 1 Temporal | 5.3 | Control 2 Parietal | 69.3 |
| Ctx | | Ctx | |
| Control 2 Temporal | 19.6 | Control 3 Parietal | 6.3 |
| Ctx | | Ctx | |
| Control 3 Temporal | 38.7 | Control (Path) 1 | 33.9 |
| Ctx | | Parietal Ctx | |
| Control 3 Temporal | 17.0 | Control (Path) 2 | 18.4 |
| Ctx | | Parietal Ctx | |
| Control (Path) 1 | 29.1 | Control (Path) 3 | 26.8 |
| Temporal Ctx | | Parietal Ctx | |
| Control (Path) 2 | 23.7 | Control (Path) 4 | 100.0 |
| Temporal Ctx | | Parietal Ctx | |

TABLE 29
General_screening_panel_v1.5

| | Rel. | | Rel. |
|---|---|---|---|
| | Exp. (%) | | Exp. (%) |
| | Ag1533, | | Ag1533, |
| | Run | | Run |
| Tissue Name | 228632846 | Tissue Name | 228632846 |
| Adipose | 12.9 | Renal ca. TK-10 | 2.4 |
| Melanoma* | 1.5 | Bladder | 43.8 |
| Hs688(A).T | | | |
| Melanoma* | 0.4 | Gastric ca. (liver | 54.7 |
| Hs688(B).T | | met.) NCI-N87 | |
| Melanoma* M14 | 0.0 | Gastric ca. KATO | 0.4 |
| | | III | |
| Melanoma* | 0.5 | Colon ca. SW-948 | 0.0 |
| LOXIMVI | | | |
| Melanoma* SK- | 0.3 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 15.1 | Colon ca. HT29 | 0.0 |
| Prostate ca.* | 1.9 | Colon ca. HCT-116 | 2.8 |
| (bone met) PC-3 | | | |
| Prostate Pool | 23.7 | Colon ca. CaCo-2 | 0.7 |
| Placenta | 2.9 | Colon cancer tissue | 1.5 |
| Uterus Pool | 11.5 | Colon ca. SW1116 | 0.3 |
| Ovarian ca. | 25.5 | Colon ca. Colo-205 | 0.0 |
| OVCAR-3 | | | |
| Ovarian ca. SK- | 87.7 | Colon ca. SW-48 | 0.0 |
| OV-3 | | | |
| Ovarian ca. | 0.0 | Colon Pool | 37.4 |
| OVCAR-4 | | | |
| Ovarian ca. | 10.1 | Small Intestine | 43.5 |
| OVCAR-5 | | Pool | |
| Ovarian ca. | 0.0 | Stomach Pool | 27.4 |
| IGROV-1 | | | |
| Ovarian ca. | 2.4 | Bone Marrow Pool | 22.1 |
| OVCAR-8 | | | |
| Ovary | 22.2 | Fetal Heart | 57.0 |
| Breast ca. MCF- | 0.0 | Heart Pool | 12.2 |
| 7 | | | |
| Breast ca. MDA- | 1.3 | Lymph Node Pool | 66.9 |
| MB-231 | | | |
| Breast ca. BT | 1.4 | Fetal Skeletal | 11.4 |
| 549 | | Muscle | |
| Breast ca. T47D | 0.8 | Skeletal Muscle | 7.5 |
| | | Pool | |
| Breast ca. MDA- | 0.7 | Spleen Pool | 21.3 |
| N | | | |
| Breast Pool | 59.5 | Thymus Pool | 38.7 |
| Trachea | 19.1 | CNS cancer (glio/ | 1.6 |
| | | astro) U87-MG | |
| Lung | 27.2 | CNS cancer (glio/ | 1.5 |
| | | astro) U-118-MG | |
| Fetal Lung | 100.0 | CNS cancer | 0.0 |
| | | (neuro;met) SK-N- | |
| | | AS | |
| Lung ca. NCI- | 0.0 | CNS cancer (astro) | 1.1 |
| N417 | | SF-539 | |
| Lung ca. LX-1 | 0.4 | CNS cancer (astro) | 4.5 |
| | | SNB-75 | |
| Lung ca. NCI- | 4.7 | CNS cancer (glio) | 1.1 |
| H146 | | SNB-19 | |
| Lung ca. SHP-77 | 0.0 | CNS cancer (glio) | 20.6 |
| | | SF-295 | |
| Lung ca. A549 | 2.9 | Brain (Amygdala) | 2.1 |
| | | Pool | |
| Lung ca. NCI- | 0.0 | Brain (cerebellum) | 0.9 |
| H526 | | | |
| Lung ca. NCI- | 6.7 | Brain (fetal) | 7.9 |
| H23 | | | |
| Lung ca. NCI- | 9.2 | Brain (Hippocampus) | 1.6 |
| H460 | | Pool | |
| Lung ca. HOP-62 | 8.4 | Cerebral Cortex | 2.6 |
| | | Pool | |
| Lung ca. NCI- | 0.0 | Brain (Substantia | 2.0 |
| H522 | | nigra) | |
| | | Pool | |
| Liver | 0.4 | Brain (Thalamus) | 2.5 |
| | | Pool | |
| Fetal Liver | 8.7 | Brain (whole) | 3.8 |
| Liver ca. HepG2 | 0.7 | Spinal Cord Pool | 5.0 |
| Kidney Pool | 67.8 | Adrenal Gland | 11.0 |
| Fetal Kidney | 94.6 | Pituitary gland | 5.3 |
| | | Pool | |
| Renal ca. 786-0 | 1.2 | Salivary Gland | 1.7 |
| Renal ca. A498 | 1.8 | Thyroid (female) | 2.5 |
| Renal ca. ACHN | 3.6 | Pancreatic ca. | 4.7 |
| | | CAPAN2 | |
| Renal ca. UO-31 | 5.8 | Pancreas Pool | 39.8 |

TABLE 30
Panel 1.2

| | Rel. Exp. (%) Ag1533, | | Rel. Exp. (%) Ag1533, |
|---|---|---|---|
| Tissue Name | Run 142223910 | Tissue Name | Run 142223910 |
| Endothelial cells | 4.4 | Renal ca. 786-0 | 1.8 |
| Heart (Fetal) | 3.9 | Renal ca. A498 | 7.5 |
| Pancreas | 4.8 | Renal ca. RXF 393 | 1.8 |
| Pancreatic ca. | 1.1 | Renal ca. ACHN | 5.0 |
| CAPAN 2 | | | |
| Adrenal Gland | 20.9 | Renal ca. UO-31 | 9.0 |
| Thyroid | 1.1 | Renal ca. TK-10 | 2.3 |
| Salivary gland | 52.1 | Liver | 9.8 |
| Pituitary gland | 0.7 | Liver (fetal) | 6.0 |
| Brain (fetal) | 0.7 | Liver ca. | 1.0 |
| | | (hepatoblast) HepG2 | |
| Brain (whole) | 0.0 | Lung | 1.5 |
| Brain (amygdala) | 1.0 | Lung (fetal) | 2.9 |
| Brain (cerebellum) | 0.3 | Lung ca. (small cell) | 0.2 |
| | | LX-1 | |
| Brain (hippocampus) | 6.3 | Lung ca. (small cell) | 5.2 |
| | | NCI-H69 | |
| Brain (thalamus) | 1.9 | Lung ca. (s. cell var.) | 0.0 |
| | | SHP-77 | |
| Cerebral Cortex | 9.2 | Lung ca. (large | 2.8 |
| | | cell) NCI-H460 | |
| Spinal cord | 2.9 | Lung ca. (non-sm. | 5.1 |
| | | cell) A549 | |
| Glio/astro U87-MG | 1.6 | Lung ca. (non-s. cell) | 10.2 |
| | | NCI-H23 | |
| Glio/astro U-118-MG | 1.1 | Lung ca. (non-s. cell) | 28.3 |
| | | HOP-62 | |
| astrocytoma SW1783 | 1.2 | Lung ca. (non-s.cl) | 2.0 |
| | | NCI-H522 | |
| Neuro*; met SK-N-AS | 0.2 | Lung ca. (squam.) | 3.6 |
| | | SW 900 | |
| astrocytoma SF-539 | 7.4 | Lung ca. (squam.) | 0.7 |
| | | NCI-H596 | |
| astrocytoma SNB-75 | 1.6 | Mammary gland | 3.4 |
| glioma SNB-19 | 8.1 | Breast ca.* (pl. ef) | 1.3 |
| | | MCF-7 | |
| glioma U251 | 5.9 | Breast ca.* (pl. ef) | 0.3 |
| | | MDA-MB-231 | |
| glioma SF-295 | 12.2 | Breast ca.* (pl. ef) | 12.8 |
| | | T47D | |
| Heart | 15.3 | Breast ca. BT-549 | 2.9 |
| Skeletal Muscle | 4.9 | Breast ca. MDA-N | 0.8 |
| Bone marrow | 4.4 | Ovary | 9.3 |
| Thymus | 0.8 | Ovarian ca. OVCAR-3 | 42.0 |
| Spleen | 5.6 | Ovarian ca. OVCAR-4 | 1.4 |
| Lymph node | 1.6 | Ovarian ca. OVCAR-5 | 18.2 |
| Colorectal | 10.9 | Ovarian ca. OVCAR-8 | 0.0 |
| Stomach | 3.2 | Ovarian ca. IGROV-1 | 0.0 |
| Small intestine | 7.8 | Ovarian ca. (ascites) | 100.0 |
| | | SK-OV-3 | |
| Colon ca. SW480 | 0.0 | Uterus | 6.5 |
| Colon ca.* SW620 | 0.0 | Placenta | 1.8 |
| (SW480 met) | | | |
| Colon ca. HT29 | 0.9 | Prostate | 23.7 |
| Colon ca. HCT-116 | 1.8 | Prostate ca.* (bone | 4.5 |
| | | met) PC-3 | |
| Colon ca. CaCo-2 | 1.5 | Testis | 2.7 |
| CC Well to Mod Diff | 2.9 | Melanoma | 0.2 |
| (ODO3866) | | Hs688 (A).T | |
| Colon ca. HCC-2998 | 11.6 | Melanoma* (met) | 0.9 |
| | | Hs688 (B).T | |
| Gastric ca. (liver met) | 44.4 | Melanoma UACC-62 | 4.5 |
| NCI-N87 | | | |
| Bladder | 98.6 | Melanoma M14 | 4.2 |
| Trachea | 1.5 | Melanoma LOX | 0.4 |
| | | IMVI | |
| Kidney | 40.1 | Melanoma* (met) | 0.0 |
| | | SK-MEL-5 | |
| Kidney (fetal) | 25.5 | | |

TABLE 31
Panel 1.3D

| | Rel. Exp. (%) Ag2617, | | Rel. Exp. (%) Ag2617, |
|---|---|---|---|
| Tissue Name | Run 167644078 | Tissue Name | Run 167644078 |
| Liver adenocarcinoma | 2.3 | Kidney (fetal) | 15.0 |
| Pancreas | 2.2 | Renal ca. 786-0 | 1.5 |
| Pancreatic ca. | 0.0 | Renal ca. A498 | 3.1 |
| CAPAN 2 | | | |
| Adrenal gland | 0.0 | Renal ca. RXF 393 | 0.0 |
| Thyroid | 1.5 | Renal ca. ACHN | 2.2 |
| Salivary gland | 3.2 | Renal ca. UO-31 | 0.0 |
| Pituitary gland | 6.1 | Renal ca. TK-10 | 2.4 |
| Brain (fetal) | 2.9 | Liver | 2.5 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 2.6 |
| Brain (hippocampus) | 3.2 | Lung (fetal) | 4.0 |
| Brain (substantia nigra) | 2.3 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 1.3 | Lung ca. (s. cell var.) | 0.0 |
| | | SHP-77 | |
| Spinal cord | 3.4 | Lung ca. (large | 0.0 |
| | | cell) NCI-H460 | |
| glio/astro U87-MG | 0.0 | Lung ca. (non-sm. | 0.0 |
| | | cell) A549 | |
| glio/astro U-118-MG | 1.5 | Lung ca. (non-s. cell) | 4.7 |
| | | NCI-H23 | |
| astrocytoma SW1783 | 2.0 | Lung ca. (non-s. cell) | 2.1 |
| | | HOP-62 | |
| Neuro*; met SK-N-AS | 0.0 | Lung ca. (non-s. cl) | 0.0 |
| | | NCI-H522 | |
| astrocytoma SF-539 | 6.3 | Lung ca. (squam.) | 3.0 |
| | | SW 900 | |
| astrocytoma SNB-75 | 1.4 | Lung ca. (squam.) | 0.0 |
| | | NCI-H596 | |
| glioma SNB-19 | 2.5 | Mammary gland | 3.3 |
| glioma U251 | 16.6 | Breast ca.* (pl. ef) | 0.0 |
| | | MCF-7 | |
| glioma SF-295 | 12.5 | Breast ca.* (pl. ef) | 0.0 |
| | | MDA-MB-231 | |
| Heart (Fetal) | 0.0 | Breast ca.* (pl. ef) | 1.4 |
| | | T47D | |
| Heart | 2.1 | Breast ca. BT-549 | 1.3 |
| Skeletal muscle (Fetal) | 3.1 | Breast ca. MDA-N | 1.7 |
| Skeletal muscle | 0.0 | Ovary | 0.0 |
| Bone marrow | 1.9 | Ovarian ca. OVCAR-3 | 9.2 |
| Thymus | 4.1 | Ovarian ca. OVCAR-4 | 0.0 |
| Spleen | 1.6 | Ovarian ca. OVCAR-5 | 9.2 |
| Lymph node | 7.1 | Ovarian ca. OVCAR-8 | 5.1 |
| Colorectal | 3.8 | Ovarian ca. IGROV-1 | 0.0 |
| Stomach | 0.0 | Ovarian ca. (ascites) | 100.0 |
| | | SK-OV-3 | |
| Small intestine | 1.4 | Uterus | 5.1 |
| Colon ca. SW480 | 1.5 | Placenta | 0.0 |
| Colon ca.* SW620 | 0.0 | Prostate | 4.5 |
| (SW480 met) | | | |
| Colon ca. HT29 | 1.0 | Prostate ca.* (bone | 1.0 |
| | | met) PC-3 | |
| Colon ca. HCT-116 | 0.0 | Testis | 5.4 |
| Colon ca. CaCo-2 | 0.0 | Melanoma | 0.0 |
| | | Hs688 (A).T | |
| CC Well to Mod Diff | 0.0 | Melanoma* (met) | 0.9 |
| (ODO3866) | | Hs688 (B).T | |
| Colon ca. HCC-2998 | 0.0 | Melanoma UACC-62 | 0.0 |
| Gastric ca. (liver met) | 12.7 | Melanoma M14 | 0.0 |
| NCI-N87 | | | |
| Bladder | 16.4 | Melanoma LOX | 0.0 |
| | | IMVI | |
| Trachea | 4.1 | Melanoma* (met) | 0.0 |
| | | SK-MEL-5 | |
| Kidney | 0.0 | Adipose | 12.3 |

TABLE 32
Panel 2D

| | Rel. Exp. (%) Ag1533, | | Rel. Exp. (%) Ag1533, |
|---|---|---|---|
| Tissue Name | Run 145165498 | Tissue Name | Run 145165498 |
| Normal Colon | 48.0 | Kidney Margin | 6.1 |
| | | 8120608 | |
| CC Well to Mod Diff | 5.6 | Kidney Cancer | 0.0 |
| (ODO3866) | | 8120613 | |
| CC Margin (ODO3866) | 4.6 | Kidney Margin | 6.9 |
| | | 8120614 | |
| CC Gr.2 rectosigmoid | 3.5 | Kidney Cancer | 10.2 |
| (ODO3868) | | 9010320 | |
| CC Margin (ODO3868) | 1.0 | Kidney Margin | 15.7 |
| | | 9010321 | |
| CC Mod Diff (ODO3920) | 4.4 | Normal Uterus | 36.3 |
| CC Margin (ODO3920) | 6.6 | Uterine Cancer | 45.7 |
| | | 064011 | |
| CC Gr.2 ascend colon | 1.6 | Normal Thyroid | 8.1 |
| (ODO3921) | | | |
| CC Margin (ODO3921) | 6.1 | Thyroid Cancer | 7.7 |
| CC from Partial | 0.0 | Thyroid Cancer | 27.0 |
| Hepatectomy (ODO4309) | | A302152 | |
| Mets | | | |
| Liver Margin (ODO4309) | 4.8 | Thyroid Margin | 29.5 |
| | | A302153 | |
| Colon mets to lung | 4.3 | Normal Breast | 40.9 |
| (OD04451-01) | | | |
| Lung Margin (OD04451- | 1.2 | Breast Cancer | 3.5 |
| 02) | | | |
| Normal Prostate 6546-1 | 26.1 | Breast Cancer | 18.9 |
| | | (OD04590-01) | |
| Prostate Cancer | 64.2 | Breast Cancer Mets | 34.6 |
| (OD04410) | | (OD04590-03) | |
| Prostate Margin | 43.8 | Breast Cancer | 19.3 |
| (OD04410) | | Metastasis | |
| Prostate Cancer | 42.0 | Breast Cancer | 21.2 |
| (OD04720-01) | | | |
| Prostate Margin | 34.2 | Breast Cancer | 35.1 |
| (OD04720-02) | | | |
| Normal Lung | 31.9 | Breast Cancer | 7.7 |
| | | 9100266 | |
| Lung Met to Muscle | 0.0 | Breast Margin | 2.9 |
| (ODO4286) | | 9100265 | |
| Muscle Margin | 1.9 | Breast Cancer | 14.8 |
| (ODO4286) | | A209073 | |
| Lung Malignant Cancer | 13.1 | Breast Margin | 23.3 |
| (OD03126) | | A2090734 | |
| Lung Margin (OD03126) | 26.8 | Normal Liver | 21.9 |
| Lung Cancer (OD04404) | 5.4 | Liver Cancer | 22.4 |
| Lung Margin (OD04404) | 37.9 | Liver Cancer 1025 | 0.0 |
| Lung Cancer (OD04565) | 2.9 | Liver Cancer 1026 | 2.1 |
| Lung Margin (OD04565) | 9.3 | Liver Cancer 6004-T | 13.5 |
| Lung Cancer (OD04237- | 24.8 | Liver Cancer 6004-N | 5.9 |
| 01) | | | |
| Lung Margin (OD04237- | 10.5 | Liver Cancer 6005-T | 0.0 |
| 02) | | | |
| Ocular Mel Met to Liver | 10.1 | Liver Tissue 6005-N | 0.0 |
| (ODO4310) | | | |
| Liver Margin (ODO4310) | 8.7 | Normal Bladder | 42.0 |
| Melanoma Metastasis | 0.0 | Bladder Cancer | 5.8 |
| Lung Margin (OD04321) | 25.0 | Bladder Cancer | 65.1 |
| Normal Kidney | 100.0 | Bladder Cancer | 5.6 |
| | | (OD04718-01) | |
| Kidney Ca, Nuclear grade | 52.1 | Bladder Normal | 32.8 |
| 2 (OD04338) | | Adjacent | |
| | | (OD04718-03) | |
| Kidney Margin | 22.1 | Normal Ovary | 5.7 |
| (OD04338) | | | |
| Kidney Ca Nuclear grade | 41.5 | Ovarian Cancer | 9.4 |
| 1/2 (OD04339) | | | |
| Kidney Margin | 46.0 | Ovarian Cancer | 8.8 |
| (OD04339) | | (OD04768-07) | |
| Kidney Ca, Clear cell | 27.0 | Ovary Margin | 3.3 |
| type (OD04340) | | (OD04768-08) | |
| Kidney Margin | 24.7 | Normal Stomach | 30.6 |
| (OD04340) | | | |
| Kidney Ca, Nuclear grade | 9.1 | Gastric Cancer | 0.0 |
| 3 (OD04348) | | 9060358 | |
| Kidney Margin | 92.7 | Stomach Margin | 0.0 |
| (OD04348) | | 9060359 | |
| Kidney Cancer | 5.0 | Gastric Cancer | 5.3 |
| (OD04622-01) | | 9060395 | |
| Kidney Margin | 2.0 | Stomach Margin | 3.1 |
| (OD04622-03) | | 9060394 | |
| Kidney Cancer | 10.9 | Gastric Cancer | 4.7 |
| (OD04450-01) | | 9060397 | |
| Kidney Margin | 17.6 | Stomach Margin | 0.0 |
| (OD04450-03) | | 9060396 | |
| Kidney Cancer 8120607 | 0.8 | Gastric Cancer | 11.3 |
| | | 064005 | |

TABLE 33
Panel 4.1D

| | Rel. Exp. (%) Ag1533, | | Rel. Exp. (%) Ag1533, |
|---|---|---|---|
| Tissue Name | Run 223794696 | Tissue Name | Run 223794696 |
| Secondary Th1 act | 0.0 | HUVEC IL-1beta | 11.8 |
| Secondary Th2 act | 20.4 | HUVEC IFN gamma | 11.5 |
| Secondary Tr1 act | 13.1 | HUVEC TNF alpha + IFN | 4.0 |
| | | gamma | |
| Secondary Th1 rest | 25.3 | HUVEC TNF alpha + IL4 | 18.7 |
| Secondary Th2 rest | 8.0 | HUVEC IL-11 | 29.1 |
| Secondary Tr1 rest | 33.9 | Lung Microvascular EC | 83.5 |
| | | none | |
| Primary Th1 act | 0.0 | Lung Microvascular EC | 14.0 |
| | | TNFalpha + IL-1beta | |
| Primary Th2 act | 25.0 | Microvascular Dermal EC | 8.5 |
| | | none | |
| Primary Tr1 act | 7.9 | Microsvasular Dermal EC | 0.0 |
| | | TNFalpha + IL-1beta | |
| Primary Th1 rest | 14.5 | Bronchial epithelium | 15.2 |
| | | TNFalpha + IL1beta | |
| Primary Th2 rest | 4.3 | Small airway epithelium | 0.0 |
| | | none | |
| Primary Tr1 rest | 20.7 | Small airway epithelium | 20.9 |
| | | TNFalpha + IL-1beta | |
| CD45RA CD4 | 21.2 | Coronery artery SMC rest | 0.0 |
| lymphocyte act | | | |
| CD45RO CD4 | 65.5 | Coronery artery SMC | 0.0 |
| lymphocyte act | | TNFalpha + IL-1beta | |
| CD8 lymphocyte act | 25.9 | Astrocytes rest | 20.3 |
| Secondary CD8 | 19.6 | Astrocytes TNFalpha + | 10.3 |
| lymphocyte rest | | IL-1beta | |
| Secondary CD8 | 4.5 | KU-812 (Basophil) rest | 10.9 |
| lymphocyte act | | | |
| CD4 lymphocyte none | 53.2 | KU-812 (Basophil) | 34.2 |
| | | PMA/ionomycin | |
| 2ry Th1/Th2/Tr1_anti- | 19.2 | CCD1106 (Keratinocytes) | 0.0 |
| CD95 CH11 | | none | |
| LAK cells rest | 22.1 | CCD1106 (Keratinocytes) | 8.5 |
| | | TNFalpha + IL-1beta | |
| LAK cells IL-2 | 51.1 | Liver cirrhosis | 26.6 |
| LAK cells IL-2 + IL-12 | 3.1 | NCI-H292 none | 19.9 |
| LAK cells IL-2 + IFN | 24.0 | NCI-H292 IL-4 | 27.5 |
| gamma | | | |
| LAK cells IL-2 + IL-18 | 14.6 | NCI-H292 IL-9 | 29.3 |
| LAK cells | 0.0 | NCI-H292 IL-13 | 16.2 |
| PMA/ionomycin | | | |
| NK Cells IL-2 rest | 38.7 | NCI-H292 IFN gamma | 44.1 |
| Two Way MLR 3 day | 61.6 | HPAEC none | 8.5 |
| Two Way MLR 5 day | 35.6 | HPAEC TNF alpha + IL-1 | 10.7 |
| | | beta | |
| Two Way MLR 7 day | 9.9 | Lung fibroblast none | 60.7 |
| PBMC rest | 26.2 | Lung fibroblast TNF alpha + | 40.6 |
| | | IL-1 beta | |
| PBMC PWM | 3.6 | Lung fibroblast IL-4 | 8.8 |
| PBMC PHA-L | 4.5 | Lung fibroblast IL-9 | 21.3 |
| Ramos (B cell) none | 0.0 | Lung fibroblast IL-13 | 4.8 |
| Ramos (B cell) | 0.0 | Lung fibroblast IFN | 4.6 |
| ionomycin | | gamma | |
| B lymphocytes PWM | 5.1 | Dermal fibroblast | 0.0 |
| | | CCD1070 rest | |
| B lymphocytes CD40L | 17.0 | Dermal fibroblast | 20.0 |
| and IL-4 | | CCD1070 TNF alpha | |
| EOL-1 dbcAMP | 10.9 | Dermal fibroblast | 4.0 |
| | | CCD1070 IL-1 beta | |
| EOL-1 dbcAMP | 0.0 | Dermal fibroblast IFN | 10.7 |
| PMA/ionomycin | | gamma | |
| Dendritic cells none | 4.1 | Dermal fibroblast IL-4 | 26.6 |
| Dendritic cells LPS | 17.7 | Dermal Fibroblasts rest | 10.4 |
| Dendritic cells anti- | 14.8 | Neutrophils TNFa + LPS | 10.0 |
| CD40 | | | |
| Monocytes rest | 10.7 | Neutrophils rest | 21.3 |
| Monocytes LPS | 16.4 | Colon | 0.0 |
| Macrophages rest | 18.6 | Lung | 4.2 |
| Macrophages LPS | 8.0 | Thymus | 100.0 |
| HUVEC none | 5.4 | Kidney | 81.8 |
| HUVEC starved | 2.6 | | |

TABLE 34
Panel 4D

| | Rel. Exp. (%) Ag2862, | | Rel. Exp. (%) Ag2862, |
|---|---|---|---|
| Tissue Name | Run 164299494 | Tissue Name | Run 164299494 |
| Secondary Th1 act | 0.0 | HUVEC IL-1beta | 0.0 |
| Secondary Th2 act | 10.9 | HUVEC IFN gamma | 4.2 |
| Secondary Tr1 act | 2.6 | HUVEC TNF alpha + IFN | 2.5 |
| | | gamma | |
| Secondary Th1 rest | 4.5 | HUVEC TNF alpha + IL4 | 4.6 |
| Secondary Th2 rest | 13.8 | HUVEC IL-11 | 2.4 |
| Secondary Tr1 rest | 9.8 | Lung Microvascular EC | 28.9 |
| | | none | |
| Primary Th1 act | 0.0 | Lung Microvascular EC | 13.2 |
| | | TNFalpha + IL-1beta | |
| Primary Th2 act | 0.0 | Microvascular Dermal EC | 11.7 |
| | | none | |
| IPrimary Tr1 act | 0.0 | Microsvasular Dermal EC | 4.3 |
| | | TNFalpha + IL-1beta | |
| Primary Th1 rest | 65.5 | Bronchial epithelium | 9.7 |
| | | TNFalpha + IL1beta | |
| Primary Th2 rest | 24.0 | Small airway epithelium | 1.7 |
| | | none | |
| Primary Tr1 rest | 6.8 | Small airway epithelium | 36.1 |
| | | TNFalpha + IL-1beta | |
| CD45RA CD4 | 12.9 | Coronery artery SMC rest | 11.1 |
| lymphocyte act | | | |
| CD45RO CD4 | 7.4 | Coronery artery SMC | 6.7 |
| lymphocyte act | | TNFalpha + IL-1beta | |
| CD8 lymphocyte act | 0.0 | Astrocytes rest | 9.4 |
| Secondary CD8 | 13.7 | Astrocytes TNFalpha + | 8.0 |
| lymphocyte rest | | IL-1beta | |
| Secondary CD8 | 0.0 | KU-812 (Basophil) rest | 5.9 |
| lymphocyte act | | | |
| CD4 lymphocyte none | 22.1 | KU-812 (Basophil) | 25.9 |
| | | PMA/ionomycin | |
| 2ry Th1/Th2/Tr1_anti- | 20.2 | CCD1106 (Keratinocytes) | 6.5 |
| CD95 CH11 | | none | |
| LAK cells rest | 20.7 | CCD1106 (Keratinocytes) | 2.9 |
| | | TNFalpha + IL-1beta | |
| LAK cells IL-2 | 53.2 | Liver cirrhosis | 17.8 |
| LAK cells IL-2 + IL-12 | 27.4 | Lupus kidney | 13.9 |
| LAK cells IL-2 + IFN | 73.2 | NCI-H292 none | 23.2 |
| gamma | | | |
| LAK cells IL-2 + IL-18 | 12.6 | NCI-H292 IL-4 | 28.3 |
| LAK cells | 0.0 | NCI-H292 IL-9 | 24.1 |
| PMA/ionomycin | | | |
| NK Cells IL-2 rest | 26.8 | NCI-H292 IL-13 | 2.3 |
| Two Way MLR 3 day | 82.9 | NCI-H292 IFN gamma | 24.1 |
| Two Way MLR 5 day | 12.1 | HPAEC none | 8.0 |
| Two Way MLR 7 day | 0.0 | HPAEC TNF alpha + IL-1 | 6.3 |
| | | beta | |
| PBMC rest | 8.9 | Lung fibroblast none | 9.9 |
| PBMC PWM | 17.4 | Lung fibroblast TNF alpha + | 18.8 |
| | | IL-1 beta | |
| PBMC PHA-L | 7.0 | Lung fibroblast IL-4 | 11.7 |
| Ramos (B cell) none | 0.0 | Lung fibroblast IL-9 | 18.7 |
| Ramos (B cell) | 4.5 | Lung fibroblast IL-13 | 20.0 |
| ionomycin | | | |
| B lymphocytes PWM | 2.5 | Lung fibroblast IFN | 17.3 |
| | | gamma | |
| B lymphocytes CD40L | 12.9 | Dermal fibroblast | 3.0 |
| and IL-4 | | CCD1070 rest | |
| EOL-1 dbcAMP | 4.7 | Dermal fibroblast | 9.9 |
| | | CCD1070 TNF alpha | |
| EOL-1 dbcAMP | 0.0 | Dermal fibroblast | 5.1 |
| PMA/ionomycin | | CCD1070 IL-1 beta | |
| Dendritic cells none | 1.8 | Dermal fibroblast IFN | 1.7 |
| | | gamma | |
| Dendritic cells LPS | 2.8 | Dermal fibroblast IL-4 | 24.5 |
| Dendritic cells anti- | 0.0 | IBD Colitis 2 | 1.8 |
| CD40 | | | |
| Monocytes rest | 6.0 | IBD Crohn's | 2.1 |
| Monocytes LPS | 0.0 | Colon | 14.9 |
| Macrophages rest | 0.0 | Lung | 11.7 |
| Macrophages LPS | 11.9 | Thymus | 82.9 |
| HUVEC none | 0.0 | Kidney | 100.0 |
| HUVEC starved | 3.1 | | |

CNS_neurodegeneration_v1.0 Summary: Ag1533 The GPCR1c gene shows widespread expression across all regions of the brain, with highest expression in the parietal cortex of a control patient (CT=33.5). This gene appears to be upregulated in the temporal cortex of patients with Alzheimer's disease. The temporal cortex is a region that shows severe degeneration in Alzheimer's disease, suggesting the expression of the GPCR1c gene may play a role in the pathogenesis of this disease. Therapeutic modulation of this gene or treatment with an antagonist to the receptor may be of benefit in treating Alzheimer's disease or dementia.

Ag2862 Expression is low/undetected in all samples in this panel (CT>35). (Data not shown.)

General_screening_panel_v1.5 Summary: Ag1533 Highest expression in the fetal lung (CT=30). Significant levels of expression are also detected in adult lung. This expression profile suggests that the GPCR1c gene product may be involved in the normal homeostasis of the lung. Therefore, therapeutic modulation of the expression or function of the GPCR1c gene may be effective in the treatment of diseases of that affect the lung including asthma, emphysema, and acute respiratory distress syndrome (ARDS).

The GPCR1c gene is also moderately expressed in a a variety of metabolic tissues including adipose, adult and fetal heart, adult and fetal skeletal muscle, adrenal, pituitary, thyroid and pancreas. Thus, the GPCR1c gene product may be a small molecule drug target for the treatment of metabolic disease, including obesity and Types 1 and 2 diabetes. Furthermore, the GPCR1c gene is differentially expressed in adult (CT value=37) versus fetal liver (CT values=33.5), and may be used to differentiate between the adult and fetal phenotype in this tissue.

There is moderate expression in some tissues of the central nervous system, including the fetal brain and the spinal cord. Please see CNS_neurodegeneration_v1.0 Summary for discussion of potential utility in the central nervous system.

There is also significant expression of the GPCR1c gene in a cell lines derived from ovarian cancer and gastric cancer. Please see Panel 1.2 for discussion of utility of this gene in the context of this expression profile.

Panel 1.2 Summary: Ag1533 The expression of the GPCR1c gene is highest in a sample derived from an ovarian cancer cell line (CT=29.1). Interestingly, this cell line was derived from a unique form of ovarian cancer, that being ascites. In addition, there appears to be substantial expression of the GPCR1c gene in samples derived from other ovarian cancer cell lines as well as normal bladder tissue, normal kidney tissue and a cell lined derived from a gastric cancer. Thus, the expression of this gene in these tissues could be used to distinguish these samples from other samples in the panel. Additionally, the expression of the GPCR1c gene could be used to distingush ascites derived samples from other samples in the panel. Furthermore, therapeutic modulation of this gene, through the use of small molecule drugs, antibodies or protein therapeutics might be of benefit in the treatment of ovarian cancer.

Panel 1.3D Summary: Ag2617 Expression is exclusive to an ovarian cancer cell line (SK-OV-3)(CT=33.1). Expression in this cell line is also detected in Panel 1.2. Interestingly, this cell line was derived from a unique form of ovarian cancer, that being ascites. Thus, the expression of the GPCR1 c gene could be used to distinguish samples derived from this cell line from other samples in the panel in addition to distingushing ascites from other samples in the panel. Moreover, therapeutic modulation of the GPCR1c gene, through the use of small molecule drugs, antibodies or protein therapeutics might be of benefit in the treatment of ovarian cancer.

Panel 2D Summary: Ag1533 The expression of the GPCR1c gene appears to be highest in a sample derived from normal kidney tissue (CT=31.9). In addition there is substantial expression in samples derived from other samples of normal kidney tissue adjacent to malignant kidney. Moreover, there also appears to be expression associated with tissues, normal or malignant, derived from uterus, prostate, breast, bladder and thyroid. Thus, the expression of this gene could be used to distinguish samples derived from these tissue types when compared to other samples in the panel. Further, therapeutic modulation of the GPCR1c gene, or gene product, through the use of small molecule drugs, antibodies or protein therapeutics might be of benefit in the treatment of cancers of the above listed tissues.

Panel 4.1D Summary: Ag1533 The GPCR1c transcript is expressed on most tissues in panel 4.1 D regardless of treatment, with highest expression in the thymus(CT=32.8). This transcript encodes a GPCR-like molecule with potential signaling activity and may important in maintaining normal cellular functions in a number of tissues. Therapies designed with the protein encoded for by this transcript could be important in regulating cellular viability or function.

Panel 4D Summary: Ag2862 The GPCR1c transcript appears to be expressed in this panel regardless of treatment, with highest expression in the kidney (CT=33.2).This transcript encodes a GPCR-like molecule with potential signaling activity and may important in maintaining normal cellular functions in a number of tissues. Therapies designed with the protein encoded for by this transcript could be important in regulating cellular viability or function.

GPCR2 (GPCR2a and GPCR2b)

Expression of the GPCR2 genes (also referred to as CG50147-01 and variant AC011711_da2) was assessed using the primer-probe sets Ag2322, Ag2365 and Ag2350, described in Tables 35, 36 and 37. Results of the RTQ-PCR runs are shown in Tables 38, 39 and 40.

TABLE 35

Probe Name Ag2322

| | | | | SEQ ID |
|---|---|---|---|---|
| Primers | Sequences | Length | Start Position | NO: |
| Forward | 5′-atcccactgtgcttcatgtatc-3′ | 22 | 119 | 134 |
| Probe | TET-5′-atcccgggcaactgcacaattctttt-3′-TAMRA | 26 | 149 | 135 |
| Reverse | 5′-agtgagcgctctgttttaatga-3′ | 22 | 177 | 136 |

TABLE 36

Probe Name Ag2365

| | | | | SEQ ID |
|---|---|---|---|---|
| Primers | Sequences | Length | Start Position | NO: |
| Forward | 5′-atcccactgtgcttcatgtatc-3′ | 22 | 119 | 137 |
| Probe | TET-5′-atcccgggcaactgcacaattctttt-3′-TAMRA | 26 | 149 | 138 |
| Reverse | 5′-agtgagcgctctgttttaatga-3′ | 22 | 177 | 139 |

TABLE 37

Probe Name Ag2350

| | | | | SEQ ID |
|---|---|---|---|---|
| Primers | Sequences | Length | Start Position | NO: |
| Forward | 5′-atcccactgtgcttcatgtatc-3′ | 22 | 119 | 140 |
| Probe | TET-5′-atcccgggcaactgcacaattcttt-3′-TAMRA | 26 | 149 | 141 |
| Reverse | 5′-agtgagcgctctgttttaatga-3′ | 22 | 177 | 142 |

TABLE 38
Panel 1.3D

| | Rel. Exp. (%) | Rel. Exp. (%) | | Rel. Exp. (%) | Rel. Exp. (%) |
|---|---|---|---|---|---|
| | Ag2322, Run | Ag2350, Run | | Ag2322, Run | Ag2350, Run |
| Tissue Name | 165627868 | 165974845 | Tissue Name | 165627868 | 165974845 |
| Liver | 0.0 | 0.0 | Kidney (fetal) | 0.0 | 0.0 |
| adenocarcinoma | | | | | |
| Pancreas | 0.0 | 0.0 | Renal ca. 786- | 0.0 | 0.0 |
| | | | 0 | | |
| Pancreatic ca. | 0.0 | 0.0 | Renal ca. | 0.0 | 0.0 |
| CAPAN 2 | | | A498 | | |
| Adrenal gland | 0.0 | 0.0 | Renal ca. RXF | 0.0 | 0.0 |
| | | | 393 | | |
| Thyroid | 0.0 | 0.0 | Renal ca. | 0.0 | 0.0 |
| | | | ACHN | | |
| Salivary gland | 0.0 | 0.0 | Renal ca. UO- | 0.0 | 0.0 |
| | | | 31 | | |
| Pituitary gland | 0.0 | 7.6 | Renal ca. TK- | 0.0 | 0.0 |
| | | | 10 | | |
| Brain (fetal) | 0.0 | 0.0 | Liver | 0.0 | 0.0 |
| Brain (whole) | 0.0 | 0.0 | Liver (fetal) | 0.0 | 0.0 |
| Brain (amygdala) | 0.0 | 0.0 | Liver ca. | 0.0 | 0.0 |
| | | | (hepatoblast) | | |
| | | | HepG2 | | |
| Brain | 0.0 | 0.0 | Lung | 0.0 | 0.0 |
| (cerebellum) | | | | | |
| Brain | 0.0 | 0.0 | Lung (fetal) | 0.0 | 0.0 |
| (hippocampus) | | | | | |
| Brain (substantia | 0.0 | 0.0 | Lung ca. | 0.0 | 0.0 |
| nigra) | | | (small cell) | | |
| | | | LX-1 | | |
| Brain (thalamus) | 0.0 | 0.0 | Lung ca. | 0.0 | 31.2 |
| | | | (small cell) | | |
| | | | NCI-H69 | | |
| Cerebral Cortex | 0.0 | 0.0 | Lung ca. | 100.0 | 100.0 |
| | | | (s. cell var.) | | |
| | | | SHP-77 | | |
| Spinal cord | 3.4 | 0.0 | Lung ca. (large | 0.0 | 0.0 |
| | | | cell) NCI-H460 | | |
| glio/astro U87- | 0.0 | 0.0 | Lung ca. (non- | 0.0 | 0.0 |
| MG | | | sm. cell) A549 | | |
| glio/astro U-118- | 0.0 | 0.0 | Lung ca. (non- | 0.0 | 0.0 |
| MG | | | s. cell) NCI- | | |
| | | | H23 | | |
| astrocytoma | 0.0 | 0.0 | Lung ca. (non- | 0.0 | 0.0 |
| SW1783 | | | s. cell) HOP-62 | | |
| neuro*; met SK- | 0.0 | 0.0 | Lung ca. (non- | 0.0 | 0.0 |
| N-AS | | | s. cl) NCI- | | |
| | | | H522 | | |
| astrocytoma SF- | 0.0 | 0.0 | Lung ca. | 0.0 | 0.0 |
| 539 | | | (squam.) SW | | |
| | | | 900 | | |
| astrocytoma | 0.0 | 0.0 | Lung ca. | 16.6 | 15.5 |
| SNB-75 | | | (squam.) NCI- | | |
| | | | H596 | | |
| Glioma SNB-19 | 2.5 | 0.0 | Mammary | 0.0 | 0.0 |
| | | | gland | | |
| Glioma U251 | 0.0 | 0.0 | Breast ca.* | 0.0 | 0.0 |
| | | | (pl. ef) MCF-7 | | |
| Glioma SF-295 | 0.0 | 0.0 | Breast ca.* | 0.0 | 0.0 |
| | | | (pl. ef) MDA- | | |
| | | | MB-231 | | |
| Heart (Fetal) | 0.0 | 0.0 | Breast ca.* (pl. | 0.0 | 0.0 |
| | | | ef) T47D | | |
| Heart | 0.0 | 0.0 | Breast ca. BT- | 0.0 | 0.0 |
| | | | 549 | | |
| Skeletal muscle | 0.0 | 0.0 | Breast ca. | 0.0 | 0.0 |
| (Fetal) | | | MDA-N | | |
| Skeletal muscle | 0.0 | 0.0 | Ovary | 0.0 | 0.0 |
| Bone marrow | 0.0 | 0.0 | Ovarian ca. | 1.7 | 0.0 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.0 | 0.0 |
| | | | OVCAR-5 | | |
| Lymph node | 0.0 | 0.0 | Ovarian ca. | 0.0 | 4.8 |
| | | | OVCAR-8 | | |
| Colorectal | 1.4 | 0.0 | Ovarian ca. | 0.0 | 0.0 |
| | | | IGROV-1 | | |
| Stomach | 0.0 | 0.0 | Ovarian ca. | 0.0 | 0.0 |
| | | | (ascites) SK- | | |
| | | | OV-3 | | |
| Small intestine | 0.0 | 0.0 | Uterus | 0.0 | 0.0 |
| Colon ca. SW480 | 0.0 | 0.0 | Placenta | 0.0 | 0.0 |
| Colon ca.* | 0.0 | 0.0 | Prostate | 0.0 | 0.0 |
| SW620 (SW480 | | | | | |
| met) | | | | | |
| Colon ca. HT29 | 0.0 | 0.0 | Prostate ca.* | 0.0 | 0.0 |
| | | | (bone met) | | |
| | | | PC-3 | | |
| Colon ca. HCT- | 0.0 | 0.0 | Testis | 0.0 | 0.0 |
| 116 | | | | | |
| Colon ca. CaCo-2 | 0.0 | 0.0 | Melanoma | 0.0 | 0.0 |
| | | | Hs688(A).T | | |
| CC Well to Mod | 0.0 | 0.0 | Melanoma* | 0.0 | 0.0 |
| Diff (ODO3866) | | | (met) | | |
| | | | Hs688(B).T | | |
| Colon ca. HCC- | 0.0 | 0.0 | Melanoma | 0.0 | 0.0 |
| 2998 | | | UACC-62 | | |
| Gastric ca. (liver | 3.3 | 0.0 | Melanoma | 0.0 | 0.0 |
| met) NCI-N87 | | | M14 | | |
| Bladder | 0.0 | 6.9 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 0.0 | 0.0 | Adipose | 0.0 | 0.0 |

TABLE 39
Panel 2D

| | Rel. Exp. (%) Ag2350, | | Rel. Exp. (%) Ag2350, |
|---|---|---|---|
| Tissue Name | Run 164079723 | Tissue Name | Run 164079723 |
| Normal Colon | 7.6 | Kidney Margin | 0.0 |
| | | 8120608 | |
| CC Well to Mod Diff | 4.1 | Kidney Cancer | 0.0 |
| (ODO3866) | | 8120613 | |
| CC Margin (ODO3866) | 1.5 | Kidney Margin | 0.0 |
| | | 8120614 | |
| CC Gr.2 rectosigmoid | 3.0 | Kidney Cancer | 0.0 |
| (ODO3868) | | 9010320 | |
| CC Margin (ODO3868) | 0.0 | Kidney Margin | 0.0 |
| | | 9010321 | |
| CC Mod Diff (ODO3920) | 0.0 | Normal Uterus | 0.0 |
| CC Margin (ODO3920) | 7.1 | Uterine Cancer | 3.7 |
| | | 064011 | |
| CC Gr.2 ascend colon | 0.0 | Normal Thyroid | 0.0 |
| (ODO3921) | | | |
| CC Margin (ODO3921) | 0.0 | Thyroid Cancer | 0.0 |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin | 0.0 | Breast Cancer | 7.6 |
| (OD04451-02) | | | |
| Normal Prostate 6546-1 | 22.4 | Breast Cancer | 0.0 |
| | | (OD04590-01) | |
| Prostate Cancer | 74.7 | Breast Cancer Mets | 0.0 |
| (OD04410) | | (OD04590-03) | |
| Prostate Margin | 100.0 | Breast Cancer | 0.0 |
| (OD04410) | | Metastasis | |
| Prostate Cancer | 30.1 | Breast Cancer | 0.0 |
| (OD04720-01) | | | |
| Prostate Margin | 38.7 | Breast Cancer | 0.0 |
| (OD04720-02) | | | |
| Normal Lung | 0.0 | Breast Cancer | 0.0 |
| | | 9100266 | |
| Lung Met to Muscle | 0.0 | Breast Margin | 1.7 |
| (ODO4286) | | 9100265 | |
| Muscle Margin | 0.0 | Breast Cancer | 7.2 |
| (ODO4286) | | A209073 | |
| Lung Malignant Cancer | 25.5 | Breast Margin | 0.0 |
| (OD03126) | | A2090734 | |
| Lung Margin (OD03126) | 0.0 | Normal Liver | 0.0 |
| Lung Cancer (OD04404) | 6.5 | Liver Cancer | 0.0 |
| Lung Margin (OD04404) | 0.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 0.0 | Liver Tissue 6004-N | 0.0 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Metastasis | 0.0 | Bladder Cancer | 0.0 |
| Lung Margin (OD04321) | 0.0 | Bladder Cancer | 6.1 |
| Normal Kidney | 0.0 | Bladder Cancer | 0.0 |
| | | (OD04718-01) | |
| Kidney Ca, Nuclear grade | 0.0 | Bladder Normal | 0.0 |
| 2 (OD04338) | | Adjacent | |
| | | (OD04718-03) | |
| Kidney Margin | 3.6 | Normal Ovary | 0.0 |
| (OD04338) | | | |
| Kidney Ca Nuclear grade | 0.0 | Ovarian Cancer | 0.0 |
| ½ (OD04339) | | | |
| Kidney Margin | 0.0 | Ovarian Cancer | 0.0 |
| (OD04339) | | (OD04768-07) | |
| Kidney Ca, Clear cell | 0.0 | Ovary Margin | 0.0 |
| type (OD04340) | | (OD04768-08) | |
| Kidney Margin | 0.0 | Normal Stomach | 0.0 |
| (OD04340) | | | |
| Kidney Ca, Nuclear grade | 1.7 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 0.0 | Stomach Margin | 0.0 |
| (OD04450-03) | | 9060396 | |
| Kidney Cancer 8120607 | 0.0 | Gastric Cancer | 3.7 |
| | | 064005 | |

TABLE 40
Panel 4D

| | Rel. Exp. (%) | Rel. Exp. (%) | | Rel. Exp. (%) | Rel. Exp. (%) |
|---|---|---|---|---|---|
| | Ag2322, Run | Ag2350, Run | | Ag2322, Run | Ag2350, Run |
| Tissue Name | 162360932 | 164145590 | Tissue Name | 162360932 | 164145590 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 29.3 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 0.0 | 0.0 |
| | | | Microvascular EC | | |
| | | | TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 48.0 |
| lymphocyte act | | | SMC TNFalpha + | | |
| | | | IL-1beta | | |
| CD8 lymphocyte | 0.0 | 23.3 | Astrocytes rest | 0.0 | 0.0 |
| act | | | | | |
| Secondary CD8 | 0.0 | 0.0 | Astrocytes | 0.0 | 0.0 |
| lymphocyte rest | | | TNFalpha + IL- | | |
| | | | 1beta | | |
| Secondary CD8 | 21.2 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 100.0 | 100.0 |
| LAK cells IL-2 + IL- | 0.0 | 0.0 | Lupus kidney | 0.0 | 0.0 |
| 12 | | | | | |
| LAK cells IL- | 0.0 | 0.0 | NCI-H292 | 0.0 | 0.0 |
| 2 + IFN gamma | | | none | | |
| LAK cells IL-2 + | 0.0 | 0.0 | NCI-H292 IL-4 | 0.0 | 0.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 21.0 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 0.0 | 0.0 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.0 |
| ionomycin | | | IL-13 | | |
| B lymphocytes | 7.1 | 0.0 | Lung fibroblast | 0.0 | 40.1 |
| PWM | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 | | |
| | | | beta | | |
| Dendritic cells | 0.0 | 0.0 | Dermal fibroblast | 0.0 | 0.0 |
| none | | | IFN gamma | | |
| Dendritic cells LPS | 0.0 | 0.0 | Dermal fibroblast | 0.0 | 0.0 |
| | | | IL-4 | | |
| Dendritic cells anti- | 0.0 | 0.0 | IBD Colitis 2 | 43.2 | 19.2 |
| CD40 | | | | | |
| Monocytes rest | 0.0 | 0.0 | IBD Crohn's | 0.0 | 15.3 |
| Monocytes LPS | 0.0 | 0.0 | Colon | 0.0 | 0.0 |
| Macrophages rest | 0.0 | 0.0 | Lung | 0.0 | 0.0 |
| Macrophages LPS | 0.0 | 0.0 | Thymus | 0.0 | 19.1 |
| HUVEC none | 0.0 | 0.0 | Kidney | 0.0 | 0.0 |
| HUVEC starved | 0.0 | 0.0 | | | |

CNS_neurodegeneration_v1.0 Summary: Ag2365 Expression is low/undetectable in all samples in this panel due to a probable probe failure. (Data not shown.)

Panel 1.3D Summary: Ag2322/2350 Expression of the GPCR2 genes is concordant in two runs with the same probe and primer set. In both runs, a sample derived from a small cell lung cancer cell line (SHP-77) showed the highest expression (CTs=32-33). There is apparent expression in other small cell lung cancer cell lines as well. Thus, the expression of the GPCR2 genes could be used to distinguish SHP-77 cells and other small cell lung cancer cell lines from other samples on the panel. Furthermore, therapeutic modulation of the expression of the GPCR2 genes, through the use of small molecule drugs, antibodies or protein therapeutics may be of use in the treatment of small cell lung cancer.

Please note that expression is low/undetectable in all samples in this panel (CTs>35) in a third experiment run with Ag2365. (Data not shown.)

Panel 2D Summary: Ag2350 The expression of the GPCR2 genes is highest in a sample derived from normal prostate tissue adjacent to a prostate cancer(CT=32.3). This pattern is true for a second matched pair of prostate cancer and normal tissue, as well. This pattern of expression appears to be highly specific and thus the expression of the GPCR2 genes could be used to distinguish prostate derived tissues from other tissues in the panel.

Moreover, therapeutic modulation of this gene, through the use of small molecule drugs, antibodies or protein therapeutics may be beneficial in the treatment of prostate cancer.

Panel 3D Summary: Ag2350 The amp plot from one experiment with this probe and primer set suggests that there were experimental difficulties with this run. (Data not shown.)

Panel 4D Summary: Ag2322/Ag2350 Significant expression of the GPCR2 genes is restricted to liver cirrhosis (CTs=34-35). Furthermore, the transcript is not expressed in normal liver in panel 1.3D or 2D. Thus, expression of the GPCR2 genes could be used for detection of liver cirrhosis. The putative GPCR encoded by this transcript may also play an important role in liver cirrhosis. Therefore, therapeutics designed with the protein encoded for by this transcript could be important for maintaining or restoring normal function to the liver undergoing cirrhosis.

GPCR3

Expression of the GPCR3 gene (also referred to as GMAC024428_A and variant CG92194_{—}01) was assessed using the primer-probe sets Ag2265, Ag2266 and Ag2277, described in Tables 41, 42 and 43. Results of the RTQ-PCR runs are shown in Tables 44 and 45.

TABLE 41

Probe Name Ag2265

| | | | | SEQ ID |
|---|---|---|---|---|
| Primers | Sequences | Length | Start Position | NO: |
| Forward | 5′-ctctcttctgtggaatcattgc-3′ | 22 | 914 | 143 |
| Probe | TET-5′-cccatgctgaatccccttatatataca-3′-TAMRA | 27 | 937 | 144 |
| Reverse | 5′-ggcttcctttacctctttgttc-3′ | 22 | 969 | 145 |

TABLE 42

Probe Name Ag2266

| | | | Start | SEQ ID |
|---|---|---|---|---|
| Primers | Sequences | Length | Position | NO: |
| Forward | 5′-agattccaccctttgtgatgtt-3′ | 22 | 161 | 146 |
| Probe | TET-5′-tcttgacaatctttggcaatctgaca-3′-TAMRA | 26 | 197 | 147 |
| Reverse | 5′-tgaaatccacatgtgacacaag-3′ | 22 | 229 | 148 |

TABLE 43

Probe Name Ag2277

| | | | Start | SEQ ID |
|---|---|---|---|---|
| Primers | Sequences | Length | Position | NO: |
| Forward | 5′-agattccaccctttgtgatgtt-3′ | 22 | 161 | 149 |
| Probe | TET-5′-tcttgacaatctttggcaatctgaca-3′ | 26 | 197 | 150 |
| Reverse | 5′-tgaaatccacatgtgacacaag-3′ | 22 | 229 | 151 |

TABLE 44
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2265, | Ag2266, | Ag2277, | | Ag2265, | Ag2266, | Ag2277, |
| | Run | Run | Run | | Run | Run | Run |
| Tissue Name | 151562862 | 151041217 | 151769293 | Tissue Name | 151562862 | 151041217 | 151769293 |
| Liver | 3.3 | 1.0 | 4.7 | Kidney | 2.7 | 0.0 | 0.0 |
| adenocarcinoma | | | | (fetal) | | | |
| Pancreas | 2.8 | 0.0 | 0.0 | Renal ca. | 3.6 | 0.0 | 2.3 |
| | | | | 786-0 | | | |
| Pancreatic ca. | 0.0 | 0.0 | 0.0 | Renal ca. | 7.6 | 8.6 | 5.0 |
| CAPAN 2 | | | | A498 | | | |
| Adrenal gland | 0.0 | 1.0 | 0.0 | Renal ca. | 1.4 | 0.0 | 0.0 |
| | | | | RXF 393 | | | |
| Thyroid | 0.0 | 1.2 | 0.0 | Renal ca. | 0.0 | 0.0 | 0.0 |
| | | | | ACHN | | | |
| Salivary gland | 0.0 | 0.0 | 0.0 | Renal ca. | 0.0 | 0.0 | 0.0 |
| | | | | UO-31 | | | |
| Pituitary gland | 0.0 | 0.0 | 0.0 | Renal ca. | 0.0 | 0.6 | 0.0 |
| | | | | TK-10 | | | |
| Brain (fetal) | 0.0 | 1.0 | 0.0 | Liver | 0.0 | 3.4 | 0.0 |
| Brain (whole) | 0.0 | 0.0 | 0.0 | Liver (fetal) | 7.7 | 2.1 | 7.3 |
| Brain | 0.0 | 0.0 | 0.0 | Liver ca. | 6.3 | 0.0 | 2.8 |
| (amygdala) | | | | (hepatoblast) | | | |
| | | | | HepG2 | | | |
| Brain | 0.0 | 0.0 | 2.0 | Lung | 0.0 | 0.0 | 0.0 |
| (cerebellum) | | | | | | | |
| Brain | 0.0 | 0.6 | 0.0 | Lung (fetal) | 1.7 | 0.6 | 0.0 |
| (hippocampus) | | | | | | | |
| Brain | 1.4 | 0.0 | 0.0 | Lung ca. | 7.9 | 1.3 | 0.7 |
| (substantia | | | | (small cell) | | | |
| nigra) | | | | LX-1 | | | |
| Brain | 0.0 | 0.0 | 0.0 | Lung ca. | 11.7 | 3.5 | 5.3 |
| (thalamus) | | | | (small cell) | | | |
| | | | | NCI-H69 | | | |
| Cerebral Cortex | 0.5 | 0.2 | 0.0 | Lung ca. | 33.0 | 22.1 | 12.8 |
| | | | | (s. cell var.) | | | |
| | | | | SHP-77 | | | |
| Spinal cord | 1.2 | 0.0 | 0.0 | Lung ca. | 1.3 | 0.0 | 0.0 |
| | | | | (large | | | |
| | | | | cell) NCI- | | | |
| | | | | H460 | | | |
| glio/astro U87- | 4.1 | 3.2 | 2.9 | Lung ca. | 0.0 | 0.0 | 0.0 |
| MG | | | | (non-sm. | | | |
| | | | | cell) A549 | | | |
| glio/astro U- | 12.2 | 9.2 | 32.8 | Lung ca. | 36.3 | 16.3 | 15.9 |
| 118-MG | | | | (non-s. cell) | | | |
| | | | | NCI-H23 | | | |
| astrocytoma | 1.0 | 6.0 | 0.0 | Lung ca. | 0.0 | 3.2 | 0.0 |
| SW1783 | | | | (non-s. cell) | | | |
| | | | | HOP-62 | | | |
| neuro*; met | 15.8 | 6.6 | 9.2 | Lung ca. | 14.6 | 3.4 | 3.0 |
| SK-N-AS | | | | (non-s. cl) | | | |
| | | | | NCI-H522 | | | |
| astrocytoma | 2.2 | 1.8 | 0.0 | Lung ca. | 0.0 | 1.2 | 1.6 |
| SF-539 | | | | (squam.) | | | |
| | | | | SW 900 | | | |
| astrocytoma | 6.3 | 13.5 | 6.0 | Lung ca. | 13.0 | 16.5 | 1.8 |
| SNB-75 | | | | (squam.) | | | |
| | | | | NCI-H596 | | | |
| Glioma SNB-19 | 0.0 | 0.0 | 0.0 | Mammary | 0.0 | 1.0 | 0.0 |
| | | | | gland | | | |
| Glioma U251 | 0.0 | 0.0 | 0.0 | Breast ca.* | 72.2 | 90.8 | 100.0 |
| | | | | (pl. ef) MCF- | | | |
| | | | | 7 | | | |
| Glioma SF-295 | 8.8 | 4.6 | 8.7 | Breast ca.* | 26.1 | 31.0 | 7.0 |
| | | | | (pl. ef) | | | |
| | | | | MDA-MB- | | | |
| | | | | 231 | | | |
| Heart (Fetal) | 0.0 | 0.0 | 0.0 | Breast ca.* | 3.4 | 6.1 | 2.4 |
| | | | | (pl. ef) | | | |
| | | | | T47D | | | |
| Heart | 0.0 | 0.0 | 1.9 | Breast ca. | 11.0 | 8.6 | 7.9 |
| | | | | BT-549 | | | |
| Skeletal muscle | 1.3 | 3.1 | 0.0 | Breast ca. | 6.6 | 16.6 | 6.9 |
| (Fetal) | | | | MDA-N | | | |
| Skeletal muscle | 0.0 | 0.0 | 2.4 | Ovary | 0.0 | 0.0 | 0.0 |
| Bone marrow | 6.4 | 4.7 | 5.9 | Ovarian ca. | 11.5 | 15.5 | 7.4 |
| | | | | OVCAR-3 | | | |
| Thymus | 0.0 | 2.7 | 2.3 | Ovarian ca. | 0.0 | 0.0 | 0.0 |
| | | | | OVCAR-4 | | | |
| Spleen | 0.0 | 1.1 | 1.3 | Ovarian ca. | 2.2 | 5.0 | 6.4 |
| | | | | OVCAR-5 | | | |
| Lymph node | 0.0 | 0.0 | 0.0 | Ovarian ca. | 1.4 | 0.8 | 0.0 |
| | | | | OVCAR-8 | | | |
| Colorectal | 2.4 | 1.8 | 1.8 | Ovarian ca. | 2.6 | 0.0 | 0.0 |
| | | | | IGROV-1 | | | |
| Stomach | 0.0 | 2.4 | 4.9 | Ovarian ca. | 0.0 | 1.1 | 0.0 |
| | | | | (ascites) SK- | | | |
| | | | | OV-3 | | | |
| Small intestine | 3.3 | 1.1 | 2.5 | Uterus | 0.0 | 0.0 | 0.0 |
| Colon ca. | 12.8 | 8.2 | 16.4 | Placenta | 5.7 | 5.8 | 12.2 |
| SW480 | | | | | | | |
| Colon ca.* | 7.2 | 3.1 | 7.5 | Prostate | 4.3 | 0.0 | 0.0 |
| SW620 | | | | | | | |
| (SW480 met) | | | | | | | |
| Colon ca. HT29 | 2.3 | 5.1 | 5.1 | Prostate ca.* | 8.2 | 7.8 | 10.9 |
| | | | | (bone met) | | | |
| | | | | PC-3 | | | |
| Colon ca. HCT- | 0.0 | 5.0 | 1.8 | Testis | 3.8 | 4.4 | 7.4 |
| 116 | | | | | | | |
| Colon ca. | 13.8 | 1.6 | 4.1 | Melanoma | 0.0 | 0.5 | 0.0 |
| CaCo-2 | | | | Hs688(A).T | | | |
| CC Well to | 0.0 | 0.0 | 0.0 | Melanoma* | 0.0 | 0.0 | 0.0 |
| Mod Diff | | | | (met) | | | |
| (ODO3866) | | | | Hs688(B).T | | | |
| Colon ca. HCC- | 6.7 | 9.0 | 11.0 | Melanoma | 0.0 | 0.0 | 0.0 |
| 2998 | | | | UACC-62 | | | |
| Gastric ca. | 100.0 | 100.0 | 64.2 | Melanoma | 3.2 | 1.1 | 1.6 |
| (liver met) NCI- | | | | M14 | | | |
| N87 | | | | | | | |
| Bladder | 2.3 | 0.7 | 2.4 | Melanoma | 1.9 | 0.0 | 0.0 |
| | | | | LOX IMVI | | | |
| Trachea | 3.7 | 1.9 | 0.0 | Melanoma* | 0.0 | 1.1 | 0.0 |
| | | | | (met) SK- | | | |
| | | | | MEL-5 | | | |
| Kidney | 0.0 | 0.0 | 0.0 | Adipose | 0.0 | 0.0 | 0.0 |

TABLE 45
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag2265, | Ag2266, | Ag2277, | | Ag2265, | Ag2266, | Ag2277, |
| | Run | Run | Run | | Run | Run | Run |
| Tissue Name | 166778536 | 166897525 | 166818127 | Tissue Name | 166778536 | 166897525 | 166818127 |
| Secondary Th1 act | 17.1 | 6.7 | 3.4 | HUVEC IL- | 0.0 | 0.0 | 0.0 |
| | | | | 1beta | | | |
| Secondary Th2 act | 7.1 | 0.0 | 2.5 | HUVEC IFN | 0.0 | 0.0 | 0.0 |
| | | | | gamma | | | |
| Secondary Tr1 act | 4.9 | 4.0 | 0.0 | HUVEC TNF | 0.0 | 0.0 | 0.0 |
| | | | | alpha + IFN | | | |
| | | | | gamma | | | |
| Secondary Th1 | 19.6 | 3.9 | 1.0 | HUVEC TNF | 0.0 | 0.0 | 0.0 |
| rest | | | | alpha + IL4 | | | |
| Secondary Th2 | 6.9 | 4.5 | 0.0 | HUVEC IL-11 | 0.0 | 0.0 | 0.0 |
| rest | | | | | | | |
| Secondary Tr1 | 7.1 | 0.0 | 6.5 | Lung | 0.0 | 0.0 | 0.0 |
| rest | | | | Microvascular | | | |
| | | | | EC none | | | |
| Primary Th1 act | 3.1 | 1.9 | 0.0 | Lung | 4.3 | 0.0 | 0.0 |
| | | | | Microvascular | | | |
| | | | | EC TNFalpha + | | | |
| | | | | IL-1beta | | | |
| Primary Th2 act | 6.0 | 3.8 | 0.0 | Microvascular | 0.0 | 0.0 | 0.0 |
| | | | | Dermal EC none | | | |
| Primary Tr1 act | 0.0 | 4.2 | 4.9 | Microsvasular | 0.0 | 0.0 | 2.1 |
| | | | | Dermal EC | | | |
| | | | | TNFalpha + IL- | | | |
| | | | | 1beta | | | |
| Primary Th1 rest | 12.9 | 10.4 | 2.6 | Bronchial | 0.0 | 0.0 | 51.8 |
| | | | | epithelium | | | |
| | | | | TNFalpha + | | | |
| | | | | IL1beta | | | |
| Primary Th2 rest | 3.4 | 3.0 | 2.9 | Small airway | 0.0 | 0.0 | 49.7 |
| | | | | epithelium none | | | |
| Primary Tr1 rest | 12.0 | 3.8 | 0.0 | Small airway | 0.0 | 0.0 | 11.4 |
| | | | | epithelium | | | |
| | | | | TNFalpha + IL- | | | |
| | | | | 1beta | | | |
| CD45RA CD4 | 5.2 | 0.0 | 7.3 | Coronery artery | 2.0 | 0.0 | 0.0 |
| lymphocyte act | | | | SMC rest | | | |
| CD45RO CD4 | 0.0 | 0.0 | 3.0 | Coronery artery | 0.0 | 0.0 | 1.8 |
| lymphocyte act | | | | SMC TNFalpha + | | | |
| | | | | IL-1beta | | | |
| CD8 lymphocyte | 2.6 | 4.0 | 3.9 | Astrocytes rest | 0.0 | 0.0 | 2.7 |
| act | | | | | | | |
| Secondary CD8 | 4.5 | 0.0 | 0.0 | Astrocytes | 0.0 | 9.6 | 0.0 |
| lymphocyte rest | | | | TNFalpha + IL- | | | |
| | | | | 1beta | | | |
| Secondary CD8 | 1.9 | 0.0 | 0.0 | KU-812 | 10.1 | 47.6 | 16.5 |
| lymphocyte act | | | | (Basophil) rest | | | |
| CD4 lymphocyte | 0.0 | 0.0 | 0.5 | KU-812 | 79.6 | 100.0 | 100.0 |
| none | | | | (Basophil) | | | |
| | | | | PMA/ionomycin | | | |
| 2ry | 10.0 | 4.6 | 2.7 | CCD1106 | 8.7 | 0.0 | 13.1 |
| Th1/Th2/Tr1_anti- | | | | (Keratinocytes) | | | |
| CD95 CH11 | | | | none | | | |
| LAK cells rest | 0.0 | 0.0 | 2.0 | CCD1106 | 2.3 | 0.0 | 0.0 |
| | | | | (Keratinocytes) | | | |
| | | | | TNFalpha + IL- | | | |
| | | | | 1beta | | | |
| LAK cells IL-2 | 12.5 | 6.7 | 8.1 | Liver cirrhosis | 5.3 | 0.0 | 4.1 |
| LAK cells IL- | 2.3 | 0.0 | 2.1 | Lupus kidney | 0.0 | 0.0 | 0.2 |
| 2 + IL-12 | | | | | | | |
| LAK cells IL- | 10.2 | 5.6 | 7.6 | NCI-H292 none | 0.0 | 9.3 | 2.9 |
| 2 + IFN gamma | | | | | | | |
| LAK cells IL-2 + | 16.5 | 9.9 | 4.4 | NCI-H292 IL-4 | 6.2 | 1.6 | 5.8 |
| IL-18 | | | | | | | |
| LAK cells | 0.0 | 0.0 | 0.0 | NCI-H292 IL-9 | 3.2 | 10.3 | 6.0 |
| PMA/ionomycin | | | | | | | |
| NK Cells IL-2 rest | 0.0 | 16.0 | 5.6 | NCI-H292 IL-13 | 0.0 | 5.0 | 12.2 |
| Two Way MLR 3 | 0.0 | 0.0 | 0.0 | NCI-H292 IFN | 2.2 | 14.0 | 7.4 |
| day | | | | gamma | | | |
| Two Way MLR 5 | 5.5 | 8.8 | 0.0 | HPAEC none | 0.0 | 0.0 | 0.0 |
| day | | | | | | | |
| Two Way MLR 7 | 0.0 | 14.7 | 2.3 | HPAEC TNF | 0.0 | 0.0 | 2.3 |
| day | | | | alpha + IL-1 | | | |
| | | | | beta | | | |
| PBMC rest | 0.0 | 0.0 | 0.0 | Lung fibroblast | 1.7 | 5.6 | 0.0 |
| | | | | none | | | |
| PBMC PWM | 57.8 | 45.1 | 40.9 | Lung fibroblast | 0.0 | 0.0 | 3.5 |
| | | | | TNF alpha + IL- | | | |
| | | | | 1 beta | | | |
| PBMC PHA-L | 5.2 | 3.6 | 0.0 | Lung fibroblast | 0.0 | 0.0 | 0.0 |
| | | | | IL-4 | | | |
| Ramos (B cell) | 6.1 | 0.0 | 2.4 | Lung fibroblast | 0.0 | 0.0 | 0.0 |
| none | | | | IL-9 | | | |
| Ramos (B cell) | 12.7 | 39.0 | 2.1 | Lung fibroblast | 2.3 | 0.0 | 0.0 |
| ionomycin | | | | IL-13 | | | |
| B lymphocytes | 25.3 | 42.3 | 29.5 | Lung fibroblast | 0.0 | 0.0 | 2.5 |
| PWM | | | | IFN gamma | | | |
| B lymphocytes | 7.9 | 17.9 | 2.3 | Dermal | 1.7 | 0.0 | 2.3 |
| CD40L and IL-4 | | | | fibroblast | | | |
| | | | | CCD1070 rest | | | |
| EOL-1 dbcAMP | 0.0 | 0.0 | 0.0 | Dermal | 8.1 | 14.5 | 11.4 |
| | | | | fibroblast | | | |
| | | | | CCD1070 TNF | | | |
| | | | | alpha | | | |
| EOL-1 dbcAMP | 0.0 | 4.4 | 0.0 | Dermal | 0.0 | 4.9 | 1.5 |
| PMA/ionomycin | | | | fibroblast | | | |
| | | | | CCD1070 IL-1 | | | |
| | | | | beta | | | |
| Dendritic cells | 0.0 | 0.0 | 0.0 | Dermal | 2.7 | 0.0 | 0.0 |
| none | | | | fibroblast IFN | | | |
| | | | | gamma | | | |
| Dendritic cells | 0.0 | 0.0 | 0.0 | Dermal | 2.0 | 7.5 | 7.7 |
| LPS | | | | fibroblast IL-4 | | | |
| Dendritic cells | 0.0 | 0.0 | 0.0 | IBD Colitis 2 | 0.0 | 0.0 | 6.8 |
| anti-CD40 | | | | | | | |
| Monocytes rest | 0.0 | 0.0 | 0.0 | IBD Crohn's | 2.9 | 4.6 | 0.0 |
| Monocytes LPS | 0.0 | 10.7 | 0.0 | Colon | 0.0 | 0.0 | 0.0 |
| Macrophages rest | 0.0 | 0.0 | 0.0 | Lung | 0.0 | 0.0 | 2.9 |
| Macrophages LPS | 0.0 | 0.0 | 0.0 | Thymus | 0.0 | 0.0 | 0.0 |
| HUVEC none | 0.0 | 0.0 | 0.0 | Kidney | 100.0 | 81.2 | 48.0 |
| HUVEC starved | 0.0 | 0.0 | 3.1 | | | | |

Panel 1.3D Summary: Ag2265/2266/2277 The expression of the GPCR3 gene was assessed in triplicate runs on panel 1.3D. There was good concordance between runs, with the highest expression seen in cell lines derived from a gastric cancer metastasis (NCI-N87) (CTs=30.5-32.5) and breast cancer (MCF-7). Thus, the expression of the GPCR3 gene could be used to distinguish NCI-N87 and MCF-7 from the other samples in the panel. Moreover, therapeutic modulation of the GPCR3 genes, through the use of small molecule drugs, antibodies or protein therapeutics might be of benefit in the treatment of gastric cancer or breast cancer.

Panel 2.2 Summary: Ag2265/2266/2277 Expression is low/undetectable in all samples in this panel. (CTs>35). (Data not shown.)

Panel 4D Summary: Ag2265/2266/2277 The GPCR3 gene is expressed in normal kidney and in a basophil cell line and is slightly induced in activated basophils. Basophils are an important component of the allergic immune response. These cells produce cytokines and mediators that can activate T cells (IL-4), induce eosinophil recruitment, smooth muscle contraction, and vascular permeability (histamines) and contribute to tissue destruction (IL-13) (1). Therefore, therapeutics designed to block the function of the protein encoded by this transcript could be important in the treatment of asthma, inflammatory bowel disease and psoriasis.

GPCR4

Expression of GPCR4 (also referred to as CG50169-01) was assessed using the primer-probe sets Ag2384, Ag2226 and Agl 828, described in Tables 46, 47 and 48. Results of the RTQ-PCR runs are shown in Tables 49 and 50.

TABLE 46

Probe Name Ag2384

| | | | Start | SEQ ID |
|---|---|---|---|---|
| Primers | Sequences | Length | Position | NO: |
| Forward | 5′-ggaaagtgtcctccctgttcta-3′ | 22 | 824 | 152 |
| Probe | TET-5′-ccataatagtccccgtgttaaaccca-3′ | 26 | 48 | 153 |
| Reverse | 5′-ctttgacatccttgttcctcaa-3′ | 22 | 886 | 154 |

TABLE 47

Probe Name Ag2226

| | | | Start | SEQ ID |
|---|---|---|---|---|
| Primers | Sequences | Length | Position | NO: |
| Forward | 5′-ggaaagtgtcctccctgttcta-3′ | 22 | 824 | 155 |
| Probe | TET-5′-ccataatagtccccgtgttaaaccca-3′-TAMRA | 26 | 848 | 156 |
| Reverse | 5′-ctttgacatccttgttcctcaa-3′ | 22 | 886 | 157 |

TABLE 48

Probe Name Ag1828

| | | | Start | SEQ ID |
|---|---|---|---|---|
| Primers | Sequences | Length | Position | NO: |
| Forward | 5′-cctctccagcattctacacaac-3′ | 22 | 675 | 158 |
| Probe | TET-5′-tctacagaaggcaggtccaaagcctt-3′-TAMRA | 26 | 700 | 159 |
| Reverse | 5′-caattatgtgggaactgcaagt-3′ | 22 | 730 | 160 |

TABLE 49
Panel 1.3D

| | Rel. Exp. (%) Ag2384, | | Rel. Exp. (%) Ag2384, |
|---|---|---|---|
| Tissue Name | Run 165629298 | Tissue Name | Run 165629298 |
| Liver adenocarcinoma | 10.5 | Kidney (fetal) | 9.5 |
| Pancreas | 1.3 | Renal ca. 786-0 | 2.9 |
| Pancreatic ca. | 3.7 | Renal ca. A498 | 5.0 |
| CAPAN 2 | | | |
| Adrenal gland | 5.8 | Renal ca. RXF 393 | 8.6 |
| Thyroid | 23.7 | Renal ca. ACHN | 6.1 |
| Salivary gland | 4.6 | Renal ca. UO-31 | 2.3 |
| Pituitary gland | 5.6 | Renal ca. TK-10 | 1.9 |
| Brain (fetal) | 7.6 | Liver | 1.3 |
| Brain (whole) | 2.4 | Liver (fetal) | 5.3 |
| Brain (amygdala) | 7.6 | Liver ca. | 0.1 |
| | | (hepatoblast) HepG2 | |
| Brain (cerebellum) | 1.1 | Lung | 17.9 |
| Brain (hippocampus) | 6.2 | Lung (fetal) | 15.0 |
| Brain (substantia nigra) | 2.8 | Lung ca. (small cell) | 2.3 |
| | | LX-1 | |
| Brain (thalamus) | 4.8 | Lung ca. (small cell) | 0.1 |
| | | NCI-H69 | |
| Cerebral Cortex | 2.8 | Lung ca. (s. cell var.) | 5.8 |
| | | SHP-77 | |
| Spinal cord | 13.9 | Lung ca. (large | 4.8 |
| | | cell) NCI-H460 | |
| glio/astro U87-MG | 1.5 | Lung ca. (non-sm. | 1.5 |
| | | cell) A549 | |
| glio/astro U-118-MG | 4.2 | Lung ca. (non-s. cell) | 3.1 |
| | | NCI-H23 | |
| astrocytoma SW1783 | 0.1 | Lung ca. (non-s. cell) | 3.9 |
| | | HOP-62 | |
| neuro*; met SK-N-AS | 4.0 | Lung ca. (non-s. cl) | 1.8 |
| | | NCI-H522 | |
| astrocytoma SF-539 | 3.5 | Lung ca. (squam.) | 9.3 |
| | | SW 900 | |
| astrocytoma SNB-75 | 12.1 | Lung ca. (squam.) | 0.0 |
| | | NCI-H596 | |
| Glioma SNB-19 | 8.3 | Mammary gland | 15.4 |
| Glioma U251 | 3.8 | Breast ca.* (pl. ef) | 7.1 |
| | | MCF-7 | |
| Glioma SF-295 | 5.2 | Breast ca.* (pl. ef) | 12.5 |
| | | MDA-MB-231 | |
| Heart (Fetal) | 15.5 | Breast ca.* (pl. ef) | 1.8 |
| | | T47D | |
| Heart | 20.6 | Breast ca. BT-549 | 2.4 |
| Skeletal muscle (Fetal) | 8.0 | Breast ca. MDA-N | 0.7 |
| Skeletal muscle | 13.2 | Ovary | 51.8 |
| Bone marrow | 1.1 | Ovarian ca. OVCAR-3 | 0.7 |
| Thymus | 2.2 | Ovarian ca. OVCAR-4 | 4.4 |
| Spleen | 11.0 | Ovarian ca. OVCAR-5 | 4.4 |
| Lymph node | 21.3 | Ovarian ca. OVCAR-8 | 0.6 |
| Colorectal | 5.4 | Ovarian ca. IGROV-1 | 0.6 |
| Stomach | 19.2 | Ovarian ca. (ascites) | 1.2 |
| | | SK-OV-3 | |
| Small intestine | 60.7 | Uterus | 100.0 |
| Colon ca. SW480 | 0.6 | Placenta | 4.5 |
| Colon ca.* SW620 | 0.4 | Prostate | 8.1 |
| (SW480 met) | | | |
| Colon ca. HT29 | 0.3 | Prostate ca.* (bone | 3.2 |
| | | met) PC-3 | |
| Colon ca. HCT-116 | 5.3 | Testis | 17.9 |
| Colon ca. CaCo-2 | 1.3 | Melanoma | 0.6 |
| | | Hs688 (A).T | |
| CC Well to Mod Diff | 2.5 | Melanoma* (met) | 0.2 |
| (ODO3866) | | Hs688 (B).T | |
| Colon ca. HCC-2998 | 2.7 | Melanoma UACC-62 | 0.9 |
| Gastric ca. (liver met) | 6.4 | Melanoma M14 | 5.3 |
| NCI-N87 | | | |
| Bladder | 2.5 | Melanoma LOX | 1.6 |
| | | IMVI | |
| Trachea | 24.5 | Melanoma* (met) | 1.8 |
| | | SK-MEL-5 | |
| Kidney | 2.2 | Adipose | 7.2 |

TABLE 50
Panel 4D

| | Rel. Exp. (%) | Rel. Exp. (%) | | Rel. Exp. (%) | Rel. Exp. (%) |
|---|---|---|---|---|---|
| | Ag1828, Run | Ag2384, Run | | Ag1828, Run | Ag2384, Run |
| Tissue Name | 165810353 | 162321123 | Tissue Name | 165810353 | 162321123 |
| Secondary Th1 act | 0.0 | 10.9 | HUVEC IL-1beta | 0.0 | 4.8 |
| Secondary Th2 act | 0.0 | 14.7 | HUVEC IFN | 0.0 | 15.6 |
| | | | gamma | | |
| Secondary Tr1 act | 0.0 | 11.6 | HUVEC TNF | 0.0 | 23.8 |
| | | | alpha + IFN | | |
| | | | gamma | | |
| Secondary Th1 rest | 0.0 | 6.6 | HUVEC TNF | 0.0 | 24.8 |
| | | | alpha + IL4 | | |
| Secondary Th2 rest | 0.0 | 12.9 | HUVEC IL-11 | 0.0 | 14.0 |
| Secondary Tr1 rest | 0.0 | 8.4 | Lung | 0.0 | 27.9 |
| | | | Microvascular EC | | |
| | | | none | | |
| Primary Th1 act | 0.0 | 8.2 | Lung | 0.0 | 17.7 |
| | | | Microvascular EC | | |
| | | | TNFalpha + IL- | | |
| | | | 1beta | | |
| Primary Th2 act | 0.0 | 20.3 | Microvascular | 0.0 | 22.1 |
| | | | Dermal EC none | | |
| Primary Tr1 act | 22.7 | 17.4 | Microsvasular | 0.0 | 15.8 |
| | | | Dermal EC | | |
| | | | TNFalpha + IL- | | |
| | | | 1beta | | |
| Primary Th1 rest | 0.0 | 22.7 | Bronchial | 0.0 | 17.4 |
| | | | epithelium | | |
| | | | TNFalpha + | | |
| | | | IL1beta | | |
| Primary Th2 rest | 3.5 | 16.0 | Small airway | 0.0 | 7.3 |
| | | | epithelium none | | |
| Primary Tr1 rest | 0.0 | 15.8 | Small airway | 0.0 | 34.6 |
| | | | epithelium | | |
| | | | TNFalpha + IL- | | |
| | | | 1beta | | |
| CD45RA CD4 | 0.0 | 19.3 | Coronery artery | 0.0 | 81.8 |
| lymphocyte act | | | SMC rest | | |
| CD45RO CD4 | 0.0 | 8.8 | Coronery artery | 0.0 | 41.8 |
| lymphocyte act | | | SMC TNFalpha + | | |
| | | | IL-1beta | | |
| CD8 lymphocyte | 0.0 | 9.0 | Astrocytes rest | 0.0 | 34.2 |
| act | | | | | |
| Secondary CD8 | 0.0 | 7.9 | Astrocytes | 0.0 | 26.1 |
| lymphocyte rest | | | TNFalpha + IL- | | |
| | | | 1beta | | |
| Secondary CD8 | 0.0 | 11.2 | KU-812 | 0.0 | 13.7 |
| lymphocyte act | | | (Basophil) rest | | |
| CD4 lymphocyte | 0.0 | 9.2 | KU-812 | 6.9 | 35.6 |
| none | | | (Basophil) | | |
| | | | PMA/ionomycin | | |
| 2ry | 0.0 | 14.8 | CCD1106 | 0.0 | 19.3 |
| Th1/Th2/Tr1_anti- | | | (Keratinocytes) | | |
| CD95 CH11 | | | none | | |
| LAK cells rest | 0.0 | 16.8 | CCD1106 | 0.0 | 23.7 |
| | | | (Keratinocytes) | | |
| | | | TNFalpha + IL- | | |
| | | | 1beta | | |
| LAK cells IL-2 | 0.0 | 5.1 | Liver cirrhosis | 100.0 | 4.1 |
| LAK cells IL-2 + IL- | 0.0 | 8.1 | Lupus kidney | 0.0 | 3.0 |
| 12 | | | | | |
| LAK cells IL- | 0.0 | 9.0 | NCI-H292 none | 0.0 | 0.0 |
| 2 + IFN gamma | | | | | |
| LAK cells IL-2 + | 0.0 | 8.7 | NCI-H292 IL-4 | 0.0 | 0.0 |
| IL-18 | | | | | |
| LAK cells | 0.0 | 27.5 | NCI-H292 IL-9 | 0.0 | 0.0 |
| PMA/ionomycin | | | | | |
| NK Cells IL-2 rest | 0.0 | 6.9 | NCI-H292 IL-13 | 0.0 | 0.0 |
| Two Way MLR 3 | 0.0 | 6.6 | NCI-H292 IFN | 0.0 | 0.0 |
| day | | | gamma | | |
| Two Way MLR 5 | 0.0 | 7.1 | HPAEC none | 0.0 | 9.7 |
| day | | | | | |
| Two Way MLR 7 | 0.0 | 6.2 | HPAEC TNF | 0.0 | 13.2 |
| day | | | alpha + IL-1 beta | | |
| PBMC rest | 0.0 | 21.2 | Lung fibroblast | 0.0 | 23.5 |
| | | | none | | |
| PBMC PWM | 0.0 | 10.7 | Lung fibroblast | 0.0 | 6.8 |
| | | | TNF alpha + IL-1 | | |
| | | | beta | | |
| PBMC PHA-L | 0.0 | 15.0 | Lung fibroblast | 0.0 | 61.1 |
| | | | IL-4 | | |
| Ramos (B cell) | 0.0 | 0.0 | Lung fibroblast | 0.0 | 29.7 |
| none | | | IL-9 | | |
| Ramos (B cell) | 0.0 | 0.0 | Lung fibroblast | 0.0 | 37.4 |
| ionomycin | | | IL-13 | | |
| B lymphocytes | 0.0 | 17.6 | Lung fibroblast | 0.0 | 64.2 |
| PWM | | | IFN gamma | | |
| B lymphocytes | 3.8 | 2.3 | Dermal fibroblast | 0.0 | 100.0 |
| CD40L and IL-4 | | | CCD1070 rest | | |
| EOL-1 dbcAMP | 0.0 | 20.3 | Dermal fibroblast | 0.0 | 78.5 |
| | | | CCD1070 TNF | | |
| | | | alpha | | |
| EOL-1 dbcAMP | 0.0 | 4.5 | Dermal fibroblast | 0.0 | 54.0 |
| PMA/ionomycin | | | CCD1070 IL-1 | | |
| | | | beta | | |
| Dendritic cells | 0.0 | 16.3 | Dermal fibroblast | 0.0 | 26.4 |
| none | | | IFN gamma | | |
| Dendritic cells LPS | 0.0 | 21.5 | Dermal fibroblast | 0.0 | 42.0 |
| | | | IL-4 | | |
| Dendritic cells anti- | 7.6 | 24.1 | IBD Colitis 2 | 8.1 | 4.3 |
| CD40 | | | | | |
| Monocytes rest | 0.0 | 44.8 | IBD Crohn's | 9.2 | 6.3 |
| Monocytes LPS | 0.0 | 3.4 | Colon | 31.9 | 30.4 |
| Macrophages rest | 3.7 | 34.2 | Lung | 3.8 | 33.0 |
| Macrophages LPS | 0.0 | 12.7 | Thymus | 0.0 | 4.0 |
| HUVEC none | 0.0 | 19.5 | Kidney | 0.0 | 16.8 |
| HUVEC starved | 0.0 | 25.5 | | | |

CNS_neurodegeneration_v1.0 Summary: Ag2226/Ag2384 Expression is low/undetectable in all the samples in this panel. (CTs>35). (Data not shown.)

Panel 1.3D Summary: Ag2384 Highest expression of the GPCR4 gene is detected in the uterus (CT=28.8). There is also substantial expression in normal ovarian and small intestine tissue. Of note is the low level of expression in cell lines derived from ovarian cancer. Thus, the expression of this gene could be used to distinguish uterine, ovarian and small intestine tissue from other tissues in the panel. In addition, the expression of this gene could be used to distinguish normal ovarian tissue from samples derived from ovarian cancer cell lines. Furthermore, therapeutic modulation of the GPCR4 gene, through the use of small molecule drugs, antibodies or protein therapeutics may be beneficial in the treatment of ovarian cancer.

The GPCR4 gene is also moderately expressed (CT values=31-33)in a variety of metabolic tissues including adrenal, thyroid, pituitary, adult and fetal heart, adult and fetal skeletal muscle, fetal liver and adipose. Thus, the GPCR4 gene product may be a small molecule target for the treatment of metabolic disease, including obesity and Types 1 and 2 diabetes.

The GPCR4 gene is expressed at low to moderate levels in all CNS regions examined. The encoded protein is a novel member of the GPCR family of receptors. Several neurotransmitter receptors are GPCRs, including the dopamine receptor family, the serotonin receptor family, the GABAB receptor, muscarinic acetylcholine receptors, and others; thus, GPCR4 may represent a novel neurotransmitter receptor. Targeting various neurotransmitter receptors (dopamine, serotonin) has proven to be an effective therapy in psychiatric illnesses such as schizophrenia, bipolar disorder and depression. In addition, other regions where the GPCR4 gene is expressed (the cerebral cortex and hippocampus) are known to play critical roles in Alzheimer's disease, seizure disorders, and in the normal process of memory formation. Thus, therapeutic modulation of the expression of this gene or its protein product may be beneficial in one or more of these diseases, as may blockade of the receptor encoded by the gene. Furthermore, significant levels of expression of the GPCR4 gene in areas outside the central nervous system (such as uterus and ovary), suggest the possibility of a wider role in intercellular signaling.

Please note that a second experiment with the probe and primer set Ag2226 showed expression to be low/undetectable in all the samples in this panel. (CTs>35). (Data not shown.)

Panel 2.2 Summary: Ag2226 Expression is low/undetectable in all the samples in this panel. (CTs>35). (Data not shown.)

Panel 4D Summary: Ag2384 The GPCR4 transcript is expressed in most tissues in this panel regardless of treatment. This transcript encodes a GPCR like molecule with potential signaling activity that may be important in maintaining normal cellular functions in a number of tissues. Therapies designed with the protein encoded by this transcript could be important in regulating cellular viability or function.

A second experiment with the probe and primer set Agl 828 is not consistent with the above results and shows low levels of transcript expression in liver cirrhosis only.

Ag2226 Expression is low/undetectable in all the samples in this panel. (CTs>35). (Data not shown.)

GPCR5

Expression of the GPCR5 gene (also referred to as AC009758_da1) was assessed using the primer-probe sets Ag2319 and Ag2337, described in Tables 51 and 52. Results of the RTQ-PCR runs are shown in Table 53.

TABLE 51

Probe Name Ag2319

| | | | Start | SEQ ID |
|---|---|---|---|---|
| Primers | Sequences | Length | Position | NO: |
| Forward | 5′-attccacacaccttttgtgaac-3′ | 22 | 591 | 161 |
| Probe | TET-5′-acattggcctagccaaatatgcatgt-3′-TAMRA | 26 | 613 | 162 |
| Reverse | 5′-ggaaaacccataccaaatgttt-3′ | 22 | 653 | 163 |

TABLE 52

Probe Name Ag2337

| | | | | SEQ ID |
|---|---|---|---|---|
| Primers | Sequences | Length | Start Position | NO: |
| Forward | 5′-attccacacaccttttgtgaac-3′ | 22 | 591 | 164 |
| Probe | TET-5′-acattggcctagccaaatatgcatgt-3′-TAMRA | 26 | 613 | 165 |
| Reverse | 5′-ggaaaacccataccaaatgttt-3′ | 22 | 653 | 166 |

TABLE 53
Panel 4.1D

| | Rel. | Rel. | | Rel. | Rel. |
|---|---|---|---|---|---|
| | Exp. (%) | Exp. (%) | | Exp. (%) | Exp. (%) |
| | Ag2319, | Ag2337, | | Ag2319, | Ag2337, |
| | Run | Run | | Run | Run |
| Tissue Name | 224781588 | 224781634 | Tissue Name | 224781588 | 224781634 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 0.0 | 0.0 |
| | | | Microvascular EC | | |
| | | | TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNFalpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 0.0 |
| lymphocyte rest | | | TNFalpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 0.0 | 0.0 |
| LAK cells IL-2 + IL- | 0.0 | 0.0 | NCI-H292 none | 0.0 | 0.0 |
| 12 | | | | | |
| LAK cells IL- | 0.0 | 0.0 | NCI-H292 IL-4 | 0.0 | 0.0 |
| 2 + IFN gamma | | | | | |
| LAK cells IL-2 + | 0.0 | 0.0 | NCI-H292 IL-9 | 0.0 | 0.0 |
| IL-18 | | | | | |
| LAK cells | 0.0 | 0.0 | NCI-H292 IL-13 | 0.0 | 0.0 |
| PMA/ionomycin | | | | | |
| NK Cells IL-2 rest | 0.0 | 0.0 | NCI-H292 IFN | 0.0 | 0.0 |
| | | | gamma | | |
| Two Way MLR 3 | 0.0 | 0.0 | HPAEC none | 0.0 | 0.0 |
| day | | | | | |
| Two Way MLR 5 | 0.0 | 0.0 | HPAEC TNF | 0.0 | 0.0 |
| day | | | alpha + IL-1 beta | | |
| Two Way MLR 7 | 0.0 | 0.0 | Lung fibroblast | 0.0 | 0.0 |
| day | | | none | | |
| PBMC rest | 0.0 | 0.0 | Lung fibroblast | 0.0 | 0.0 |
| | | | TNF alpha + IL-1 | | |
| | | | beta | | |
| PBMC PWM | 0.0 | 0.0 | Lung fibroblast | 0.0 | 0.0 |
| | | | IL-4 | | |
| PBMC PHA-L | 0.0 | 0.0 | Lung fibroblast | 0.0 | 0.0 |
| | | | IL-9 | | |
| Ramos (B cell) | 0.0 | 0.0 | Lung fibroblast | 0.0 | 0.0 |
| none | | | IL-13 | | |
| Ramos (B cell) | 0.0 | 0.0 | Lung fibroblast | 0.0 | 0.0 |
| ionomycin | | | IFN gamma | | |
| B lymphocytes | 0.0 | 0.0 | Dermal fibroblast | 0.0 | 0.0 |
| PWM | | | CCD1070 rest | | |
| B lymphocytes | 0.0 | 0.0 | Dermal fibroblast | 0.0 | 0.0 |
| CD40L and IL-4 | | | CCD1070 TNF | | |
| | | | alpha | | |
| EOL-1 dbcAMP | 0.0 | 0.0 | Dermal fibroblast | 0.0 | 0.0 |
| | | | CCD1070 IL-1 | | |
| | | | beta | | |
| EOL-1 dbcAMP | 0.0 | 0.0 | Dermal fibroblast | 0.0 | 1.0 |
| PMA/ionomycin | | | IFN gamma | | |
| Dendritic cells | 0.0 | 0.0 | Dermal fibroblast | 0.0 | 0.0 |
| none | | | IL-4 | | |
| Dendritic cells LPS | 0.0 | 0.0 | Dermal | 0.0 | 0.0 |
| | | | Fibroblasts rest | | |
| Dendritic cells anti- | 0.0 | 0.0 | Neutrophils | 0.0 | 1.2 |
| CD40 | | | TNFa + LPS | | |
| Monocytes rest | 0.0 | 0.0 | Neutrophils rest | 0.0 | 1.1 |
| Monocytes LPS | 0.0 | 0.0 | Colon | 0.0 | 0.0 |
| Macrophages rest | 0.0 | 0.0 | Lung | 1.7 | 1.8 |
| Macrophages LPS | 0.0 | 0.0 | Thymus | 9.6 | 14.5 |
| HUVEC none | 0.0 | 0.0 | Kidney | 100.0 | 100.0 |
| HUVEC starved | 0.0 | 0.0 | | | |

CNS_neurodegeneration_v1.0 Summary Ag2319/Ag2337 Expression of the GPCR5 gene is low/undetectable (CTs>35) across all of the samples on this panel (data not shown).

General_screening_panel_v1.5 Summary Ag2337 Expression of the GPCR5 gene is low/undetectable (CTs>35) across all of the samples on this panel due to a probable probe failure. (data not shown).

Panel 1.3D Summary Ag2319/Ag2337 Expression of the GPCR5 gene is low/undetectable (CTs>35) across all of the samples on this panel due to a probable probe failure. (data not shown).

Panel 2.2 Summary Ag2319 Expression of the GPCR5 gene is low/undetectable (CTs >35) across all of the samples on this panel due to a probable probe failure. (data not shown-Run 174309682). A suspicious amp plot from a second experiment with the same probe and primer set suggests that there were experimental difficulties with that run. (Data not shown-Run 174310428.)

Panel 4.1D Summary: Ag2319/Ag2337 Expression of the GPCR5 gene is limited to kidney. Therefore, expression of this gene could be used to distinguish kidney from other tissues. Furthermore, the restriction of expression of the GPCR5 gene to the kidney suggests that the protein encoded by this gene may be involved in the homeostasis of the kidney. Therefore, therapeutic modulation of the expression or function of the GPCR5 gene may be effective in the treatment of diseases of the kidney such as diabetes, autoimmune disease, renal artery stenosis, interstitial nephritis, glomerulonephritis, polycystic kidney disease, systemic lupus erythematosus, renal tubular acidosis, IgA nephropathy, hypercalceimia, and Lesch-Nyhan syndrome.

Panel 4D Summary Ag2319/Ag2337 Expression of the GPCR5 gene is low/undetectable (CTs>35) in all of the tissues on this panel (data not shown). (Nouri-Aria et al., Basophil recruitment and IL-4 production during human allergen-induced late asthma. J Allergy Clin Immunol 108(2):205-11, 2001).

GPCR6

Expression of the GPCR6 gene (also referred to as CG50149-01) was assessed using the primer-probe sets Ag2364 and Agl725, described in Tables 54 and 55. Results of the RTQ-PCR runs are shown in Table 56.

TABLE 54

Probe Name Ag2364

| | | | Start | SEQ ID |
|---|---|---|---|---|
| Primers | Sequences | Length | Position | NO: |
| Forward | 5′-caaccagccacagagatagttg-3′ | 22 | 224 | 167 |
| Probe | TET-5′-cttcttgcaggaagccctccagcatt-3′-TAMRA | 26 | 198 | 168 |
| Reverse | 5′-tctctacacctccgacagtgat-3′ | 21 | 171 | 169 |

TABLE 55

Probe Name Ag1725

| | | | | SEQ ID |
|---|---|---|---|---|
| Primers | Sequences | Length | Start Position | NO: |
| Forward | 5′-gctcaggtgacaactctcattc-3′ | 22 | 538 | 170 |
| Probe | TET-5′-tgtgttctgcctcactattccttttgga-3′-TAMRA | 28 | 564 | 171 |
| Reverse | 5′-caccacaattctggcataagat-3′ | 22 | 603 | 172 |

TABLE 56
Panel 4D

| | Rel. | Rel. | | Rel. | Rel. |
|---|---|---|---|---|---|
| | Exp. (%) | Exp. (%) | | Exp. (%) | Exp. (%) |
| | Ag1725, | Ag2364, | | Ag1725, | Ag2364, |
| | Run | Run | | Run | Run |
| Tissue Name | 165767161 | 162361133 | Tissue Name | 165767161 | 162361133 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 2.1 | 0.0 | Lung | 2.3 | 0.0 |
| | | | Microvascular EC | | |
| | | | none | | |
| Primary Th1 act | 0.0 | 0.0 | Lung | 0.0 | 0.0 |
| | | | Microvascular EC | | |
| | | | TNFalpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 0.0 | 0.0 |
| | | | epithelium | | |
| | | | TNFalpha + | | |
| | | | IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNFalpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNFalpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 0.0 |
| lymphocyte rest | | | TNFalpha + IL- | | |
| | | | 1beta | | |
| Secondary CD8 | 0.0 | 0.0 | KU-812 | 0.0 | 13.0 |
| lymphocyte act | | | (Basophil) rest | | |
| CD4 lymphocyte | 0.0 | 0.0 | KU-812 | 3.7 | 0.0 |
| none | | | (Basophil) | | |
| | | | PMA/ionomycin | | |
| 2ry | 0.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 3.0 | 0.0 |
| | | | (Keratinocytes) | | |
| | | | TNFalpha + IL- | | |
| | | | 1beta | | |
| LAK cells IL-2 | 0.0 | 0.0 | Liver cirrhosis | 100.0 | 100.0 |
| LAK cells IL-2 + IL- | 0.0 | 0.0 | Lupus kidney | 2.0 | 0.0 |
| 12 | | | | | |
| LAK cells IL- | 0.0 | 0.0 | NCI-H292 none | 0.0 | 0.0 |
| 2 + IFN gamma | | | | | |
| LAK cells IL-2 + | 0.0 | 0.0 | NCI-H292 IL-4 | 0.0 | 0.0 |
| IL-18 | | | | | |
| LAK cells | 0.0 | 0.0 | NCI-H292 IL-9 | 0.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 | 0.0 | 0.0 | NCI-H292 IFN | 0.0 | 0.0 |
| day | | | gamma | | |
| Two Way MLR 5 | 0.0 | 0.0 | HPAEC none | 0.0 | 0.0 |
| day | | | | | |
| Two Way MLR 7 | 0.0 | 0.0 | HPAEC TNF | 0.0 | 0.0 |
| day | | | alpha + IL-1 beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 0.0 | 0.0 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.0 |
| ionomycin | | | IL-13 | | |
| B lymphocytes | 0.0 | 0.0 | Lung fibroblast | 0.0 | 0.0 |
| PWM | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF | | |
| | | | alpha | | |
| EOL-1 dbcAMP | 0.0 | 0.0 | Dermal fibroblast | 0.0 | 0.0 |
| PMA/ionomycin | | | CCD1070 IL-1 | | |
| | | | beta | | |
| Dendritic cells | 0.0 | 0.0 | Dermal fibroblast | 0.0 | 0.0 |
| none | | | IFN gamma | | |
| Dendritic cells LPS | 0.0 | 0.0 | Dermal fibroblast | 0.0 | 12.0 |
| | | | IL-4 | | |
| Dendritic cells anti- | 0.0 | 0.0 | IBD Colitis 2 | 6.3 | 20.7 |
| CD40 | | | | | |
| Monocytes rest | 0.0 | 0.0 | IBD Crohn's | 2.9 | 0.0 |
| Monocytes LPS | 0.0 | 0.0 | Colon | 9.7 | 0.0 |
| Macrophages rest | 0.0 | 0.0 | Lung | 0.0 | 0.0 |
| Macrophages LPS | 0.0 | 0.0 | Thymus | 0.0 | 25.3 |
| HUVEC none | 0.0 | 0.0 | Kidney | 0.0 | 0.0 |
| HUVEC starved | 4.5 | 0.0 | | | |

CNS_neurodegeneration_v1.0 Summary: Ag2364 Expression is low/undetected in all samples in this panel. (Data not shown.)

Panel 1.3D Summary: Ag2364 Expression is low/undetected in all samples in this panel. (CTs>35)(Data not shown.)

Panel 4D Summary: Agl725/Ag2364 The GPCR6 transcript is only detected in liver cirrhosis. Furthermore, this transcript is not detected in normal liver in Panel 1.3D, suggesting that this gene expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR6 could also be used for the diagnosis of liver cirrhosis.

GPCR7

Expression of the GPCR7 gene (also referred to as GM_330202597_A_da1) was assessed using the primer-probe set Ag1173, described in Table 57.

TABLE 57

Probe Name Ag1173

| | | | Start | SEQ ID |
|---|---|---|---|---|
| Primers | Sequences | Length | Position | NO: |
| Forward | 5′-gcgcaatagaatacgtcaattt-3′ | 22 | 527 | 173 |
| Probe | TET-5′-cagctcctaagcctcttagaccccaa-3′-TAMRA | 26 | 564 | 174 |
| Reverse | 5′-aaccatgactccaatctcaatg-3′ | 22 | 599 | 175 |

CNS_neurodegeneration_v1.0 Summary: Ag1173 The amp plot from one experiment with this probe and primer suggests that there were experimental difficulties with this run.

Panel 1.2 Summary: Ag1173 Expression is low/undetectable in all samples in this panel. (CTs>35). (Data not shown.)

Panel 4D Summary: Ag1173 Expression is low/undetectable with questionable amp plots for multiple runs with this probe and primer set. (Data not shown.)

GPCR8

Expression of the GPCR8 gene(also referred to as AC076959_da2) was assessed using the primer-probe sets Ag2308, Ag1510, Ag4494, and Ag1538, described in Tables 58, 59, 60 and 61. Results of the RTQ-PCR runs are shown in Tables 62, 63, 64 and 65.

TABLE 58

Probe Name Ag2308

| | | | | |
|---|---|---|---|---|
| | | | Start | SEQ ID |
| Primers | Sequences | Length | Position | NO: |

| | | | | |
|---|---|---|---|---|
| Forward | 5′-taccgatcatagcacatcatca-3′ | 22 | 591 | 176 |
| Probe | TET-5′-tcagacactctgtaatagcaaacgcca-3′-TAMRA | 27 | 619 | 177 |
| Reverse | 5′-tgctccttgcatacttcagact-3′ | 22 | 656 | 178 |

TABLE 59

Probe Name Ag1510

| | | | Start | SEQ ID |
|---|---|---|---|---|
| Primers | Sequences | Length | Position | NO: |
| Forward | 5′-attctcaagaacggaggaagat-3′ | 22 | 141 | 179 |
| Probe | TET-5′-tttacagccttttcaacccgatcctg-3′-TAMRA | 26 | 104 | 180 |
| Reverse | 5′-tctgcattcctaaggctgtaga-3′ | 22 | 72 | 181 |

TABLE 60

Probe Name Ag4494

| | | | Start | SEQ ID |
|---|---|---|---|---|
| Primers | Sequences | Length | Position | NO: |
| Forward | 5′-attctcaagaacggaggaagat-3′ | 22 | 141 | 182 |
| Probe | TET-5′-tttacagccttttcaacccgatcctg-3′-TAMRA | 26 | 104 | 183 |
| Reverse | 5′-tctgcattcctaaggctgtaga-3′ | 22 | 72 | 184 |

TABLE 61

Probe Name Ag1538

| | | | Start | SEQ ID |
|---|---|---|---|---|
| Primers | Sequences | Length | Position | NO: |
| Forward | 5′-AGGAAGATCCTTTCCCTGTTT-3′ | 21 | 171 | 185 |
| Probe | TET-5′-TACAGCCTTTTCAACCCGATCCTGAA-3′-TAMRA | 26 | 192 | 186 |
| Reverse | 5′-CTCTCTTTAGAGCCCCTTTCAC-3′ | 22 | 249 | 187 |

TABLE 62
CNS_neurodegeneration_v1.0

| | Rel. Exp. (%) Ag2308, | | Rel. Exp. (%) Ag2308, |
|---|---|---|---|
| Tissue Name | Run 207970871 | Tissue Name | Run 207970871 |
| AD 1 Hippo | 0.0 | Control (Path) 3 | 0.0 |
| | | Temporal Ctx | |
| AD 2 Hippo | 9.8 | Control (Path) 4 | 10.7 |
| | | Temporal Ctx | |
| AD 3 Hippo | 0.0 | AD 1 Occipital Ctx | 24.0 |
| AD 4 Hippo | 18.2 | AD 2 Occipital Ctx | 0.0 |
| | | (Missing) | |
| AD 5 Hippo | 16.6 | AD 3 Occipital Ctx | 0.0 |
| AD 6 Hippo | 81.8 | AD 4 Occipital Ctx | 1.2 |
| Control 2 Hippo | 0.0 | AD 5 Occipital Ctx | 1.8 |
| Control 4 Hippo | 38.7 | AD 5 Occipital Ctx | 1.9 |
| Control (Path) 3 | 1.2 | Control 1 Occipital | 17.6 |
| Hippo | | Ctx | |
| AD 1 Temporal Ctx | 16.4 | Control 2 Occipital | 0.0 |
| | | Ctx | |
| AD 2 Temporal Ctx | 14.6 | Control 3 Occipital | 17.2 |
| | | Ctx | |
| AD 3 Temporal Ctx | 0.0 | Control 4 Occipital | 30.8 |
| | | Ctx | |
| AD 4 Temporal Ctx | 20.4 | Control (Path) 1 | 0.0 |
| | | Occipital Ctx | |
| AD 5 Inf Temporal | 15.1 | Control (Path) 2 | 11.7 |
| Ctx | | Occipital Ctx | |
| AD 5 Sup Temporal | 27.9 | Control (Path) 3 | 0.0 |
| Ctx | | Occipital Ctx | |
| AD 6 Inf Temporal | 13.0 | Control (Path) 4 | 16.7 |
| Ctx | | Occipital Ctx | |
| AD 6 Sup Temporal | 1.6 | Control 1 Parietal | 0.0 |
| Ctx | | Ctx | |
| Control 1 Temporal | 0.0 | Control 2 Parietal | 0.0 |
| Ctx | | Ctx | |
| Control 2 Temporal | 1.2 | Control 3 Parietal | 0.0 |
| Ctx | | Ctx | |
| Control 3 Temporal | 100.0 | Control (Path) 1 | 4.0 |
| Ctx | | Parietal Ctx | |
| Control 3 Temporal | 38.7 | Control (Path) 2 | 0.0 |
| Ctx | | Parietal Ctx | |
| Control (Path) 1 | 16.2 | Control (Path) 3 | 10.3 |
| Temporal Ctx | | Parietal Ctx | |
| Control (Path) 2 | 0.0 | Control (Path) 4 | 31.0 |
| Temporal Ctx | | Parietal Ctx | |

TABLE 63
General_screening_panel_v1.4

| | Rel. | Rel. | | Rel. | Rel. |
|---|---|---|---|---|---|
| | Exp. (%) | Exp. (%) | | Exp. (%) | Exp. (%) |
| | Ag1510, | Ag4494, | | Ag1510, | Ag4494, |
| | Run | Run | | Run | Run |
| Tissue Name | 222653849 | 222666589 | Tissue Name | 222653849 | 222666589 |
| Adipose | 14.2 | 6.1 | Renal ca. TK-10 | 8.3 | 13.8 |
| Melanoma* | 9.0 | 0.0 | Bladder | 100.0 | 24.3 |
| Hs688 (A).T | | | | | |
| Melanoma* | 4.2 | 6.7 | Gastric ca. (liver | 3.7 | 9.7 |
| Hs688 (B).T | | | met.) NCI-N87 | | |
| Melanoma* | 0.0 | 0.0 | Gastric ca. | 1.7 | 8.7 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 0.0 | Colon ca. SW480 | 0.0 | 0.0 |
| SK-MEL-5 | | | | | |
| Squamous cell | 0.0 | 0.0 | Colon ca.* | 0.0 | 8.2 |
| carcinoma | | | (SW480 met) | | |
| SCC-4 | | | SW620 | | |
| Testis Pool | 6.9 | 0.0 | Colon ca. HT29 | 2.4 | 0.0 |
| Prostate ca.* | 0.0 | 0.0 | Colon ca. HCT- | 0.0 | 12.8 |
| (bone met) | | | 116 | | |
| PC-3 | | | | | |
| Prostate Pool | 10.6 | 7.2 | Colon ca. CaCo-2 | 11.0 | 8.4 |
| Placenta | 2.5 | 2.3 | Colon cancer | 19.3 | 10.3 |
| | | | tissue | | |
| Uterus Pool | 4.0 | 2.5 | Colon ca. | 0.0 | 0.0 |
| | | | SW1116 | | |
| Ovarian ca. | 12.5 | 8.6 | Colon ca. Colo-205 | 0.0 | 0.0 |
| OVCAR-3 | | | | | |
| Ovarian ca. | 2.2 | 3.6 | Colon ca. SW-48 | 0.0 | 0.0 |
| SK-OV-3 | | | | | |
| Ovarian ca. | 0.0 | 0.0 | Colon Pool | 3.0 | 5.5 |
| OVCAR-4 | | | | | |
| Ovarian ca. | 0.0 | 17.2 | Small Intestine | 11.0 | 6.5 |
| OVCAR-5 | | | Pool | | |
| Ovarian ca. | 0.0 | 0.0 | Stomach Pool | 21.6 | 24.8 |
| IGROV-1 | | | | | |
| Ovarian ca. | 5.7 | 0.0 | Bone Marrow | 57.4 | 22.4 |
| OVCAR-8 | | | Pool | | |
| Ovary | 13.5 | 15.7 | Fetal Heart | 0.0 | 0.0 |
| Breast ca. | 0.0 | 3.0 | Heart Pool | 19.1 | 7.3 |
| MCF-7 | | | | | |
| Breast ca. | 4.0 | 2.2 | Lymph Node | 27.9 | 29.5 |
| MDA-MB- | | | Pool | | |
| 231 | | | | | |
| Breast ca. BT | 0.0 | 0.0 | Fetal Skeletal | 0.0 | 0.0 |
| 549 | | | Muscle | | |
| Breast ca. | 16.2 | 2.2 | Skeletal Muscle | 0.0 | 0.0 |
| T47D | | | Pool | | |
| Breast ca. | 0.0 | 7.1 | Spleen Pool | 4.3 | 5.4 |
| MDA-N | | | | | |
| Breast Pool | 8.3 | 0.0 | Thymus Pool | 7.9 | 13.1 |
| Trachea | 7.1 | 2.7 | CNS cancer | 0.0 | 0.0 |
| | | | (glio/astro) U87- | | |
| | | | MG | | |
| Lung | 3.4 | 7.2 | CNS cancer | 6.7 | 0.0 |
| | | | (glio/astro) U- | | |
| | | | 118-MG | | |
| Fetal Lung | 29.9 | 34.9 | CNS cancer | 0.0 | 0.0 |
| | | | (neuro;met) SK- | | |
| | | | N-AS | | |
| Lung ca. NCI- | 0.0 | 0.0 | CNS cancer | 0.0 | 0.0 |
| N417 | | | (astro) SF-539 | | |
| Lung ca. LX- | 9.2 | 14.7 | CNS cancer | 0.0 | 0.0 |
| 1 | | | (astro) SNB-75 | | |
| Lung ca. NCI- | 0.0 | 0.0 | CNS cancer | 0.0 | 0.0 |
| H146 | | | (glio) SNB-19 | | |
| Lung ca. | 0.0 | 0.0 | CNS cancer | 0.0 | 2.8 |
| SHP-77 | | | (glio) SF-295 | | |
| Lung ca. | 0.0 | 0.0 | Brain | 0.0 | 0.0 |
| A549 | | | (Amygdala) Pool | | |
| Lung ca. NCI- | 0.0 | 0.0 | Brain | 0.0 | 0.0 |
| H526 | | | (cerebellum) | | |
| Lung ca. NCI- | 3.4 | 4.1 | Brain (fetal) | 0.0 | 6.5 |
| H23 | | | | | |
| Lung ca. NCI- | 0.0 | 0.0 | Brain | 0.0 | 0.0 |
| H460 | | | (Hippocampus) | | |
| | | | Pool | | |
| Lung ca. | 3.1 | 0.0 | Cerebral Cortex | 0.0 | 0.0 |
| HOP-62 | | | Pool | | |
| Lung ca. NCI- | 0.0 | 0.0 | Brain (Substantia | 0.0 | 0.0 |
| H522 | | | nigra) Pool | | |
| Liver | 0.0 | 0.0 | Brain (Thalamus) | 0.0 | 8.5 |
| | | | Pool | | |
| Fetal Liver | 6.9 | 9.9 | Brain (whole) | 0.0 | 0.0 |
| Liver ca. | 8.8 | 14.1 | Spinal Cord Pool | 0.0 | 9.7 |
| HepG2 | | | | | |
| Kidney Pool | 19.2 | 4.5 | Adrenal Gland | 3.7 | 3.9 |
| Fetal Kidney | 70.7 | 100.0 | Pituitary gland | 0.0 | 3.5 |
| | | | Pool | | |
| Renal ca. 786- | 34.6 | 32.8 | Salivary Gland | 3.2 | 0.0 |
| 0 | | | | | |
| Renal ca. | 16.2 | 5.9 | Thyroid (female) | 3.2 | 3.1 |
| A498 | | | | | |
| Renal ca. | 3.4 | 2.8 | Pancreatic ca. | 2.4 | 0.0 |
| ACHN | | | CAPAN2 | | |
| Renal ca. UO- | 1.9 | 2.6 | Pancreas Pool | 17.9 | 18.3 |
| 31 | | | | | |

TABLE 64
Panel 1.2

| | Rel. Exp. (%) Ag1510, | | Rel. Exp. (%) Ag1510, |
|---|---|---|---|
| Tissue Name | Run 141938638 | Tissue Name | Run 141938638 |
| Endothelial cells | 0.0 | Renal ca. 786-0 | 11.9 |
| Heart (Fetal) | 0.0 | Renal ca. A498 | 24.3 |
| Pancreas | 0.6 | Renal ca. RXF 393 | 22.2 |
| Pancreatic ca. | 0.3 | Renal ca. ACHN | 2.6 |
| CAPAN 2 | | | |
| Adrenal Gland | 2.7 | Renal ca. UO-31 | 43.8 |
| Thyroid | 1.0 | Renal ca. TK-10 | 8.2 |
| Salivary gland | 49.7 | Liver | 11.2 |
| Pituitary gland | 0.0 | Liver (fetal) | 3.1 |
| Brain (fetal) | 0.0 | Liver ca. | 55.1 |
| | | (hepatoblast) HepG2 | |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) | 4.6 |
| | | LX-1 | |
| Brain (hippocampus) | 0.0 | Lung ca. (small cell) | 61.1 |
| | | NCI-H69 | |
| Brain (thalamus) | 0.0 | Lung ca. (s. cell var.) | 0.0 |
| | | SHP-77 | |
| Cerebral Cortex | 0.0 | Lung ca. (large | 46.7 |
| | | cell) NCI-H460 | |
| Spinal cord | 0.0 | Lung ca. (non-sm. | 23.0 |
| | | cell) A549 | |
| glio/astro U87-MG | 0.0 | Lung ca. (non-s. cell) | 6.1 |
| | | NCI-H23 | |
| glio/astro U-118-MG | 2.3 | Lung ca. (non-s. cell) | 51.1 |
| | | HOP-62 | |
| astrocytoma SW1783 | 14.7 | Lung ca. (non-s. cl) | 0.0 |
| | | NCI-H522 | |
| neuro*; met SK-N-AS | 0.0 | Lung ca. (squam.) | 37.9 |
| | | SW 900 | |
| astrocytoma SF-539 | 4.5 | Lung ca. (squam.) | 27.7 |
| | | NCI-H596 | |
| astrocytoma SNB-75 | 0.0 | Mammary gland | 15.4 |
| glioma SNB-19 | 13.8 | Breast ca.* (pl. ef) | 2.5 |
| | | MCF-7 | |
| glioma U251 | 0.0 | Breast ca.* (pl. ef) | 0.0 |
| | | MDA-MB-231 | |
| glioma SF-295 | 7.3 | Breast ca.* (pl. ef) | 1.8 |
| | | T47D | |
| Heart | 2.8 | Breast ca. BT-549 | 6.4 |
| Skeletal Muscle | 0.0 | Breast ca. MDA-N | 19.9 |
| Bone marrow | 2.9 | Ovary | 1.7 |
| Thymus | 0.0 | Ovarian ca. OVCAR-3 | 6.8 |
| Spleen | 0.0 | Ovarian ca. OVCAR-4 | 11.8 |
| Lymph node | 1.3 | Ovarian ca. OVCAR-5 | 100.0 |
| Colorectal | 14.0 | Ovarian ca. OVCAR-8 | 42.3 |
| Stomach | 3.6 | Ovarian ca. IGROV-1 | 0.0 |
| Small intestine | 0.3 | Ovarian ca. (ascites) | 0.0 |
| | | SK-OV-3 | |
| Colon ca. SW480 | 0.0 | Uterus | 0.3 |
| Colon ca.* SW620 | 0.6 | Placenta | 3.8 |
| (SW480 met) | | | |
| Colon ca. HT29 | 27.0 | Prostate | 12.1 |
| Colon ca. HCT-116 | 7.2 | Prostate ca.* (bone | 6.3 |
| | | met) PC-3 | |
| Colon ca. CaCo-2 | 0.0 | Testis | 7.1 |
| CC Well to Mod Diff | 30.8 | Melanoma | 11.9 |
| (ODO3866) | | Hs688 (A).T | |
| Colon ca. HCC-2998 | 27.5 | Melanoma* (met) | 27.5 |
| | | Hs688 (B).T | |
| Gastric ca. (liver met) | 12.6 | Melanoma UACC-62 | 0.0 |
| NCI-N87 | | | |
| Bladder | 83.5 | Melanoma M14 | 67.4 |
| Trachea | 0.0 | Melanoma LOX | 0.0 |
| | | IMVI | |
| Kidney | 100.0 | Melanoma* (met) | 0.0 |
| | | SK-MEL-5 | |
| Kidney (fetal) | 14.2 | | |

TABLE 65
Panel 4D

| | Rel. Exp. (%) Ag2308, | | Rel. Exp. (%) Ag2308, |
|---|---|---|---|
| Tissue Name | Run 158927487 | Tissue Name | Run 158927487 |
| Secondary Th1 act | 0.0 | HUVEC IL-1beta | 0.0 |
| Secondary Th2 act | 0.0 | HUVEC IFN gamma | 17.4 |
| Secondary Tr1 act | 0.0 | HUVEC TNF alpha + IFN | 0.0 |
| | | gamma | |
| Secondary Th1 rest | 17.2 | HUVEC TNF alpha + IL4 | 0.0 |
| Secondary Th2 rest | 0.0 | HUVEC IL-11 | 4.4 |
| Secondary Tr1 rest | 0.0 | Lung Microvascular EC | 0.0 |
| | | none | |
| Primary Th1 act | 11.3 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 24.0 | Bronchial epithelium | 0.0 |
| | | TNFalpha + IL1beta | |
| Primary Th2 rest | 14.5 | Small airway epithelium | 0.0 |
| | | none | |
| Primary Tr1 rest | 0.0 | Small airway epithelium | 15.1 |
| | | TNFalpha + IL-1beta | |
| CD45RA CD4 | 0.0 | Coronery artery SMC rest | 0.0 |
| lymphocyte act | | | |
| CD45RO CD4 | 6.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 29.1 |
| LAK cells IL-2 + IL-12 | 0.0 | Lupus kidney | 9.9 |
| LAK cells IL-2 + IFN | 21.8 | NCI-H292 none | 26.6 |
| gamma | | | |
| LAK cells IL-2 + IL-18 | 0.0 | NCI-H292 IL-4 | 28.7 |
| LAK cells | 0.0 | NCI-H292 IL-9 | 37.9 |
| PMA/ionomycin | | | |
| NK Cells IL-2 rest | 0.0 | NCI-H292 IL-13 | 0.0 |
| Two Way MLR 3 day | 10.6 | NCI-H292 IFN gamma | 13.3 |
| Two Way MLR 5 day | 21.5 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 11.3 |
| PBMC PWM | 0.0 | Lung fibroblast TNF alpha + | 0.0 |
| | | IL-1 beta | |
| PBMC PHA-L | 14.6 | Lung fibroblast IL-4 | 18.6 |
| Ramos (B cell) none | 0.0 | Lung fibroblast IL-9 | 0.0 |
| Ramos (B cell) | 0.0 | Lung fibroblast IL-13 | 15.9 |
| ionomycin | | | |
| B lymphocytes PWM | 0.0 | Lung fibroblast IFN | 22.2 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 6.7 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 27.5 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 8.7 |
| Macrophages rest | 7.5 | Lung | 47.0 |
| Macrophages LPS | 0.0 | Thymus | 100.0 |
| HUVEC none | 0.0 | Kidney | 5.8 |
| HUVEC starved | 0.0 | | |

CNS_neurodegeneration_v1.0 Summary: Ag2308 Highest expression of the GPCR8 gene is detected in the temporal cortex of a control patient (CT=33.34) with expression also detected in the hippocampus in both a control brain and a brain from a patient with Alzheimer's disease. The GPCR8 gene encodes a putative GPCR. Several neurotransmitter receptors are GPCrs, including the dopamine receptor family, the serotonin receptor family, the GABAB receptor, muscarinic acetylcholine receptors, and others; thus the GPCR8 gene product may represent a novel neurotransmitter receptor. Targeting various neurotransmitter receptors (dopamine, serotonin) has proven to be an effective therapy in psychiatric illnesses such as schizophrenia, bipolar disorder, and depression. Furthermore, the cerebral cortex and hippocampus are regions of the brain that are known to be involved in Alzheimer's disease, seizure disorders, and in the normal process of memory formation. Therefore, therapeutic modulation of the GPCR8 gene or its protein product may be beneficial in the treatment of one or more of these diseases, as may stimulation and/or blockade of the receptor coded for by the GPCR8 gene.

Ag1510/Ag4494 (identical sequence) Please note that two experiments with a second probe and primer set showed low/undetectable levels of expression across all samples in this panel. (CT>35). (Data not shown.)

General_screening_panel_v1.4 Summary: Ag1510/Ag4494 (identical sequence) two runs with same probe and primer set produce results that are in good agreement with highest expression in the bladder and fetal kidney (CTs=32.5-33.5). Of note is the difference in expression between the fetal kidney (CTs=32.5-34) and the adult kidney (CTs=36-37). Thus, expression of the GPCR8 gene could be used to differentiate between adult and fetal kidney tissue.

Panel 1.2 Summary: Ag1510 Moderate expression of the the GPCR8 gene is detected in both adult kidney tissue and ovarian cancer cell lines (CTs=31.4). This result suggests that therapeutic modulation of the transcript of the GPCR8 gene may be effective in the treatment of ovarian cancer. Furthermore, the overexpression of this gene in adult kidney as compared to the lower expression level in fetal kidney (CT=34.3) indicates that this gene could be used to differentiate between adult and fetal kidney tissue. The GPCR8 gene is expressed at low levels in a wide variety of both healthy tissues and cancerous cell lines. Cancerous cell lines demonstrating expression of the gene include lung, kidney, colon and other ovarian cancer cell lines. Thus, expression of the GPCR8 gene could potentially be used to distinguish cancer cells from their normal counterparts. Therefore, therapeutic modulation of the protein product of the gene may be of utility in the treatment of lung, kidney or colon cancer. Healthy tissues demonstrating significant expression of gene include bladder and salivary gland tissue. Ag1538 Expression of the gene is low/undetectable (CT values>35) across all of the samples on this panel (data not shown).

Panel 1.3D Summary Ag2308 Expression is low/undet. (CT>35) in all samples shown in this Panel. (Data not shown.)

Panel 4.1D Summary Ag1510/Ag4494 (identical sequence) Expression is low/undet. (CT>35) in all samples in this panel. (Data not shown.)

Panel 4D Summary: Ag1538/Ag2308 Expression of the GPCR8 gene is detected in the thymus (CT=33.3) and lung (CT=34.4) using the probe/primer set Ag2308. This observation suggests that the GPCR8 gene could be used as a marker to detect the presence of thymus or lung tissue. The putative GPCR encoded for by the GPCR8 gene may also play an important role in the normal homeostasis of these tissues. Therapeutics designed with the GPCR8 protein product could be important for maintaining or restoring normal function to these organs during inflammation. Ag1538 Expression of the gene is low/undetectable (CT values>35) across all of the samples on this panel (data not shown).

GPCR9

Expression of GPCR9 gene (also referred to as AC073364_da1) was assessed using the primer-probe set Ag2310, described in Table 66. Results of the RTQ-PCR runs are shown in Table 67.

TABLE 66

Probe Name Ag2310

| | | | | SEQ ID |
|---|---|---|---|---|
| Primers | Sequences | Length | Start Position | NO: |
| Forward | 5′-catgctgtgaccatcatcatta-3′ | 22 | 619 | 188 |
| Probe | TET-5′-ccttcctaatcattgccctgtcctatg-3′-TAMRA | 27 | 641 | 189 |
| Reverse | 5′-cttcagaagagggaatcctcaa-3′ | 22 | 688 | 190 |

TABLE 67
Panel 4D

| | Rel. Exp. (%) | | Rel. Exp. (%) |
|---|---|---|---|
| | Ag2310, Run | | Ag2310, Run |
| Tissue Name | 162308429 | Tissue Name | 162308429 |
| Secondary Th1 act | 0.0 | HUVEC IL-1beta | 0.0 |
| Secondary Th2 act | 0.0 | HUVEC IFN gamma | 0.0 |
| Secondary Tr1 act | 16.8 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 2.5 |
| | | TNFalpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 24.3 |
| | | PMA/ionomycin | |
| 2ry Th1/Th2/Tr1_anti- | 0.0 | CCD1106 (Keratinocytes) | 14.3 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 10.8 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 15.7 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 2.8 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 20.3 | Colon | 0.0 |
| Macrophages rest | 100.0 | Lung | 3.9 |
| Macrophages LPS | 0.0 | Thymus | 11.2 |
| HUVEC none | 0.0 | Kidney | 12.0 |
| HUVEC starved | 0.0 | | |

Panel 1.3D Summary Ag2310 Expression low/undetectable (CT values>34.5) across the samples on this panel.

Panel 2.2 Summary Ag2310 Expression low/undetectable (CT values>34.5) across the samples on this panel.

Panel 4.1D Summary Ag3210 Expression low/undetectable (CT values>34.5) across the samples on this panel.

Panel 4D Summary: Ag2310 Teh GPCR9 transcript is detected at significant levels in resting macrophates. The putative GPCR encoded for by this gene is down regulated in macrophages after LPS stimulation. Therefore, the GPCR9 gene may function to respond to inflammatory stimuli and become down regulated after 12-24 hr exposure. Antibody or small molecule therapeutics designe againts this putative GPCR could thus reduce or inhibit inflammation and be important in diseases such as asthma, IBD, psoriasis, arthritis and allergy Furthermore, agonistic (lingand-like) therapeutics designed with the putative GPCR could “jump start” the immune response and improve the efficacy of vaccines and antiviral or antibacterial treatments.

GPCR10

Expression of GPCR10 gene(also refered to as CG55732

-01) was assessed using the primer-probe set Ag2588, described in Table 68. Results of the RTQ-PCR runs are shown in Table 69.

TABLE 68

Probe Name Ag2588

| | | | Start | SEQ ID |
|---|---|---|---|---|
| Primers | Sequences | Length | Position | NO: |
| Forward | 5′-gaagaggatctgtatggttgca-3′ | 22 | 532 | 191 |
| Probe | TET-5′-tcctacccatgacactcccactagca-3′-TAMRA | 26 | 565 | 192 |
| Reverse | 5′-tgcactataccaccatcatgaa-3′ | 22 | 608 | 193 |

TABLE 69
Panel 4D

| | Rel. Exp. (%) | | Rel. Exp. (%) |
|---|---|---|---|
| | Ag2588, Run | | Ag2588, Run |
| Tissue Name | 164327719 | Tissue Name | 164327719 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 13.7 | HUVEC TNF alpha + IL4 | 0.0 |
| Secondary Th2 rest | 0.0 | HUVEC IL-11 | 2.5 |
| Secondary Tr1 rest | 13.7 | Lung Microvascular EC | 0.0 |
| | | none | |
| Primary Th1 act | 0.0 | Lung Microvascular EC | 5.5 |
| | | TNFalpha + IL-1beta | |
| Primary Th2 act | 0.0 | Microvascular Dermal EC | 3.7 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 0.0 | Small airway epithelium | 16.8 |
| | | none | |
| Primary Tr1 rest | 0.0 | Small airway epithelium | 17.4 |
| | | TNFalpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 6.2 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 4.4 | CCD1106 (Keratinocytes) | 0.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 100.0 |
| LAK cells IL-2 + IL-12 | 13.3 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 11.0 |
| PMA/ionomycin | | | |
| NK Cells IL-2 rest | 0.0 | NCI-H292 IL-13 | 6.6 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 9.3 |
| | | CCD1070 TNF alpha | |
| EOL-1 dbcAMP | 2.6 | Dermal fibroblast | 0.0 |
| PMA/ionomycin | | CCD1070 IL-1 beta | |
| Dendritic cells none | 0.0 | Dermal fibroblast IFN | 6.0 |
| | | gamma | |
| Dendritic cells LPS | 0.0 | Dermal fibroblast IL-4 | 17.7 |
| Dendritic cells anti- | 0.0 | IBD Colitis 2 | 0.0 |
| CD40 | | | |
| Monocytes rest | 0.0 | IBD Crohn's | 7.7 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag2588 Expression is low/undetected in all samples in this panel (CTh35). (Data not shown.)

Panel 1.3D Summary: Ag2588 Expression is low/undetected in all samples in this panel (CT>35). (Data not shown.)

Panel 4D Summary: Ag2588 The GPCR10 transcript is only detected in liver cirrhosis. Furthermore, this transcript is not detected in normal liver in Panel 1.3D, suggesting that this gene expression is unique to liver cirrhosis. The GPCR10 gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

GPCR11

Expression of the GPCR11 gene (also referred to as sggc_draft_ba656o22_20000731_da1) was assessed using the primer-probe set Ag1898, described in Table 70. Results of the RTQ-PCR runs are shown in Table 71.

TABLE 70

Probe Name Ag1898

| | | | | |
|---|---|---|---|---|
| | | | | SEQ ID |
| Primers | Sequences | Length | Start Position | NO: |

| | | | | |
|---|---|---|---|---|
| Forward | 5′-ggctgtggtgtctctgttttac-3′ | 122 | 740 | 194 |
| Probe | TET-5′-catcttcatgtatctccagccagcca-3′-TAMRA | 26 | 770 | 195 |
| Reverse | 5′-ctatgaacttgccctgctcat-3′ | 21 | 808 | 196 |

TABLE 71
Panel 1.3D

| | Rel. Exp. (%) | | Rel. Exp. (%) |
|---|---|---|---|
| | Ag1898, Run | | Ag1898, Run |
| Tissue Name | 165544705 | Tissue Name | 165544705 |
| Liver adenocarcinoma | 0.0 | Kidney (fetal) | 0.0 |
| Pancreas | 0.0 | Renal ca. 786-0 | 0.0 |
| Pancreatic ca. | 0.0 | Renal ca. A498 | 0.0 |
| CAPAN 2 | | | |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 1.7 | Liver (fetal) | 0.0 |
| Brain (amygdala) | 0.0 | Liver ca. | 0.0 |
| | | (hepatoblast) HepG2 | |
| Brain (cerebellum) | 29.9 | Lung | 0.0 |
| Brain (hippocampus) | 0.0 | Lung (fetal) | 0.0 |
| Brain (substantia nigra) | 1.2 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 0.3 |
| | | SHP-77 | |
| Spinal cord | 0.0 | Lung ca. (large | 0.0 |
| | | cell) NCI-H460 | |
| glio/astro U87-MG | 0.0 | Lung ca. (non-sm. | 0.0 |
| | | cell) A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 2.6 | Lung ca. (squam.) SW 900 | 0.0 |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl. ef) | 0.0 |
| | | MCF-7 | |
| glioma SF-295 | 0.0 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 4.1 |
| Skeletal muscle | 0.0 | Ovary | 0.8 |
| Bone marrow | 0.0 | Ovarian ca. OVCAR-3 | 0.0 |
| Thymus | 1.3 | Ovarian ca. OVCAR-4 | 0.0 |
| Spleen | 0.0 | Ovarian ca. OVCAR-5 | 0.0 |
| Lymph node | 2.2 | Ovarian ca. OVCAR-8 | 0.0 |
| Colorectal | 0.0 | Ovarian ca. IGROV-1 | 0.0 |
| Stomach | 0.0 | Ovarian ca. (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 4.2 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 31.9 |
| Colon ca. CaCo-2 | 0.0 | Melanoma | 0.0 |
| | | Hs688(A).T | |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 2.3 |
| Gastric ca. (liver met) | 0.0 | Melanoma M14 | 6.0 |
| NCI-N87 | | | |
| Bladder | 0.0 | Melanoma LOX | 0.0 |
| | | IMVI | |
| Trachea | 0.0 | Melanoma* (met) | 100.0 |
| | | SK-MEL-5 | |
| Kidney | 0.0 | Adipose | 0.0 |

Panel 1.3D Summary: Ag1898 Highest expression of the GPCR11 gene is detected in a melanoma cell line (CT=31) with low but significant expression also seen in the cerebellum and testis. Thus, the expression of this gene could be used to distinguish samples derived from this melanoma cell line from other samples. In addition, therapeutic modulation of the expression or function of the GPCR11 gene, through the use of small molecule drugs or antibodies, might be of use in the treatment of melanoma.

The GPCR11 gene is also expressed differentially in the cerebellum. Cerebellar G protein function is known to be defective in Alzheimer's disease cerebella, suggesting this cerebellum-preferential GPCR may have utility as a drug target to counter the G-protein signaling deficit in Alzheimer's disease (Fowler et al., Receptor-effector coupling dysfunctions in Alzheimer's disease. Ann N Y Acad Sci. 786:294-304, 1996; Cowburn et al., Adenylyl cyclase activity in postmortem human brain: evidence of altered G protein mediation in Alzheimer's disease. J Neurochem. 58:1409-19, 1992).

Panel 4D Summary: Ag1898 Expression of this gene is low/undetectable (Ct values >35) in all samples on this panel (data not shown).

GPCR12

Expression of the GPCR12 gene (also referred to as ba407h12_da1) was assessed using the primer-probe set Ag1726, described in Table 72. Results of the RTQ-PCR runs are shown in Tables 73 and 74.

TABLE 72

Probe Name Ag1726

| | | | Start | SEQ ID |
|---|---|---|---|---|
| Primers | Sequences | Length | Position | NO: |
| Forward | 5′-acctcccaacaaccttctgtag-3′ | 22 | 903 | 197 |
| Probe | 5′-ccgtgacatccttgttcctaaggctg-3′-TAMRA | 26 | 872 | 198 |
| Reverse | 5′-ccatgctcaatccactcattta-3′ | 22 | 850 | 199 |

TABLE 73
Panel 2.2

| | Rel. Exp. (%) | | Rel. Exp. (%) |
|---|---|---|---|
| | Ag1726, Run | | Ag1726, Run |
| Tissue Name | 173761836 | Tissue Name | 173761836 |
| Normal Colon | 0.0 | Kidney Margin | 20.3 |
| | | (OD04348) | |
| Colon cancer | 0.0 | Kidney malignant | 3.1 |
| (OD06064) | | cancer (OD06204B) | |
| Colon Margin | 0.0 | Kidney normal adjacent | 0.0 |
| (OD06064) | | tissue (OD06204E) | |
| Colon cancer | 0.0 | Kidney Cancer | 4.0 |
| (OD06159) | | (OD04450-01) | |
| Colon Margin | 3.3 | Kidney Margin | 0.0 |
| (OD06159) | | (OD04450-03) | |
| Colon cancer | 0.0 | Kidney Cancer 8120613 | 0.0 |
| (OD06297-04) | | | |
| Colon Margin | 0.0 | Kidney Margin | 2.6 |
| (OD06297-015) | | 8120614 | |
| CC Gr.2 ascend colon | 0.0 | Kidney Cancer 9010320 | 0.0 |
| (ODO3921) | | | |
| CC Margin (ODO3921) | 0.0 | Kidney Margin | 0.0 |
| | | 9010321 | |
| Colon cancer metastasis | 0.0 | Kidney Cancer 8120607 | 0.0 |
| (OD06104) | | | |
| Lung Margin | 0.0 | Kidney Margin | 0.0 |
| (OD06104) | | 8120608 | |
| Colon mets to lung | 0.0 | Normal Uterus | 0.0 |
| (OD04451-01) | | | |
| Lung Margin | 0.0 | Uterine Cancer 064011 | 0.0 |
| (OD04451-02) | | | |
| Normal Prostate | 0.0 | Normal Thyroid | 0.0 |
| Prostate Cancer | 0.0 | Thyroid Cancer | 0.0 |
| (OD04410) | | | |
| Prostate Margin | 0.0 | Thyroid Cancer | 0.0 |
| (OD04410) | | A302152 | |
| Normal Ovary | 0.0 | Thyroid Margin | 0.0 |
| | | A302153 | |
| Ovarian cancer | 0.0 | Normal Breast | 0.0 |
| (OD06283-03) | | | |
| Ovarian Margin | 0.0 | Breast Cancer | 3.3 |
| (OD06283-07) | | | |
| Ovarian Cancer | 100.0 | Breast Cancer | 0.0 |
| Ovarian cancer | 0.0 | Breast Cancer | 0.0 |
| (OD06145) | | (OD04590-01) | |
| Ovarian Margin | 0.0 | Breast Cancer Mets | 0.0 |
| (OD06145) | | (OD04590-03) | |
| Ovarian cancer | 0.0 | Breast Cancer | 0.0 |
| (OD06455-03) | | Metastasis | |
| Ovarian Margin | 0.0 | Breast Cancer | 0.0 |
| (OD06455-07) | | | |
| Normal Lung | 0.0 | Breast Cancer 9100266 | 0.0 |
| Invasive poor diff. lung | 0.0 | Breast Margin 9100265 | 0.0 |
| adeno (ODO4945-01 | | | |
| Lung Margin | 0.0 | Breast Cancer A209073 | 0.0 |
| (ODO4945-03) | | | |
| Lung Malignant Cancer | 0.0 | Breast Margin | 0.0 |
| (OD03126) | | A2090734 | |
| Lung Margin | 0.0 | Breast cancer | 0.0 |
| (OD03126) | | (OD06083) | |
| Lung Cancer | 0.0 | Breast cancer node | 0.0 |
| (OD05014A) | | metastasis (OD06083) | |
| Lung Margin | 0.0 | Normal Liver | 0.0 |
| (OD05014B) | | | |
| Lung cancer (OD06081) | 0.0 | Liver Cancer 1026 | 0.0 |
| Lung Margin | 0.0 | Liver Cancer 1025 | 11.3 |
| (OD06081) | | | |
| Lung Cancer | 0.0 | Liver Cancer 6004-T | 0.0 |
| (OD04237-01) | | | |
| Lung Margin | 0.0 | Liver Tissue 6004-N | 0.0 |
| (OD04237-02) | | | |
| Ocular Mel Met to Liver | 0.0 | Liver Cancer 6005-T | 0.0 |
| (ODO4310) | | | |
| Liver Margin | 0.0 | Liver Tissue 6005-N | 0.0 |
| (ODO4310) | | | |
| Melanoma Metastasis | 0.0 | Liver Cancer | 0.0 |
| Lung Margin | 0.0 | Normal Bladder | 0.0 |
| (OD04321) | | | |
| Normal Kidney | 0.0 | Bladder Cancer | 0.0 |
| Kidney Ca, Nuclear | 0.0 | Bladder Cancer | 0.0 |
| grade 2 (OD04338) | | | |
| Kidney Margin | 0.7 | Normal Stomach | 0.0 |
| (OD04338) | | | |
| Kidney Ca Nuclear | 0.0 | Gastric Cancer 9060397 | 0.0 |
| grade ½ (OD04339) | | | |
| Kidney Margin | 4.3 | Stomach Margin | 12.9 |
| (OD04339) | | 9060396 | |
| Kidney Ca, Clear cell | 0.0 | Gastric Cancer 9060395 | 13.3 |
| type (OD04340) | | | |
| Kidney Margin | 0.0 | Stomach Margin | 0.0 |
| (OD04340) | | 9060394 | |
| Kidney Ca, Nuclear | 0.0 | Gastric Cancer 064005 | 0.0 |
| grade 3 (OD04348) | | | |

TABLE 74
Panel 4D

| | Rel. Exp. (%) | | Rel. Exp. (%) |
|---|---|---|---|
| | Ag1726, Run | | Ag1726, Run |
| Tissue Name | 165364124 | Tissue Name | 165364124 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNFalpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNFalpha + IL-1beta | |
| Primary Th1 rest | 0.0 | Bronchial epithelium | 0.0 |
| | | TNFalpha + IL1beta | |
| Primary Th2 rest | 7.2 | Small airway epithelium | 0.0 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 0.0 |
| lymphocyte act | | | |
| CD45RO CD4 | 6.0 | Coronery artery SMC | 0.0 |
| lymphocyte act | | TNFalpha + IL-1beta | |
| CD8 lymphocyte act | 0.0 | Astrocytes rest | 0.0 |
| Secondary CD8 | 0.0 | Astrocytes TNFalpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 6.7 | CCD1106 (Keratinocytes) | 7.0 |
| CD95 CH11 | | none | |
| LAK cells rest | 0.0 | CCD1106 (Keratinocytes) | 0.0 |
| | | TNFalpha + IL-1beta | |
| LAK cells IL-2 | 4.7 | Liver cirrhosis | 100.0 |
| LAK cells IL-2 + IL-12 | 0.0 | Lupus kidney | 5.4 |
| LAK cells IL-2 + IFN | 7.1 | NCI-H292 none | 0.0 |
| gamma | | | |
| LAK cells IL-2 + IL-18 | 12.7 | NCI-H292 IL-4 | 0.0 |
| LAK cells | 0.0 | NCI-H292 IL-9 | 0.0 |
| PMA/ionomycin | | | |
| NK Cells IL-2 rest | 3.8 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 2.4 | HPAEC TNF alpha + IL-1 | 0.0 |
| | | beta | |
| PBMC rest | 0.0 | Lung fibroblast none | 0.0 |
| PBMC PWM | 10.3 | Lung fibroblast TNF alpha + | 0.0 |
| | | IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 6.4 | Lung fibroblast IFN | 0.0 |
| | | gamma | |
| B lymphocytes CD40L | 31.2 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 3.3 |
| Dendritic cells anti- | 6.1 | IBD Colitis 2 | 5.3 |
| CD40 | | | |
| Monocytes rest | 0.0 | IBD Crohn's | 7.8 |
| Monocytes LPS | 0.0 | Colon | 6.7 |
| Macrophages rest | 0.0 | Lung | 0.0 |
| Macrophages LPS | 0.0 | Thymus | 19.6 |
| HUVEC none | 0.0 | Kidney | 0.0 |
| HUVEC starved | 0.0 | | |

Panel 1.3D Summary Ag1726 Expression of the GPCR12 gene is low/undetectable (CT values>35) across all of the samples on this panel (data not shown).

Panel 2.2 Summary: Ag1726 The GPCR12 gene is expressed at moderate levels in a sample derived from ovarian cancer (CT=31.4). Thus, expression of this gene could be used to distinguish ovarian cancer from other tissues. In addition, low level of gene expression is observed in a tissue sample from a normal kidney.

Panel 4D Summary: Summary Ag1726 Expression of the GPCR12 gene is detected at low levels (CT=33.3) in liver cirrhosis, but not in normal liver (no expression in normal liver is detected on Panel 1.3D). The putative GPCR encoded for by this gene could potentially allow cells within the liver to respond to specific microenvironmental signals. Therefore, therapies designed with the protein encoded for by this gene may potentially modulate liver function and play a role in the identification and treatment of inflammatory or autoimmune diseases which effect the liver including liver cirrhosis and fibrosis.

References

1. Mark M D, Wittemann S, Herlitze S (2000) G protein modulation of recombinant P/Q-type calcium channels by regulators of G protein signalling proteins. J Physiol. 528 Pt 1:65-77.

Fast synaptic transmission is triggered by the activation of presynaptic Ca2+channels which can be inhibited by Gbetagamma subunits via G protein-coupled receptors (GPCR).

Regulators of G protein signalling (RGS) proteins are GTPase-accelerating proteins (GAPs), which are responsible for>100-fold increases in the GTPase activity of G proteins and might be involved in the regulation of presynaptic Ca2+ channels. In this study we investigated the effects of RGS2 on G protein modulation of recombinant P/Q-type channels expressed in a human embryonic kidney (HEK293) cell line using whole-cell recordings. 2. RGS2 markedly accelerates transmitter-mediated inhibition and recovery from inhibition of Ba2+ currents (IBa) through P/Q-type channels heterologously expressed with the muscarinic acetylcholine receptor M2 (mAChR M2). 3. Both RGS2 and RGS4 modulate the prepulse facilitation properties of P/Q-type Ca2+ channels. G protein reinhibition is accelerated, while release from inhibition is slowed. These kinetics depend on the availability of G protein alpha and betagamma subunits which is altered by RGS proteins. 4. RGS proteins unmask the Ca2+ channel beta subunit modulation of Ca2+ channel G protein inhibition. In the presence of RGS2, P/Q-type channels containing the beta2a and beta3 subunits reveal significantly altered kinetics of G protein modulation and increased facilitation compared to Ca2+channels coexpressed with the beta1b or beta4 subunit. PMID: 11018106

GPCR13

Expression of the GPCR13 gene (also referred to as AC074365_da1) was assessed using the primer-probe sets Ag1478 and Ag2501, described in Tables 75 and 76. Results of the RTQ-PCR runs are shown in Tables 77, 78 and 79.

TABLE 75

Probe Name Ag1478

| | | | Start | SEQ ID |
|---|---|---|---|---|
| Primers | Sequences | Length | Position | NO: |
| Forward | 5′-ctattttggggaataccaccat-3′ | 22 | 181 | 200 |
| Probe | TET-5′-tctcgtctggaacccaagcctcatat-3′-TAMRA | 26 | 213 | 201 |
| Reverse | 5′-ggaaggagagatgagaaaggaa-3′ | 22 | 252 | 202 |

TABLE 76

Probe Name Ag2501

| | | | Start | SEQ ID |
|---|---|---|---|---|
| Primers | Sequences | Length | Position | NO: |
| Forward | 5′-acgcagtgttgaggattaagtc-3′ | 22 | 736 | 203 |
| Probe | TET-5′-acagaaagcattcgggacctgcttct-3′-TAMRA | 26 | 770 | 204 |
| Reverse | 5′-tgatggttccataaaagatggt-3′ | 22 | 813 | 205 |

TABLE 77
Panel 1.2

| | Rel. Exp. (%) | | Rel. Exp. (%) |
|---|---|---|---|
| | Ag1478, Run | | Ag1478, Run |
| Tissue Name | 139460019 | Tissue Name | 139460019 |
| Endothelial cells | 0.0 | Renal ca. 786-0 | 0.0 |
| Heart (Fetal) | 0.0 | Renal ca. A498 | 9.4 |
| Pancreas | 0.0 | Renal ca. RXF 393 | 0.0 |
| Pancreatic ca. | 0.0 | Renal ca. ACHN | 8.8 |
| CAPAN 2 | | | |
| Adrenal Gland | 0.0 | Renal ca. UO-31 | 7.0 |
| Thyroid | 0.0 | Renal ca. TK-10 | 5.6 |
| Salivary gland | 3.6 | Liver | 0.0 |
| Pituitary gland | 0.0 | Liver (fetal) | 0.0 |
| Brain (fetal) | 0.0 | Liver ca. | 0.0 |
| | | (hepatoblast) HepG2 | |
| Brain (whole) | 2.5 | Lung | 0.0 |
| Brain (amygdala) | 0.0 | Lung (fetal) | 0.0 |
| Brain (cerebellum) | 10.9 | Lung ca. (small cell) | 0.0 |
| | | LX-1 | |
| Brain (hippocampus) | 0.0 | Lung ca. (small cell) | 100.0 |
| | | NCI-H69 | |
| Brain (thalamus) | 0.0 | Lung ca. (s. cell var.) | 1.3 |
| | | SHP-77 | |
| Cerebral Cortex | 0.0 | Lung ca. (large | 24.5 |
| | | cell) NCI-H460 | |
| Spinal cord | 0.0 | Lung ca. (non-sm. | 22.7 |
| | | cell) A549 | |
| glio/astro U87-MG | 3.2 | Lung ca. (non-s. cell) | 3.1 |
| | | NCI-H23 | |
| glio/astro U-118-MG | 3.8 | Lung ca. (non-s. cell) | 40.9 |
| | | HOP-62 | |
| astrocytoma SW1783 | 0.0 | Lung ca. (non-s. cl) | 4.1 |
| | | NCI-H522 | |
| neuro*; met SK-N-AS | 11.0 | Lung ca. (squam.) | 5.5 |
| | | SW 900 | |
| astrocytoma SF-539 | 6.0 | Lung ca. (squam.) | 24.7 |
| | | NCI-H596 | |
| astrocytoma SNB-75 | 2.1 | Mammary gland | 0.0 |
| glioma SNB-19 | 24.8 | Breast ca.* (pl. ef) | 0.0 |
| | | MCF-7 | |
| Glioma U251 | 0.0 | Breast ca.* (pl. ef) | 0.0 |
| | | MDA-MB-231 | |
| glioma SF-295 | 1.6 | Breast ca.* (pl. ef) | 36.1 |
| | | T47D | |
| Heart | 2.0 | Breast ca. BT-549 | 8.8 |
| Skeletal Muscle | 2.7 | Breast ca. MDA-N | 12.2 |
| Bone marrow | 4.8 | Ovary | 0.0 |
| Thymus | 0.0 | Ovarian ca. OVCAR-3 | 2.7 |
| Spleen | 0.0 | Ovarian ca. OVCAR-4 | 0.0 |
| Lymph node | 0.0 | Ovarian ca. OVCAR-5 | 59.9 |
| Colorectal | 21.9 | Ovarian ca. OVCAR-8 | 9.5 |
| Stomach | 0.0 | Ovarian ca. IGROV-1 | 17.9 |
| Small intestine | 10.2 | Ovarian ca. (ascites) | 13.3 |
| | | SK-OV-3 | |
| Colon ca. SW480 | 0.0 | Uterus | 0.0 |
| Colon ca.* SW620 | 0.0 | Placenta | 0.0 |
| (SW480 met) | | | |
| Colon ca. HT29 | 8.1 | Prostate | 16.3 |
| Colon ca. HCT-116 | 0.0 | Prostate ca.* (bone | 9.0 |
| | | met) PC-3 | |
| Colon ca. CaCo-2 | 0.0 | Testis | 14.2 |
| CC Well to Mod Diff | 24.0 | Melanoma | 5.4 |
| (ODO3866) | | Hs688(A).T | |
| Colon ca. HCC-2998 | 4.2 | Melanoma* (met) | 17.0 |
| | | Hs688(B).T | |
| Gastric ca. (liver met) | 1.7 | Melanoma UACC-62 | 0.0 |
| NCI-N87 | | | |
| Bladder | 8.6 | Melanoma M14 | 61.6 |
| Trachea | 0.0 | Melanoma LOX | 0.0 |
| | | IMVI | |
| Kidney | 0.0 | Melanoma* (met) | 14.4 |
| | | SK-MEL-5 | |
| Kidney (fetal) | 0.0 | | |

TABLE 78D
Panel 2D

| | Rel. Exp. (%) | | Rel. Exp. (%) |
|---|---|---|---|
| | Ag1478, Run | | Ag1478, Run |
| Tissue Name | 146936352 | Tissue Name | 146936352 |
| Normal Colon | 0.2 | Kidney Margin | 0.0 |
| | | 8120608 | |
| CC Well to Mod Diff | 0.0 | Kidney Cancer | 0.0 |
| (ODO3866) | | 8120613 | |
| CC Margin (ODO3866) | 0.2 | Kidney Margin | 0.0 |
| | | 8120614 | |
| CC Gr.2 rectosigmoid | 0.0 | Kidney Cancer | 0.1 |
| (ODO3868) | | 9010320 | |
| CC Margin (ODO3868) | 0.0 | Kidney Margin | 0.0 |
| | | 9010321 | |
| CC Mod Diff (ODO3920) | 0.0 | Normal Uterus | 0.0 |
| CC Margin (ODO3920) | 0.0 | Uterine Cancer | 0.0 |
| | | 064011 | |
| CC Gr.2 ascend colon | 0.2 | Normal Thyroid | 0.0 |
| (ODO3921) | | | |
| CC Margin (ODO3921) | 0.0 | Thyroid Cancer | 0.0 |
| CC from Partial | 0.0 | Thyroid Cancer | 0.0 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 0.0 | Normal Breast | 0.0 |
| (OD04451-01) | | | |
| Lung Margin | 0.0 | Breast Cancer | 0.0 |
| (OD04451-02) | | | |
| Normal Prostate 6546-1 | 0.0 | Breast Cancer | 0.0 |
| | | (OD04590-01) | |
| Prostate Cancer | 0.1 | Breast Cancer Mets | 0.0 |
| (OD04410) | | (OD04590-03) | |
| Prostate Margin | 0.2 | Breast Cancer | 0.0 |
| (OD04410) | | Metastasis | |
| Prostate Cancer | 0.1 | Breast Cancer | 0.0 |
| (OD04720-01) | | | |
| Prostate Margin | 100.0 | Breast Cancer | 0.0 |
| (OD04720-02) | | | |
| Normal Lung | 0.1 | Breast Cancer | 0.1 |
| | | 9100266 | |
| Lung Met to Muscle | 0.3 | Breast Margin | 0.0 |
| (ODO4286) | | 9100265 | |
| Muscle Margin | 0.0 | Breast Cancer | 0.2 |
| (ODO4286) | | A209073 | |
| Lung Malignant Cancer | 0.0 | Breast Margin | 0.0 |
| (OD03126) | | A2090734 | |
| Lung Margin (OD03126) | 0.2 | Normal Liver | 0.0 |
| Lung Cancer (OD04404) | 0.0 | Liver Cancer | 0.1 |
| Lung Margin (OD04404) | 0.1 | Liver Cancer 1025 | 0.0 |
| Lung Cancer (OD04565) | 0.1 | Liver Cancer 1026 | 0.0 |
| Lung Margin (OD04565) | 0.1 | Liver Cancer 6004-T | 0.0 |
| Lung Cancer | 0.0 | Liver Tissue 6004-N | 0.0 |
| (OD04237-01) | | | |
| Lung Margin | 0.0 | Liver Cancer 6005-T | 0.0 |
| (OD04237-02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Metastasis | 0.0 | Bladder Cancer | 0.0 |
| Lung Margin (OD04321) | 0.0 | Bladder Cancer | 0.2 |
| Normal Kidney | 0.0 | Bladder Cancer | 0.0 |
| | | (OD04718-01) | |
| Kidney Ca, Nuclear | 0.0 | Bladder Normal | 0.0 |
| grade 2 (OD04338) | | Adjacent | |
| | | (OD04718-03) | |
| Kidney Margin | 0.0 | Normal Ovary | 0.0 |
| (OD04338) | | | |
| Kidney Ca Nuclear grade | 0.0 | Ovarian Cancer | 0.0 |
| ½ (OD04339) | | | |
| Kidney Margin | 0.0 | Ovarian Cancer | 0.0 |
| (OD04339) | | (OD04768-07) | |
| Kidney Ca, Clear cell | 0.1 | Ovary Margin | 0.0 |
| type (OD04340) | | (OD04768-08) | |
| Kidney Margin | 0.0 | Normal Stomach | 0.0 |
| (OD04340) | | | |
| Kidney Ca, Nuclear grade | 0.0 | Gastric Cancer | 0.0 |
| 3 (OD04348) | | 9060358 | |
| Kidney Margin | 0.0 | Stomach Margin | 0.0 |
| (OD04348) | | 9060359 | |
| Kidney Cancer | 0.0 | Gastric Cancer | 0.0 |
| (OD04622-01) | | 9060395 | |
| Kidney Margin | 0.0 | Stomach Margin | 0.0 |
| (OD04622-03) | | 9060394 | |
| Kidney Cancer | 0.0 | Gastric Cancer | 0.0 |
| (OD04450-01) | | 9060397 | |
| Kidney Margin | 0.0 | Stomach Margin | 0.0 |
| (OD04450-03) | | 9060396 | |
| Kidney Cancer 8120607 | 0.0 | Gastric Cancer | 0.0 |
| | | 064005 | |

TABLE 79
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1478, | Ag1478, | Ag2501, | | Ag1478, | Ag1478, | Ag2501, |
| | Run | Run | Run | | Run | Run | Run |
| Tissue Name | 145909066 | 146946828 | 164318131 | Tissue Name | 145909066 | 146946828 | 164318131 |
| Secondary Th1 act | 0.0 | 0.0 | 0.0 | HUVEC IL- | 0.0 | 0.0 | 0.0 |
| | | | | 1beta | | | |
| Secondary Th2 act | 0.0 | 0.0 | 0.0 | HUVEC IFN | 0.0 | 0.0 | 0.0 |
| | | | | gamma | | | |
| Secondary Tr1 act | 0.0 | 0.0 | 0.0 | HUVEC TNF | 0.0 | 0.0 | 0.0 |
| | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 0.0 | 0.0 |
| rest | | | | | | | |
| Secondary Tr1 | 0.0 | 0.0 | 0.0 | Lung | 0.0 | 0.0 | 0.0 |
| rest | | | | Microvascular | | | |
| | | | | EC none | | | |
| Primary Th1 act | 0.0 | 0.0 | 0.0 | Lung | 0.0 | 0.0 | 0.0 |
| | | | | Microvascular | | | |
| | | | | EC TNFalpha + | | | |
| | | | | IL-1beta | | | |
| Primary Th2 act | 0.0 | 0.0 | 0.0 | Microvascular | 0.0 | 0.0 | 0.0 |
| | | | | Dermal EC none | | | |
| Primary Tr1 act | 0.0 | 0.0 | 0.0 | Microsvasular | 0.0 | 0.0 | 0.0 |
| | | | | Dermal EC | | | |
| | | | | TNFalpha + IL- | | | |
| | | | | 1beta | | | |
| Primary Th1 rest | 0.0 | 0.0 | 0.0 | Bronchial | 0.0 | 0.0 | 0.0 |
| | | | | epithelium | | | |
| | | | | TNFalpha + | | | |
| | | | | IL1beta | | | |
| Primary Th2 rest | 0.0 | 0.0 | 0.0 | Small airway | 0.0 | 0.0 | 0.0 |
| | | | | epithelium none | | | |
| Primary Tr1 rest | 0.0 | 1.0 | 0.0 | Small airway | 0.0 | 0.0 | 0.0 |
| | | | | epithelium | | | |
| | | | | TNFalpha + IL- | | | |
| | | | | 1beta | | | |
| CD45RA CD4 | 0.0 | 0.0 | 0.0 | Coronery artery | 0.0 | 0.0 | 0.0 |
| lymphocyte act | | | | SMC rest | | | |
| CD45RO CD4 | 0.0 | 0.0 | 0.0 | Coronery artery | 0.0 | 0.0 | 0.0 |
| lymphocyte act | | | | SMC TNFalpha + | | | |
| | | | | IL-1beta | | | |
| CD8 lymphocyte | 0.0 | 0.0 | 0.0 | Astrocytes rest | 0.0 | 0.0 | 0.0 |
| act | | | | | | | |
| Secondary CD8 | 0.0 | 0.0 | 0.0 | Astrocytes | 0.0 | 0.0 | 0.0 |
| lymphocyte rest | | | | TNFalpha + IL- | | | |
| | | | | 1beta | | | |
| Secondary CD8 | 0.0 | 0.0 | 0.0 | KU-812 | 10.0 | 15.1 | 13.4 |
| lymphocyte act | | | | (Basophil) rest | | | |
| CD4 lymphocyte | 0.9 | 0.0 | 0.0 | KU-812 | 100.0 | 100.0 | 100.0 |
| none | | | | (Basophil) | | | |
| | | | | PMA/ionomycin | | | |
| 2ry | 0.0 | 0.0 | 0.0 | CCD1106 | 0.0 | 0.0 | 0.0 |
| Th1/Th2/Tr1_anti- | | | | (Keratinocytes) | | | |
| CD95 CH11 | | | | none | | | |
| LAK cells rest | 0.0 | 1.0 | 1.3 | CCD1106 | 0.0 | 0.0 | 0.0 |
| | | | | (Keratinocytes) | | | |
| | | | | TNFalpha + IL- | | | |
| | | | | 1beta | | | |
| LAK cells IL-2 | 0.0 | 0.0 | 0.0 | Liver cirrhosis | 4.1 | 2.5 | 2.3 |
| LAK cells IL- | 0.0 | 0.0 | 0.0 | Lupus kidney | 0.0 | 0.0 | 0.0 |
| 2 + IL-12 | | | | | | | |
| LAK cells IL- | 0.0 | 0.0 | 1.6 | NCI-H292 none | 0.0 | 0.0 | 0.0 |
| 2 + IFN gamma | | | | | | | |
| LAK cells IL-2 + | 0.0 | 0.0 | 0.0 | NCI-H292 IL-4 | 0.0 | 0.0 | 0.0 |
| IL-18 | | | | | | | |
| LAK cells | 1.4 | 0.0 | 1.3 | NCI-H292 IL-9 | 0.0 | 0.0 | 0.0 |
| PMA/ionomycin | | | | | | | |
| NK Cells IL-2 rest | 0.0 | 0.0 | 0.9 | NCI-H292 IL- | 0.0 | 0.0 | 0.0 |
| | | | | 13 | | | |
| Two Way MLR 3 | 0.0 | 0.0 | 1.2 | NCI-H292 IFN | 0.0 | 0.0 | 0.0 |
| day | | | | gamma | | | |
| Two Way MLR 5 | 0.0 | 0.0 | 0.0 | HPAEC none | 0.0 | 0.0 | 0.0 |
| day | | | | | | | |
| Two Way MLR 7 | 0.0 | 0.0 | 0.0 | HPAEC TNF | 0.0 | 0.0 | 0.0 |
| day | | | | alpha + IL-1 | | | |
| | | | | beta | | | |
| PBMC rest | 0.0 | 1.9 | 0.0 | Lung fibroblast | 0.0 | 0.0 | 0.0 |
| | | | | none | | | |
| PBMC PWM | 7.4 | 0.0 | 2.0 | Lung fibroblast | 0.0 | 0.0 | 0.0 |
| | | | | TNF alpha + IL- | | | |
| | | | | 1 beta | | | |
| PBMC PHA-L | 0.0 | 0.0 | 2.6 | Lung fibroblast | 0.0 | 0.0 | 0.0 |
| | | | | IL-4 | | | |
| Ramos (B cell) | 0.0 | 0.0 | 0.0 | Lung fibroblast | 0.0 | 0.0 | 0.0 |
| none | | | | IL-9 | | | |
| Ramos (B cell) | 0.0 | 0.0 | 0.0 | Lung fibroblast | 0.0 | 0.0 | 0.0 |
| ionomycin | | | | IL-13 | | | |
| B lymphocytes | 0.0 | 0.0 | 0.0 | Lung fibroblast | 0.0 | 0.0 | 0.0 |
| PWM | | | | IFN gamma | | | |
| B lymphocytes | 0.0 | 0.0 | 0.0 | Dermal | 0.0 | 0.0 | 0.0 |
| CD40L and IL-4 | | | | fibroblast | | | |
| | | | | CCD1070 rest | | | |
| EOL-1 dbcAMP | 0.0 | 0.0 | 0.0 | Dermal | 0.0 | 0.0 | 0.0 |
| | | | | fibroblast | | | |
| | | | | CCD1070 TNF | | | |
| | | | | alpha | | | |
| EOL-1 dbcAMP | 0.0 | 0.0 | 0.0 | Dermal | 0.0 | 0.0 | 0.0 |
| PMA/ionomycin | | | | fibroblast | | | |
| | | | | CCD1070 IL-1 | | | |
| | | | | beta | | | |
| Dendritic cells | 0.0 | 0.0 | 0.0 | Dermal | 0.0 | 0.0 | 0.0 |
| none | | | | fibroblast IFN | | | |
| | | | | gamma | | | |
| Dendritic cells | 0.0 | 0.0 | 0.0 | Dermal | 0.0 | 0.0 | 0.0 |
| LPS | | | | fibroblast IL-4 | | | |
| Dendritic cells | 0.0 | 0.0 | 0.0 | IBD Colitis 2 | 0.0 | 1.2 | 0.0 |
| anti-CD40 | | | | | | | |
| Monocytes rest | 0.0 | 0.0 | 0.0 | IBD Crohn's | 0.0 | 0.0 | 0.0 |
| Monocytes LPS | 0.0 | 0.0 | 0.0 | Colon | 0.0 | 1.3 | 0.0 |
| Macrophages rest | 0.0 | 0.0 | 0.0 | Lung | 0.0 | 0.0 | 0.0 |
| Macrophages LPS | 1.7 | 0.0 | 0.0 | Thymus | 1.1 | 0.0 | 0.0 |
| HUVEC none | 0.0 | 0.0 | 0.0 | Kidney | 0.6 | 0.0 | 1.6 |
| HUVEC starved | 0.0 | 0.0 | 0.0 | | | | |

CNS_neurodegeneration_v1.0 Summary Ag1478/Ag2501 Expression is low to undetectable (CT values>35) in all of the samples on this panel (data not shown).

Panel 1.3D Summary Ag1478/Ag2501 Expression is low to undetectable (CT values>35) in all of the examples on this panel (data not shown).

Panel 1.2 Summary: Ag1478 Highest expression is in a sample derive from a lung cancer cell line (CT=33.1). There is also significant expression in samples derived from an ovarian cancer cell line and a melanoma cell line. Thus, the GPCR13 gene may be involved in melanoma, or ovarianor lung cancer. Therefore, inhibition of the function of the protein encoded by this gene, through the use of antibodies or small molecule drugs, might be of use for the treatment of these diseases.

Panel 2D Summary: Ag1478 The expression of the GPCR13 gene is expressed highest and exclusively in normal prostate tissue. Thus, the expression of this gene could be used to distinguish normal prostate tissue from other tissues. A second run with the same probe and primer set showed low/undetectable levels of expression in all the samples in this panel.

Panel 4D Summary: Ag1478/Ag2501 Replicate experiments using different probe/primer sets all show that the GPCR13 transcript is induced in the PMA and ionomycin-treated basophil cell line KU-812. Basophils release histamines and other biological modifiers in repose to allergens and play an important role in the pathology of asthma and hypersensitivity reactions. Small molecule or antibody therapeutics designed against the putative GPCR encoded for by this gene could therefore reduce or inhibit inflammation by blocking basophil function in these diseases.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32 and 34;
(b) a variant of a mature form of an amino acid sequence selected from the group consisting of SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2, 4, 6, 8, 10, 12, 14, 16, 18, 20, 22, 24, 26, 28, 30, 32 and 34; and
(d) a variant of an amino acid sequence selected from the group consisting of SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS: 2, 4, 6, 8, 10, 12, 14, 16, 18, 20, 22, 24, 26, 28, 30, 32 and 34.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31 and 33.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32 and 34;
(b) a variant of a mature form of an amino acid sequence selected from the group consisting of SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32 and 34;
(d) a variant of an amino acid sequence selected from the group consisting SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32 and 34, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31 and 33.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, 27, 29, 31 and 33;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31 and 33, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, 23, 25, 27, 29, 31 and 33, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a GPCRX-associated disorder, said method comprising administering to a subject in which such treatment or prevention is desired the polypeptide of claim 1 in an amount sufficient to treat or prevent said GPCRX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a GPCRX-associated disorder, said method comprising administering to a subject in which such treatment or prevention is desired the nucleic acid of claim 5 in an amount sufficient to treat or prevent said GPCRX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a GPCRX-associated disorder, said method comprising administering to a subject in which such treatment or prevention is desired the antibody of claim 15 in an amount sufficient to treat or prevent said GPCRX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32 and, or a biologically active fragment thereof.

49. A method of treating a pathological state in a mammal, the method comprising administering to the mammal the antibody of claim 15 in an amount sufficient to alleviate the pathological state.
